(19) United States
(12) Patent Application Publication
(10) Pub. No.: US 2020/0370016 A1
(43) Pub. Date: Nov. 26, 2020

(54) METHODS OF GENERATING ENUCLEATED ERYTHROID CELLS

(71) Applicant: Rubius Therapeutics, Inc., Cambridge, MA (US)

(72) Inventors: Jonathan Lipsitz, Cambridge, MA (US); Brian Joseph Pereira, Watertown, MA (US); Bryan Wang, Atlanta, GA (US); Alan Gilbert, Arlington, MA (US)

(73) Assignee: Rubius Therapeutics, Inc., Cambridge, MA (US)

(21) Appl. No.: 16/882,099

(22) Filed: May 22, 2020

Related U.S. Application Data
(60) Provisional application No. 62/852,930, filed on May 24, 2019.

(51) Int. Cl. C12N 5/078; C12M 1/34; C12M 1/42; C12M 1/24; C12M 1/00; C12N 5/00

(52) U.S. Cl. CPC C12N 5/0641; C12N 2501/39; C12M 35/04; C12M 23/08; C12M 23/14; C12N 5/0018; C12M 29/10; C12N 2521/00; C12N 2500/25; C12N 2500/33; C12N 2500/36; C12N 2501/26; C12N 2501/125; C12N 2501/2303; C12N 2501/2306; C12N 2501/14; C12M 41/32

(57) ABSTRACT

Provided herein are methods of generating a population of enucleated erythroid cells.

### CROSS-REFERENCE TO RELATED APPLICATION

This application claims priority to U.S. Provisional Patent Application Ser. No. 62/852,930, filed May 24, 2019; the entire contents of which are herein incorporated by reference.

### TECHNICAL FIELD

The present invention relates generally to methods of generating enucleated erythroid cells.

### BACKGROUND

Red blood cells are transfused to patients who have experienced blood loss. In addition, engineered enucleated erythroid cells, including red blood cells, are in development as therapeutic agents which carry or present exogenous protein(s) to patients in need thereof.

### SUMMARY

The present invention is based on the discovery that the perfusion culture of erythroid progenitor cells during differentiation and/or maturation result in an increased cell proliferation, increased rate of enucleation, thus resulting in an increased number of enucleated erythroid cells (e.g., as compared to methods that do not include the use of perfusion culture during differentiation or maturation of erythroid progenitor cells), and/or increasing the length of time over which erythroid progenitor cells can continue to grow and/or enucleate.

In view of this discovery, provided herein are methods of generating a population of enucleated erythroid cells, the methods include: (a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium comprised within a perfusion bioreactor to provide a third cell culture with an initial cell density of about 1×10⁵ to about 1×10⁷ cells/mL; (d) perfusion culturing the third cell culture of step (c) for about 5 days to 20 days, where after step (d) the third culture medium includes a population of enucleated erythroid cells. Also provided herein are methods of generating a population of enucleated erythroid cells, the methods include: (a) disposing a volume of a first cell culture of erythroid progenitor cells into a second culture medium comprised within a vessel to provide a second cell culture with an initial cell density of about 0.1×10⁵ cells/mL to about 1×10⁷ cells/mL; (b) batch or fed batch culturing the second cell culture for about 2 days to about 15 days; (c) disposing a volume of the second cell culture of step (b) into a third culture medium comprised within a perfusion bioreactor to provide a third cell culture with an initial cell density of about 1×10⁵ to about 1×10⁷ cells/mL; (d) perfusion culturing the third cell culture of step (c) for about 5 days to 20 days, where after step (d) the third culture medium comprises a population of enucleated erythroid cells. Also provided herein are methods of generating a population of enucleated erythroid cells, the methods include (a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium comprised within a vessel to provide a third cell culture with an initial cell density of about 1×10⁵ to about 1×10⁷ cells/mL; (d) batch or fed batch culturing the third cell culture of step (c) for about 5 days to 20 days, where after step (d) the third culture medium comprises a population of enucleated erythroid cells.

Provided herein are methods of generating a population of enucleated erythroid cells that include: (a) disposing a volume of a first cell culture of erythroid progenitor cells into a second culture medium included within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium included within a perfusion bioreactor to provide a third cell culture with an initial cell density of about 1×10⁵ to about 1×10⁷ cells/mL; (d) perfusion culturing the third cell culture of step (c) for about 5 days to 20 days, wherein after step (d) the third culture medium includes a population of enucleated erythroid cells.

In some embodiments of any of the methods described herein, the method further includes prior to step (a): (i) disposing a plurality of erythroid progenitor cells in a first culture medium included within a vessel to provide the first cell culture with an initial cell density of about of about 0.1×10⁵ cells/mL to about 2×10⁶ cells/mL; and (ii) batch or fed batch culturing the first cell culture for about 1 day to about 15 days.

In some embodiments, the vessel in step (i) is a shake flask. In some embodiments, the shake flask has a volume of about 15 mL to about 5 L. In some embodiments, the shake flask has a volume of about 50 mL to about 1.5 L. In some embodiments, the shake flask has a volume of about 100 mL to about 500 mL. In some embodiments of any of the methods described herein, step (ii) includes incubating the shake flask at 0.1×g to about 4.1×g. In some embodiments, step (ii) includes incubating the shake flask at about 0.23×g to about 1.78×g.

In some embodiments, the vessel in step (i) is a shake tube. In some embodiments, the shake tube has a volume of about 2 mL to about 500 mL. In some embodiments, the shake tube has a volume of about 10 mL to about 250 mL. In some embodiments, the shake tube has as volume of about 50 mL to about 200 mL.

In some embodiments, the shake tube is a conical container. In some embodiments, step (ii) includes incubating the shake tube at about 0.1×g to about 4.1×g. In some embodiments, step (ii) includes incubating the shake tube at about 0.23×g to about 1.78×g.

In some embodiments, the vessel in step (i) is a culture bag. In some embodiments, the culture bag has a volume of about 50 mL to about 25 L. In some embodiments, the culture bag has a volume of about 50 mL to about 5 L. In some embodiments, the culture bag has a volume of about 50 mL to about 500 mL.

In some embodiments of any of the methods described herein, step (ii) further includes incubating the culture bag at a rocking rate of about 10 rock cycles per minute to about 50 rock cycles per minute. In some embodiments, step (ii) includes incubating the culture bag at a rocking rate of about 10 rock cycles per minute to about 25 rock cycles per minute.

In some embodiments of any of the methods described herein, step (ii) includes batch culturing the first cell culture.

In some embodiments of any of the methods described herein, step (ii) includes fed batch culturing the first cell culture. In some embodiments, fed batch culturing includes adding an additional volume of the first culture medium to the first cell culture over time. In some embodiments, the additional volume of the first culture medium is added continuously to the first cell culture over time. In some embodiments, the additional volume of the first cell culture medium is added periodically to the first cell culture over time.

In some embodiments of any of the methods described herein, the first culture medium includes one or more of Flt-3 ligand, stem cell factor (SCF), IL-3, and IL-6. In some embodiments, the first culture medium includes each of Flt-3 ligand, SCF, IL-3, and IL-6.

In some embodiments of any of the methods described herein, the first culture medium includes about 0.1 ng/mL to about 200 ng/mL Flt-3 ligand. In some embodiments, the first culture medium includes about 50 ng/mL to about 150 ng/mL Flt-3 ligand.

In some embodiments of any of the methods described herein, the first culture medium includes about 1 ng/mL to about 1 μg/mL SCF. In some embodiments, the first culture medium includes about 50 ng/mL to about 500 ng/mL SCF.

In some embodiments of any of the methods described herein, the first culture medium includes about 0.1 ng/mL to about 200 ng/mL IL-3.

In some embodiments of any of the methods described herein, the first culture medium includes about 0.1 ng/mL to about 200 ng/mL IL-6. In some embodiments, the first culture medium includes about 10 ng/mL to about 100 ng/mL IL-6.

In some embodiments of any of the methods described herein, the first culture medium includes about 1 μg/mL to about 20 μg/mL insulin. In some embodiments, the first culture medium includes about 8 μg/mL to about 12 μg/mL insulin.

In some embodiments of any of the methods described herein, the first culture medium includes about 1 mM to about 10 mM of L-glutamine, L-alanyl-L-glutamine, L-glycyl-L-glutamine, N-acetyl-L-glutamine, or a combination thereof. In some embodiments of any of the methods described herein, the first culture medium includes about 5 mM to about 7 mM of L-glutamine. In some embodiments of any of the methods described herein, the first culture medium includes about 5 mM to about 7 mM of L-alanyl-L-glutamine. In some embodiments of any of the methods described herein, the first culture medium comprises about 5 mM to about 7 mM of L-glycyl-L-glutamine. In some embodiments of any of the methods described herein, the first culture medium comprises about 5 mM to about 7 mM of N-acetyl-L-glutamine.

In some embodiments of any of the methods described herein, the first culture medium includes lipid (e.g., lipid mixture).

In some embodiments of any of the methods described herein, the first culture medium includes about 50 μg/mL to about 400 μg/mL transferrin. In some embodiments, the first culture medium includes about 150 μg/mL to about 250 μg/mL transferrin. In some embodiments of any of the methods described herein, the first culture medium comprises about 160 μg/mL to about 240 μg/mL transferrin. In some embodiments of any of the methods described herein, the first culture medium comprises about 180 μg/mL to about 220 μg/mL transferrin.

In some embodiments of any of the methods described herein, the batch or fed batch culturing in step (ii) is performed for about 5 days to about 12 days. In some embodiments, the batch or fed batch culturing in step (ii) is performed for about 5 days to about 9 days.

In some embodiments of any of the methods described herein, the initial cell density in step (i) is about 0.1×10⁵ cells/mL to about 2×10⁵ cells/mL. In some embodiments, the initial cell density in step (i) is about 0.5×10⁵ cells/mL to about 1.5×10⁵ cells/mL.

In some embodiments of any of the methods described herein, the perfusion bioreactor in step (a) has a volume of about 500 mL to about 15,000 L. In some embodiments, the perfusion bioreactor in step (a) has a volume of 5 L to about 5,000 L.

In some embodiments, the perfusion bioreactor in step (a) has a volume of about 5 L to about 2,500 L. In some embodiments, the perfusion bioreactor in step (a) has a volume of about 5 L to about 100 L. In some embodiments of any of the methods described herein, step (b) includes agitating the second cell culture at about 10 W/m³ to about 200 W/m³. In some embodiments, step (b) includes agitating the second cell culture at about 10 W/m³ to about 100 W/m³. In some embodiments of any of the methods described herein, the perfusion culturing in step (b) is performed using a perfusion rate of about 0.5 nL/cell/day to about 40 nL/cell/day. In some embodiments, the perfusion culturing in step (b) is performed using a perfusion rate of about 5 nL/cell/day to about 35 nL/cell/day. In some embodiments, the perfusion culturing in step (b) is performed using a perfusion rate of about 10 nL/cell/day to about 25 nL/cell/day.

In some embodiments of any of the methods described herein, the perfusion culturing in step (b) includes adding an additional volume of the second culture medium to the second cell culture over time. In some embodiments, the additional volume of the second culture medium is added continuously to the second cell culture over time. In some embodiments, the additional volume of the second culture medium is added periodically to the second cell culture over time.

In some embodiments of any of the methods described herein, the second culture medium includes one or more of: transferrin, IL-3, SCF, dexamethasone, erythropoietin (EPO), and insulin. In some embodiments, the second culture medium includes three or more of: transferrin, IL-3, SCF, dexamethasone, EPO, and insulin. In some embodiments, the second culture medium includes each of: transferrin, IL-3, SCF, dexamethasone, EPO, and insulin.

In some embodiments of any of the methods described herein, the second culture medium includes about 1 μg/mL transferrin to about 500 μg/mL transferrin. In some embodiments, the second culture medium includes about 100 μg/mL transferrin to about 300 μg/mL transferrin.

In some embodiments of any of the methods described herein, the second culture medium includes about 0.1 ng/mL to about 200 ng/mL IL-3. In some embodiments, the second culture medium includes about 0.1 ng/mL to about 100 ng/mL IL-3. In some embodiments, the second culture medium includes about 0.1 ng/mL to about 10 ng/mL IL-3.

In some embodiments of any of the methods described herein, the second culture medium includes about 1 ng/mL to about 1 μg/mL SCF. In some embodiments, the second culture medium includes about 1 ng/mL to about 500 ng/mL SCF. In some embodiments, the second culture medium includes about 10 ng/mL to about 200 ng/nL SCF.

In some embodiments of any of the methods described herein, the second culture medium includes about 0.1 nM to about 200 nM dexamethasone. In some embodiments, the second culture medium includes about 0.1 nM to about 100 nM dexamethasone. In some embodiments, the second culture medium includes about 0.1 nM to about 25 nM dexamethasone.

In some embodiments of any of the methods described herein, the second culture medium includes about 1 ng/mL to about 500 ng/mL EPO. In some embodiments, the second culture medium includes about 1 ng/mL to about 200 ng/mL EPO. In some embodiments, the second culture medium includes about 1 ng/mL to about 100 mg/mL EPO.

In some embodiments of any of the methods described herein, the second culture medium includes about 0.1 μg/mL to about 50 μg/mL insulin. In some embodiments, the second culture medium includes about 0.1 μg/mL to about 20 μg/mL insulin.

In some embodiments of any of the methods described herein, the second culture medium includes about 1 mM to about 10 mM of L-glutamine, L-alanyl-L-glutamine, L-glycyl-L-glutamine, N-acetyl-L-glutamine, or a combination thereof. In some embodiments, the second culture medium includes about 5 mM to about 7 mM L-glutamine. In some embodiments, the second culture medium includes about 5 mM to about 7 mM L-alanyl-L-glutamine. In some embodiments, the second culture medium includes about 5 mM to about 7 mM of L-glycyl-L-glutamine. In some embodiments, the second culture medium includes about 5 mM to about 7 mM N-acetyl-L-glutamine.

In some embodiments of any of the methods described herein, the second culture medium includes lipid (e.g., lipid mixture).

In some embodiments of any of the methods described herein, the perfusion culturing of step (b) is performed for about 4 days to about 10 days. In some embodiments, the perfusion culturing of step (b) is performed for about 5 days to about 8 days.

In some embodiments of any of the methods described herein, the initial cell density in step (a) is about 0.1×10⁵ cells/mL to about 2×10⁵ cells/mL. In some embodiments of any of the methods described herein, the initial cell density in step (a) is about 0.5×10⁵ cells/mL to about 1.5×10⁵ cells/mL.

In some embodiments of any of the methods described herein, the perfusion bioreactor in step (c) has a volume of about 500 mL to about 15,000 L. In some embodiments, the perfusion bioreactor in step (c) has a volume of 5 L to about 5,000 L. In some embodiments, the perfusion bioreactor in step (c) has a volume of about 5 L to about 2,500 L. In some embodiments, the perfusion bioreactor in step (c) has a volume of about 5 L to about 100 L. In some embodiments of any of the methods described herein, step (d) includes agitating the third cell culture at about 10 W/m³ to about 200 W/m³. In some embodiments, step (d) includes agitating the third cell culture at about 10 W/m³ to about 100 W/m³. In some embodiments of any of the methods described herein, the perfusion culturing in step (d) is performed using a perfusion rate of about 0.5 nL/cell/day to about 40 nL/cell/day. In some embodiments, the perfusion culturing in step (d) is performed using a perfusion rate of about 5 nL/cell/day to about 35 nL/cell/day. In some embodiments, the perfusion culturing in step (d) is performed using a perfusion rate of about 10 nL/cell/day to about 25 nL/cell/day.

In some embodiments of any of the methods described herein, the perfusion culturing in step (d) includes: (i) adding an additional volume of the third culture medium to the third cell culture for a first period of time, and (ii) adding an additional volume of a fourth culture medium to the third cell culture for a second period of time.

In some embodiments, the additional volume of the third culture medium in (i) is added continuously to the third cell culture for the first period of time; and/or the additional volume of the fourth culture medium in (ii) is added continuously to the third cell culture for the second period of time.

In some embodiments, the additional volume of the third culture medium in (i) is added periodically to the third cell culture for the first period of time; and/or the additional volume of the fourth culture medium in (ii) is added periodically to the third cell culture for the second period of time.

In some embodiments of any of the methods described herein, the first period of time in (i) is about 1 day to about 7 days. In some embodiments, the first period of time in (i) is about 3 days to about 5 days. In some embodiments of any of the methods described herein, the second period of time in (ii) is about 1 day to about 10 days. In some embodiments, the second period of time in (ii) is about 3 days to about 7 days.

In some embodiments of any of the methods described herein, the third culture medium includes one or more of: transferrin, insulin, SCF, and EPO. In some embodiments, the third culture medium includes two or more of: transferrin, insulin, SCF, and EPO. In some embodiments, the third culture medium includes each of: transferrin, insulin, SCF, and EPO.

In some embodiments of any of the methods described herein, the third culture medium includes about 1 transferrin to about 500 transferrin. In some embodiments, the third culture medium includes about 100 μg/mL transferrin to about 300 μg/mL transferrin.

In some embodiments of any of the methods described herein, the third culture medium includes about 1 ng/mL to about 1 SCF. In some embodiments, the third culture medium includes about 1 ng/mL to about 500 ng/mL SCF. In some embodiments, the third culture medium includes about 10 ng/mL to about 200 ng/nL SCF.

In some embodiments of any of the methods described herein, the third culture medium includes about 1 ng/mL to about 500 ng/mL EPO. In some embodiments, the third culture medium includes about 1 ng/mL to about 200 ng/mL EPO. In some embodiments, the third culture medium includes about 1 ng/mL to about 100 mg/mL EPO.

In some embodiments of any of the methods described herein, the third culture medium includes about 0.1 μg/mL to about 50 μg/mL insulin. In some embodiments, the third culture medium includes about 0.1 μg/mL to about 20 μg/mL insulin.

In some embodiments of any of the methods described herein, the third culture medium includes about 1 mM to about 8 mM of L-glutamine, L-alanyl-L-glutamine, L-glycyl-L-glutamine, N-acetyl-L-glutamine, or a combination thereof. In some embodiments, the third culture medium includes about 3 mM to about 5 mM of L-glutamine, L-alanyl-L-glutamine, L-glycyl-L-glutamine, N-acetyl-L-glutamine, or a combination thereof. In some embodiments, the third culture medium includes about 3 mM to about 5 mM L-glutamine. In some embodiments, the third culture medium includes about 3 mM to about 5 mM L-alanyl-L-glutamine.

In some embodiments of any of the methods described herein, the third culture medium includes about 0.5% v/v to about 10% v/v serum. In some embodiments, the third culture medium includes about 4% v/v to about 6% v/v serum.

In some embodiments of any of the methods described herein, the fourth culture medium includes one or more of: transferrin, insulin, and EPO. In some embodiments, the fourth culture medium includes two or more of: transferrin, insulin, and EPO. In some embodiments, the fourth culture medium includes each of: transferrin, insulin, and EPO.

In some embodiments of any of the methods described herein, the fourth culture medium includes about 100 μg/mL transferrin to about 2 mg/mL transferrin. In some embodiments, the fourth culture medium includes about 100 μg/mL transferrin to about 1.5 mg/mL transferrin.

In some embodiments of any of the methods described herein, the fourth culture medium includes about 1 ng/mL to about 500 ng/mL EPO. In some embodiments, the fourth culture medium includes about 1 ng/mL to about 200 ng/mL EPO. In some embodiments, the fourth culture medium includes about 1 ng/mL to about 100 mg/mL EPO.

In some embodiments of any of the methods described herein, the fourth culture medium includes about 0.1 μg/mL to about 50 μg/mL insulin. In some embodiments, the fourth culture medium includes about 0.1 μg/mL to about 20 μg/mL insulin.

In some embodiments of any of the methods described herein, the fourth culture medium includes about 1 mM to about 8 mM of L-glutamine, L-alanyl-L-glutamine, L-glycyl-L-glutamine, N-acetyl-L-glutamine, or a combination thereof. In some embodiments, the fourth culture medium includes about 3 mM to about 5 mM of L-glutamine, L-alanyl-L-glutamine, L-glycyl-L-glutamine, N-acetyl-L-glutamine, or a combination thereof. In some embodiments, the fourth culture medium includes about 3 mM to about 5 mM L-glutamine. In some embodiments, the fourth culture medium includes about 3 mM to about 5 mM L-alanyl-L-glutamine.

In some embodiments of any of the methods described herein, the fourth culture medium includes about 0.5% v/v to about 10% v/v serum. In some embodiments, the fourth culture medium includes about 4% v/v to about 6% v/v serum.

In some embodiments of any of the methods described herein, the perfusion culturing of step (d) is performed for about 8 days to about 15 days. In some embodiments, the perfusion culturing of step (d) is performed for about 9 days to about 13 days.

In some embodiments of any of the methods described herein, the initial cell density in step (c) is about 2.0×10⁵ cells/mL to about 8.0×10⁵ cells/mL. In some embodiments, the initial cell density in step (c) is about 4.0×10⁵ cells/mL to about 6.0×10⁵ cells/mL.

In some embodiments of any of the methods described herein, the perfusion culturing in steps (b) and (d) includes the use of tangential filtration. In some embodiments, the tangential filtration is alternating tangential filtration (ATF). In some embodiments of any of the methods described herein, the tangential filtration includes the use of a filter that has an average pore size of about 10 nm to about 6.0 μm. In some embodiments, the filter has an average pore size of about 0.1 μm to about 3.0 μm.

In some embodiments of any of the methods described herein, step (d) results in a cell density of about 5×10⁶ enucleated erythroid cells/mL to about 2×10⁸ enucleated erythroid cells/mL. In some embodiments, step (d) results in a cell density of about 1×10⁷ enucleated erythroid cells/mL to about 2×10⁸ enucleated erythroid cells/mL. In some embodiments, step (d) results in a cell density of about 5×10⁷ enucleated erythroid cells/mL to about 2×10⁸ enucleated erytroid cells/mL.

Provided herein are methods of generating a population of enucleated erythroid cells that include: (a) disposing a volume of a first cell culture of erythroid progenitor cells into a second culture medium included within a vessel to provide a second cell culture with an initial cell density of about 0.1×10⁵ cells/mL to about 1×10⁷ cells/mL; (b) batch or fed batch culturing the second cell culture for about 2 days to about 15 days; (c) disposing a volume of the second cell culture of step (b) into a third culture medium included within a perfusion bioreactor to provide a third cell culture with an initial cell density of about 1×10⁵ to about 1×10⁷ cells/mL; (d) perfusion culturing the third cell culture of step (c) for about 5 days to 20 days, wherein after step (d) the third culture medium includes a population of enucleated erythroid cells.

In some embodiments of any of the methods described herein, the method further includes prior to step (a): (i) disposing a plurality of erythroid progenitor cells in a first culture medium included within a vessel to provide the first cell culture with an initial cell density of about of about 0.1×10⁵ cells/mL to about 2×10⁶ cells/mL; and (ii) batch or fed batch culturing the first cell culture for about 1 day to about 15 days.

In some embodiments, the vessel in step (i) is a shake flask. In some embodiments, the shake flask has a volume of about 15 mL to about 5 L. In some embodiments, the shake flask has a volume of about 50 mL to about 1.5 L. In some embodiments, the shake flask has a volume of about 100 mL to about 500 mL. In some embodiments of any of the methods described herein, step (ii) includes incubating the shake flask at about 0.1×g to about 4.1×g. In some embodiments, step (ii) includes incubating the shake flask at about 0.23×g to about 1.78×g. In some embodiments, the vessel in step (i) is a shake tube. In some embodiments, the shake tube has a volume of about 2 mL to about 500 mL. In some embodiments, the shake tube has a volume of about 10 mL to about 250 mL. In some embodiments, the shake tube has as volume of about 50 mL to about 200 mL. In some embodiments of any of the methods described herein, the shake tube is a conical container. In some embodiments of any of the methods described herein, step (ii) includes incubating the shake tube at about 0.1×g to about 4.1×g. In some embodiments, step (ii) includes incubating the shake tube at about 0.23×g to about 1.78×g.

In some embodiments, the vessel in step (i) is a culture bag. In some embodiments, the culture bag has a volume of about 50 mL to about 25 L. In some embodiments, the culture bag has a volume of about 50 mL to about 5 L. In some embodiments, the culture bag has a volume of about 50 mL to about 500 mL. In some embodiments of any of the methods described herein, step (ii) further includes incubating the culture bag at a rocking rate of about 10 rock cycles per minute to about 50 rock cycles per minute. In some embodiments, step (ii) includes incubating the culture bag at a rocking rate of about 10 rock cycles per minute to about 25 rock cycles per minute.

In some embodiments of any of the methods described herein, step (ii) includes batch culturing the first cell culture. In some embodiments of any of the methods described herein, step (ii) includes fed batch culturing the first cell culture. In some embodiments, fed batch culturing includes adding an additional volume of the first culture medium to the first cell culture over time. In some embodiments, the additional volume of the first culture medium is added continuously to the first cell culture over time. In some embodiments, the additional volume of the first cell culture medium is added periodically to the first cell culture over time.

In some embodiments of any of the methods described herein, the first culture medium includes one or more of Flt-3 ligand, stem cell factor (SCF), IL-3, and IL-6. In some embodiments, the first culture medium includes each of Flt-3 ligand, SCF, IL-3, and IL-6.

In some embodiments, the first culture medium includes about 0.1 ng/mL to about 200 ng/mL Flt-3 ligand. In some embodiments, the first culture medium includes about 50 ng/mL to about 150 ng/mL Flt-3 ligand.

In some embodiments of any of the methods described herein, the first culture medium includes about 1 ng/mL to about 1 μg/mL SCF. In some embodiments, the first culture medium includes about 50 ng/mL to about 500 ng/mL SCF.

In some embodiments of any of the methods described herein, the first culture medium includes about 0.1 ng/mL to about 200 ng/mL IL-3.

In some embodiments of any of the methods described herein, the first culture medium includes about 0.1 ng/mL to about 200 ng/mL IL-6. In some embodiments, the first culture medium includes about 10 ng/mL to about 100 ng/mL IL-6.

In some embodiments of any of the methods described herein, the first culture medium includes about 1 μg/mL to about 20 μg/mL insulin. In some embodiments, the first culture medium includes about 8 μg/mL to about 12 μg/mL insulin.

In some embodiments of any of the methods described herein, the first culture medium includes about 1 mM to about 10 mM of L-glutamine, L-alanyl-L-glutamine, L-glycyl-L-glutamine, N-acetyl-L-glutamine, or a combination thereof. In some embodiments, the first culture medium includes about 5 mM to about 7 mM of L-glutamine, L-alanyl-L-glutamine, L-glycyl-L-glutamine, N-acetyl-L-glutamine, or a combination thereof. In some embodiments, the first culture medium includes about 5 mM to about 7 mM L-glutamine. In some embodiments, the first culture medium includes about 5 mM to about 7 mM L-alanyl-L-glutamine. In some embodiments, the first culture medium includes about 5 mM to about 7 mM L-glycyl-L-glutamine. In some embodiments, the first culture medium includes about 5 mM to about 7 mM N-acetyl-L-glutamine.

In some embodiments of any of the methods described herein, the first culture medium includes lipid (e.g., lipid mixture).

In some embodiments of any of the methods described herein, the first culture medium includes about 50 μg/mL to about 400 μg/mL transferrin. In some embodiments, the first culture medium includes about 150 μg/mL to about 250 μg/mL transferrin. In some embodiments, the first culture medium includes about 160 μg/mL to about 240 μg/mL transferrin. In some embodiments, the first culture medium includes about 180 μg/mL to about 220 μg/mL transferrin.

In some embodiments of any of the methods described herein, the batch or fed batch culturing in step (ii) is performed for about 5 days to about 12 days. In some embodiments, the batch or fed batch culturing in step (ii) is performed for about 5 days to about 9 days.

In some embodiments of any of the methods described herein, the initial cell density in step (i) is about 0.1×10⁵ cells/mL to about 2×10⁵ cells/mL. In some embodiments, the initial cell density in step (i) is about 0.5×10⁵ cells/mL to about 1.5×10⁵ cells/mL.

In some embodiments of any of the methods described herein, the vessel in step (a) is a shake flask. In some embodiments, the shake flask has a volume of about 15 mL to about 3 L. In some embodiments, the shake flask has a volume of about 50 mL to about 1.5 L. In some embodiments, the shake flask has a volume of about 100 mL to about 500 mL. In some embodiments of any of the methods described herein, step (b) includes incubating the shake flask at 0.1×g to about 4.1×g. In some embodiments of any of the methods described herein, step (b) includes incubating the shake flask at about 0.23×g to about 1.78×g. In some embodiments of any of the methods described herein, the vessel in step (a) is a shake tube. In some embodiments, the shake tube has a volume of about 2 mL to about 500 mL. In some embodiments, the shake tube has a volume of about 10 mL to about 250 mL. In some embodiments, the shake tube has as volume of about 50 mL to about 200 mL. In some embodiments of any of the methods described herein, the shake tube is a conical container.

In some embodiments of any of the methods described herein, step (b) includes incubating the shake tube at about 0.1×g to about 4.1×g. In some embodiments, step (b) includes incubating the shake tube at about 0.23×g to about 1.78×g.

In some embodiments of any of the methods described herein, the vessel in step (a) is a culture bag. In some embodiments, the culture bag has a volume of about 50 mL to about 25 L. In some embodiments, the culture bag has a volume of about 50 mL to about 5 L. In some embodiments, the culture bag has a volume of about 50 mL to about 500 mL. In some embodiments of any of the methods described herein, step (b) further includes incubating the culture bag at a rocking rate of about 10 rock cycles per minute to about 50 rock cycles per minute. In some embodiments, step (b) includes incubating the culture bag at a rocking rate of about 10 rock cycles per minute to about 25 rock cycles per minute.

In some embodiments of any of the methods described herein, the vessel in step (a) is a bioreactor. In some embodiments, the bioreactor has a volume of 1 L to about 15,000 L. In some embodiments, the bioreactor in step (a) has a volume of 5 L to about 5,000 L. In some embodiments, the bioreactor in step (a) has a volume of about 5 L to about 2,500 L. In some embodiments, the bioreactor in step (a) has a volume of about 5 L to about 100 L.

In some embodiments of any of the methods described herein, step (b) includes agitating the second cell culture with a P/V value of about 10 W/m³ to about 200 W/m³. In some embodiments, step (b) includes agitating the second cell culture with a P/V value of about 10 W/m³ to about 100 W/m³.

In some embodiments of any of the methods described herein, step (b) includes batch culturing the second cell culture.

In some embodiments of any of the methods described herein, step (b) includes fed batch culturing the second cell culture. In some embodiments, fed batch culturing includes adding an additional volume of the second culture medium to the second cell culture over time. In some embodiments, the additional volume of the second culture medium is added continuously to the second cell culture over time. In some embodiments, the additional volume of the second cell culture medium is added periodically to the second cell culture over time.

In some embodiments of any of the methods described herein, the second culture medium includes one or more of: transferrin, IL-3, SCF, dexamethasone, erythropoietin (EPO), and insulin. In some embodiments, the second culture medium includes three or more of: transferrin, IL-3, SCF, dexamethasone, EPO, and insulin. In some embodiments, the second culture medium includes each of: transferrin, IL-3, SCF, dexamethasone, EPO, and insulin.

In some embodiments of any of the methods described herein, the second culture medium includes about 1 μg/mL transferrin to about 500 μg/mL transferrin. In some embodiments, the second culture medium includes about 100 μg/mL transferrin to about 300 μg/mL transferrin.

In some embodiments of any of the methods described herein, the second culture medium includes about 0.1 ng/mL to about 200 ng/mL IL-3. In some embodiments, the second culture medium includes about 0.1 ng/mL to about 100 ng/mL IL-3. In some embodiments, the second culture medium includes about 0.1 ng/mL to about 10 ng/mL IL-3.

In some embodiments of any of the methods described herein, the second culture medium includes about 1 ng/mL to about 1 μg/mL SCF. In some embodiments, the second culture medium includes about 1 ng/mL to about 500 ng/mL SCF. In some embodiments, the second culture medium includes about 10 ng/mL to about 200 ng/nL SCF.

In some embodiments of any of the methods described herein, the second culture medium includes about 0.1 nM to about 200 nM dexamethasone. In some embodiments, the second culture medium includes about 0.1 nM to about 100 nM dexamethasone. In some embodiments, the second culture medium includes about 0.1 nM to about 25 nM dexamethasone.

In some embodiments of any of the methods described herein, the second culture medium includes about 1 ng/mL to about 500 ng/mL EPO. In some embodiments, the second culture medium includes about 1 ng/mL to about 200 ng/mL EPO. In some embodiments, the second culture medium includes about 1 ng/mL to about 100 mg/mL EPO.

In some embodiments of any of the methods described herein, the second culture medium includes about 0.1 μg/mL to about 50 μg/mL insulin. In some embodiments, the second culture medium includes about 0.1 μg/mL to about 20 μg/mL insulin.

In some embodiments of any of the methods described herein, the second culture medium includes about 1 mM to about 10 mM of L-glutamine, L-alanyl-L-glutamine, L-glycyl-L-glutamine, N-acetyl-L-glutamine, or a combination thereof. In some embodiments, the second culture medium includes about 5 mM to about 7 mM L-glutamine. In some embodiments, the second culture medium includes about 5 mM to about 7 mM L-alanyl-L-glutamine. In some embodiments, the second culture medium includes about 5 mM to about 7 mM L-glycyl-L-glutamine. In some embodiments, the second culture medium includes about 5 mM to about 7 mM N-acetyl-L-glutamine.

In some embodiments of any of the methods described herein, the second culture medium includes lipid (e.g., lipid mixture).

In some embodiments of any of the methods described herein, the batch or fed batch culturing of step (b) is performed for about 4 days to about 10 days. In some embodiments, the batch or fed batch culturing of step (b) is performed for about 5 days to about 8 days.

In some embodiments of any of the methods described herein, the initial cell density in step (a) is about 0.1×10⁵ cells/mL to about 2×10⁵ cells/mL. In some embodiments, the initial cell density in step (a) is about 0.5×10⁵ cells/mL to about 1.5×10⁵ cells/mL.

In some embodiments of any of the methods described herein, the perfusion bioreactor in step (c) has a volume of about 500 mL to about 15,000 L. In some embodiments, the perfusion bioreactor in step (c) has a volume of 5 L to about 5,000 L. In some embodiments, the perfusion bioreactor in step (c) has a volume of about 5 L to about 2,500 L. In some embodiments, the perfusion bioreactor in step (c) has a volume of about 5 L to about 100 L.

In some embodiments of any of the methods described herein, step (d) includes agitating the third cell culture with a P/V value of about 10 W/m³ to about 200 W/m³. In some embodiments, step (d) includes agitating the third cell culture with a P/V value of about 10 W/m³ to about 100 W/m³.

In some embodiments of any of the methods described herein, the perfusion culturing in step (d) is performed using a perfusion rate of about 0.5 nL/cell/day to about 40 nL/cell/day. In some embodiments, the perfusion culturing in step (d) is performed using a perfusion rate of about 5 nL/cell/day to about 35 nL/cell/day. In some embodiments, the perfusion culturing in step (d) is performed using a perfusion rate of about 10 nL/cell/day to about 25 nL/cell/day.

In some embodiments of any of the methods described herein, the perfusion culturing in step (d) includes: (i) adding an additional volume of the third culture medium to the third cell culture for a first period of time, and (ii) adding an additional volume of a fourth culture medium to the third cell culture for a second period of time.

In some embodiments, the additional volume of the third culture medium in (i) is added continuously to the third cell culture for the first period of time; and/or the additional volume of the fourth culture medium in (ii) is added continuously to the third cell culture for the second period of time.

In some embodiments, the additional volume of the third culture medium in (i) is added periodically to the third cell culture for the first period of time; and/or the additional volume of the fourth culture medium in (ii) is added periodically to the third cell culture for the second period of time.

In some embodiments of any of the methods described herein, the first period of time in (i) is about 1 day to about 7 days. In some embodiments, the first period of time in (i) is about 3 days to about 5 days. In some embodiments of any of the methods described herein, the second period of time in (ii) is about 1 day to about 10 days. In some embodiments, the second period of time in (ii) is about 3 days to about 7 days.

In some embodiments of any of the methods described herein, the third culture medium includes one or more of: transferrin, insulin, SCF, and EPO. In some embodiments, the third culture medium includes two or more of: transferrin, insulin, SCF, and EPO. In some embodiments, the third culture medium includes each of: transferrin, insulin, SCF, and EPO.

In some embodiments of any of the methods described herein, the third culture medium includes about 1 μg/mL transferrin to about 500 μg/mL transferrin. In some embodiments, the third culture medium includes about 100 μg/mL transferrin to about 300 μg/mL transferrin.

In some embodiments of any of the methods described herein, the third culture medium includes about 1 ng/mL to about 1 μg/mL SCF. In some embodiments, the third culture medium includes about 1 ng/mL to about 500 ng/mL SCF. In some embodiments, the third culture medium includes about 10 ng/mL to about 200 ng/nL SCF.

In some embodiments of any of the methods described herein, the third culture medium includes about 1 ng/mL to about 500 ng/mL EPO. In some embodiments, the third culture medium includes about 1 ng/mL to about 200 ng/mL EPO. In some embodiments, the third culture medium includes about 1 ng/mL to about 100 mg/mL EPO.

In some embodiments of any of the methods described herein, the third culture medium includes about 0.1 μg/mL to about 50 μg/mL insulin. In some embodiments, the third culture medium includes about 0.1 μg/mL to about 20 μg/mL insulin.

In some embodiments of any of the methods described herein, the third culture medium includes about 1 mM to about 8 mM of L-glutamine, L-alanyl-L-glutamine, L-glycyl-L-glutamine, N-acetyl-L-glutamine, or a combination thereof. In some embodiments, the third culture medium includes about 3 mM to about 5 mM of L-glutamine, L-alanyl-L-glutamine, L-glycyl-L-glutamine, N-acetyl-L-glutamine, or a combination thereof. In some embodiments, the third culture medium includes about 3 mM to about 5 mM L-glutamine. In some embodiments, the third culture medium includes about 3 mM to about 5 mM L-alanyl-L-glutamine.

In some embodiments of any of the methods described herein, the third culture medium includes about 0.5% v/v to about 10% v/v serum. In some embodiments, the third culture medium includes about 4% v/v to about 6% v/v serum.

In some embodiments of any of the methods described herein, the fourth culture medium includes one or more of: transferrin, insulin, and EPO. In some embodiments, the fourth culture medium includes two or more of: transferrin, insulin, and EPO. In some embodiments, the fourth culture medium includes each of: transferrin, insulin, and EPO.

In some embodiments of any of the methods described herein, the fourth culture medium includes about 100 μg/mL transferrin to about 2 mg/mL transferrin. In some embodiments, the fourth culture medium includes about 100 μg/mL transferrin to about 1.5 mg/mL transferrin.

In some embodiments of any of the methods described herein, the fourth culture medium includes about 1 ng/mL to about 500 ng/mL EPO. In some embodiments, the fourth culture medium includes about 1 ng/mL to about 200 ng/mL EPO. In some embodiments, the fourth culture medium includes about 1 ng/mL to about 100 mg/mL EPO.

In some embodiments of any of the methods described herein, the fourth culture medium includes about 0.1 μg/mL to about 50 μg/mL insulin. In some embodiments, the fourth culture medium includes about 0.1 μg/mL to about 20 μg/mL insulin.

In some embodiments of any of the methods described herein, the fourth culture medium includes about 1 mM to about 8 mM of L-glutamine, L-alanyl-L-glutamine, L-glycyl-L-glutamine, N-acetyl-L-glutamine, or a combination thereof. In some embodiments, the fourth culture medium includes about 3 mM to about 5 mM of L-glutamine, L-alanyl-L-glutamine, L-glycyl-L-glutamine, N-acetyl-L-glutamine, or a combination thereof. In some embodiments, the fourth culture medium includes about 3 mM to about 5 mM L-glutamine. In some embodiments, the fourth culture medium includes about 3 mM to about 5 mM L-alanyl-L-glutamine.

In some embodiments of any of the methods described herein, the fourth culture medium includes about 0.5% v/v to about 10% v/v serum. In some embodiments, the fourth culture medium includes about 4% v/v to about 6% v/v serum.

In some embodiments of any of the methods described herein, the perfusion culturing of step (d) is performed for about 8 days to about 15 days. In some embodiments, the perfusion culturing of step (d) is performed for about 9 days to about 13 days.

In some embodiments of any of the methods described herein, the initial cell density in step (c) is about 2.0×10⁵ cells/mL to about 8.0×10⁵ cells/mL. In some embodiments, the initial cell density in step (c) is about 4.0×10⁵ cells/mL to about 6.0×10⁵ cells/mL.

In some embodiments of any of the methods described herein, the perfusion culturing in step (d) includes the use of tangential filtration. In some embodiments, the tangential filtration is alternating tangential filtration (ATF). In some embodiments of any of the methods described herein, the tangential filtration includes the use of a filter that has an average pore size of about 10 nm to about 6.0 μm. In some embodiments, the filter has an average pore size of about 0.1 μm to about 3.0 μm.

In some embodiments of any of the methods described herein, step (d) results in a cell density of about 5×10⁶ enucleated erythroid cells/mL to about 2×10⁸ enucleated erythroid cells/mL. In some embodiments, step (d) results in a cell density of about 1×10⁷ enucleated erythroid cells/mL to about 2×10⁸ enucleated erythroid cells/mL. In some embodiments, step (d) results in a cell density of about 5×10⁷ enucleated erythroid cells/mL to about 2×10⁸ enucleated erythroid cells/mL.

Also provided herein are methods of generating a population of enucleated erythroid cells that include: (a) disposing a volume of a first cell culture of erythroid progenitor cells into a second culture medium included within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium included within a vessel to provide a third cell culture with an initial cell density of about 1×10⁵ to about 1×10⁷ cells/mL; (d) batch or fed batch culturing the third cell culture of step (c) for about 5 days to 20 days, wherein after step (d) the third culture medium includes a population of enucleated erythroid cells.

In some embodiments of any of the methods described herein, the method further includes prior to step (a): (i) disposing a plurality of erythroid progenitor cells in a first culture medium included within a vessel to provide the first cell culture with an initial cell density of about of about 0.1×10⁵ cells/mL to about 2×10⁶ cells/mL; and (ii) batch or fed batch culturing the first cell culture for about 1 day to about 15 days.

In some embodiments, the vessel in step (i) is a shake flask. In some embodiments, the shake flask has a volume of about 15 mL to about 3 L. In some embodiments, the shake flask has a volume of about 50 mL to about 1.5 L. In some embodiments, the shake flask has a volume of about 100 mL to about 500 mL. In some embodiments of any of the methods described herein, step (ii) includes incubating the shake flask at about 0.1×g to about 4.1×g. In some embodiments, step (ii) includes incubating the shake flask at about 0.23×g to about 1.78×g.

In some embodiments, the vessel in step (i) is a shake tube. In some embodiments, the shake tube has a volume of about 2 mL to about 500 mL. In some embodiments, the shake tube has a volume of about 10 mL to about 250 mL. In some embodiments, the shake tube has a volume of about 50 mL to about 200 mL. In some embodiments of any of the methods described herein, the shake tube is a conical container. In some embodiments of any of the methods described herein, step (ii) includes incubating the shake tube at about 0.1×g to about 4.1×g. In some embodiments, step (ii) includes incubating the shake tube at about 0.23×g to about 1.78×g.

In some embodiments, the vessel in step (i) is a culture bag. In some embodiments, the culture bag has a volume of about 50 mL to about 25 L. In some embodiments, the culture bag has a volume of about 50 mL to about 5 L. In some embodiments, the culture bag has a volume of about 50 mL to about 500 mL. In some embodiments of any of the methods described herein, step (ii) further includes incubating the culture bag at a rocking rate of about 10 rock cycles per minute to about 50 rock cycles per minute. In some embodiments, step (ii) includes incubating the culture bag at a rocking rate of about 10 rock cycles per minute to about 25 rock cycles per minute.

In some embodiments of any of the methods described herein, step (ii) includes batch culturing the first cell culture.

In some embodiments of any of the methods described herein, step (ii) includes fed batch culturing the first cell culture. In some embodiments, fed batch culturing includes adding an additional volume of the first culture medium to the first cell culture over time. In some embodiments, the additional volume of the first culture medium is added continuously to the first cell culture over time. In some embodiments, the additional volume of the first cell culture medium is added periodically to the first cell culture over time.

In some embodiments of any of the methods described herein, the first culture medium includes one or more of Flt-3 ligand, stem cell factor (SCF), IL-3, and IL-6. In some embodiments, the first culture medium includes each of Flt-3 ligand, SCF, IL-3, and IL-6.

In some embodiments of any of the methods described herein, the first culture medium includes about 0.1 ng/mL to about 200 ng/mL Flt-3 ligand. In some embodiments, the first culture medium includes about 50 ng/mL to about 150 ng/mL Flt-3 ligand.

In some embodiments of any of the methods described herein, the first culture medium includes about 1 ng/mL to about 1 μg/mL SCF. In some embodiments, the first culture medium includes about 50 ng/mL to about 500 ng/mL SCF. In some embodiments of any of the methods described herein, the first culture medium includes about 0.1 ng/mL to about 200 ng/mL IL-3.

In some embodiments of any of the methods described herein, the first culture medium includes about 0.1 ng/mL to about 200 ng/mL IL-6. In some embodiments, the first culture medium includes about 10 ng/mL to about 100 ng/mL IL-6.

In some embodiments of any of the methods described herein, the first culture medium includes about 1 μg/mL to about 20 μg/mL insulin. In some embodiments, the first culture medium includes about 8 μg/mL to about 12 μg/mL insulin.

In some embodiments of any of the methods described herein, the first culture medium includes about 1 mM to about 10 mM of L-glutamine, L-alanyl-L-glutamine, L-glycyl-L-glutamine, N-acetyl-L-glutamine, or a combination thereof. In some embodiments, the first culture medium includes about 5 mM to about 7 mM of L-glutamine, L-alanyl-L-glutamine, L-glycyl-L-glutamine, N-acetyl-L-glutamine, or a combination thereof. In some embodiments of any of the methods described herein, wherein the first culture medium comprises about 5 mM to about 7 mM of L-glutamine. In some embodiments of any of the methods described herein, wherein the first culture medium comprises about 5 mM to about 7 mM of L-alanyl-L-glutamine. In some embodiments of any of the methods described herein, wherein the first culture medium comprises about 5 mM to about 7 mM of L-glycyl-L-glutamine. In some embodiments of any of the methods described herein, wherein the first culture medium comprises about 5 mM to about 7 mM of N-acetyl-L-glutamine.

In some embodiments of any of the methods described herein, the first culture medium includes lipid (e.g., lipid mixture).

In some embodiments of any of the methods described herein, the first culture medium includes about 50 μg/mL to about 400 μg/mL transferrin. In some embodiments, the first culture medium includes about 150 μg/mL to about 250 μg/mL transferrin. In some embodiments, the first culture medium includes about 160 μg/mL to about 240 μg/mL transferrin. In some embodiments, the first culture medium includes about 180 μg/mL to about 220 μg/mL transferrin.

In some embodiments of any of the methods described herein, the batch or fed batch culturing in step (ii) is performed for about 5 days to about 12 days. In some embodiments, the batch or fed batch culturing in step (ii) is performed for about 5 days to about 9 days.

In some embodiments of any of the methods described herein, the initial cell density in step (i) is about 0.1×10⁵ cells/mL to about 2×10⁵ cells/mL. In some embodiments, the initial cell density in step (i) is about 0.5×10⁵ cells/mL to about 1.5×10⁵ cells/mL.

In some embodiments of any of the methods described herein, the perfusion bioreactor in step (a) has a volume of about 500 mL to about 15,000 L. In some embodiments, the perfusion bioreactor in step (a) has a volume of 5 L to about 5,000 L. In some embodiments, the perfusion bioreactor in step (a) has a volume of about 5 L to about 2,500 L. In some embodiments, the perfusion bioreactor in step (a) has a volume of about 5 L to about 100 L. In some embodiments of any of the methods described herein, step (b) includes agitating the second cell culture with a P/V value of about 10 W/m³ to about 200 W/m³. In some embodiments, step (b) includes agitating the second cell culture with a P/V value of about 10 W/m³ to about 100 W/m³. In some embodiments of any of the methods described herein, the perfusion culturing in step (b) is performed using a perfusion rate of about 0.5 nL/cell/day to about 40 nL/cell/day. In some embodiments, the perfusion culturing in step (b) is performed using a perfusion rate of about 5 nL/cell/day to about 35 nL/cell/day. In some embodiments, the perfusion culturing in step (b) is performed using a perfusion rate of about 10 nL/cell/day to about 25 nL/cell/day.

In some embodiments of any of the methods described herein, the perfusion culturing in step (b) includes adding an additional volume of the second culture medium to the second cell culture over time. In some embodiments, the additional volume of the second culture medium is added continuously to the second cell culture over time. In some embodiments, the additional volume of the second culture medium is added periodically to the second cell culture over time.

In some embodiments of any of the methods described herein, the second culture medium includes one or more of: transferrin, IL-3, SCF, dexamethasone, erythropoietin (EPO), and insulin. In some embodiments, the second culture medium includes three or more of: transferrin, IL-3, SCF, dexamethasone, EPO, and insulin. In some embodiments, the second culture medium includes each of: transferrin, IL-3, SCF, dexamethasone, EPO, and insulin.

In some embodiments of any of the methods described herein, the second culture medium includes about 1 μg/mL transferrin to about 500 μg/mL transferrin. In some embodiments, the second culture medium includes about 100 μg/mL transferrin to about 300 μg/mL transferrin.

In some embodiments of any of the methods described herein, the second culture medium includes about 0.1 ng/mL to about 200 ng/mL IL-3. In some embodiments, the second culture medium includes about 0.1 ng/mL to about 100 ng/mL IL-3. In some embodiments, the second culture medium includes about 0.1 ng/mL to about 10 ng/mL IL-3.

In some embodiments of any of the methods described herein, the second culture medium includes about 1 ng/mL to about 1 μg/mL SCF. In some embodiments, the second culture medium includes about 1 ng/mL to about 500 ng/mL SCF. In some embodiments, the second culture medium includes about 10 ng/mL to about 200 ng/nL SCF.

In some embodiments of any of the methods described herein, the second culture medium includes about 0.1 nM to about 200 nM dexamethasone. In some embodiments, the second culture medium includes about 0.1 nM to about 100 nM dexamethasone. In some embodiments, the second culture medium includes about 0.1 nM to about 25 nM dexamethasone.

In some embodiments of any of the methods described herein, the second culture medium includes about 1 ng/mL to about 500 ng/mL EPO. In some embodiments, the second culture medium includes about 1 ng/mL to about 200 ng/mL EPO. In some embodiments, the second culture medium includes about 1 ng/mL to about 100 mg/mL EPO.

In some embodiments of any of the methods described herein, the second culture medium includes about 0.1 μg/mL to about 50 μg/mL insulin. In some embodiments, the second culture medium includes about 0.1 μg/mL to about 20 μg/mL insulin.

In some embodiments of any of the methods described herein, the second culture medium includes about 1 mM to about 10 mM of L-glutamine, L-alanyl-L-glutamine, L-glycyl-L-glutamine, N-acetyl-L-glutamine, or a combination thereof. In some embodiments, the second culture medium includes about 5 mM to about 7 mM L-glutamine. In some embodiments, the second culture medium includes about 5 mM to about 7 mM L-alanyl-L-glutamine. In some embodiments, the second culture medium includes about 5 mM to about 7 mM L-glycyl-L-glutamine. In some embodiments, the second culture medium includes about 5 mM to about 7 mM N-acetyl-L-glutamine.

In some embodiments of any of the methods described herein, the second culture medium includes about lipid (e.g., lipid mixture).

In some embodiments of any of the methods described herein, the perfusion culturing of step (b) is performed for about 4 days to about 10 days. In some embodiments, the perfusion culturing of step (b) is performed for about 5 days to about 8 days.

In some embodiments of any of the methods described herein, the initial cell density in step (a) is about 0.1×10⁵ cells/mL to about 2×10⁵ cells/mL. In some embodiments, the initial cell density in step (a) is about 0.5×10⁵ cells/mL to about 1.5×10⁵ cells/mL.

In some embodiments of any of the methods described herein, the vessel in step (c) is a shake flask. In some embodiments, the shake flask has a volume of about 15 mL to about 3 L. In some embodiments, the shake flask has a volume of about 50 mL to about 1.5 L. In some embodiments, the shake flask has a volume of about 100 mL to about 500 mL. In some embodiments of any of the methods described herein, step (d) includes incubating the shake flask at about 0.1×g to about 4.1×g. In some embodiments, step (d) includes incubating the shake flask at about 0.23×g to about 1.78×g.

In some embodiments of any of the methods described herein, the vessel in step (c) is a shake tube. In some embodiments, the shake tube has a volume of about 2 mL to about 500 mL. In some embodiments, the shake tube has a volume of about 10 mL to about 250 mL. In some embodiments, the shake tube has as volume of about 50 mL to about 200 mL. In some embodiments of any of the methods described herein, the shake tube is a conical container. In some embodiments of any of the methods described herein, step (d) includes incubating the shake tube at about 0.1×g to about 4.1×g. In some embodiments, step (d) includes incubating the shake tube at about 0.23×g to about 1.78×g.

In some embodiments of any of the methods described herein, the vessel in step (c) is a culture bag. In some embodiments, the culture bag has a volume of about 50 mL to about 25 L. In some embodiments, the culture bag has a volume of about 50 mL to about 5 L. In some embodiments, the culture bag has a volume of about 50 mL to about 500 mL. In some embodiments of any of the methods described herein, step (d) further includes incubating the culture bag at a rocking rate of about 10 rock cycles per minute to about 50 rock cycles per minute. In some embodiments, step (d) includes incubating the culture bag at a rocking rate of about 10 rock cycles per minute to about 25 rock cycles per minute.

In some embodiments of any of the methods described herein, the vessel in step (c) is a bioreactor. In some embodiments, the bioreactor has a volume of 1L to about 15,000L. In some embodiments, the bioreactor has a volume of 5 L to about 5,000 L. In some embodiments, the bioreactor has a volume of about 5 L to about 2,500 L. In some embodiments, the bioreactor has a volume of about 5 L to about 100 L. In some embodiments of any of the methods described herein, step (b) includes agitating the second cell culture with a P/V value of about 10 W/m³ to about 200 W/m³. In some embodiments, step (b) includes agitating the second cell culture with a P/V value of about 10 W/m³ to about 100 W/m³. In some embodiments of any of the methods described herein, step (d) includes batch culturing the third cell culture.

In some embodiments of any of the methods described herein, step (d) includes fed batch culturing the third cell culture. In some embodiments, fed batch culturing includes adding an additional volume of the third culture medium to the third cell culture over time. In some embodiments, the additional volume of the third culture medium is added continuously to the third cell culture over time. In some embodiments, the additional volume of the third cell culture medium is added periodically to the third cell culture over time.

In some embodiments of any of the methods described herein, the batch and fed batch culturing in step (d) includes: (i) adding an additional volume of the third culture medium to the third cell culture for a first period of time, and (ii) adding an additional volume of a fourth culture medium to the third cell culture for a second period of time.

In some embodiments, the additional volume of the third culture medium in (i) is added continuously to the third cell culture for the first period of time; and/or the additional volume of the fourth culture medium in (ii) is added continuously to the third cell culture for the second period of time.

In some embodiments, the additional volume of the third culture medium in (i) is added periodically to the third cell culture for the first period of time; and/or the additional volume of the fourth culture medium in (ii) is added periodically to the third cell culture for the second period of time.

In some embodiments of any of the methods described herein, the first period of time in (i) is about 1 day to about 7 days. In some embodiments, the first period of time in (i) is about 3 days to about 5 days. In some embodiments of any of the methods described herein, the second period of time in (ii) is about 1 day to about 10 days. In some embodiments, the second period of time in (ii) is about 3 days to about 7 days.

In some embodiments of any of the methods described herein, the third culture medium includes one or more of: transferrin, insulin, SCF, and EPO. In some embodiments, the third culture medium includes two or more of: transferrin, insulin, SCF, and EPO. In some embodiments, the third culture medium includes each of: transferrin, insulin, SCF, and EPO.

In some embodiments of any of the methods described herein, the third culture medium includes about 1 μg/mL transferrin to about 500 μg/mL transferrin. In some embodiments, the third culture medium includes about 100 μg/mL transferrin to about 300 μg/mL transferrin.

In some embodiments of any of the methods described herein, the third culture medium includes about 1 ng/mL to about 1 μg/mL SCF. In some embodiments, the third culture medium includes about 1 ng/mL to about 500 ng/mL SCF. In some embodiments, the third culture medium includes about 10 ng/mL to about 200 ng/nL SCF. In some embodiments of any of the methods described herein, the third culture medium includes about 1 ng/mL to about 500 ng/mL EPO. In some embodiments, the third culture medium includes about 1 ng/mL to about 200 ng/mL EPO. In some embodiments, the third culture medium includes about 1 ng/mL to about 100 mg/mL EPO.

In some embodiments of any of the methods described herein, the third culture medium includes about 0.1 μg/mL to about 50 μg/mL insulin. In some embodiments, the third culture medium includes about 0.1 μg/mL to about 20 μg/mL insulin.

In some embodiments of any of the methods described herein, the third culture medium includes about 1 mM to about 8 mM of L-glutamine, L-alanyl-L-glutamine, L-glycyl-L-glutamine, N-acetyl-L-glutamine, or a combination thereof. In some embodiments, the third culture medium includes about 3 mM to about 5 mM of L-glutamine, L-alanyl-L-glutamine, L-glycyl-L-glutamine, N-acetyl-L-glutamine, or a combination thereof. In some embodiments, the third culture medium includes about 3 mM to about 5 mM L-glutamine. In some embodiments, the third culture medium includes about 3 mM to about 5 mM L-alanyl-L-glutamine.

In some embodiments of any of the methods described herein, the third culture medium includes about 0.5% v/v to about 10% v/v serum. In some embodiments, the third culture medium includes about 4% v/v to about 6% v/v serum.

In some embodiments of any of the methods described herein, the fourth culture medium includes one or more of: transferrin, insulin, and EPO. In some embodiments, the fourth culture medium includes two or more of: transferrin, insulin, and EPO. In some embodiments, the fourth culture medium includes each of: transferrin, insulin, and EPO.

In some embodiments of any of the methods described herein, the fourth culture medium includes about 100 μg/mL transferrin to about 2 mg/mL transferrin. In some embodiments, the fourth culture medium includes about 100 μg/mL transferrin to about 1.5 mg/mL transferrin.

In some embodiments of any of the methods described herein, the fourth culture medium includes about 1 ng/mL to about 500 ng/mL EPO. In some embodiments, the fourth culture medium includes about 1 ng/mL to about 200 ng/mL EPO. In some embodiments, the fourth culture medium includes about 1 ng/mL to about 100 mg/mL EPO.

In some embodiments of any of the methods described herein, the fourth culture medium includes about 0.1 μg/mL to about 50 μg/mL insulin. In some embodiments, the fourth culture medium includes about 0.1 μg/mL to about 20 μg/mL insulin.

In some embodiments of any of the methods described herein, the fourth culture medium includes about 1 mM to about 8 mM of L-glutamine, L-alanyl-L-glutamine, L-glycyl-L-glutamine, N-acetyl-L-glutamine, or a combination thereof. In some embodiments, the fourth culture medium includes about 3 mM to about 5 mM of L-glutamine, L-alanyl-L-glutamine, L-glycyl-L-glutamine, N-acetyl-L-glutamine, or a combination thereof. In some embodiments, the fourth culture medium includes about 3 nM to about 5 mM L-glutamine. In some embodiments, the fourth culture medium includes about 3 mM to about 5 mM L-alanyl-L-glutamine.

In some embodiments of any of the methods described herein, the fourth culture medium includes about 0.5% v/v to about 10% v/v serum. In some embodiments, the fourth culture medium includes about 4% v/v to about 6% v/v serum.

In some embodiments of any of the methods described herein, the batch or fed batch culturing of step (d) is performed for about 8 days to about 15 days. In some embodiments, the batch or fed batch culturing of step (d) is performed for about 9 days to about 13 days.

In some embodiments of any of the methods described herein, the initial cell density in step (c) is about 2.0×10⁵ cells/mL to about 8.0×10⁵ cells/mL. In some embodiments, the initial cell density in step (c) is about 4.0×10⁵ cells/mL to about 6.0×10⁵ cells/mL.

In some embodiments of any of the methods described herein, the perfusion culturing in step (b) includes the use of tangential filtration. In some embodiments, the tangential filtration is alternating tangential filtration (ATF). In some embodiments of any of the methods described herein, the tangential filtration includes the use of a filter that has an average pore size of about 10 nm to about 6.0 μm. In some embodiments, the filter has an average pore size of about 0.1 μm to about 3.0 μm.

In some embodiments of any of the methods described herein, step (d) results in a cell density of about 5×10⁶ enucleated erythroid cells/mL to about 2×10⁸ enucleated erythroid cells/mL. In some embodiments, step (d) results in a cell density of about 1×10⁷ enucleated erythroid cells/mL to about 2×10⁸ enucleated erythroid cells/mL. In some embodiments, step (d) results in a cell density of about 5×10⁷ enucleated erythroid cells/mL to about 2×10⁸ enucleated erythroid cells/mL.

In some embodiments of any of the methods described herein, the erythroid progenitor cells are human erythroid progenitor cells. In some embodiments, the erythroid progenitor cells are obtained from an O negative human donor. In some embodiments, the erythroid progenitor cells are obtained from an O negative and a Kell negative human donor.

In some embodiments of any of the methods described herein, the population of enucleated erythroid cells are a population of enucleated human erythroid cells.

In some embodiments of any of the methods described herein, the method further includes: (e) isolating the population of enucleated erythroid cells from the third cell culture in step (d).

In some embodiments of any of the methods described herein, the method further includes: (f) formulating the population of enucleated erythroid cells isolated in step (e).

In some embodiments of any of the methods described herein, the method further includes: (g) administering the formulated population of enucleated erythroid cells in step (f) to a subject in need thereof.

In some embodiments of any of the methods described herein, the method further includes: prior to step (a), introducing a nucleic acid into the erythroid progenitor cells in the first cell culture, and the method results in the production of a population of engineered enucleated erythroid cells.

In some embodiments, the population of engineered enucleated erythroid cells are engineered enucleated human erythroid cells.

In some embodiments of any of the methods described herein, the nucleic acid encodes one or more exogenous polypeptides.

In some embodiments of any of the methods described herein, the engineered human enucleated erythroid cells include one or more exogenous protein (s).

In some embodiments of any of the methods described herein, the method further includes click-conjugating one or more exogenous proteins to the cells. In some embodiments of any of the methods described herein, the method further includes hypotonically loading the cells. In some embodiments of any of the methods described herein, the method further includes loading the cells via physical manipulation.

In some embodiments of any of the methods described herein, one of the one or more exogenous protein(s) is present in the cytosol of the engineered human enucleated erythroid cells.

In some embodiments of any of the methods described herein, one of the one or more exogenous protein is a protein present on the membrane of the engineered human enucleated erythroid cells.

In some embodiments of any of the methods described herein, one of the one or more exogenous protein(s) is phenylalanine ammonia lyase, wherein the phenylalanine ammonia lyase is present in the cytosol of the engineered human enucleated erythroid cell.

In some embodiments of any of the methods described herein, the one or more exogenous protein(s) includes an immunomodulatory molecule. In some embodiments of any of the methods described herein, the one or more exogenous protein(s) includes an antigen-presenting molecule.

In some embodiments of any of the methods described herein, the method further includes: (e) isolating the population of engineered enucleated erythroid cells from the third cell culture in step (d).

In some embodiments of any of the methods described herein, the method further includes: (f) formulating the population of engineered enucleated erythroid cells isolated in step (e).

In some embodiments of any of the methods described herein, the method further includes: (g) administering the formulated population of engineered enucleated erythroid cells in step (f) to a subject in need thereof.

Also provided herein are populations of enucleated erythroid cells produced by any of the methods described herein.

Also provided herein are formulations produced by any of the methods described herein.

The term “population” means two or more of a given article (e.g., any of the exemplary enucleated erythroid cells described herein).

The term “engineered enucleated erythroid cell” means an enucleated erythroid cell (e.g., a human enucleated erythroid cell) that comprises one or more (e.g., two, three, four, five, or six) exogenous protein(s) (e.g., any combination of the exemplary exogenous proteins described herein or known in the art). For example, an engineered enucleated erythroid cell can have one or more exogenous protein(s) present in its cytosol. In some examples, an engineered enucleated erythroid cell can have one or more exogenous protein(s) present on its plasma membrane. In some examples, an engineered red blood cell can have (i) one or more exogenous protein(s) present in its cytosol and (ii) one or more exogenous proteins present on its plasma membrane. Non-limiting examples of engineered enucleated erythroid cells include click-conjugated enucleated erythroid cells, enucleated erythroid cell that have been hypotonically loaded, and enucleated erythroid cells that have been loaded by physical manipulation (e.g., any of the exemplary types of physical manipulation described herein or known in the art). Additional non-limiting aspects of engineered enucleated erythroid cells are describered herein.

The term “click-conjugated enucleated erythroid cell” means an engineered enucleated erythroid cell that has at least one exogenous protein conjugated to another protein (e.g., an endogenous protein of an enucleated red blood cell or different exogenous protein) present on the plasma membrane of an engineered enucleated erythroid cells through a chemical reaction.

The term “hypotonically-loaded enucleated erythroid cell” means an engineered enucleated erythroid cell that was generated, at least in part, by exposing an enucleated erythroid cell or an erythroid progenitor cell to a low ionic strength buffer (e.g., any of the exemplary low ionic strength buffers described herein) comprising one or more exogenous protein(s). Non-limiting examples of methods that can be used to generate a hypotonically-loaded enucleated erythroid cell are described herein. Additional methods for generating a hypotonically-loaded enucleated erythroid cell are known in the art.

The term “enucleated erythroid cell loaded by physical manipulation” means an enucleated erythroid cell that was generated, at least in part, by physically manipulating an erythroid progenitor cell in a manner that results in the introduction of one or more exogenous proteins (e.g., any of the exemplary exogenous proteins described herein or known in the art) and/or a nucleic acid encoding one or more exogenous protein(s) (e.g., any of the exemplary exogenous proteins described herein or known in the art) into the erythroid progenitor cell. Non-limiting examples of physical manipulation that can be used to introduce a nucleic acid encoding one or more exogenous protein(s) into an erythroid progenitor cell include electroporation and particle-mediated transfection. Additional examples of physical manipulation that can be used to introduce a nucleic acid encoding one or more exogenous protein(s) into an erythroid progenitor are known in the art.

The term “exogenous protein” refers to a protein that is introduced into or onto a cell, or is caused to be expressed by the cell by introducing an exogenous nucleic acid encoding the protein into the cell or into a progenitor of the cell. In some embodiments, an exogenous protein is a protein encoded by an exogenous nucleic acid that was introduced into the cell or a progenitor of the cell, which nucleic acid is optionally not retained by the cell. In some embodiments, an exogenous protein is a protein conjugated to the surface of the cell by chemical or enzymatic means. Non-limiting classes of exogenous proteins include enzymes, interleukins, cytokine receptors, Fc-binding molecules, T-cell activating ligands, T-cell receptors, immune inhibitory molecules, MHC molecules, APC-binding molecules, autoantigens, allergens, toxins, targeting agents, receptor ligands (e.g., receptor agonists or receptor antagonists), and antibodies or antibody fragments. Additional examples of exogenous proteins that can be present in an engineered enucleated erythroid cell are described herein (see, e.g., Tables A-D). Additional examples of exogenous proteins that can be present in engineered enucleated erythroid cells are known in the art.

The term “protein present on the membrane” means a (1) a protein that is physically attached to or at least partially embedded in the membrane of an enucleated erythroid cell (e.g., a transmembrane protein, a peripheral membrane protein, a lipid-anchored protein (e.g., a GPI-anchor, an N-myristolyated protein, or a S-palmitoylated protein)) or (2) a protein that is stably bound to its cognate receptor, where the cognate receptor is physically attached to the membrane of an enucleated erythroid cell (e.g., a ligand bound to its cognate receptor, where the cognate receptor is physically attached to the membrane of the enucleated erythroid cell). Non-limiting methods for determining the presence of protein on the membrane of a mammalian cell include fluorescence-activated cell sorting (FACS), immunohistochemistry, cell-fractionation assays and Western blotting.

The term “erythroid progenitor cells” means a mammalian cell that is capable of eventually differentiating/developing into an enucleated erythroid cell. In some embodiments, the erythroid progenitor cell is a cord blood stem cell, a CD34⁺ cell, a hematopoietic stem/progenitor cell (HSC, HSPC), a spleen colony forming (CFU-S) cell, a common myeloid progenitor (CMP) cell, a blastocyte colony-forming cell, a burst forming unit-erythroid/erythrocyte (BFU-E), a megakaryocyte-erythroid progenitor (MEP) cell, an erythroid colony-forming unit, or colony-forming unit erythrocyte (CFU-E), an induced pluripotent stem cell (iPSC), a mesenchymal stem cell (MSC), or a combination thereof. In some examples, an erythroid progenitor cell is a human erythroid progenitor cell. In some embodiments, an erythroid progenitor cell can be obtained from an O negative human donor. In some embodiments, an erythroid progenitor cell can be obtained from an O negative and a Kell negative human donor.

The term “subject” refers to any mammal. In some embodiments, the subject or “subject in need of treatment” can be a primate (e.g., a human, a simian (e.g., a monkey (e.g., marmoset or baboon), or an ape (e.g., a gorilla, chimpanzee, orangutan, or gibbon)), a rodent (e.g., a mouse, a guinea pig, a hamster, or a rat), a rabbit, a dog, a cat, a horse, a sheep, a cow, a pig, or a goat. In some embodiments, the subject or “subject suitable for treatment” may be a non-human mammal, especially mammals that are conventionally used as models for demonstrating therapeutic efficacy in humans (e.g., a mouse, a pig, a rat, or a non-human primate) may be employed. In some examples, a subject can be previously diagnosed or identified as being in need of treatment by a medical professional (e.g., a physician, a laboratory technician, a physician's assistant, a nurse, or a clinical laboratory technician).

As used herein, “treating” means a reduction in the number, severity, frequency, and/or duration of one or more symptoms of a medical disease or condition in a subject (e.g., any of the exemplary subjects described herein).

The term “shake flask” means a vessel (e.g., a sterile vessel) that can hold a volume of liquid culture medium that has at least one gas permeable surface (e.g., an end that has a gas-permeable element, e.g., a membrane, which may also act as a sterile barrier) and/or at least one vent cap, and at least a portion of its shape is approximately frustoconical. For example, a shake flask can be a cell culture flask, such as a T-flask, an Erlenmeyer flask, or any art-recognized modified version thereof.

The term “shake tube” means a vessel (e.g., a sterile vessel) that can retain liquid culture medium that has at least one gas permeable surface (e.g., an end that has a gas-permeable element, e.g., a membrane, which may also act as a sterile barrier) and/or at least one vent cap, and is capable of retaining liquid culture medium within the vessel upon agitation (e.g., rotary agitation), and at least a portion of its shape is approximately cylindrical. For example, a shake tube can be an Eppendorf™ tube (e.g., a 50-mL or 15-mL Eppendorf™ tube), or any art-recognized equivalent or modified version thereof In some examples, a shake tube can be a well (e.g., a round-bottomed well or a flat-bottomed well) in a multi-well plate.

The term “feed-batch culture” means a culturing method that includes the incremental or continuous addition of a second liquid culture medium to an initial cell culture without substantial or significant removal of the first liquid culture medium from the cell culture. In some instances, the second liquid culture medium includes the same components at substantially the same concentration as the first liquid culture medium. In other instances, the second liquid culture medium is a concentrated form of the first liquid culture medium and/or is added as a dry powder.

The term “perfusion culturing” means a culturing method that includes both the addition (incremental or continuous) of a liquid culture medium to a cell culture and removal of liquid culture medium from the cell culture. Removal and addition can be performed simultaneously or sequentially, or a combination of the two. Further, removal and addition can be performed continuously.

The volume of the liquid culture medium removed and the volume of the liquid culture medium added can in some instances be held approximately the same over each 24-hour period (or, alternatively, an incremental time period of about 1 hour to about 24 hours or an incremental time period of greater than 24 hours) over the entire or part of the culturing period. As is known in the art, the rate at which the volume of the liquid culture medium is removed (volume/unit of time) and the rate at which the volume of the liquid culture medium is added (volume/unit of time) can be varied. The rate at which the volume of the liquid culture medium is removed (volume/unit of time) and the rate at which the volume of the liquid culture medium is added (volume/unit of time) can be about the same or can be different.

Alternatively, the volume removed and added can change (e.g., gradually increase) over each 24-hour period (or alternatively, an incremental time period of between 1 hour and about 24 hours or an incremental time period of greater than 24hours) during the culturing period. For example the volume of the liquid culture medium removed and the volume of the liquid culture medium added within each 24-hour period (or alternatively, an incremental time period of between about 1 hour and above 24 hours or an incremental time period of greater than 24 hours) over the culturing period can be increased (e.g., gradually or through staggered increments) over the culturing period.

The volume of the liquid culture medium can be removed, e.g., by a mechanical system that can remove the volume of the liquid culture medium from the vessel (e.g., bioreactor), by allowing the cells to settle and removing the volume of the liquid culture medium using pipetting, or by a method that can at least partially include the use of centrifugal force). Alternatively, or in addition, the volume of the liquid culture medium can be removed by seeping or gravity flow of the volume of the liquid culture medium through a sterile membrane with a molecular weight cut-off that excludes the erythroid progenitor cells and enucleated erythroid cells.

The volume of the liquid culture medium can be added to the vessel (e.g., bioreactor) in an automated fashion, e.g., by a peristaltic pump or a perfusion pump.

The term “animal component-free liquid culture medium” means a liquid culture medium that does not contain any components (e.g., proteins or serum) derived from a mammal.

The term “serum-free liquid culture medium” means a liquid culture medium that does not contain the serum of a mammal.

The term “chemically-defined liquid culture medium” means a liquid culture medium in which all of the chemical components are known. For example, a chemically-defined liquid culture medium does not contain fetal serum, serum albumin, or serum albumin, as these preparations typically contain a complex mix of albumins and lipids.

The term “agitation” means the movement of a cell culture (e.g., a cell culture including any of the exemplary cells described herein) in a vessel. Agitation can be performed using any art known method, e.g., an instrument that moves a vessel containing a cell culture in a circular or ellipsoidal motion, such as a rotary shaker. Alternatively, or in addition, agitation can be performed by tilting the container or rolling a vessel containing a cell culture. In some embodiments, agitation of a cell culture can occur through the use of an impeller in a bioreactor containing the cell culture.

In some examples of any of the methods described herein, cell culturing does not include agitation of the cell culture for at least part of any of the methods described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the percentages of enucleation of perfusion cultured M-phase erythroid cells and M-phase erythroid cells cultured under the control condition. M-phase corresponds to step (d) in the methods described herein.

FIG. 2 shows viable cell densities of perfusion cultured M-phase erythroid cells and erythroid cells cultured under the control condition.

FIG. 3 shows viable cell density and viability of perfusion cultured D-phase erythroid cells. D-phase corresponds to step (b) in the methods described herein.

### DETAILED DESCRIPTION

Provided herein are methods of generating a population of enucleated erythroid cells, the methods include: (a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium comprised within a perfusion bioreactor to provide a third cell culture with an initial cell density of about 1×10⁵ to about 1×10⁷ cells/mL; (d) perfusion culturing the third cell culture of step (c) for about 5 days to 20 days, where after step (d) the third culture medium includes a population of enucleated erythroid cells. Also provided herein are methods of generating a population of enucleated erythroid cells, the methods include: (a) disposing a volume of a first cell culture of erythroid progenitor cells into a second culture medium comprised within a vessel to provide a second cell culture with an initial cell density of about 0.1×10⁵ cells/mL to about 1×10⁷ cells/mL; (b) batch or fed batch culturing the second cell culture for about 2 days to about 15 days; (c) disposing a volume of the second cell culture of step (b) into a third culture medium comprised within a perfusion bioreactor to provide a third cell culture with an initial cell density of about 1×10⁵ to about 1×10⁷ cells/mL; (d) perfusion culturing the third cell culture of step (c) for about 5 days to 20 days, where after step (d) the third culture medium comprises a population of enucleated erythroid cells. Also provided herein are methods of generating a population of enucleated erythroid cells, the methods include (a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL; (b) perfusion culturing the second cell culture for about 2 days to about 15 days; (c) disposing a volume of the second cell culture of step (b) into a third culture medium comprised within a vessel to provide a third cell culture with an initial cell density of about 1×10⁵ to about 1×10⁷ cells/mL; (d) batch or fed batch culturing the third cell culture of step (c) for about 5 days to 20 days, where after step (d) the third culture medium comprises a population of enucleated erythroid cells.

In some examples, the methods described herein provide for an increase (e.g., at least a 1% to about a 300% increase (e.g., about 1% to about a 280% increase, about a 1% to about 260% increase, about a 1% to about a 240% increase, about a 1% to about a 220% increase, about a 1% to about a 200% increase, about a 1% to about a 180% increase, about a 1% to about a 160% increase, about a 1% increase to about a 140% increase, about a 1% to about a 120% increase, about a 1% increase to about a 100% increase, about a 1% increase to about a 80% increase, about a 1% increase to about a 60% increase, about a 1% increase to about a 40% increase, about a 1% increase to about a 20% increase, about a 1% increase to about a 10% increase, about a 1% increase to about a 5% increase, about a 5% increase to about a 300% increase, about 5% to about a 280% increase, about a 5% to about 260% increase, about a 5% to about a 240% increase, about a 5% to about a 220% increase, about a 5% to about a 200% increase, about a 5% to about a 180% increase, about a 5% to about a 160% increase, about a 5% increase to about a 140% increase, about a 5% to about a 120% increase, about a 5% increase to about a 100% increase, about a 5% increase to about a 80% increase, about a 5% increase to about a 60% increase, about a 5% increase to about a 40% increase, about a 5% increase to about a 20% increase, about a 5% increase to about a 10% increase, about a 10% increase to about a 300% increase, about 10% to about a 280% increase, about a 10% to about 260% increase, about a 10% to about a 240% increase, about a 10% to about a 220% increase, about a 10% to about a 200% increase, about a 10% to about a 180% increase, about a 10% to about a 160% increase, about a 10% increase to about a 140% increase, about a 10% to about a 120% increase, about a 10% increase to about a 100% increase, about a 10% increase to about a 80% increase, about a 10% increase to about a 60% increase, about a 10% increase to about a 40% increase, about a 10% increase to about a 20% increase, about a 20% increase to about a 300% increase, about 20% to about a 280% increase, about a 20% to about 260% increase, about a 20% to about a 240% increase, about a 20% to about a 220% increase, about a 20% to about a 200% increase, about a 20% to about a 180% increase, about a 20% to about a 160% increase, about a 20% increase to about a 140% increase, about a 20% to about a 120% increase, about a 20% increase to about a 100% increase, about a 20% increase to about a 80% increase, about a 20% increase to about a 60% increase, about a 20% increase to about a 40% increase, about a 40% increase to about a 300% increase, about 40% to about a 280% increase, about a 40% to about 260% increase, about a 40% to about a 240% increase, about a 40% to about a 220% increase, about a 40% to about a 200% increase, about a 40% to about a 180% increase, about a 40% to about a 160% increase, about a 40% increase to about a 140% increase, about a 40% to about a 120% increase, about a 40% increase to about a 100% increase, about a 40% increase to about a 80% increase, about a 40% increase to about a 60% increase, about a 60% increase to about a 300% increase, about 60% to about a 280% increase, about a 60% to about 260% increase, about a 60% to about a 240% increase, about a 60% to about a 220% increase, about a 60% to about a 200% increase, about a 60% to about a 180% increase, about a 60% to about a 160% increase, about a 60% increase to about a 140% increase, about a 60% to about a 120% increase, about a 60% increase to about a 100% increase, about a 60% increase to about a 80% increase, about a 80% increase to about a 300% increase, about 80% to about a 280% increase, about a 80% to about 260% increase, about a 80% to about a 240% increase, about a 80% to about a 220% increase, about a 80% to about a 200% increase, about a 80% to about a 180% increase, about a 80% to about a 160% increase, about a 80% increase to about a 140% increase, about a 80% to about a 120% increase, about a 80% increase to about a 100% increase, about a 100% increase to about a 300% increase, about 100% to about a 280% increase, about a 100% to about 260% increase, about a 100% to about a 240% increase, about a 100% to about a 220% increase, about a 100% to about a 200% increase, about a 100% to about a 180% increase, about a 100% to about a 160% increase, about a 100% increase to about a 140% increase, about a 100% to about a 120% increase, about a 120% increase to about a 300% increase, about 120% to about a 280% increase, about a 120% to about 260% increase, about a 120% to about a 240% increase, about a 120% to about a 220% increase, about a 120% to about a 200% increase, about a 120% to about a 180% increase, about a 120% to about a 160% increase, about a 120% increase to about a 140% increase, about a 140% increase to about a 300% increase, about 140% to about a 280% increase, about a 140% to about 260% increase, about a 140% to about a 240% increase, about a 140% to about a 220% increase, about a 140% to about a 200% increase, about a 140% to about a 180% increase, about a 140% to about a 160% increase, about a 160% increase to about a 300% increase, about 160% to about a 280% increase, about a 160% to about 260% increase, about a 160% to about a 240% increase, about a 160% to about a 220% increase, about a 160% to about a 200% increase, about a 160% to about a 180% increase, about a 180% increase to about a 300% increase, about 180% to about a 280% increase, about a 180% to about 260% increase, about a 180% to about a 240% increase, about a 180% to about a 220% increase, about a 180% to about a 200% increase, about a 200% increase to about a 300% increase, about 200% to about a 280% increase, about a 200% to about 260% increase, about a 200% to about a 240% increase, about a 200% to about a 220% increase, about a 220% increase to about a 300% increase, about 220% to about a 280% increase, about a 220% to about 260% increase, about a 220% to about a 240% increase, about a 240% increase to about a 300% increase, about 240% to about a 280% increase, about a 240% to about 260% increase, about a 260% increase to about a 300% increase, about 260% to about a 280% increase, or about a 280% increase to about a 300% increase)) in the rate of enucleation or the percentage of enucleated cells at the end of any of the methods described herein, e.g., as compared to a similar method that does not include the use of perfusion culturing during differentiation or maturation.

In some examples, the methods described herein provide for an about 1% increase to about a 300% increase (or any of the subranges of this range described herein) in the concentration, number, or yield of enucleated erythroid cells (e.g., any of the enucleated erythroid cells described herein) at the end of any of the methods described herein, e.g., as compared to a similar method that does not include the use of perfusion culturing during differentiation or maturation.

In some examples, the method described herein provide for an about 1% increase to about a 300% increase (or any of the subranges of this range described herein) in the average number of cell divisions per progenitor erythroid cell during any of the methods described herein, e.g., as compared to a similar method that does not include the use of perfusion culturing during differentiation or maturation.

Non-limiting aspects of these methods are described below. As can be appreciated by those in the field, the exemplary aspects listed below can be used in any combination, and can be combined with other aspects known in the field.

### Shake Flasks

Some aspects of the methods described herein include the use of a shake flask. In some examples, a shake flask can have a volume of about 2 mL to about 5 L, about 2 mL to about 4.5 L, about 2 mL to about 4 L, about 2 mL to about 3.5 L, about 2 mL to about 3 L, about 2 mL to about 2.5 L, about 2 mL to about 2 L, about 2 mL to about 1.5 L, about 2 mL to about 1.0 L, about 2 mL to about 950 mL, about 2 mL to about 900 mL, about 2 mL to about 850 mL, about 2 mL to about 800 mL, about 2 mL to about 750 mL, about 2 mL to about 700 mL, about 2 mL to about 650 mL, about 2 mL to about 600 mL, about 2 mL to about 550 mL, about 2 mL to about 500 mL, about 2 mL to about 450 mL, about 2 mL to about 400 mL, about 2 mL to about 350 mL, about 2 mL to about 300 mL, about 2 mL to about 250 mL, about 2 mL to about 200 mL, about 2 mL to about 180 mL, about 2 mL to about 160 mL, about 2 mL to about 140 mL, about 2 mL to about 120 mL, about 2 mL to about 100 mL, about 2 mL to about 80 mL, about 2 mL to about 60 mL, about 2 mL to about 40 mL, about 2 mL to about 20 mL, about 2 mL to about 10 mL, about 2 mL to about 5 mL, about 5 mL to about 5 L, about 5 mL to about 4.5 L, about 5 mL to about 4 L, about 5 mL to about 3.5 L, about 5mL to about 3L, about 5 mL to about 2.5 L, about 5 mL to about 2 L, about 5 mL to about 1.5 L, about 5 mL to about 1.0 L, about 5 mL to about 950 mL, about 5 mL to about 900 mL, about 5 mL to about 850 mL, about 5 mL to about 800 mL, about 5 mL to about 750 mL, about 5 mL to about 700 mL, about 5 mL to about 650 mL, about 5 mL to about 600 mL, about 5 mL to about 550 mL, about 5 mL to about 500 mL, about 5 mL to about 450 mL, about 5 mL to about 400 mL, about 5 mL to about 350 mL, about 5 mL to about 300 mL, about 5 mL to about 250 mL, about 5 mL to about 200 mL, about 5 mL to about 180 mL, about 5 mL to about 160 mL, about 5 mL to about 140 mL, about 5 mL to about 120 mL, about 5 mL to about 100 mL, about 5 mL to about 80 mL, about 5 mL to about 60 mL, about 5 mL to about 40 mL, about 5 mL to about 20 mL, about 5 mL to about 10 mL, about 10 mL to about 5 L, about 10 mL to about 4.5 L, about 10 mL to about 4 L, about 10 mL to about 3.5 L, about 10 mL to about 3 L, about 10 mL to about 2.5 L, about 10 mL to about 2 L, about 10 mL to about 1.5 L, about 10 mL to about 1.0 L, about 10 mL to about 950 mL, about 10 mL to about 900 mL, about 10 mL to about 850 mL, about 10 mL to about 800 mL, about 10 mL to about 750 mL, about 10 mL to about 700 mL, about 10 mL to about 650 mL, about 10 mL to about 600 mL, about 10 mL to about 550 mL, about 10 mL to about 500 mL, about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 180 mL, about 10 mL to about 160 mL, about 10 mL to about 140 mL, about 10 mL to about 120 mL, about 10 mL to about 100 mL, about 10 mL to about 80 mL, about 10 mL to about 60 mL, about 10 mL to about 40 mL, about 10 mL to about 20 mL, about 20 mL to about 5 L, about 20 mL to about 4.5 L, about 20 mL to about 4 L, about 20 mL to about 3.5 L, about 20 mL to about 3 L, about 20 mL to about 2.5 L, about 20 mL to about 2 L, about 20 mL to about 1.5 L, about 20 mL to about 1.0 L, about 20 mL to about 950 mL, about 20 mL to about 900 mL, about 20 mL to about 850 mL, about 20 mL to about 800 mL, about 20 mL to about 750 mL, about 20 mL to about 700 mL, about 20 mL to about 650 mL, about 20 mL to about 600 mL, about 20 mL to about 550 mL, about 20 mL to about 500 mL, about 20 mL to about 450 mL, about 20 mL to about 400 mL, about 20 mL to about 350 mL, about 20 mL to about 300 mL, about 20 mL to about 250 mL, about 20 mL to about 200 mL, about 20 mL to about 180 mL, about 20 mL to about 160 mL, about 20 mL to about 140 mL, about 20 mL to about 120 mL, about 20 mL to about 100 mL, about 20 mL to about 80 mL, about 20 mL to about 60 mL, about 20 mL to about 40 mL, about 40 mL to about 5 L, about 40 mL to about 4.5 L, about 40 mL to about 4 L, about 40 mL to about 3.5 L, about 40 mL to about 3 L, about 40 mL to about 2.5 L, about 40 mL to about 2 L, about 40 mL to about 1.5 L, about 40 mL to about 1.0 L, about 40 mL to about 950 mL, about 40 mL to about 900 mL, about 40 mL to about 850 mL, about 40 mL to about 800 mL, about 40 mL to about 750 mL, about 40 mL to about 700 mL, about 40 mL to about 650 mL, about 40 mL to about 600 mL, about 40 mL to about 550 mL, about 40 mL to about 500 mL, about 40 mL to about 450 mL, about 40 mL to about 400 mL, about 40 mL to about 350 mL, about 40 mL to about 300 mL, about 40 mL to about 250 mL, about 40 mL to about 200 mL, about 40 mL to about 180 mL, about 40 mL to about 160 mL, about 40 mL to about 140 mL, about 40 mL to about 120 mL, about 40 mL to about 100 mL, about 40 mL to about 80 mL, about 40 mL to about 60 mL, about 60 mL to about 5 L, about 60 mL to about 4.5 L, about 60 mL to about 4 L, about 60 mL to about 3.5 L, about 60 mL to about 3 L, about 60 mL to about 2.5 L, about 60 mL to about 2 L, about 60 mL to about 1.5 L, about 60 mL to about 1.0 L, about 60 mL to about 950 mL, about 60 mL to about 900 mL, about 60 mL to about 850 mL, about 60 mL to about 800 mL, about 60 mL to about 750 mL, about 60 mL to about 700 mL, about 60 mL to about 650 mL, about 60 mL to about 600 mL, about 60 mL to about 550 mL, about 60 mL to about 500 mL, about 60 mL to about 450 mL, about 60 mL to about 400 mL, about 60 mL to about 350 mL, about 60 mL to about 300 mL, about 60 mL to about 250 mL, about 60 mL to about 200 mL, about 60 mL to about 180 mL, about 60 mL to about 160 mL, about 60 mL to about 140 mL, about 60 mL to about 120 mL, about 60 mL to about 100 mL, about 60 mL to about 80 mL, about 80 mL to about 5 L, about 80 mL to about 4.5 L, about 80 mL to about 4 L, about 80 mL to about 3.5 L, about 80 mL to about 3 L, about 80 mL to about 2.5 L, about 80 mL to about 2 L, about 80 mL to about 1.5 L, about 80 mL to about 1.0 L, about 80 mL to about 950 mL, about 80 mL to about 900 mL, about 80 mL to about 850 mL, about 80 mL to about 800 mL, about 80 mL to about 750 mL, about 80 mL to about 700 mL, about 80 mL to about 650 mL, about 80 mL to about 600 mL, about 80 mL to about 550 mL, about 80 mL to about 500 mL, about 80 mL to about 450 mL, about 80 mL to about 400 mL, about 80 mL to about 350 mL, about 80 mL to about 300 mL, about 80 mL to about 250 mL, about 80 mL to about 200 mL, about 80 mL to about 180 mL, about 80 mL to about 160 mL, about 80 mL to about 140 mL, about 80 mL to about 120 mL, about 80 mL to about 100 mL, about 100 mL to about 5 L, about 100 mL to about 4.5 L, about 100 mL to about 4 L, about 100 mL to about 3.5 L, about 100 mL to about 3 L, about 100 mL to about 2.5 L, about 100 mL to about 2 L, about 100 mL to about 1.5 L, about 100 mL to about 1.0 L, about 100 mL to about 950 mL, about 100 mL to about 900 mL, about 100 mL to about 850 mL, about 100 mL to about 800 mL, about 100 mL to about 750 mL, about 100 mL to about 700 mL, about 100 mL to about 650 mL, about 100 mL to about 600 mL, about 100 mL to about 550 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 180 mL, about 100 mL to about 160 mL, about 100 mL to about 140 mL, about 100 mL to about 120 mL, about 120 mL to about 5 L, about 120 mL to about 4.5 L, about 120 mL to about 4 L, about 120 mL to about 3.5 L, about 120 mL to about 3 L, about 120 mL to about 2.5 L, about 120 mL to about 2 L, about 120 mL to about 1.5 L, about 120 mL to about 1.0 L, about 120 mL to about 950 mL, about 120 mL to about 900 mL, about 120 mL to about 850 mL, about 120 mL to about 800 mL, about 120 mL to about 750 mL, about 120 mL to about 700 mL, about 120 mL to about 650 mL, about 120 mL to about 600 mL, about 120 mL to about 550 mL, about 120 mL to about 500 mL, about 120 mL to about 450 mL, about 120 mL to about 400 mL, about 120 mL to about 350 mL, about 120 mL to about 300 mL, about 120 mL to about 250 mL, about 120 mL to about 200 mL, about 120 mL to about 180 mL, about 120 mL to about 160 mL, about 120 mL to about 140 mL, about 140 mL to about 5 L, about 140 mL to about 4.5 L, about 140 mL to about 4 L, about 140 mL to about 3.5 L, about 140 mL to about 3 L, about 140 mL to about 2.5 L, about 140 mL to about 2 L, about 140 mL to about 1.5 L, about 140 mL to about 1.0 L, about 140 mL to about 950 mL, about 140 mL to about 900 mL, about 140 mL to about 850 mL, about 140 mL to about 800 mL, about 140 mL to about 750 mL, about 140 mL to about 700 mL, about 140 mL to about 650 mL, about 140 mL to about 600 mL, about 140 mL to about 550 mL, about 140 mL to about 500 mL, about 140 mL to about 450 mL, about 140 mL to about 400 mL, about 140 mL to about 350 mL, about 140 mL to about 300 mL, about 140 mL to about 250 mL, about 140 mL to about 200 mL, about 140 mL to about 180 mL, about 140 mL to about 160 mL, about 160 mL to about 5 L, about 160 mL to about 4.5 L, about 160 mL to about 4 L, about 160 mL to about 3.5 L, about 160 mL to about 3 L, about 160 mL to about 2.5 L, about 160 mL to about 2 L, about 160 mL to about 1.5 L, about 160 mL to about 1.0 L, about 160 mL to about 950 mL, about 160 mL to about 900 mL, about 160 mL to about 850 mL, about 160 mL to about 800 mL, about 160 mL to about 750 mL, about 160 mL to about 700 mL, about 160 mL to about 650 mL, about 160 mL to about 600 mL, about 160 mL to about 550 mL, about 160 mL to about 500 mL, about 160 mL to about 450 mL, about 160 mL to about 400 mL, about 160 mL to about 350 mL, about 160 mL to about 300 mL, about 160 mL to about 250 mL, about 160 mL to about 200 mL, about 160 mL to about 180 mL, about 180 mL to about 5 L, about 180 mL to about 4.5 L, about 180 mL to about 4 L, about 180 mL to about 3.5 L, about 180 mL to about 3 L, about 180 mL to about 2.5 L, about 180 mL to about 2 L, about 180 mL to about 1.5 L, about 180 mL to about 1.0 L, about 180 mL to about 950 mL, about 180 mL to about 900 mL, about 180 mL to about 850 mL, about 180 mL to about 800 mL, about 180 mL to about 750 mL, about 180 mL to about 700 mL, about 180 mL to about 650 mL, about 180 mL to about 600 mL, about 180 mL to about 550 mL, about 180 mL to about 500 mL, about 180 mL to about 450 mL, about 180 mL to about 400 mL, about 180 mL to about 350 mL, about 180 mL to about 300 mL, about 180 mL to about 250 mL, about 180 mL to about 200 mL, about 200 mL to about 5 L, about 200 mL to about 4.5 L, about 200 mL to about 4 L, about 200 mL to about 3.5 L, about 200 mL to about 3 L, about 200 mL to about 2.5 L, about 200 mL to about 2 L, about 200 mL to about 1.5 L, about 200 mL to about 1.0 L, about 200 mL to about 950 mL, about 200 mL to about 900 mL, about 200 mL to about 850 mL, about 200 mL to about 800 mL, about 200 mL to about 750 mL, about 200 mL to about 700 mL, about 200 mL to about 650 mL, about 200 mL to about 600 mL, about 200 mL to about 550 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 5 L, about 250 mL to about 4.5 L, about 250 mL to about 4 L, about 250 mL to about 3.5 L, about 250 mL to about 3 L, about 250 mL to about 2.5 L, about 250 mL to about 2 L, about 250 mL to about 1.5 L, about 250 mL to about 1.0 L, about 250 mL to about 950 mL, about 250 mL to about 900 mL, about 250 mL to about 850 mL, about 250 mL to about 800 mL, about 250 mL to about 750 mL, about 250 mL to about 700 mL, about 250 mL to about 650 mL, about 250 mL to about 600 mL, about 250 mL to about 550 mL, about 250 mL to about 500 mL, about 250 mL to about 450 mL, about 250 mL to about 400 mL, about 250 mL to about 350 mL, about 250 mL to about 300 mL, about 300 mL to about 5 L, about 300 mL to about 4.5 L, about 300 mL to about 4 L, about 300 mL to about 3.5 L, about 300 mL to about 3 L, about 300 mL to about 2.5 L, about 300 mL to about 2 L, about 300 mL to about 1.5 L, about 300 mL to about 1.0 L, about 300 mL to about 950 mL, about 300 mL to about 900 mL, about 300 mL to about 850 mL, about 300 mL to about 800 mL, about 300 mL to about 750 mL, about 300 mL to about 700 mL, about 300 mL to about 650 mL, about 300 mL to about 600 mL, about 300 mL to about 550 mL, about 300 mL to about 500 mL, about 300 mL to about 450 mL, about 300 mL to about 400 mL, about 300 mL to about 350 mL, about 350 mL to about 3 L, about 350 mL to about 2.5 L, about 350 mL to about 2 L, about 350 mL to about 1.5 L, about 350 mL to about 1.0 L, about 350 mL to about 950 mL, about 350 mL to about 900 mL, about 350 mL to about 850 mL, about 350 mL to about 800 mL, about 350 mL to about 750 mL, about 350 mL to about 700 mL, about 350 mL to about 650 mL, about 350 mL to about 600 mL, about 350 mL to about 550 mL, about 350 mL to about 500 mL, about 350 mL to about 450 mL, about 350 mL to about 400 mL, about 400 mL to about 5 L, about 400 mL to about 4.5 L, about 400 mL to about 4 L, about 400 mL to about 3.5 L, about 400 mL to about 3 L, about 400 mL to about 2.5 L, about 400 mL to about 2 L, about 400 mL to about 1.5 L, about 400 mL to about 1.0 L, about 400 mL to about 950 mL, about 400 mL to about 900 mL, about 400 mL to about 850 mL, about 400 mL to about 800 mL, about 400 mL to about 750 mL, about 400 mL to about 700 mL, about 400 mL to about 650 mL, about 400 mL to about 600 mL, about 400 mL to about 550 mL, about 400 mL to about 500 mL, about 400 mL to about 450 mL, about 450 mL to about 3 L, about 450 mL to about 2.5 L, about 450 mL to about 2 L, about 450 mL to about 1.5 L, about 450 mL to about 1.0 L, about 450 mL to about 950 mL, about 450 mL to about 900 mL, about 450 mL to about 850 mL, about 450 mL to about 800 mL, about 450 mL to about 750 mL, about 450 mL to about 700 mL, about 450 mL to about 650 mL, about 450 mL to about 600 mL, about 450 mL to about 550 mL, about 450 mL to about 500 mL, about 500 mL to about 5 L, about 500 mL to about 4.5 L, about 500 mL to about 4 L, about 500 mL to about 3.5 L, about 500 mL to about 3 L, about 500 mL to about 2.5 L, about 500 mL to about 2 L, about 500 mL to about 1.5 L, about 500 mL to about 1.0 L, about 500 mL to about 950 mL, about 500 mL to about 900 mL, about 500 mL to about 850 mL, about 500 mL to about 800 mL, about 500 mL to about 750 mL, about 500 mL to about 700 mL, about 500 mL to about 650 mL, about 500 mL to about 600 mL, about 500 mL to about 550 mL, about 550 mL to about 5 L, about 550 mL to about 4.5 L, about 550 mL to about 4 L, about 550 mL to about 3.5 L, about 550 mL to about 3 L, about 550 mL to about 2.5 L, about 550 mL to about 2 L, about 550 mL to about 1.5 L, about 550 mL to about 1.0 L, about 550 mL to about 950 mL, about 550 mL to about 900 mL, about 550 mL to about 850 mL, about 550 mL to about 800 mL, about 550 mL to about 750 mL, about 550 mL to about 700 mL, about 550 mL to about 650 mL, about 550 mL to about 600 mL, about 600 mL to about 5 L, about 600 mL to about 4.5 L, about 600 mL to about 4 L, about 600 mL to about 3.5 L, about 600 mL to about 3 L, about 600 mL to about 2.5 L, about 600 mL to about 2 L, about 600 mL to about 1.5 L, about 600 mL to about 1.0 L, about 600 mL to about 950 mL, about 600 mL to about 900 mL, about 600 mL to about 850 mL, about 600 mL to about 800 mL, about 600 mL to about 750 mL, about 600 mL to about 700 mL, about 600 mL to about 650 mL, about 650 mL to about 5 L, about 650 mL to about 4.5 L, about 650 mL to about 4 L, about 650 mL to about 3.5 L, about 650 mL to about 3 L, about 650 mL to about 2.5 L, about 650 mL to about 2 L, about 650 mL to about 1.5 L, about 650 mL to about 1.0 L, about 650 mL to about 950 mL, about 650 mL to about 900 mL, about 650 mL to about 850 mL, about 650 mL to about 800 mL, about 650 mL to about 750 mL, about 650 mL to about 700 mL, about 700 mL to about 5 L, about 700 mL to about 4.5 L, about 700 mL to about 4 L, about 700 mL to about 3.5 L, about 700 mL to about 3 L, about 700 mL to about 2.5 L, about 700 mL to about 2 L, about 700 mL to about 1.5 L, about 700 mL to about 1.0 L, about 700 mL to about 950 mL, about 700 mL to about 900 mL, about 700 mL to about 850 mL, about 700 mL to about 800 mL, about 700 mL to about 750 mL, about 750 mL to about 5 L, about 750 mL to about 4.5 L, about 750 mL to about 4 L, about 750 mL to about 3.5 L, about 750 mL to about 3 L, about 750 mL to about 2.5 L, about 750 mL to about 2 L, about 750 mL to about 1.5 L, about 750 mL to about 1.0 L, about 750 mL to about 950 mL, about 750 mL to about 900 mL, about 750 mL to about 850 mL, about 750 mL to about 800 mL, about 800 mL to about 5 L, about 800 mL to about 4.5 L, about 800 mL to about 4 L, about 800 mL to about 3.5 L, about 800 mL to about 3 L, about 800 mL to about 2.5 L, about 800 mL to about 2 L, about 800 mL to about 1.5 L, about 800 mL to about 1.0 L, about 800 mL to about 950 mL, about 800 mL to about 900 mL, about 800 mL to about 850 mL, about 850 mL to about 5 L, about 850 mL to about 4.5 L, about 850 mL to about 4 L, about 850 mL to about 3.5 L, about 850 mL to about 3 L, about 850 mL to about 2.5 L, about 850 mL to about 2 L, about 850 mL to about 1.5 L, about 850 mL to about 1.0 L, about 850 mL to about 950 mL, about 850 mL to about 900 mL, about 900 mL to about 5 L, about 900 mL to about 4.5 L, about 900 mL to about 4 L, about 900 mL to about 3.5 L, about 900 mL to about 3 L, about 900 mL to about 2.5 L, about 900 mL to about 2 L, about 900 mL to about 1.5 L, about 900 mL to about 1.0 L, about 900 mL to about 950 mL, about 950 mL to about 5 L, about 950 mL to about 4.5 L, about 950 mL to about 4 L, about 950 mL to about 3.5 L, about 950 mL to about 3 L, about 950 mL to about 2.5 L, about 950 mL to about 2 L, about 950 mL to about 1.5 L, about 950 mL to about 1.0 L, about 1.0 L to about 5 L, about 1.0 L to about 4.5 L, about 1.0 L to about 4 L, about 1.0 L to about 3.5 L, about 1.0 L to about 3 L, 1.0 L to about 2.5 L, 1.0 L to about 2 L, 1.0 L to about 1.5 L, about 1.5 L to about 5 L, about 1.5 L to about 4.5 L, about 1.5 L to about 4 L, about 1.5 L to about 3.5 L, about 1.5 L to about 3 L, about 1.5 L to about 2.5 L, about 1.5 L to about 2 L, about 2 L to about 5 L, about 2 L to about 4.5 L, about 2 L to about 4 L, about 2 L to about 3.5 L, about 2 L to about 3 L, about 2 L to about 2.5 L, about 2.5 L to about 5 L, about 2.5 L to about 4.5 L, about 2.5 L to about 4 L, about 2.5 L to about 3.5 L, about 2.5 L to about 3 L, about 3 L to about 5 L, about 3 L to about 4.5 L, about 3 L to about 4 L, about 3 L to about 3.5 L, about 3.5 L to about 5 L, about 3.5 L to about 4.5 L, about 3.5 L to about 4 L, about 4 L to about 5 L, about 4 L to about 4.5 L, or about 4.5 L to about 5 L.

In some examples, a shake flask can be incubated with an agitation of about 0.1×g to about 50×g (e.g., about 0.1×g to about 40×g, about 0.1×g to about 30×g, about 0.1×g to about 20×g, about 0.1×g to about 15×g, about 0.1×g to about 10×g, about 0.1×g to about 9.5×g, about 0.1×g to about 9.0×g, about 0.1×g to about 8.5×g, about 0.1×g to about 8.0×g, about 0.1×g to about 7.5×g, about 0.1×g to about 7.0×g, about 0.1×g to about 6.5×g, about 0.1×g to about 6.0×g, about 0.1×g to about 5.5×g, about 0.1×g to about 5.0×g, about 0.1×g to about 4.5×g, about 0.1×g to about 4.1×g, about 0.1×g to about 4.0×g, about 0.1×g to about 3.5×g, about 0.1×g to about 3.0×g, about 0.1×g to about 2.5×g, about 0.1×g to about 2.0×g, about 0.1×g to about 1.78×g, about 0.1×g to about 1.6×g, about 0.1×g to about 1.4×g, about 0.1×g to about 1.2×g, about 0.1×g to about 1.0×g, about 0.1×g to about 0.8×g, about 0.1×g to about 0.6×g, about 0.1×g to about 0.4×g, about 0.1×g to about 0.23×g, about 0.23×g to about 50×g, about 0.23×g to about 40×g, about 0.23×g to about 30×g, about 0.23×g to about 20×g, about 0.23×g to about 15×g, about 0.23×g to about 10×g, about 0.23×g to about 9.5×g, about 0.23×g to about 9.0×g, about 0.23×g to about 8.5×g, about 0.23×g to about 8.0×g, about 0.23×g to about 7.5×g, about 0.23×g to about 7.0×g, about 0.23×g to about 6.5×g, about 0.23×g to about 6.0×g, about 0.23×g to about 5.5×g, about 0.23×g to about 5.0×g, about 0.23×g to about 4.5×g, about 0.23×g to about 4.1×g, about 0.23×g to about 4.0×g, about 0.23×g to about 3.5×g, about 0.23×g to about 3.0×g, about 0.23×g to about 2.5×g, about 0.23×g to about 2.0×g, about 0.23×g to about 1.78×g, about 0.23×g to about 1.6×g, about 0.23×g to about 1.4×g, about 0.23×g to about 1.2×g, about 0.23×g to about 1.0×g, about 0.23×g to about 0.8×g, about 0.23×g to about 0.6×g, about 0.23×g to about 0.4×g, about 0.4×g to about 50×g, about 0.4×g to about 40×g, about 0.4×g to about 30×g, about 0.4×g to about 20×g, about 0.4×g to about 15×g, about 0.4×g to about 10×g, about 0.4×g to about 9.5×g, about 0.4×g to about 9.0×g, about 0.4×g to about 8.5×g, about 0.4×g to about 8.0×g, about 0.4×g to about 7.5×g, about 0.4×g to about 7.0×g, about 0.4×g to about 6.5×g, about 0.4×g to about 6.0×g, about 0.4×g to about 5.5×g, about 0.4×g to about 5.0×g, about 0.4×g to about 4.5×g, about 0.4×g to about 4.1×g, about 0.4×g to about 4.0×g, about 0.4×g to about 3.5×g, about 0.4×g to about 3.0×g, about 0.4×g to about 2.5×g, about 0.4×g to about 2.0×g, about 0.4×g to about 1.78×g, about 0.4×g to about 1.6×g, about 0.4×g to about 1.4×g, about 0.4×g to about 1.2×g, about 0.4×g to about 1.0×g, about 0.4×g to about 0.8×g, about 0.4×g to about 0.6×g, about 0.6×g to about 50×g, about 0.6×g to about 40×g, about 0.6×g to about 30×g, about 0.6×g to about 20×g, about 0.6×g to about 15×g, about 0.6×g to about 10×g, about 0.6×g to about 9.5×g, about 0.6×g to about 9.0×g, about 0.6×g to about 8.5×g, about 0.6×g to about 8.0×g, about 0.6×g to about 7.5×g, about 0.6×g to about 7.0×g, about 0.6×g to about 6.5×g, about 0.6×g to about 6.0×g, about 0.6×g to about 5.5×g, about 0.6×g to about 5.0×g, about 0.6×g to about 4.5×g, about 0.6×g to about 4.1×g, about 0.6×g to about 4.0×g, about 0.6×g to about 3.5×g, about 0.6×g to about 3.0×g, about 0.6×g to about 2.5×g, about 0.6×g to about 2.0×g, about 0.6×g to about 1.78×g, about 0.6×g to about 1.6×g, about 0.6×g to about 1.4×g, about 0.6×g to about 1.2×g, about 0.6×g to about 1.0×g, about 0.6×g to about 0.8×g, about 0.8×g to about 50×g, about 0.8×g to about 40×g, about 0.8×g to about 30×g, about 0.8×g to about 20×g, about 0.8×g to about 15×g, about 0.8×g to about 10×g, about 0.8×g to about 9.5×g, about 0.8×g to about 9.0×g, about 0.8×g to about 8.5×g, about 0.8×g to about 8.0×g, about 0.8×g to about 7.5×g, about 0.8×g to about 7.0×g, about 0.8×g to about 6.5×g, about 0.8×g to about 6.0×g, about 0.8×g to about 5.5×g, about 0.8×g to about 5.0×g, about 0.8×g to about 4.5×g, about 0.8×g to about 4.1×g, about 0.8×g to about 4.0×g, about 0.8×g to about 3.5×g, about 0.8×g to about 3.0×g, about 0.8×g to about 2.5×g, about 0.8×g to about 2.0×g, about 0.8×g to about 1.78×g, about 0.8×g to about 1.6×g, about 0.8×g to about 1.4×g, about 0.8×g to about 1.2×g, about 0.8×g to about 1.0×g, about 1.0×g to about 50×g, about 1.0×g to about 40×g, about 1.0×g to about 30×g, about 1.0×g to about 20×g, about 1.0×g to about 15×g, about 1.0×g to about 10×g, about 1.0×g to about 9.5×g, about 1.0×g to about 9.0×g, about 1.0×g to about 8.5×g, about 1.0×g to about 8.0×g, about 1.0×g to about 7.5×g, about 1.0×g to about 7.0×g, about 1.0×g to about 6.5×g, about 1.0×g to about 6.0×g, about 1.0×g to about 5.5×g, about 1.0×g to about 5.0×g, about 1.0×g to about 4.5×g, about 1.0×g to about 4.1×g, about 1.0×g to about 4.0×g, about 1.0×g to about 3.5×g, about 1.0×g to about 3.0×g, about 1.0×g to about 2.5×g, about 1.0×g to about 2.0×g, about 1.0×g to about 1.78×g, about 1.0×g to about 1.6×g, about 1.0×g to about 1.4×g, about 1.0×g to about 1.2×g, about 1.2×g to about 50×g, about 1.2×g to about 40×g, about 1.2×g to about 30×g, about 1.2×g to about 20×g, about 1.2×g to about 15×g, about 1.2×g to about 10×g, about 1.2×g to about 9.5×g, about 1.2×g to about 9.0×g, about 1.2×g to about 8.5×g, about 1.2×g to about 8.0×g, about 1.2×g to about 7.5×g, about 1.2×g to about 7.0×g, about 1.2×g to about 6.5×g, about 1.2×g to about 6.0×g, about 1.2×g to about 5.5×g, about 1.2×g to about 5.0×g, about 1.2×g to about 4.5×g, about 1.2×g to about 4.1×g, about 1.2×g to about 4.0×g, about 1.2×g to about 3.5×g, about 1.2×g to about 3.0×g, about 1.2×g to about 2.5×g, about 1.2×g to about 2.0×g, about 1.2×g to about 1.78×g, about 1.2×g to about 1.6×g, about 1.2×g to about 1.4×g, about 1.4×g to about 50×g, about 1.4×g to about 40×g, about 1.4×g to about 30×g, about 1.4×g to about 20×g, about 1.4×g to about 15×g, about 1.4×g to about 10×g, about 1.4×g to about 9.5×g, about 1.4×g to about 9.0×g, about 1.4×g to about 8.5×g, about 1.4×g to about 8.0×g, about 1.4×g to about 7.5×g, about 1.4×g to about 7.0×g, about 1.4×g to about 6.5×g, about 1.4×g to about 6.0×g, about 1.4×g to about 5.5×g, about 1.4×g to about 5.0×g, about 1.4×g to about 4.5×g, about 1.4×g to about 4.1×g, about 1.4×g to about 4.0×g, about 1.4×g to about 3.5×g, about 1.4×g to about 3.0×g, about 1.4×g to about 2.5×g, about 1.4×g to about 2.0×g, about 1.4×g to about 1.78×g, about 1.4×g to about 1.6×g, about 1.6×g to about 50×g, about 1.6×g to about 40×g, about 1.6×g to about 30×g, about 1.6×g to about 20×g, about 1.6×g to about 15×g, about 1.6×g to about 10×g, about 1.6×g to about 9.5×g, about 1.6×g to about 9.0×g, about 1.6×g to about 8.5×g, about 1.6×g to about 8.0×g, about 1.6×g to about 7.5×g, about 1.6×g to about 7.0×g, about 1.6×g to about 6.5×g, about 1.6×g to about 6.0×g, about 1.6×g to about 5.5×g, about 1.6×g to about 5.0×g, about 1.6×g to about 4.5×g, about 1.6×g to about 4.1×g, about 1.6×g to about 4.0×g, about 1.6×g to about 3.5×g, about 1.6×g to about 3.0×g, about 1.6×g to about 2.5×g, about 1.6×g to about 2.0×g, about 1.6×g to about 1.78×g, about 1.78×g to about 50×g, about 1.78×g to about 40×g, about 1.78×g to about 30×g, about 1.78×g to about 20×g, about 1.78×g to about 15×g, about 1.78×g to about 10×g, about 1.78×g to about 9.5×g, about 1.78×g to about 9.0×g, about 1.78×g to about 8.5×g, about 1.78×g to about 8.0×g, about 1.78×g to about 7.5×g, about 1.78×g to about 7.0×g, about 1.78×g to about 6.5×g, about 1.78×g to about 6.0×g, about 1.78×g to about 5.5×g, about 1.78×g to about 5.0×g, about 1.78×g to about 4.5×g, about 1.78×g to about 4.1×g, about 1.78×g to about 4.0×g, about 1.78×g to about 3.5×g, about 1.78×g to about 3.0×g, about 1.78×g to about 2.5×g, about 1.78×g to about 2.0×g, about 2.0×g to about 50×g, about 2.0×g to about 40×g, about 2.0×g to about 30×g, about 2.0×g to about 20×g, about 2.0×g to about 15×g, about 2.0×g to about 10×g, about 2.0×g to about 9.5×g, about 2.0×g to about 9.0×g, about 2.0×g to about 8.5×g, about 2.0×g to about 8.0×g, about 2.0×g to about 7.5×g, about 2.0×g to about 7.0×g, about 2.0×g to about 6.5×g, about 2.0×g to about 6.0×g, about 2.0×g to about 5.5×g, about 2.0×g to about 5.0×g, about 2.0×g to about 4.5×g, about 2.0×g to about 4.1×g, about 2.0×g to about 4.0×g, about 2.0×g to about 3.5×g, about 2.0×g to about 3.0×g, about 2.0×g to about 2.5×g, about 2.5×g to about 50×g, about 2.5×g to about 40×g, about 2.5×g to about 30×g, about 2.5×g to about 20×g, about 2.5×g to about 15×g, about 2.5×g to about 10×g, about 2.5×g to about 9.5×g, about 2.5×g to about 9.0×g, about 2.5×g to about 8.5×g, about 2.5×g to about 8.0×g, about 2.5×g to about 7.5×g, about 2.5×g to about 7.0×g, about 2.5×g to about 6.5×g, about 2.5×g to about 6.0×g, about 2.5×g to about 5.5×g, about 2.5×g to about 5.0×g, about 2.5×g to about 4.5×g, about 2.5×g to about 4.1×g, about 2.5×g to about 4.0×g, about 2.5×g to about 3.5×g, about 2.5×g to about 3.0×g, about 3.0×g to about 50×g, about 3.0×g to about 40×g, about 3.0×g to about 30×g, about 3.0×g to about 20×g, about 3.0×g to about 15×g, about 3.0×g to about 10×g, about 3.0×g to about 9.5×g, about 3.0×g to about 9.0×g, about 3.0×g to about 8.5×g, about 3.0×g to about 8.0×g, about 3.0×g to about 7.5×g, about 3.0×g to about 7.0×g, about 3.0×g to about 6.5×g, about 3.0×g to about 6.0×g, about 3.0×g to about 5.5×g, about 3.0×g to about 5.0×g, about 3.0×g to about 4.5×g, about 3.0×g to about 4.1×g, about 3.0×g to about 4.0×g, about 3.0×g to about 3.5×g, about 3.5×g to about 50×g, about 3.5×g to about 40×g, about 3.5×g to about 30×g, about 3.5×g to about 20×g, about 3.5×g to about 15×g, about 3.5×g to about 10×g, about 3.5×g to about 9.5×g, about 3.5×g to about 9.0×g, about 3.5×g to about 8.5×g, about 3.5×g to about 8.0×g, about 3.5×g to about 7.5×g, about 3.5×g to about 7.0×g, about 3.5×g to about 6.5×g, about 3.5×g to about 6.0×g, about 3.5×g to about 5.5×g, about 3.5×g to about 5.0×g, about 3.5×g to about 4.5×g, about 3.5×g to about 4.1×g, about 3.5×g to about 4.0×g, about 4.0×g to about 50×g, about 4.0×g to about 40×g, about 4.0×g to about 30×g, about 4.0×g to about 20×g, about 4.0×g to about 15×g, about 4.0×g to about 10×g, about 4.0×g to about 9.5×g, about 4.0×g to about 9.0×g, about 4.0×g to about 8.5×g, about 4.0×g to about 8.0×g, about 4.0×g to about 7.5×g, about 4.0×g to about 7.0×g, about 4.0×g to about 6.5×g, about 4.0×g to about 6.0×g, about 4.0×g to about 5.5×g, about 4.0×g to about 5.0×g, about 4.0×g to about 4.5×g, about 4.0×g to about 4.1×g, about 4.1×g to about 50×g, about 4.1×g to about 40×g, about 4.1×g to about 30×g, about 4.1×g to about 20×g, about 4.1×g to about 15×g, about 4.1×g to about 10×g, about 4.1×g to about 9.5×g, about 4.1×g to about 9.0×g, about 4.1×g to about 8.5×g, about 4.1×g to about 8.0×g, about 4.1×g to about 7.5×g, about 4.1×g to about 7.0×g, about 4.1×g to about 6.5×g, about 4.1×g to about 6.0×g, about 4.1×g to about 5.5×g, about 4.1×g to about 5.0×g, about 4.1×g to about 4.5×g, about 4.5×g to about 50×g, about 4.5×g to about 40×g, about 4.5×g to about 30×g, about 4.5×g to about 20×g, about 4.5×g to about 15×g, about 4.5×g to about 10×g, about 4.5×g to about 9.5×g, about 4.5×g to about 9.0×g, about 4.5×g to about 8.5×g, about 4.5×g to about 8.0×g, about 4.5×g to about 7.5×g, about 4.5×g to about 7.0×g, about 4.5×g to about 6.5×g, about 4.5×g to about 6.0×g, about 4.5×g to about 5.5×g, about 4.5×g to about 5.0×g, about 5.0×g to about 50×g, about 5.0×g to about 40×g, about 5.0×g to about 30×g, about 5.0×g to about 20×g, about 5.0×g to about 15×g, about 5.0×g to about 10×g, about 5.0×g to about 9.5×g, about 5.0×g to about 9.0×g, about 5.0×g to about 8.5×g, about 5.0×g to about 8.0×g, about 5.0×g to about 7.5×g, about 5.0×g to about 7.0×g, about 5.0×g to about 6.5×g, about 5.0×g to about 6.0×g, about 5.0×g to about 5.5×g, about 5.5×g to about 50×g, about 5.5×g to about 40×g, about 5.5×g to about 30×g, about 5.5×g to about 20×g, about 5.5×g to about 15×g, about 5.5×g to about 10×g, about 5.5×g to about 9.5×g, about 5.5×g to about 9.0×g, about 5.5×g to about 8.5×g, about 5.5×g to about 8.0×g, about 5.5×g to about 7.5×g, about 5.5×g to about 7.0×g, about 5.5×g to about 6.5×g, about 5.5×g to about 6.0×g, about 6.0×g to about 50×g, about 6.0×g to about 40×g, about 6.0×g to about 30×g, about 6.0×g to about 20×g, about 6.0×g to about 15×g, about 6.0×g to about 10×g, about 6.0×g to about 9.5×g, about 6.0×g to about 9.0×g, about 6.0×g to about 8.5×g, about 6.0×g to about 8.0×g, about 6.0×g to about 7.5×g, about 6.0×g to about 7.0×g, about 6.0×g to about 6.5×g, about 6.5×g to about 50×g, about 6.5×g to about 40×g, about 6.5×g to about 30×g, about 6.5×g to about 20×g, about 6.5×g to about 15×g, about 6.5×g to about 10×g, about 6.5×g to about 9.5×g, about 6.5×g to about 9.0×g, about 6.5×g to about 8.5×g, about 6.5×g to about 8.0×g, about 6.5×g to about 7.5×g, about 6.5×g to about 7.0×g, about 7.0×g to about 50×g, about 7.0×g to about 40×g, about 7.0×g to about 30×g, about 7.0×g to about 20×g, about 7.0×g to about 15×g, about 7.0×g to about 10×g, about 7.0×g to about 9.5×g, about 7.0×g to about 9.0×g, about 7.0×g to about 8.5×g, about 7.0×g to about 8.0×g, about 7.0×g to about 7.5×g, about 7.5×g to about 50×g, about 7.5×g to about 40×g, about 7.5×g to about 30×g, about 7.5×g to about 20×g, about 7.5×g to about 15×g, about 7.5×g to about 10×g, about 7.5×g to about 9.5×g, about 7.5×g to about 9.0×g, about 7.5×g to about 8.5×g, about 7.5×g to about 8.0×g, about 8.0×g to about 50×g, about 8.0×g to about 40×g, about 8.0×g to about 30×g, about 8.0×g to about 20×g, about 8.0×g to about 15×g, about 8.0×g to about 10×g, about 8.0×g to about 9.5×g, about 8.0×g to about 9.0×g, about 8.0×g to about 8.5×g, about 8.5×g to about 50×g, about 8.5×g to about 40×g, about 8.5×g to about 30×g, about 8.5×g to about 20×g, about 8.5×g to about 15×g, about 8.5×g to about 10×g, about 8.5×g to about 9.5×g, about 8.5×g to about 9.0×g, about 9.0×g to about 50×g, about 9.0×g to about 40×g, about 9.0×g to about 30×g, about 9.0×g to about 20×g, about 9.0×g to about 15×g, about 9.0×g to about 10×g, about 9.0×g to about 9.5×g, about 9.5×g to about 50×g, about 9.5×g to about 40×g, about 9.5×g to about 30×g, about 9.5×g to about 20×g, about 9.5×g to about 15×g, about 9.5×g to about 10×g, about 10×g to about 50×g, about 10×g to about 40×g, about 10×g to about 30×g, about 10×g to about 20×g, about 10×g to about 15×g, about 15×g to about 50×g, about 15×g to about 40×g, about 15×g to about 30×g, about 15×g to about 20×g, about 20×g to about 50×g, about 20×g to about 40×g, about 20×g to about 30×g, about 30×g to about 50×g, about 30×g to about 40×g, or about 40×g to about 50×g).

A variety of shake flasks are commercially available. Additional non-limiting aspects of shake flasks are known in the art.

### Shake Tubes

Some aspects of the methods described herein include the use of a shake tube. In some examples, a shake tube can have a volume of about 1 mL to about 500 mL, about 1 mL to about 450 mL, about 1 mL to about 400 mL, about 1 mL to about 350 mL, about 1 mL to about 300 mL, about 1 mL to about 250 mL, about 1 mL to about 200 mL, about 1 mL to about 180 mL, about 1 mL to about 160 mL, about 1 mL to about 140 mL, about 1 mL to about 120 mL, about 1 mL to about 100 mL, about 1 mL to about 80 mL, about 1 mL to about 60 mL, about 1 mL to about 40 mL, about 1 mL to about 20 mL, about 1 mL to about 10 mL, about 1 mL to about 5 mL, about 1 mL to about 2 mL, about 2 mL to about 500 mL, about 2 mL to about 450 mL, about 2 mL to about 400 mL, about 2 mL to about 350 mL, about 2 mL to about 300 mL, about 2 mL to about 250 mL, about 2 mL to about 200 mL, about 2 mL to about 180 mL, about 2 mL to about 160 mL, about 2 mL to about 140 mL, about 2 mL to about 120 mL, about 2 mL to about 100 mL, about 2 mL to about 80 mL, about 2 mL to about 60 mL, about 2 mL to about 40 mL, about 2 mL to about 20 mL, about 2 mL to about 10 mL, about 2 mL to about 5 mL, about 5 mL to about 500 mL, about 5 mL to about 450 mL, about 5 mL to about 400 mL, about 5 mL to about 350 mL, about 5 mL to about 300 mL, about 5 mL to about 250 mL, about 5 mL to about 200 mL, about 5 mL to about 180 mL, about 5 mL to about 160 mL, about 5 mL to about 140 mL, about 5 mL to about 120 mL, about 5 mL to about 100 mL, about 5 mL to about 80 mL, about 5 mL to about 60 mL, about 5 mL to about 40 mL, about 5 mL to about 20 mL, about 5 mL to about 10 mL, about 10 mL to about 500 mL, about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 180 mL, about 10 mL to about 160 mL, about 10 mL to about 140 mL, about 10 mL to about 120 mL, about 10 mL to about 100 mL, about 10 mL to about 80 mL, about 10 mL to about 60 mL, about 10 mL to about 40 mL, about 10 mL to about 20 mL, about 20 mL to about 500 mL, about 20 mL to about 450 mL, about 20 mL to about 400 mL, about 20 mL to about 350 mL, about 20 mL to about 300 mL, about 20 mL to about 250 mL, about 20 mL to about 200 mL, about 20 mL to about 180 mL, about 20 mL to about 160 mL, about 20 mL to about 140 mL, about 20 mL to about 120 mL, about 20 mL to about 100 mL, about 20 mL to about 80 mL, about 20 mL to about 60 mL, about 20 mL to about 40 mL, about 40 mL to about 500 mL, about 40 mL to about 450 mL, about 40 mL to about 400 mL, about 40 mL to about 350 mL, about 40 mL to about 300 mL, about 40 mL to about 250 mL, about 40 mL to about 200 mL, about 40 mL to about 180 mL, about 40 mL to about 160 mL, about 40 mL to about 140 mL, about 40 mL to about 120 mL, about 40 mL to about 100 mL, about 40 mL to about 80 mL, about 40 mL to about 60 mL, about 60 mL to about 500 mL, about 60 mL to about 450 mL, about 60 mL to about 400 mL, about 60 mL to about 350 mL, about 60 mL to about 300 mL, about 60 mL to about 250 mL, about 60 mL to about 200 mL, about 60 mL to about 180 mL, about 60 mL to about 160 mL, about 60 mL to about 140 mL, about 60 mL to about 120 mL, about 60 mL to about 100 mL, about 60 mL to about 80 mL, about 80 mL to about 500 mL, about 80 mL to about 450 mL, about 80 mL to about 400 mL, about 80 mL to about 350 mL, about 80 mL to about 300 mL, about 80 mL to about 250 mL, about 80 mL to about 200 mL, about 80 mL to about 180 mL, about 80 mL to about 160 mL, about 80 mL to about 140 mL, about 80 mL to about 120 mL, about 80 mL to about 100 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 180 mL, about 100 mL to about 160 mL, about 100 mL to about 140 mL, about 100 mL to about 120 mL, about 120 mL to about 500 mL, about 120 mL to about 450 mL, about 120 mL to about 400 mL, about 120 mL to about 350 mL, about 120 mL to about 300 mL, about 120 mL to about 250 mL, about 120 mL to about 200 mL, about 120 mL to about 180 mL, about 120 mL to about 160 mL, about 120 mL to about 140 mL, about 140 mL to about 500 mL, about 140 mL to about 450 mL, about 140 mL to about 400 mL, about 140 mL to about 350 mL, about 140 mL to about 300 mL, about 140 mL to about 250 mL, about 140 mL to about 200 mL, about 140 mL to about 180 mL, about 140 mL to about 160 mL, about 160 mL to about 500 mL, about 160 mL to about 450 mL, about 160 mL to about 400 mL, about 160 mL to about 350 mL, about 160 mL to about 300 mL, about 160 mL to about 250 mL, about 160 mL to about 200 mL, about 160 mL to about 180 mL, about 180 mL to about 500 mL, about 180 mL to about 450 mL, about 180 mL to about 400 mL, about 180 mL to about 350 mL, about 180 mL to about 300 mL, about 180 mL to about 250 mL, about 180 mL to about 200 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 500 mL, about 250 mL to about 450 mL, about 250 mL to about 400 mL, about 250 mL to about 350 mL, about 250 mL to about 300 mL, about 300 mL to about 500 mL, about 300 mL to about 450 mL, about 300 mL to about 400 mL, about 300 mL to about 350 mL, about 350 mL to about 500 mL, about 350 mL to about 450 mL, about 350 mL to about 400 mL, about 400 mL to about 500 mL, about 400 mL to about 450 mL, or about 450 mL to about 500 mL.

In some examples, a shake tube can be incubated with an agitation of about 0.1×g to about 50×g (or any of the subranges of this range described herein).

A variety of shake tubes are commercially available. Additional non-limiting aspects of shake tubes are known in the art.

### Culture Bag

Some aspects of the methods described herein include the use of a culture bag. In some examples, a culture bag can have a volume of about 50 mL to about 500 L, about 50 mL to about 400 L, about 50 mL to about 300 L, about 50 mL to about 200 L, about 50 mL to about 100 L, about 50 mL to about 50 L, 50 mL to about 25 L, about 50 mL to about 20 L, about 50 mL to about 15 L, about 50 mL to about 10 L, about 50 mL to about 8 L, about 50 mL to about 6 L, about 50 mL to about 5 L, about 50 mL to about 4 L, about 50 mL to about 3 L, about 50 mL to about 2.5 L, about 50 mL to about 2.0 L, about 50 mL to about 1.8 L, about 50 mL to about 1.6 L, about 50 mL to about 1.4 L, about 50 mL to about 1.2 L, about 50 mL to about 1.0 L, about 50 mL to about 900 mL, about 50 mL to about 800 mL, about 50 mL to about 700 mL, about 50 mL to about 600 mL, about 50 mL to about 500 mL, about 50 mL to about 450 mL, about 50 mL to about 400 mL, about 50 mL to about 350 mL, about 50 mL to about 300 mL, about 50 mL to about 250 mL, about 50 mL to about 200 mL, about 50 mL to about 150 mL, about 50 mL to about 100 mL, about 50 mL to about 75 mL, about 75 mL to about 500 L, about 75 mL to about 400 L, about 75 mL to about 300 L, about 75 mL to about 200 L, about 75 mL to about 100 L, about 75 mL to about 50 L, 75 mL to about 25 L, about 75 mL to about 20 L, about 75 mL to about 15 L, about 75 mL to about 10 L, about 75 mL to about 8 L, about 75 mL to about 6 L, about 75 mL to about 5 L, about 75 mL to about 4 L, about 75 mL to about 3 L, about 75 mL to about 2.5 L, about 75 mL to about 2.0 L, about 75 mL to about 1.8 L, about 75 mL to about 1.6 L, about 75 mL to about 1.4 L, about 75 mL to about 1.2 L, about 75 mL to about 1.0 L, about 75 mL to about 900 mL, about 75 mL to about 800 mL, about 75 mL to about 700 mL, about 75 mL to about 600 mL, about 75 mL to about 500 mL, about 75 mL to about 450 mL, about 75 mL to about 400 mL, about 75 mL to about 350 mL, about 75 mL to about 300 mL, about 75 mL to about 250 mL, about 75 mL to about 200 mL, about 75 mL to about 150 mL, about 75 mL to about 100 mL, about 100 mL to about 500 L, about 100 mL to about 400 L, about 100 mL to about 300 L, about 100 mL to about 200 L, about 100 mL to about 100 L, about 100 mL to about 50 L, 100 mL to about 25 L, about 100 mL to about 20 L, about 100 mL to about 15 L, about 100 mL to about 10 L, about 100 mL to about 8 L, about 100 mL to about 6 L, about 100 mL to about 5 L, about 100 mL to about 4 L, about 100 mL to about 3 L, about 100 mL to about 2.5 L, about 100 mL to about 2.0 L, about 100 mL to about 1.8 L, about 100 mL to about 1.6 L, about 100 mL to about 1.4 L, about 100 mL to about 1.2 L, about 100 mL to about 1.0 L, about 100 mL to about 900 mL, about 100 mL to about 800 mL, about 100 mL to about 700 mL, about 100 mL to about 600 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 150 mL, about 150 mL to about 500 L, about 150 mL to about 400 L, about 150 mL to about 300 L, about 150 mL to about 200 L, about 150 mL to about 100 L, about 150 mL to about 50 L, 150 mL to about 25 L, about 150 mL to about 20 L, about 150 mL to about 15 L, about 150 mL to about 10 L, about 150 mL to about 8 L, about 150 mL to about 6 L, about 150 mL to about 5 L, about 150 mL to about 4 L, about 150 mL to about 3 L, about 150 mL to about 2.5 L, about 150 mL to about 2.0 L, about 150 mL to about 1.8 L, about 150 mL to about 1.6 L, about 150 mL to about 1.4 L, about 150 mL to about 1.2 L, about 150 mL to about 1.0 L, about 150 mL to about 900 mL, about 150 mL to about 800 mL, about 150 mL to about 700 mL, about 150 mL to about 600 mL, about 150 mL to about 500 mL, about 150 mL to about 450 mL, about 150 mL to about 400 mL, about 150 mL to about 350 mL, about 150 mL to about 300 mL, about 150 mL to about 250 mL, about 150 mL to about 200 mL, about 200 mL to about 500 L, about 200 mL to about 400 L, about 200 mL to about 300 L, about 200 mL to about 200 L, about 200 mL to about 100 L, about 200 mL to about 50 L, 200 mL to about 25 L, about 200 mL to about 20 L, about 200 mL to about 15 L, about 200 mL to about 10 L, about 200 mL to about 8 L, about 200 mL to about 6 L, about 200 mL to about 5 L, about 200 mL to about 4 L, about 200 mL to about 3 L, about 200 mL to about 2.5 L, about 200 mL to about 2.0 L, about 200 mL to about 1.8 L, about 200 mL to about 1.6 L, about 200 mL to about 1.4 L, about 200 mL to about 1.2 L, about 200 mL to about 1.0 L, about 200 mL to about 900 mL, about 200 mL to about 800 mL, about 200 mL to about 700 mL, about 200 mL to about 600 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 500 L, about 250 mL to about 400 L, about 250 mL to about 300 L, about 250 mL to about 200 L, about 250 mL to about 100 L, about 250 mL to about 50 L, 250 mL to about 25 L, about 250 mL to about 20 L, about 250 mL to about 15 L, about 250 mL to about 10 L, about 250 mL to about 8 L, about 250 mL to about 6 L, about 250 mL to about 5 L, about 250 mL to about 4 L, about 250 mL to about 3 L, about 250 mL to about 2.5 L, about 250 mL to about 2.0 L, about 250 mL to about 1.8 L, about 250 mL to about 1.6 L, about 250 mL to about 1.4 L, about 250 mL to about 1.2 L, about 250 mL to about 1.0 L, about 250 mL to about 900 mL, about 250 mL to about 800 mL, about 250 mL to about 700 mL, about 250 mL to about 600 mL, about 250 mL to about 500 mL, about 250 mL to about 450 mL, about 250 mL to about 400 mL, about 250 mL to about 350 mL, about 250 mL to about 300 mL, about 300 mL to about 500 L, about 300 mL to about 400 L, about 300 mL to about 300 L, about 300 mL to about 200 L, about 300 mL to about 100 L, about 300 mL to about 50 L, 300 mL to about 25 L, about 300 mL to about 20 L, about 300 mL to about 15 L, about 300 mL to about 10 L, about 300 mL to about 8 L, about 300 mL to about 6 L, about 300 mL to about 5 L, about 300 mL to about 4 L, about 300 mL to about 3 L, about 300 mL to about 2.5 L, about 300 mL to about 2.0 L, about 300 mL to about 1.8 L, about 300 mL to about 1.6 L, about 300 mL to about 1.4 L, about 300 mL to about 1.2 L, about 300 mL to about 1.0 L, about 300 mL to about 900 mL, about 300 mL to about 800 mL, about 300 mL to about 700 mL, about 300 mL to about 600 mL, about 300 mL to about 500 mL, about 300 mL to about 450 mL, about 300 mL to about 400 mL, about 300 mL to about 350 mL, about 350 mL to about 500 L, about 350 mL to about 400 L, about 350 mL to about 300 L, about 350 mL to about 200 L, about 350 mL to about 100 L, about 350 mL to about 50 L, 350 mL to about 25 L, about 350 mL to about 20 L, about 350 mL to about 15 L, about 350 mL to about 10 L, about 350 mL to about 8 L, about 350 mL to about 6 L, about 350 mL to about 5 L, about 350 mL to about 4 L, about 350 mL to about 3 L, about 350 mL to about 2.5 L, about 350 mL to about 2.0 L, about 350 mL to about 1.8 L, about 350 mL to about 1.6 L, about 350 mL to about 1.4 L, about 350 mL to about 1.2 L, about 350 mL to about 1.0 L, about 350 mL to about 900 mL, about 350 mL to about 800 mL, about 350 mL to about 700 mL, about 350 mL to about 600 mL, about 350 mL to about 500 mL, about 350 mL to about 450 mL, about 350 mL to about 400 mL, about 400 mL to about 500 L, about 400 mL to about 400 L, about 400 mL to about 300 L, about 400 mL to about 200 L, about 400 mL to about 100 L, about 400 mL to about 50 L, 400 mL to about 25 L, about 400 mL to about 20 L, about 400 mL to about 15 L, about 400 mL to about 10 L, about 400 mL to about 8 L, about 400 mL to about 6 L, about 400 mL to about 5 L, about 400 mL to about 4 L, about 400 mL to about 3 L, about 400 mL to about 2.5 L, about 400 mL to about 2.0 L, about 400 mL to about 1.8 L, about 400 mL to about 1.6 L, about 400 mL to about 1.4 L, about 400 mL to about 1.2 L, about 400 mL to about 1.0 L, about 400 mL to about 900 mL, about 400 mL to about 800 mL, about 400 mL to about 700 mL, about 400 mL to about 600 mL, about 400 mL to about 500 mL, about 400 mL to about 450 mL, about 450 mL to about 500 L, about 450 mL to about 400 L, about 450 mL to about 300 L, about 450 mL to about 200 L, about 450 mL to about 100 L, about 450 mL to about 50 L, 450 mL to about 25 L, about 450 mL to about 20 L, about 450 mL to about 15 L, about 450 mL to about 10 L, about 450 mL to about 8 L, about 450 mL to about 6 L, about 450 mL to about 5 L, about 450 mL to about 4 L, about 450 mL to about 3 L, about 450 mL to about 2.5 L, about 450 mL to about 2.0 L, about 450 mL to about 1.8 L, about 450 mL to about 1.6 L, about 450 mL to about 1.4 L, about 450 mL to about 1.2 L, about 450 mL to about 1.0 L, about 450 mL to about 900 mL, about 450 mL to about 800 mL, about 450 mL to about 700 mL, about 450 mL to about 600 mL, about 450 mL to about 500 mL, about 500 mL to about 500 L, about 500 mL to about 400 L, about 500 mL to about 300 L, about 500 mL to about 200 L, about 500 mL to about 100 L, about 500 mL to about 50 L, 500 mL to about 25 L, about 500 mL to about 20 L, about 500 mL to about 15 L, about 500 mL to about 10 L, about 500 mL to about 8 L, about 500 mL to about 6 L, about 500 mL to about 5 L, about 500 mL to about 4 L, about 500 mL to about 3 L, about 500 mL to about 2.5 L, about 500 mL to about 2.0 L, about 500 mL to about 1.8 L, about 500 mL to about 1.6 L, about 500 mL to about 1.4 L, about 500 mL to about 1.2 L, about 500 mL to about 1.0 L, about 500 mL to about 900 mL, about 500 mL to about 800 mL, about 500 mL to about 700 mL, about 500 mL to about 600 mL, about 600 mL to about 500 L, about 600 mL to about 400 L, about 600 mL to about 300 L, about 600 mL to about 200 L, about 600 mL to about 100 L, about 600 mL to about 50 L, 600 mL to about 25 L, about 600 mL to about 20 L, about 600 mL to about 15 L, about 600 mL to about 10 L, about 600 mL to about 8 L, about 600 mL to about 6 L, about 600 mL to about 5 L, about 600 mL to about 4 L, about 600 mL to about 3 L, about 600 mL to about 2.5 L, about 600 mL to about 2.0 L, about 600 mL to about 1.8 L, about 600 mL to about 1.6 L, about 600 mL to about 1.4 L, about 600 mL to about 1.2 L, about 600 mL to about 1.0 L, about 600 mL to about 900 mL, about 600 mL to about 800 mL, about 600 mL to about 700 mL, about 700 mL to about 500 L, about 700 mL to about 400 L, about 700 mL to about 300 L, about 700 mL to about 200 L, about 700 mL to about 100 L, about 700 mL to about 50 L, 700 mL to about 25 L, about 700 mL to about 20 L, about 700 mL to about 15 L, about 700 mL to about 10 L, about 700 mL to about 8 L, about 700 mL to about 6 L, about 700 mL to about 5 L, about 700 mL to about 4 L, about 700 mL to about 3 L, about 700 mL to about 2.5 L, about 700 mL to about 2.0 L, about 700 mL to about 1.8 L, about 700 mL to about 1.6 L, about 700 mL to about 1.4 L, about 700 mL to about 1.2 L, about 700 mL to about 1.0 L, about 700 mL to about 900 mL, about 700 mL to about 800 mL, about 800 mL to about 500 L, about 800 mL to about 400 L, about 800 mL to about 300 L, about 800 mL to about 200 L, about 800 mL to about 100 L, about 800 mL to about 50 L, 800 mL to about 25 L, about 800 mL to about 20 L, about 800 mL to about 15 L, about 800 mL to about 10 L, about 800 mL to about 8 L, about 800 mL to about 6 L, about 800 mL to about 5 L, about 800 mL to about 4 L, about 800 mL to about 3 L, about 800 mL to about 2.5 L, about 800 mL to about 2.0 L, about 800 mL to about 1.8 L, about 800 mL to about 1.6 L, about 800 mL to about 1.4 L, about 800 mL to about 1.2 L, about 800 mL to about 1.0 L, about 800 mL to about 900 mL, about 900 mL to about 500 L, about 900 mL to about 400 L, about 900 mL to about 300 L, about 900 mL to about 200 L, about 900 mL to about 100 L, about 900 mL to about 50 L, 900 mL to about 25 L, about 900 mL to about 20 L, about 900 mL to about 15 L, about 900 mL to about 10 L, about 900 mL to about 8 L, about 900 mL to about 6 L, about 900 mL to about 5 L, about 900 mL to about 4 L, about 900 mL to about 3 L, about 900 mL to about 2.5 L, about 900 mL to about 2.0 L, about 900 mL to about 1.8 L, about 900 mL to about 1.6 L, about 900 mL to about 1.4 L, about 900 mL to about 1.2 L, about 900 mL to about 1.0 L, about 1.0 L to about 500 L, about 1.0 L to about 400 L, about 1.0 L to about 300 L, about 1.0 L to about 200 L, about 1.0 L to about 100 L, about 1.0 L to about 50 L, 1.0 L to about 25 L, about 1.0 L to about 20 L, about 1.0 L to about 15 L, about 1.0 L to about 10 L, about 1.0 L to about 8 L, about 1.0 L to about 6 L, about 1.0 L to about 5 L, about 1.0 L to about 4 L, about 1.0 L to about 3 L, about 1.0 L to about 2.5 L, about 1.0 L to about 2.0 L, about 1.0 L to about 1.8 L, about 1.0 L to about 1.6 L, about 1.0 L to about 1.4 L, about 1.0 L to about 1.2 L, about 1.2 L to about 500 L, about 1.2 L to about 400 L, about 1.2 L to about 300 L, about 1.2 L to about 200 L, about 1.2 L to about 100 L, about 1.2 L to about 50 L, 1.2 L to about 25 L, about 1.2 L to about 20 L, about 1.2 L to about 15 L, about 1.2 L to about 10 L, about 1.2 L to about 8 L, about 1.2 L to about 6 L, about 1.2 L to about 5 L, about 1.2 L to about 4 L, about 1.2 L to about 3 L, about 1.2 L to about 2.5 L, about 1.2 L to about 2.0 L, about 1.2 L to about 1.8 L, about 1.2 L to about 1.6 L, about 1.2 L to about 1.4 L, about 1.4 L to about 500 L, about 1.4 L to about 400 L, about 1.4 L to about 300 L, about 1.4 L to about 200 L, about 1.4 L to about 100 L, about 1.4 L to about 50 L, 1.4 L to about 25 L, about 1.4 L to about 20 L, about 1.4 L to about 15 L, about 1.4 L to about 10 L, about 1.4 L to about 8 L, about 1.4 L to about 6 L, about 1.4 L to about 5 L, about 1.4 L to about 4 L, about 1.4 L to about 3 L, about 1.4 L to about 2.5 L, about 1.4 L to about 2.0 L, about 1.4 L to about 1.8 L, about 1.4 L to about 1.6 L, about 1.6 L to about 500 L, about 1.6 L to about 400 L, about 1.6 L to about 300 L, about 1.6 L to about 200 L, about 1.6 L to about 100 L, about 1.6 L to about 50 L, 1.6 L to about 25 L, about 1.6 L to about 20 L, about 1.6 L to about 15 L, about 1.6 L to about 10 L, about 1.6 L to about 8 L, about 1.6 L to about 6 L, about 1.6 L to about 5 L, about 1.6 L to about 4 L, about 1.6 L to about 3 L, about 1.6 L to about 2.5 L, about 1.6 L to about 2.0 L, about 1.6 L to about 1.8 L, about 1.8 L to about 500 L, about 1.8 L to about 400 L, about 1.8 L to about 300 L, about 1.8 L to about 200 L, about 1.8 L to about 100 L, about 1.8 L to about 50 L, 1.8 L to about 25 L, about 1.8 L to about 20 L, about 1.8 L to about 15 L, about 1.8 L to about 10 L, about 1.8 L to about 8 L, about 1.8 L to about 6 L, about 1.8 L to about 5 L, about 1.8 L to about 4 L, about 1.8 L to about 3 L, about 1.8 L to about 2.5 L, about 1.8 L to about 2.0 L, about 2.0 L to about 500 L, about 2.0 L to about 400 L, about 2.0 L to about 300 L, about 2.0 L to about 200 L, about 2.0 L to about 100 L, about 2.0 L to about 50 L, 2.0 L to about 25 L, about 2.0 L to about 20 L, about 2.0 L to about 15 L, about 2.0 L to about 10 L, about 2.0 L to about 8 L, about 2.0 L to about 6 L, about 2.0 L to about 5 L, about 2.0 L to about 4 L, about 2.0 L to about 3 L, about 2.0 L to about 2.5 L, about 2.5 L to about 500 L, about 2.5 L to about 400 L, about 2.5 L to about 300 L, about 2.5 L to about 200 L, about 2.5 L to about 100 L, about 2.5 L to about 50 L, 2.5 L to about 25 L, about 2.5 L to about 20 L, about 2.5 L to about 15 L, about 2.5 L to about 10 L, about 2.5 L to about 8 L, about 2.5 L to about 6 L, about 2.5 L to about 5 L, about 2.5 L to about 4 L, about 2.5 L to about 3 L, about 3 L to about 500 L, about 3 L to about 400 L, about 3 L to about 300 L, about 3 L to about 200 L, about 3 L to about 100 L, about 3 L to about 50 L, 3 L to about 25 L, about 3 L to about 20 L, about 3 L to about 15 L, about 3 L to about 10 L, about 3 L to about 8 L, about 3 L to about 6 L, about 3 L to about 5 L, about 3 L to about 4 L, about 4 L to about 500 L, about 4 L to about 400 L, about 4 L to about 300 L, about 4 L to about 200 L, about 4 L to about 100 L, about 4 L to about 50 L, 4 L to about 25 L, about 4 L to about 20 L, about 4 L to about 15 L, about 4 L to about 10 L, about 4 L to about 8 L, about 4 L to about 6 L, about 4 L to about 5 L, about 5 L to about 500 L, about 5 L to about 400 L, about 5 L to about 300 L, about 5 L to about 200 L, about 5 L to about 100 L, about 5 L to about 50 L, 5 L to about 25 L, about 5 L to about 20 L, about 5 L to about 15 L, about 5 L to about 10 L, about 5 L to about 8 L, about 5 L to about 6 L, about 6 L to about 500 L, about 6 L to about 400 L, about 6 L to about 300 L, about 6 L to about 200 L, about 6 L to about 100 L, about 6 L to about 50 L, 6 L to about 25 L, about 6 L to about 20 L, about 6 L to about 15 L, about 6 L to about 10 L, about 6 L to about 8 L, about 8 L to about 500 L, about 8 L to about 400 L, about 8 L to about 300 L, about 8 L to about 200 L, about 8 L to about 100 L, about 8 L to about 50 L, 8 L to about 25 L, about 8 L to about 20 L, about 8 L to about 15 L, about 8 L to about 10 L, about 10 L to about 500 L, about 10 L to about 400 L, about 10 L to about 300 L, about 10 L to about 200 L, about 10 L to about 100 L, about 10 L to about 50 L, 10 L to about 25 L, about 10 L to about 20 L, about 10 L to about 15 L, about 15 L to about 500 L, about 15 L to about 400 L, about 15 L to about 300 L, about 15 L to about 200 L, about 15 L to about 100 L, about 15 L to about 50 L, 15 L to about 25 L, about 15 L to about 20 L, about 20 L to about 500 L, about 20 L to about 400 L, about 20 L to about 300 L, about 20 L to about 200 L, about 20 L to about 100 L, about 20 L to about 50 L, 20 L to about 25 L, about 25 L to about 500 L, about 25 L to about 400 L, about 25 L to about 300 L, about 25 L to about 200 L, about 25 L to about 100 L, about 25 L to about 50 L, about 50 L to about 500 L, about 50 L to about 400 L, about 50 L to about 300 L, about 50 L to about 200 L, about 50 L to about 100 L, about 100 L to about 500 L, about 100 L to about 400 L, about 100 L to about 300 L, about 100 L to about 200 L, about 200 L to about 500 L, about 200 L to about 400 L, about 200 L to about 300 L, about 300 L to about 500 L, about 300 L to about 400 L, or about 400 L to about 500 L.

In some examples, a culture bag can be incubated at a rocking rate of about 5 rock cycles per minute to about 40 rock cycles per minute, about 5 rock cycles per minute to about 35 rock cycles per minute, about 5 rock cycles per minute to about 30 rock cycles per minute, about 5 rock cycles per minute to about 25 rock cycles per minute, about 5 rock cycles per minute to about 20 rock cycles per minute, about 5 rock cycles per minute to about 15 rock cycles per minute, about 5 rock cycles per minute to about 10 rock cycles per minute, about 10 rock cycles per minute to about 40 rock cycles per minute, about 10 rock cycles per minute to about 35 rock cycles per minute, about 10 rock cycles per minute to about 30 rock cycles per minute, about 10 rock cycles per minute to about 25 rock cycles per minute, about 10 rock cycles per minute to about 20 rock cycles per minute, about 10 rock cycles per minute to about 15 rock cycles per minute, about 15 rock cycles per minute to about 40 rock cycles per minute, about 15 rock cycles per minute to about 35 rock cycles per minute, about 15 rock cycles per minute to about 30 rock cycles per minute, about 15 rock cycles per minute to about 25 rock cycles per minute, about 15 rock cycles per minute to about 20 rock cycles per minute, about 20 rock cycles per minute to about 40 rock cycles per minute, about 20 rock cycles per minute to about 35 rock cycles per minute, about 20 rock cycles per minute to about 30 rock cycles per minute, about 20 rock cycles per minute to about 25 rock cycles per minute, about 25 rock cycles per minute to about 40 rock cycles per minute, about 25 rock cycles per minute to about 35 rock cycles per minute, about 25 rock cycles per minute to about 30 rock cycles per minute, about 30 rock cycles per minute to about 40 rock cycles per minute, about 30 rock cycles per minute to about 35 rock cycles per minute, or about 35 rock cycles per minute to about 40 rock cycles per minute.

A variety of culture bags are commercially available. Additional non-limiting aspects of culture bags are known in the art.

### Bioreactors

Some aspects of the methods described herein include the use of a bioreactor (e.g., a perfusion bioreactor). In some examples, a bioreactor (e.g., a perfusion bioreactor) can have a volume of about 500 mL to about 15,000 L, about 500 mL to about 12,500 L, about 500 mL to about 10,000 L, about 500 mL to about 8,000 L, about 500 mL to about 6,000 L, about 500 mL to about 4,000 L, about 500 mL to about 2,000 L, about 500 mL to about 1,000 L, about 500 mL to about 800 L, about 500 mL to about 600 L, about 500 mL to about 400 L, about 500 mL to about 200 L, about 500 mL to about 100 L, about 500 mL to about 50 L, about 500 mL to about 20 L, about 500 mL to about 10 L, about 500 mL to about 5 L, about 500 mL to about 1 L, about 500 mL to about 750 mL, about 750 mL to about 15,000 L, about 750 mL to about 12,500 L, about 750 mL to about 10,000 L, about 750 mL to about 8,000 L, about 750 mL to about 6,000 L, about 750 mL to about 4,000 L, about 750 mL to about 2,000 L, about 750 mL to about 1,000 L, about 750 mL to about 800 L, about 750 mL to about 600 L, about 750 mL to about 400 L, about 750 mL to about 200 L, about 750 mL to about 100 L, about 750 mL to about 50 L, about 750 mL to about 20 L, about 750 mL to about 10 L, about 750 mL to about 5 L, about 750 mL to about 1 L, about 1 L to about 15,000 L, about 1 L to about 12,500 L, about 1 L to about 10,000 L, about 1 L to about 8,000 L, about 1 L to about 6,000 L, about 1 L to about 4,000 L, about 1 L to about 2,000 L, about 1 L to about 1,000 L, about 1 L to about 800 L, about 1 L to about 600 L, about 1 L to about 400 L, about 1 L to about 200 L, about 1 L to about 100 L, about 1 L to about 50 L, about 1 L to about 20 L, about 1 L to about 10 L, about 1 L to about 5 L, about 5 L to about 15,000 L, about 5 L to about 12,500 L, about 5 L to about 10,000 L, about 5 L to about 8,000 L, about 5 L to about 6,000 L, about 5 L to about 4,000 L, about 5 L to about 2,000 L, about 5 L to about 1,000 L, about 5 L to about 800 L, about 5 L to about 600 L, about 5 L to about 400 L, about 5 L to about 200 L, about 5 L to about 100 L, about 5 L to about 50 L, about 5 L to about 20 L, about 5 L to about 10 L, about 10 L to about 15,000 L, about 10 L to about 12,500 L, about 10 L to about 10,000 L, about 10 L to about 8,000 L, about 10 L to about 6,000 L, about 10 L to about 4,000 L, about 10 L to about 2,000 L, about 10 L to about 1,000 L, about 10 L to about 800 L, about 10 L to about 600 L, about 10 L to about 400 L, about 10 L to about 200 L, about 10 L to about 100 L, about 10 L to about 50 L, about 10 L to about 20 L, about 20 L to about 15,000 L, about 20 L to about 12,500 L, about 20 L to about 10,000 L, about 20 L to about 8,000 L, about 20 L to about 6,000 L, about 20 L to about 4,000 L, about 20 L to about 2,000 L, about 20 L to about 1,000 L, about 20 L to about 800 L, about 20 L to about 600 L, about 20 L to about 400 L, about 20 L to about 200 L, about 20 L to about 100 L, about 20 L to about 50 L, about 50 L to about 15,000 L, about 50 L to about 12,500 L, about 50 L to about 10,000 L, about 50 L to about 8,000 L, about 50 L to about 6,000 L, about 50 L to about 4,000 L, about 50 L to about 2,000 L, about 50 L to about 1,000 L, about 50 L to about 800 L, about 50 L to about 600 L, about 50 L to about 400 L, about 50 L to about 200 L, about 50 L to about 100 L, about 100 L to about 15,000 L, about 100 L to about 12,500 L, about 100 L to about 10,000 L, about 100 L to about 8,000 L, about 100 L to about 6,000 L, about 100 L to about 4,000 L, about 100 L to about 2,000 L, about 100 L to about 1,000 L, about 100 L to about 800 L, about 100 L to about 600 L, about 100 L to about 400 L, about 100 L to about 200 L, about 200 L to about 15,000 L, about 200 L to about 12,500 L, about 200 L to about 10,000 L, about 200 L to about 8,000 L, about 200 L to about 6,000 L, about 200 L to about 4,000 L, about 200 L to about 2,000 L, about 200 L to about 1,000 L, about 200 L to about 800 L, about 200 L to about 600 L, about 200 L to about 400 L, about 400 L to about 15,000 L, about 400 L to about 12,500 L, about 400 L to about 10,000 L, about 400 L to about 8,000 L, about 400 L to about 6,000 L, about 400 L to about 4,000 L, about 400 L to about 2,000 L, about 400 L to about 1,000 L, about 400 L to about 800 L, about 400 L to about 600 L, about 600 L to about 15,000 L, about 600 L to about 12,500 L, about 600 L to about 10,000 L, about 600 L to about 8,000 L, about 600 L to about 6,000 L, about 600 L to about 4,000 L, about 600 L to about 2,000 L, about 600 L to about 1,000 L, about 600 L to about 800 L, about 800 L to about 15,000 L, about 800 L to about 12,500 L, about 800 L to about 10,000 L, about 800 L to about 8,000 L, about 800 L to about 6,000 L, about 800 L to about 4,000 L, about 800 L to about 2,000 L, about 800 L to about 1,000 L, about 1,000 L to about 15,000 L, about 1,000 L to about 12,500 L, about 1,000 L to about 10,000 L, about 1,000 L to about 8,000 L, about 1,000 L to about 6,000 L, about 1,000 L to about 4,000 L, about 1,000 L to about 2,000 L, about 2,000 L to about 15,000 L, about 2,000 L to about 12,500 L, about 2,000 L to about 10,000 L, about 2,000 L to about 8,000 L, about 2,000 L to about 6,000 L, about 2,000 L to about 4,000 L, about 4,000 L to about 15,000 L, about 4,000 L to about 12,500 L, about 4,000 L to about 10,000 L, about 4,000 L to about 8,000 L, about 2,000 L to about 6,000 L, about 6,000 L to about 15,000 L, about 6,000 L to about 12,500 L, about 6,000 L to about 10,000 L, about 6,000 L to about 8,000 L, about 8,000 L to about 15,000 L, about 8,000 L to about 12,500 L, about 8,000 L to about 10,000 L, about 10,000 L to about 15,000 L, about 10,000 L to about 12,500 L, or about 12,500 L to about 15,000 L.

A variety of bioreactors (e.g., perfusion bioreactors) are commercially available. Additional non-limiting aspects of bioreactors (e.g., perfusion bioreactors) are known in the art.

### Providing a Second Cell Culture (Step A)

In any of the methods described herein, step (a) comprises disposing a volume of a first cell culture of erythroid progenitor cells (e.g., any of the exemplary erythroid progenitor cells described herein or known in the art) into a second culture medium (e.g., any of the exemplary second culture media described herein or known in the art) contained within a vessel (e.g., a bioreactor (e.g., a perfusion bioreactor), a shake flask, a shake tube, or a culture bag) to provide a second cell culture with an initial cell density of about 0.1×10⁴ cells/mL to about 1×10⁷ cells/mL, about 0.1×10⁴ cells/mL to about 0.5×10⁷ cells/mL, about 0.1×10⁴ cells/mL to about 1×10⁶ cells/mL, about 0.1×10⁴ cells/mL to about 0.5×10⁶ cells/mL, about 0.1×10⁴ cells/mL to about 4×10⁵ cells/mL, about 0.1×10⁴ cells/mL to about 2×10⁵ cells/mL, about 0.1×10⁴ cells/mL to about 1.5×10⁵ cells/mL, about 0.1×10⁴ cells/mL to about 1×10⁵ cells/mL, about 0.1×10⁴ cells/mL to about 0.5×10⁵ cells/mL, about 0.1×10⁴ cells/mL to about 1×10⁴ cells/mL, about 0.1×10⁴ cells/mL to about 0.5×10⁴ cells/mL, 0.5×10⁴ cells/mL to about 1×10⁷ cells/mL, about 0.5×10⁴ cells/mL to about 0.5×10⁷ cells/mL, about 0.5×10⁴ cells/mL to about 1×10⁶ cells/mL, about 0.5×10⁴ cells/mL to about 0.5×10⁶ cells/mL, about 0.5×10⁴ cells/mL to about 4×10⁵ cells/mL, about 0.5×10⁴ cells/mL to about 2×10⁵ cells/mL, about 0.5×10⁴ cells/mL to about 1.5×10⁵ cells/mL, about 0.5×10⁴ cells/mL to about 1×10⁵ cells/mL, about 0.5×10⁴ cells/mL to about 0.5×10⁵ cells/mL, about 0.5×10⁴ cells/mL to about 1×10⁴ cells/mL, 1×10⁴ cells/mL to about 1×10⁷ cells/mL, about 1×10⁴ cells/mL to about 0.5×10⁷ cells/mL, about 1×10⁴ cells/mL to about 1×10⁶ cells/mL, about 1×10⁴ cells/mL to about 0.5×10⁶ cells/mL, about 1×10⁴ cells/mL to about 4×10⁵ cells/mL, about 1×10⁴ cells/mL to about 2×10⁵ cells/mL, about 1×10⁴ cells/mL to about 1.5×10⁵ cells/mL, about 1×10⁴ cells/mL to about 1×10⁵ cells/mL, about 1×10⁴ cells/mL to about 0.5×10⁵ cells/mL, about 0.5×10⁵ cells/mL to about 1×10⁷ cells/mL, about 0.5×10⁵ cells/mL to about 0.5×10⁷ cells/mL, about 0.5×10⁵ cells/mL to about 1×10⁶ cells/mL, about 0.5×10⁵ cells/mL to about 0.5×10⁶ cells/mL, about 0.5×10⁵ cells/mL to about 4×10⁵ cells/mL, about 0.5×10⁵ cells/mL to about 2×10⁵ cells/mL, about 0.5×10⁵ cells/mL to about 1.5×10⁵ cells/mL, about 0.5×10⁵ cells/mL to about 1×10⁵ cells/mL, about 1×10⁵ cells/mL to about 1×10⁷ cells/mL, about 1×10⁵ cells/mL to about 0.5×10⁷ cells/mL, about 1×10⁵ cells/mL to about 1×10⁶ cells/mL, about 1×10⁵ cells/mL to about 0.5×10⁶ cells/mL, about 1×10⁵ cells/mL to about 4×10⁵ cells/mL, about 1×10⁵ cells/mL to about 2×10⁵ cells/mL, about 1×10⁵ cells/mL to about 1.5×10⁵ cells/mL, about 1.5×10⁵ cells/mL to about 1×10⁷ cells/mL, about 1.5×10⁵ cells/mL to about 0.5×10⁷ cells/mL, about 1.5×10⁵ cells/mL to about 1×10⁶ cells/mL, about 1.5×10⁵ cells/mL to about 0.5×10⁶ cells/mL, about 1.5×10⁵ cells/mL to about 4×10⁵ cells/mL, about 1.5×10⁵ cells/mL to about 2×10⁵ cells/mL, about 2×10⁵ cells/mL to about 1×10⁷ cells/mL, about 2×10⁵ cells/mL to about 0.5×10⁷ cells/mL, about 2×10⁵ cells/mL to about 1×10⁶ cells/mL, about 2×10⁵ cells/mL to about 0.5×10⁶ cells/mL, about 2×10⁵ cells/mL to about 4×10⁵ cells/mL, about 4×10⁵ cells/mL to about 1×10⁷ cells/mL, about 4×10⁵ cells/mL to about 0.5×10⁷ cells/mL, about 4×10⁵ cells/mL to about 1×10⁶ cells/mL, about 4×10⁵ cells/mL to about 0.5×10⁶ cells/mL, about 0.5×10⁶ cells/mL to about 1×10⁷ cells/mL, about 0.5×10⁶ cells/mL to about 0.5×10⁷ cells/mL, about 0.5×10⁶ cells/mL to about 1×10⁶ cells/mL, about 1×10⁶ cells/mL to about 1×10⁷ cells/mL, about 1×10⁶ cells/mL to about 0.5×10⁷ cells/mL, or about 0.5×10⁷ cells/mL to about 1×10⁷ cells/mL.

In some embodiments, step (a) includes disposing a volume of about 0.1 mL to about 5,000 L, about 0.1 mL to about 4,500 L, about 0.1 mL to about 4,000 L, about 0.1 mL to about 3,500 L, about 0.1 mL to about 3,000 L, about 0.1 mL to about 2,500 L, about 0.1 mL to about 2,000 L, about 0.1 mL to about 1,500 L, about 1 mL to about 1,000 L, about 0.1 mL to about 800 mL, about 0.1 mL to about 600 mL, about 0.1 mL to about 500 mL, about 0.1 mL to about 450 mL, about 0.1 mL to about 400 mL, about 0.1 mL to about 350 mL, about 0.1 mL to about 300 mL, about 0.1 mL to about 250 mL, about 0.1 mL to about 200 mL, about 0.1 mL to about 150 mL, about 0.1 mL to about 100 mL, about 0.1 mL to about 50 mL, about 0.1 mL to about 20 mL, about 0.1 mL to about 10 mL, about 0.1 mL to about 5 mL, about 5 mL to about 5,000 L, about 5 mL to about 4,500 L, about 5 mL to about 4,000 L, about 5 mL to about 3,500 L, about 5 mL to about 3,000 L, about 5 mL to about 2,500 L, about 5 mL to about 2,000 L, about 5 mL to about 1,500 L, about 5 mL to about 1,000 L, about 5 mL to about 800 mL, about 5 mL to about 600 mL, about 5 mL to about 500 mL, about 5 mL to about 450 mL, about 5 mL to about 400 mL, about 5 mL to about 350 mL, about 5 mL to about 300 mL, about 5 mL to about 250 mL, about 5 mL to about 200 mL, about 5 mL to about 150 mL, about 5 mL to about 100 mL, about 5 mL to about 50 mL, about 5 mL to about 20 mL, about 5 mL to about 10 mL, about 10 mL to about 5,000 L, about 10 mL to about 4,500 L, about 10 mL to about 4,000 L, about 10 mL to about 3,500 L, about 10 mL to about 3,000 L, about 10 mL to about 2,500 L, about 10 mL to about 2,000 L, about 10 mL to about 1,500 L, about 10 mL to about 1,000 L, about 10 mL to about 800 mL, about 10 mL to about 600 mL, about 10 mL to about 500 mL, about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 150 mL, about 10 mL to about 100 mL, about 10 mL to about 50 mL, about 10 mL to about 20 mL, about 20 mL to about 5,000 L, about 20 mL to about 4,500 L, about 20 mL to about 4,000 L, about 20 mL to about 3,500 L, about 20 mL to about 3,000 L, about 20 mL to about 2,500 L, about 20 mL to about 2,000 L, about 20 mL to about 1,500 L, about 20 mL to about 1,000 L, about 20 mL to about 800 mL, about 20 mL to about 600 mL, about 20 mL to about 500 mL, about 20 mL to about 450 mL, about 20 mL to about 400 mL, about 20 mL to about 350 mL, about 20 mL to about 300 mL, about 20 mL to about 250 mL, about 20 mL to about 200 mL, about 20 mL to about 150 mL, about 20 mL to about 100 mL, about 20 mL to about 50 mL, about 50 mL to about 5,000 L, about 50 mL to about 4,500 L, about 50 mL to about 4,000 L, about 50 mL to about 3,500 L, about 50 mL to about 3,000 L, about 50 mL to about 2,500 L, about 50 mL to about 2,000 L, about 50 mL to about 1,500 L, about 50 mL to about 1,000 L, about 50 mL to about 800 mL, about 50 mL to about 600 mL, about 50 mL to about 500 mL, about 50 mL to about 450 mL, about 50 mL to about 400 mL, about 50 mL to about 350 mL, about 50 mL to about 300 mL, about 50 mL to about 250 mL, about 50 mL to about 200 mL, about 50 mL to about 150 mL, about 50 mL to about 100 mL, about 100 mL to about 5,000 L, about 100 mL to about 4,500 L, about 100 mL to about 4,000 L, about 100 mL to about 3,500 L, about 100 mL to about 3,000 L, about 100 mL to about 2,500 L, about 100 mL to about 2,000 L, about 100 mL to about 1,500 L, about 100 mL to about 1,000 L, about 100 mL to about 800 mL, about 100 mL to about 600 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 150 mL, about 150 mL to about 5,000 L, about 150 mL to about 4,500 L, about 150 mL to about 4,000 L, about 150 mL to about 3,500 L, about 150 mL to about 3,000 L, about 150 mL to about 2,500 L, about 150 mL to about 2,000 L, about 150 mL to about 1,500 L, about 150 mL to about 1,000 L, about 150 mL to about 800 mL, about 150 mL to about 600 mL, about 150 mL to about 500 mL, about 150 mL to about 450 mL, about 150 mL to about 400 mL, about 150 mL to about 350 mL, about 150 mL to about 300 mL, about 150 mL to about 250 mL, about 150 mL to about 200 mL, about 200 mL to about 5,000 L, about 200 mL to about 4,500 L, about 200 mL to about 4,000 L, about 200 mL to about 3,500 L, about 200 mL to about 3,000 L, about 200 mL to about 2,500 L, about 200 mL to about 2,000 L, about 200 mL to about 1,500 L, about 200 mL to about 1,000 L, about 200 mL to about 800 mL, about 200 mL to about 600 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 5,000 L, about 250 mL to about 4,500 L, about 250 mL to about 4,000 L, about 250 mL to about 3,500 L, about 250 mL to about 3,000 L, about 250 mL to about 2,500 L, about 250 mL to about 2,000 L, about 250 mL to about 1,500 L, about 250 mL to about 1,000 L, about 250 mL to about 800 mL, about 250 mL to about 600 mL, about 250 mL to about 500 mL, about 250 mL to about 450 mL, about 250 mL to about 400 mL, about 250 mL to about 350 mL, about 250 mL to about 300 mL, about 300 mL to about 5,000 L, about 300 mL to about 4,500 L, about 300 mL to about 4,000 L, about 300 mL to about 3,500 L, about 300 mL to about 3,000 L, about 300 mL to about 2,500 L, about 300 mL to about 2,000 L, about 300 mL to about 1,500 L, about 300 mL to about 1,000 L, about 300 mL to about 800 mL, about 300 mL to about 600 mL, about 300 mL to about 500 mL, about 300 mL to about 450 mL, about 300 mL to about 400 mL, about 300 mL to about 350 mL, about 350 mL to about 5,000 L, about 350 mL to about 4,500 L, about 350 mL to about 4,000 L, about 350 mL to about 3,500 L, about 350 mL to about 3,000 L, about 350 mL to about 2,500 L, about 350 mL to about 2,000 L, about 350 mL to about 1,500 L, about 350 mL to about 1,000 L, about 350 mL to about 800 mL, about 350 mL to about 600 mL, about 350 mL to about 500 mL, about 350 mL to about 450 mL, about 350 mL to about 400 mL, about 400 mL to about 5,000 L, about 400 mL to about 4,500 L, about 400 mL to about 4,000 L, about 400 mL to about 3,500 L, about 400 mL to about 3,000 L, about 400 mL to about 2,500 L, about 400 mL to about 2,000 L, about 400 mL to about 1,500 L, about 400 mL to about 1,000 L, about 400 mL to about 800 mL, about 400 mL to about 600 mL, about 400 mL to about 500 mL, about 400 mL to about 450 mL, about 450 mL to about 5,000 L, about 450 mL to about 4,500 L, about 450 mL to about 4,000 L, about 450 mL to about 3,500 L, about 450 mL to about 3,000 L, about 450 mL to about 2,500 L, about 450 mL to about 2,000 L, about 450 mL to about 1,500 L, about 450 mL to about 1,000 L, about 450 mL to about 800 mL, about 450 mL to about 600 mL, about 450 mL to about 500 mL, about 500 mL to about 5,000 L, about 500 mL to about 4,500 L, about 500 mL to about 4,000 L, about 500 mL to about 3,500 L, about 500 mL to about 3,000 L, about 500 mL to about 2,500 L, about 500 mL to about 2,000 L, about 500 mL to about 1,500 L, about 500 mL to about 1,000 L, about 500 mL to about 800 mL, about 500 mL to about 600 mL, about 600 mL to about 5,000 L, about 600 mL to about 4,500 L, about 600 mL to about 4,000 L, about 600 mL to about 3,500 L, about 600 mL to about 3,000 L, about 600 mL to about 2,500 L, about 600 mL to about 2,000 L, about 600 mL to about 1,500 L, about 600 mL to about 1,000 L, about 600 mL to about 800 mL, about 800 mL to about 5,000 L, about 800 mL to about 4,500 L, about 800 mL to about 4,000 L, about 800 mL to about 3,500 L, about 800 mL to about 3,000 L, about 800 mL to about 2,500 L, about 800 mL to about 2,000 L, about 800 mL to about 1,500 L, about 800 mL to about 1,000 L, about 1,000 L to about 5,000 L, about 1,000 L to about 4,500 L, about 1,000 L to about 4,000 L, about 1,000 L to about 3,500 L, about 1,000 L to about 3,000 L, about 1,000 L to about 2,500 L, about 1,000 L to about 2,000 L, about 1,000 L to about 1,500 L, about 1,500 L to about 5,000 L, about 1,500 L to about 4,500 L, about 1,500 L to about 4,000 L, about 1,500 L to about 3,500 L, about 1,500 L to about 3,000 L, about 1,500 L to about 2,500 L, about 1,500 L to about 2,000 L, about 2,000 L to about 5,000 L, about 2,000 L to about 4,500 L, about 2,000 L to about 4,000 L, about 2,000 L to about 3,500 L, about 2,000 L to about 3,000 L, about 2,000 L to about 2,500 L, about 2,500 L to about 5,000 L, about 2,500 L to about 4,500 L, about 2,500 L to about 4,000 L, about 2,500 L to about 3,500 L, about 2,500 L to about 3,000 L, about 3,000 L to about 5,000 L, about 3,000 L to about 4,500 L, about 3,000 L to about 4,000 L, about 3,000 L to about 3,500 L, about 3,500 L to about 5,000 L, about 3,500 L to about 4,500 L, about 3,500 L to about 4,000 L, about 4,000 L to about 5,000 L, about 4,000 L to about 4,500 L, about 4,500 L to about 5,000 L.

In some embodiments, step (a) includes disposing a volume of about 10 μL to about 0.1 mL, about 10 μL to about 80 μL, about 10 μL to about 60 μL, about 10 μL to about 40 μL, about 10 μL to about 20 μL, about 20 μL to about 0.1 mL, about 20 μL to about 80 μL, about 20 μL to about 60 μL, about 20 μL to about 40 μL, about 40 μL to about 0.1 mL, about 40 μL to about 80 μL, about 40 μL to about 60 μL, about 60 μL to about 0.1 mL, about 60 μL to about 80 μL, or about 60 μL to about 0.1 mL.

In some embodiments, the vessel in step (a) is a bioreactor (e.g., a perfusion bioreactor) (e.g., any of the exemplary bioreactors described herein having any of the exemplary volumes described herein). Additional examples and aspects of bioreactors that can be used in step (a) are known in the art.

In some embodiments, the vessel in step (a) is a shake flask (e.g., any of the exemplary shake flasks described herein having any of the exemplary volumes described herein). Additional examples and aspects of shake flasks that can be used in step (a) are known in the art.

In some embodiments, the vessel in step (a) is a shake tube (e.g., any of the exemplary shake tubes described herein or known in the art). Additional examples and aspects of shake tubes that can be used in step (a) are known in the art. In some embodiments, the vessel in step (a) is a culture bag (e.g., any of the exemplary culture bags described herein).

In some examples, the second culture medium includes one or more (e.g., one, two, three, four, five, or six) of: transferrin (e.g., about 1 μg/mL to about 500 μg/mL transferrin (e.g., apotransferrin, holo transferrin, or a combination thereof) or any of the subranges of this range described herein), IL-3 (e.g., about 0.1 ng/mL to about 200 ng/mL IL-3 or any of the subranges of this range described herein), stem cell factor (SCF) (e.g., about 1 ng/mL to about 1 μg/mL SCF or any of the subranges of this range described herein), dexamethasone (e.g., about 0.1 nM to about 200 nM dexamethasone or any of the subranges of this range described herein), erythropoietin (EPO) or an EPO-mimetic peptide (e.g., about 1 ng/mL to about 500 ng/mL of EPO or an EPO-mimetic peptide or any of the subranges of this range described herein), and insulin (e.g., about 0.1 μg/mL to about 50 μg/mL insulin or any of the subranges of this range described herein). In some embodiments of any of the second culture media described herein, the second culture medium includes Iscove's modified Dulbecco's medium (IMDM). In some embodiments of any of the second culture media described herein, the second culture medium includes lipid (e.g., a lipid mixture). In some embodiments of any of the second culture media described herein, the second culture medium includes about 0.1 mM to about 10 mM (or any of the subranges of this range described herein) of L-glutamine, L-alanyl-L-glutamine, L-glycyl-L-glutamine, N-acetyl-L-glutamine, or a combination thereof. In some embodiments of any of the second culture media described herein, the second culture medium includes 0.1% w/v to about 4% w/v (e.g., about 0.1% w/v to about 3.5% w/v, about 0.1% w/v to about 3.0% w/v, about 0.1% w/v to about 2.5% w/v, about 0.1% w/v to about 2.0% w/v, about 0.1% w/v to about 1.5% w/v, about 0.1% w/v to about 1.0% w/v, about 0.1% w/v to about 0.5% w/v, about 0.2% w/v to about 4% w/v, about 0.2% w/v to about 3.5% w/v, about 0.2% w/v to about 3.0% w/v, about 0.2% w/v to about 2.5% w/v, about 0.2% w/v to about 2.0% w/v, about 0.2% w/v to about 1.5% w/v, about 0.2% w/v to about 1.0% w/v, about 0.2% w/v to about 0.5% w/v, about 0.5% w/v to about 4% w/v, about 0.5% w/v to about 3.5% w/v, about 0.5% w/v to about 3.0% w/v, about 0.5% w/v to about 2.5% w/v, about 0.5% w/v to about 2.0% w/v, about 0.5% w/v to about 1.5% w/v, about 0.5% w/v to about 1.0% w/v, about 1.0% w/v to about 4% w/v, about 1.0% w/v to about 3.5% w/v, about 1.0% w/v to about 3.0% w/v, about 1.0% w/v to about 2.5% w/v, about 1.0% w/v to about 2.0% w/v, about 1.0% w/v to about 1.5% w/v, about 1.5% w/v to about 4% w/v, about 1.5% w/v to about 3.5% w/v, about 1.5% w/v to about 3.0% w/v, about 1.5% w/v to about 2.5% w/v, about 1.5% w/v to about 2.0% w/v, about 2.0% w/v to about 4% w/v, about 2.0% w/v to about 3.5% w/v, about 2.0% w/v to about 3.0% w/v, about 2.0% w/v to about 2.5% w/v, about 2.5% w/v to about 4% w/v, about 2.5% w/v to about 3.5% w/v, about 2.5% w/v to about 3.0% w/v, about 3.0% w/v to about 4% w/v, about 3.0% w/v to about 3.5% w/v, or about 3.5% w/v to about 4.0% w/v) human serum albumin. In some embodiments of any of the second culture media described herein, the second culture medium includes about 0.1% w/v to about 3% w/v (e.g., about 0.1% w/v to about 2.5% w/v, about 0.1% w/v to about 2.0% w/v, about 0.1% w/v to about 1.5% w/v, about 0.1% w/v to about 1.0% w/v, about 0.1% w/v to about 0.5% w/v, about 0.5% w/v to about 3% w/v, about 0.5% w/v to about 2.5% w/v, about 0.5% w/v to about 2.0% w/v, about 0.5% w/v to about 1.5% w/v, about 0.5% w/v to about 1.0% w/v, about 1.0% w/v to about 3% w/v, about 1.0% w/v to about 2.5% w/v, about 1.0% w/v to about 2.0% w/v, about 1.0% w/v to about 1.5% w/v, about 1.5% w/v to about 3% w/v, about 1.5% w/v to about 2.5% w/v, about 1.5% w/v to about 2.0% w/v, about 2.0% w/v to about 3% w/v, about 2.0% w/v to about 2.5% w/v, or about 2.5% w/v to about 3% w/v) Poloxamer-188 (P188).

In some examples, the second culture medium comprises about 1 μg/mL to about 500 μg/mL, about 1 μg/mL to about 480 μg/mL, about 1 μg/mL to about 460 μg/mL, about 1 μg/mL to about 440 μg/mL, about 1 μg/mL to about 420 μg/mL, about 1 μg/mL to about 400 μg/mL, about 1 μg/mL to about 380 μg/mL, about 1 μg/mL to about 360 μg/mL, about 1 μg/mL to about 340 μg/mL, about 1 μg/mL to about 320 μg/mL, about 1 μg/mL to about 300 μg/mL, about 1 μg/mL to about 280 μg/mL, about 1 μg/mL to about 260 μg/mL, about 1 μg/mL to about 240 μg/mL, about 1 μg/mL to about 220 μg/mL, about 1 μg/mL to about 200 μg/mL, about 1 μg/mL to about 180 μg/mL, about 1 μg/mL to about 160 μg/mL, about 1 μg/mL to about 140 μg/mL, about 1 μg/mL to about 120 μg/mL, about 1 μg/mL to about 100 μg/mL, about 1 μg/mL to about 80 μg/mL, about 1 μg/mL to about 60 μg/mL, about 1 μg/mL to about 40 μg/mL, about 1 μg/mL to about 20 μg/mL, about 1 μg/mL to about 10 μg/mL, about 10 μg/mL to about 500 μg/mL, about 10 μg/mL to about 480 μg/mL, about 10 μg/mL to about 460 μg/mL, about 10 μg/mL to about 440 μg/mL, about 10 μg/mL to about 420 μg/mL, about 10 μg/mL to about 400 μg/mL, about 10 μg/mL to about 380 μg/mL, about 10 μg/mL to about 360 μg/mL, about 10 μg/mL to about 340 μg/mL, about 10 μg/mL to about 320 μg/mL, about 10 μg/mL to about 300 μg/mL, about 10 μg/mL to about 280 μg/mL, about 10 μg/mL to about 260 μg/mL, about 10 μg/mL to about 240 μg/mL, about 10 μg/mL to about 220 μg/mL, about 10 μg/mL to about 200 μg/mL, about 10 μg/mL to about 180 μg/mL, about 10 μg/mL to about 160 μg/mL, about 10 μg/mL to about 140 μg/mL, about 10 μg/mL to about 120 μg/mL, about 10 μg/mL to about 100 μg/mL, about 10 μg/mL to about 80 μg/mL, about 10 μg/mL to about 60 μg/mL, about 10 μg/mL to about 40 μg/mL, about 10 μg/mL to about 20 μg/mL, about 20 μg/mL to about 500 μg/mL, about 20 μg/mL to about 480 μg/mL, about 20 μg/mL to about 460 μg/mL, about 20 μg/mL to about 440 μg/mL, about 20 μg/mL to about 420 μg/mL, about 20 μg/mL to about 400 μg/mL, about 20 μg/mL to about 380 μg/mL, about 20 μg/mL to about 360 μg/mL, about 20 μg/mL to about 340 μg/mL, about 20 μg/mL to about 320 μg/mL, about 20 μg/mL to about 300 μg/mL, about 20 μg/mL to about 280 μg/mL, about 20 μg/mL to about 260 μg/mL, about 20 μg/mL to about 240 μg/mL, about 20 μg/mL to about 220 μg/mL, about 20 μg/mL to about 200 μg/mL, about 20 μg/mL to about 180 μg/mL, about 20 μg/mL to about 160 μg/mL, about 20 μg/mL to about 140 μg/mL, about 20 μg/mL to about 120 μg/mL, about 20 μg/mL to about 100 μg/mL, about 20 μg/mL to about 80 μg/mL, about 20 μg/mL to about 60 μg/mL, about 20 μg/mL to about 40 μg/mL, about 40 μg/mL to about 500 μg/mL, about 40 μg/mL to about 480 μg/mL, about 40 μg/mL to about 460 μg/mL, about 40 μg/mL to about 440 μg/mL, about 40 μg/mL to about 420 μg/mL, about 40 μg/mL to about 400 μg/mL, about 40 μg/mL to about 380 μg/mL, about 40 μg/mL to about 360 μg/mL, about 40 μg/mL to about 340 μg/mL, about 40 μg/mL to about 320 μg/mL, about 40 μg/mL to about 300 μg/mL, about 40 μg/mL to about 280 μg/mL, about 40 μg/mL to about 260 μg/mL, about 40 μg/mL to about 240 μg/mL, about 40 μg/mL to about 220 μg/mL, about 40 μg/mL to about 200 μg/mL, about 40 μg/mL to about 180 μg/mL, about 40 μg/mL to about 160 μg/mL, about 40 μg/mL to about 140 μg/mL, about 40 μg/mL to about 120 μg/mL, about 40 μg/mL to about 100 μg/mL, about 40 μg/mL to about 80 μg/mL, about 40 μg/mL to about 60 μg/mL, about 60 μg/mL to about 500 μg/mL, about 60 μg/mL to about 480 μg/mL, about 60 μg/mL to about 460 μg/mL, about 60 μg/mL to about 440 μg/mL, about 60 μg/mL to about 420 μg/mL, about 60 μg/mL to about 400 μg/mL, about 60 μg/mL to about 380 μg/mL, about 60 μg/mL to about 360 μg/mL, about 60 μg/mL to about 340 μg/mL, about 60 μg/mL to about 320 μg/mL, about 60 μg/mL to about 300 μg/mL, about 60 μg/mL to about 280 μg/mL, about 60 μg/mL to about 260 μg/mL, about 60 μg/mL to about 240 μg/mL, about 60 μg/mL to about 220 μg/mL, about 60 μg/mL to about 200 μg/mL, about 60 μg/mL to about 180 μg/mL, about 60 μg/mL to about 160 μg/mL, about 60 μg/mL to about 140 μg/mL, about 60 μg/mL to about 120 μg/mL, about 60 μg/mL to about 100 μg/mL, about 60 μg/mL to about 80 μg/mL, about 80 μg/mL to about 500 μg/mL, about 80 μg/mL to about 480 μg/mL, about 80 μg/mL to about 460 μg/mL, about 80 μg/mL to about 440 μg/mL, about 80 μg/mL to about 420 μg/mL, about 80 μg/mL to about 400 μg/mL, about 80 μg/mL to about 380 μg/mL, about 80 μg/mL to about 360 μg/mL, about 80 μg/mL to about 340 μg/mL, about 80 μg/mL to about 320 μg/mL, about 80 μg/mL to about 300 μg/mL, about 80 μg/mL to about 280 μg/mL, about 80 μg/mL to about 260 μg/mL, about 80 μg/mL to about 240 μg/mL, about 80 μg/mL to about 220 μg/mL, about 80 μg/mL to about 200 μg/mL, about 80 μg/mL to about 180 μg/mL, about 80 μg/mL to about 160 μg/mL, about 80 μg/mL to about 140 μg/mL, about 80 μg/mL to about 120 μg/mL, about 80 μg/mL to about 100 μg/mL, about 100 μg/mL to about 500 μg/mL, about 100 μg/mL to about 480 μg/mL, about 100 μg/mL to about 460 μg/mL, about 100 μg/mL to about 440 μg/mL, about 100 μg/mL to about 420 μg/mL, about 100 μg/mL to about 400 μg/mL, about 100 μg/mL to about 380 μg/mL, about 100 μg/mL to about 360 μg/mL, about 100 μg/mL to about 340 μg/mL, about 100 μg/mL to about 320 μg/mL, about 100 μg/mL to about 300 μg/mL, about 100 μg/mL to about 280 μg/mL, about 100 μg/mL to about 260 μg/mL, about 100 μg/mL to about 240 μg/mL, about 100 μg/mL to about 220 μg/mL, about 100 μg/mL to about 200 μg/mL, about 100 μg/mL to about 180 μg/mL, about 100 μg/mL to about 160 μg/mL, about 100 μg/mL to about 140 μg/mL, about 100 μg/mL to about 120 μg/mL, about 120 μg/mL to about 500 μg/mL, about 120 μg/mL to about 480 μg/mL, about 120 μg/mL to about 460 μg/mL, about 120 μg/mL to about 440 μg/mL, about 120 μg/mL to about 420 μg/mL, about 120 μg/mL to about 400 μg/mL, about 120 μg/mL to about 380 μg/mL, about 120 μg/mL to about 360 μg/mL, about 120 μg/mL to about 340 μg/mL, about 120 μg/mL to about 320 μg/mL, about 120 μg/mL to about 300 μg/mL, about 120 μg/mL to about 280 μg/mL, about 120 μg/mL to about 260 μg/mL, about 120 μg/mL to about 240 μg/mL, about 120 μg/mL to about 220 μg/mL, about 120 μg/mL to about 200 μg/mL, about 120 μg/mL to about 180 μg/mL, about 120 μg/mL to about 160 μg/mL, about 120 μg/mL to about 140 μg/mL, about 140 μg/mL to about 500 μg/mL, about 140 μg/mL to about 480 μg/mL, about 140 μg/mL to about 460 μg/mL, about 140 μg/mL to about 440 μg/mL, about 140μg/mL to about 420 μg/mL, about 140 μg/mL to about 400 μg/mL, about 140 μg/mL to about 380 μg/mL, about 140 μg/mL to about 360 μg/mL, about 140 μg/mL to about 340 μg/mL, about 140 μg/mL to about 320 μg/mL, about 140 μg/mL to about 300 μg/mL, about 140 μg/mL to about 280 μg/mL, about 140 μg/mL to about 260 μg/mL, about 140 μg/mL to about 240 μg/mL, about 140 μg/mL to about 220 μg/mL, about 140 μg/mL to about 200 μg/mL, about 140 μg/mL to about 180 μg/mL, about 140 μg/mL to about 160 μg/mL, about 160 μg/mL to about 500 μg/mL, about 160 μg/mL to about 480 μg/mL, about 160 μg/mL to about 460 μg/mL, about 160 μg/mL to about 440 μg/mL, about 160 μg/mL to about 420 μg/mL, about 160 μg/mL to about 400 μg/mL, about 160 μg/mL to about 380 μg/mL, about 160 μg/mL to about 360 μg/mL, about 160 μg/mL to about 340 μg/mL, about 160 μg/mL to about 320 μg/mL, about 160 μg/mL to about 300 μg/mL, about 160 μg/mL to about 280 μg/mL, about 160 μg/mL to about 260 μg/mL, about 160 μg/mL to about 240 μg/mL, about 160 μg/mL to about 220 μg/mL, about 160 μg/mL to about 200 μg/mL, about 160 μg/mL to about 180 μg/mL, about 180 μg/mL to about 500 μg/mL, about 180 μg/mL to about 480 μg/mL, about 180 μg/mL to about 460 μg/mL, about 180 μg/mL to about 440 μg/mL, about 180 μg/mL to about 420 μg/mL, about 180 μg/mL to about 400 μg/mL, about 180 μg/mL to about 380 μg/mL, about 180 μg/mL to about 360 μg/mL, about 180 μg/mL to about 340 μg/mL, about 180 μg/mL to about 320 μg/mL, about 180 μg/mL to about 300 μg/mL, about 180 μg/mL to about 280 μg/mL, about 180 μg/mL to about 260 μg/mL, about 180 μg/mL to about 240 μg/mL, about 180 μg/mL to about 220 μg/mL, about 180 μg/mL to about 200 μg/mL, about 200 μg/mL to about 500 μg/mL, about 200 μg/mL to about 480 μg/mL, about 200 μg/mL to about 460 μg/mL, about 200 μg/mL to about 440 μg/mL, about 200 μg/mL to about 420 μg/mL, about 200 μg/mL to about 400 μg/mL, about 200 μg/mL to about 380 μg/mL, about 200 μg/mL to about 360 μg/mL, about 200 μg/mL to about 340 μg/mL, about 200 μg/mL to about 320 μg/mL, about 200 μg/mL to about 300 μg/mL, about 200 μg/mL to about 280 μg/mL, about 200 μg/mL to about 260 μg/mL, about 200 μg/mL to about 240 μg/mL, about 200 μg/mL to about 220 μg/mL, about 220 μg/mL to about 500 μg/mL, about 220 μg/mL to about 480 μg/mL, about 220 μg/mL to about 460 μg/mL, about 220 μg/mL to about 440 μg/mL, about 220 μg/mL to about 420 μg/mL, about 220 μg/mL to about 400 μg/mL, about 220 μg/mL to about 380 μg/mL, about 220 μg/mL to about 360 μg/mL, about 220 μg/mL to about 340 μg/mL, about 220 μg/mL to about 320 μg/mL, about 220 μg/mL to about 300 μg/mL, about 220 μg/mL to about 280 μg/mL, about 220 μg/mL to about 260 μg/mL, about 220 μg/mL to about 240 μg/mL, about 240 μg/mL to about 500 μg/mL, about 240 μg/mL to about 480 μg/mL, about 240 μg/mL to about 460 μg/mL, about 240 μg/mL to about 440 μg/mL, about 240 μg/mL to about 420 μg/mL, about 240 μg/mL to about 400 μg/mL, about 240 μg/mL to about 380 μg/mL, about 240 μg/mL to about 360 μg/mL, about 240 μg/mL to about 340 μg/mL, about 240 μg/mL to about 320 μg/mL, about 240 μg/mL to about 300 μg/mL, about 240 μg/mL to about 280 μg/mL, about 240 μg/mL to about 260 μg/mL, about 260 μg/mL to about 500 μg/mL, about 260 μg/mL to about 480 μg/mL, about 260 μg/mL to about 460 μg/mL, about 260 μg/mL to about 440 μg/mL, about 260 μg/mL to about 420 μg/mL, about 260 μg/mL to about 400 μg/mL, about 260 μg/mL to about 380 μg/mL, about 260 μg/mL to about 360 μg/mL, about 260 μg/mL to about 340 μg/mL, about 260 μg/mL to about 320 μg/mL, about 260 μg/mL to about 300 μg/mL, about 260 μg/mL to about 280 μg/mL, about 280 μg/mL to about 500 μg/mL, about 280 μg/mL to about 480 μg/mL, about 280 μg/mL to about 460 μg/mL, about 280 μg/mL to about 440 μg/mL, about 280 μg/mL to about 420 μg/mL, about 280 μg/mL to about 400 μg/mL, about 280 μg/mL to about 380 μg/mL, about 280 μg/mL to about 360 μg/mL, about 280 μg/mL to about 340 μg/mL, about 280 μg/mL to about 320 μg/mL, about 280 μg/mL to about 300 μg/mL, about 300 μg/mL to about 500 μg/mL, about 300 μg/mL to about 480 μg/mL, about 300 μg/mL to about 460 μg/mL, about 300 μg/mL to about 440 μg/mL, about 300 μg/mL to about 420 μg/mL, about 300 μg/mL to about 400 μg/mL, about 300 μg/mL to about 380 μg/mL, about 300 μg/mL to about 360 μg/mL, about 300 μg/mL to about 340 μg/mL, about 300 μg/mL to about 320 μg/mL, about 320 μg/mL to about 500 μg/mL, about 320 μg/mL to about 480 μg/mL, about 320 μg/mL to about 460 μg/mL, about 320 μg/mL to about 440 μg/mL, about 320 μg/mL to about 420 μg/mL, about 320 μg/mL to about 400 μg/mL, about 320 μg/mL to about 380 μg/mL, about 320 μg/mL to about 360 μg/mL, about 320 μg/mL to about 340 μg/mL, about 340 μg/mL to about 500 μg/mL, about 340 μg/mL to about 480 μg/mL, about 340 μg/mL to about 460 μg/mL, about 340 μg/mL to about 440 μg/mL, about 340 μg/mL to about 420 μg/mL, about 340 μg/mL to about 400 μg/mL, about 340 μg/mL to about 380 μg/mL, about 340 μg/mL to about 360 μg/mL, about 360 μg/mL to about 500 μg/mL, about 360 μg/mL to about 480 μg/mL, about 360 μg/mL to about 460 μg/mL, about 360 μg/mL to about 440 μg/mL, about 360 μg/mL to about 420 μg/mL, about 360 μg/mL to about 400 μg/mL, about 360 μg/mL to about 380 μg/mL, about 380 μg/mL to about 500 μg/mL, about 380 μg/mL to about 480 μg/mL, about 380 μg/mL to about 460 μg/mL, about 380 μg/mL to about 440 μg/mL, about 380 μg/mL to about 420 μg/mL, about 380 μg/mL to about 400 μg/mL, about 400 μg/mL to about 500 μg/mL, about 400 μg/mL to about 480 μg/mL, about 400 μg/mL to about 460 μg/mL, about 400 μg/mL to about 440 μg/mL, about 400 μg/mL to about 420 μg/mL, about 420 μg/mL to about 500 μg/mL, about 420 μg/mL to about 480 μg/mL, about 420 μg/mL to about 460 μg/mL, about 420 μg/mL to about 440 μg/mL, about 440 μg/mL to about 500 μg/mL, about 440 μg/mL to about 480 μg/mL, about 440 μg/mL to about 460 μg/mL, about 460 μg/mL to about 500 μg/mL, about 460 μg/mL to about 480 μg/mL, or about 480 μg/mL to about 500 μg/mL, of transferrin (e.g., human apotransferrin, human holo transferrin, or a combination thereof).

In some embodiments, the second culture medium comprises about 0.1 ng/mL to about 200 ng/mL, about 0.1 ng/mL to about 180 ng/mL, about 0.1 ng/mL to about 160 ng/mL, about 0.1 ng/mL to about 140 ng/mL, about 0.1 ng/mL to about 120 ng/mL, about 0.1 ng/mL to about 100 ng/mL, about 0.1 ng/mL to about 80 ng/mL, about 0.1 ng/mL to about 60 ng/mL, about 0.1 ng/mL to about 40 ng/mL, about 0.1 ng/mL to about 20 ng/mL, about 0.1 ng/mL to about 10 ng/mL, about 0.1 ng/mL to about 5 ng/mL, about 5 ng/mL to about 200 ng/mL, about 5 ng/mL to about 180 ng/mL, about 5 ng/mL to about 160 ng/mL, about 5 ng/mL to about 140 ng/mL, about 5 ng/mL to about 120 ng/mL, about 5 ng/mL to about 100 ng/mL, about 5 ng/mL to about 80 ng/mL, about 5 ng/mL to about 60 ng/mL, about 5 ng/mL to about 40 ng/mL, about 5 ng/mL to about 20 ng/mL, about 5 ng/mL to about 10 ng/mL, about 10 ng/mL to about 200 ng/mL, about 10 ng/mL to about 180 ng/mL, about 10 ng/mL to about 160 ng/mL, about 10 ng/mL to about 140 ng/mL, about 10 ng/mL to about 120 ng/mL, about 10 ng/mL to about 100 ng/mL, about 10 ng/mL to about 80 ng/mL, about 10 ng/mL to about 60 ng/mL, about 10 ng/mL to about 40 ng/mL, about 10 ng/mL to about 20 ng/mL, about 20 ng/mL to about 200 ng/mL, about 20 ng/mL to about 180 ng/mL, about 20 ng/mL to about 160 ng/mL, about 20 ng/mL to about 140 ng/mL, about 20 ng/mL to about 120 ng/mL, about 20 ng/mL to about 100 ng/mL, about 20 ng/mL to about 80 ng/mL, about 20 ng/mL to about 60 ng/mL, about 20 ng/mL to about 40 ng/mL, about 40 ng/mL to about 200 ng/mL, about 40 ng/mL to about 180 ng/mL, about 40 ng/mL to about 160 ng/mL, about 40 ng/mL to about 140 ng/mL, about 40 ng/mL to about 120 ng/mL, about 40 ng/mL to about 100 ng/mL, about 40 ng/mL to about 80 ng/mL, about 40 ng/mL to about 60 ng/mL, about 60 ng/mL to about 200 ng/mL, about 60 ng/mL to about 180 ng/mL, about 60 ng/mL to about 160 ng/mL, about 60 ng/mL to about 140 ng/mL, about 60 ng/mL to about 120 ng/mL, about 60 ng/mL to about 100 ng/mL, about 60 ng/mL to about 80 ng/mL, about 80 ng/mL to about 200 ng/mL, about 80 ng/mL to about 180 ng/mL, about 80 ng/mL to about 160 ng/mL, about 80 ng/mL to about 140 ng/mL, about 80 ng/mL to about 120 ng/mL, about 80 ng/mL to about 100 ng/mL, about 100 ng/mL to about 200 ng/mL, about 100 ng/mL to about 180 ng/mL, about 100 ng/mL to about 160 ng/mL, about 100 ng/mL to about 140 ng/mL, about 100 ng/mL to about 120 ng/mL, about 120 ng/mL to about 200 ng/mL, about 120 ng/mL to about 180 ng/mL, about 120 ng/mL to about 160 ng/mL, about 120 ng/mL to about 140 ng/mL, about 140 ng/mL to about 200 ng/mL, about 140 ng/mL to about 180 ng/mL, about 140 ng/mL to about 160 ng/mL, about 160 ng/mL to about 200 ng/mL, about 160 ng/mL to about 180 ng/mL, or about 180 ng/mL to about 200 ng/mL of IL-3 (e.g., human IL-3).

In some embodiments, the second culture medium comprises about 1 ng/mL to about 1 μg/mL, about 1 ng/mL to about 950 ng/mL, about 1 ng/mL to about 900 ng/mL, about 1 ng/mL to about 850 ng/mL, about 1 ng/mL to about 800 ng/mL, about 1 ng/mL to about 750 ng/mL, about 1 ng/mL to about 700 ng/mL, about 1 ng/mL to about 650 ng/mL, about 1 ng/mL to about 600 ng/mL, about 1 ng/mL to about 550 ng/mL, about 1 ng/mL to about 500 ng/mL, about 1 ng/mL to about 450 ng/mL, about 1 ng/mL to about 400 ng/mL, about 1 ng/mL to about 350 ng/mL, about 1 ng/mL to about 300 ng/mL, about 1 ng/mL to about 250 ng/mL, about 1 ng/mL to about 200 ng/mL, about 1 ng/mL to about 150 ng/mL, about 1 ng/mL to about 100 ng/mL, about 1 ng/mL to about 80 ng/mL, about 1 ng/mL to about 60 ng/mL, about 1 ng/mL to about 40 ng/mL, about 1 ng/mL to about 20 ng/mL, about 1 ng/mL to about 10 ng/mL, about 10 ng/mL to about 1 μg/mL, about 10 ng/mL to about 950 ng/mL, about 10 ng/mL to about 900 ng/mL, about 10 ng/mL to about 850 ng/mL, about 10 ng/mL to about 800 ng/mL, about 10 ng/mL to about 750 ng/mL, about 10 ng/mL to about 700 ng/mL, about 10 ng/mL to about 650 ng/mL, about 10 ng/mL to about 600 ng/mL, about 10 ng/mL to about 550 ng/mL, about 10 ng/mL to about 500 ng/mL, about 10 ng/mL to about 450 ng/mL, about 10 ng/mL to about 400 ng/mL, about 10 ng/mL to about 350 ng/mL, about 10 ng/mL to about 300 ng/mL, about 10 ng/mL to about 250 ng/mL, about 10 ng/mL to about 200 ng/mL, about 10 ng/mL to about 150 ng/mL, about 10 ng/mL to about 100 ng/mL, about 10 ng/mL to about 80 ng/mL, about 10 ng/mL to about 60 ng/mL, about 10 ng/mL to about 40 ng/mL, about 10 ng/mL to about 20 ng/mL, about 20 ng/mL to about 1 μg/mL, about 20 ng/mL to about 950 ng/mL, about 20 ng/mL to about 900 ng/mL, about 20 ng/mL to about 850 ng/mL, about 20 ng/mL to about 800 ng/mL, about 20 ng/mL to about 750 ng/mL, about 20 ng/mL to about 700 ng/mL, about 20 ng/mL to about 650 ng/mL, about 20 ng/mL to about 600 ng/mL, about 20 ng/mL to about 550 ng/mL, about 20 ng/mL to about 500 ng/mL, about 20 ng/mL to about 450 ng/mL, about 20 ng/mL to about 400 ng/mL, about 20 ng/mL to about 350 ng/mL, about 20 ng/mL to about 300 ng/mL, about 20 ng/mL to about 250 ng/mL, about 20 ng/mL to about 200 ng/mL, about 20 ng/mL to about 150 ng/mL, about 20 ng/mL to about 100 ng/mL, about 20 ng/mL to about 80 ng/mL, about 20 ng/mL to about 60 ng/mL, about 20 ng/mL to about 40 ng/mL, about 40 ng/mL to about 1 μg/mL, about 40 ng/mL to about 950 ng/mL, about 40 ng/mL to about 900 ng/mL, about 40 ng/mL to about 850 ng/mL, about 40 ng/mL to about 800 ng/mL, about 40 ng/mL to about 750 ng/mL, about 40 ng/mL to about 700 ng/mL, about 40 ng/mL to about 650 ng/mL, about 40 ng/mL to about 600 ng/mL, about 40 ng/mL to about 550 ng/mL, about 40 ng/mL to about 500 ng/mL, about 40 ng/mL to about 450 ng/mL, about 40 ng/mL to about 400 ng/mL, about 40 ng/mL to about 350 ng/mL, about 40 ng/mL to about 300 ng/mL, about 40 ng/mL to about 250 ng/mL, about 40 ng/mL to about 200 ng/mL, about 40 ng/mL to about 150 ng/mL, about 40 ng/mL to about 100 ng/mL, about 40 ng/mL to about 80 ng/mL, about 40 ng/mL to about 60 ng/mL, about 60 ng/mL to about 1 μg/mL, about 60 ng/mL to about 950 ng/mL, about 60 ng/mL to about 900 ng/mL, about 60 ng/mL to about 850 ng/mL, about 60 ng/mL to about 800 ng/mL, about 60 ng/mL to about 750 ng/mL, about 60 ng/mL to about 700 ng/mL, about 60 ng/mL to about 650 ng/mL, about 60 ng/mL to about 600 ng/mL, about 60 ng/mL to about 550 ng/mL, about 60 ng/mL to about 500 ng/mL, about 60 ng/mL to about 450 ng/mL, about 60 ng/mL to about 400 ng/mL, about 60 ng/mL to about 350 ng/mL, about 60 ng/mL to about 300 ng/mL, about 60 ng/mL to about 250 ng/mL, about 60 ng/mL to about 200 ng/mL, about 60 ng/mL to about 150 ng/mL, about 60 ng/mL to about 100 ng/mL, about 60 ng/mL to about 80 ng/mL, about 80 ng/mL to about 1 μg/mL, about 80 ng/mL to about 950 ng/mL, about 80 ng/mL to about 900 ng/mL, about 80 ng/mL to about 850 ng/mL, about 80 ng/mL to about 800 ng/mL, about 80 ng/mL to about 750 ng/mL, about 80 ng/mL to about 700 ng/mL, about 80 ng/mL to about 650 ng/mL, about 80 ng/mL to about 600 ng/mL, about 80 ng/mL to about 550 ng/mL, about 80 ng/mL to about 500 ng/mL, about 80 ng/mL to about 450 ng/mL, about 80 ng/mL to about 400 ng/mL, about 80 ng/mL to about 350 ng/mL, about 80 ng/mL to about 300 ng/mL, about 80 ng/mL to about 250 ng/mL, about 80 ng/mL to about 200 ng/mL, about 80 ng/mL to about 150 ng/mL, about 80 ng/mL to about 100 ng/mL, about 100 ng/mL to about 1 μg/mL, about 100 ng/mL to about 950 ng/mL, about 100 ng/mL to about 900 ng/mL, about 100 ng/mL to about 850 ng/mL, about 100 ng/mL to about 800 ng/mL, about 100 ng/mL to about 750 ng/mL, about 100 ng/mL to about 700 ng/mL, about 100 ng/mL to about 650 ng/mL, about 100 ng/mL to about 600 ng/mL, about 100 ng/mL to about 550 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 100 ng/mL to about 250 ng/mL, about 100 ng/mL to about 200 ng/mL, about 100 ng/mL to about 150 ng/mL, about 150 ng/mL to about 1 μg/mL, about 150 ng/mL to about 950 ng/mL, about 150 ng/mL to about 900 ng/mL, about 150 ng/mL to about 850 ng/mL, about 150 ng/mL to about 800 ng/mL, about 150 ng/mL to about 750 ng/mL, about 150 ng/mL to about 700 ng/mL, about 150 ng/mL to about 650 ng/mL, about 150 ng/mL to about 600 ng/mL, about 150 ng/mL to about 550 ng/mL, about 150 ng/mL to about 500 ng/mL, about 150 ng/mL to about 450 ng/mL, about 150 ng/mL to about 400 ng/mL, about 150 ng/mL to about 350 ng/mL, about 150 ng/mL to about 300 ng/mL, about 150 ng/mL to about 250 ng/mL, about 150 ng/mL to about 200 ng/mL, about 200 ng/mL to about 1 μg/mL, about 200 ng/mL to about 950 ng/mL, about 200 ng/mL to about 900 ng/mL, about 200 ng/mL to about 850 ng/mL, about 200 ng/mL to about 800 ng/mL, about 200 ng/mL to about 750 ng/mL, about 200 ng/mL to about 700 ng/mL, about 200 ng/mL to about 650 ng/mL, about 200 ng/mL to about 600 ng/mL, about 200 ng/mL to about 550 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 200 ng/mL to about 250 ng/mL, about 250 ng/mL to about 1 μg/mL, about 250 ng/mL to about 950 ng/mL, about 250 ng/mL to about 900 ng/mL, about 250 ng/mL to about 850 ng/mL, about 250 ng/mL to about 800 ng/mL, about 250 ng/mL to about 750 ng/mL, about 250 ng/mL to about 700 ng/mL, about 250 ng/mL to about 650 ng/mL, about 250 ng/mL to about 600 ng/mL, about 250 ng/mL to about 550 ng/mL, about 250 ng/mL to about 500 ng/mL, about 250 ng/mL to about 450 ng/mL, about 250 ng/mL to about 400 ng/mL, about 250 ng/mL to about 350 ng/mL, about 250 ng/mL to about 300 ng/mL, about 300 ng/mL to about 1 μg/mL, about 300 ng/mL to about 950 ng/mL, about 300 ng/mL to about 900 ng/mL, about 300 ng/mL to about 850 ng/mL, about 300 ng/mL to about 800 ng/mL, about 300 ng/mL to about 750 ng/mL, about 300 ng/mL to about 700 ng/mL, about 300 ng/mL to about 650 ng/mL, about 300 ng/mL to about 600 ng/mL, about 300 ng/mL to about 550 ng/mL, about 300 ng/mL to about 500 ng/mL, about 300 ng/mL to about 450 ng/mL, about 300 ng/mL to about 400 ng/mL, about 300 ng/mL to about 350 ng/mL, about 350 ng/mL to about 1 μg/mL, about 350 ng/mL to about 950 ng/mL, about 350 ng/mL to about 900 ng/mL, about 350 ng/mL to about 850 ng/mL, about 350 ng/mL to about 800 ng/mL, about 350 ng/mL to about 750 ng/mL, about 350 ng/mL to about 700 ng/mL, about 350 ng/mL to about 650 ng/mL, about 350 ng/mL to about 600 ng/mL, about 350 ng/mL to about 550 ng/mL, about 350 ng/mL to about 500 ng/mL, about 350 ng/mL to about 450 ng/mL, about 350 ng/mL to about 400 ng/mL, about 400 ng/mL to about 1 μg/mL, about 400 ng/mL to about 950 ng/mL, about 400 ng/mL to about 900 ng/mL, about 400 ng/mL to about 850 ng/mL, about 400 ng/mL to about 800 ng/mL, about 400 ng/mL to about 750 ng/mL, about 400 ng/mL to about 700 ng/mL, about 400 ng/mL to about 650 ng/mL, about 400 ng/mL to about 600 ng/mL, about 400 ng/mL to about 550 ng/mL, about 400 ng/mL to about 500 ng/mL, about 400 ng/mL to about 450 ng/mL, about 450 ng/mL to about 1 μg/mL, about 450 ng/mL to about 950 ng/mL, about 450 ng/mL to about 900 ng/mL, about 450 ng/mL to about 850 ng/mL, about 450 ng/mL to about 800 ng/mL, about 450 ng/mL to about 750 ng/mL, about 450 ng/mL to about 700 ng/mL, about 450 ng/mL to about 650 ng/mL, about 450 ng/mL to about 600 ng/mL, about 450 ng/mL to about 550 ng/mL, about 450 ng/mL to about 500 ng/mL, about 500 ng/mL to about 1 μg/mL, about 500 ng/mL to about 950 ng/mL, about 500 ng/mL to about 900 ng/mL, about 500 ng/mL to about 850 ng/mL, about 500 ng/mL to about 800 ng/mL, about 500 ng/mL to about 750 ng/mL, about 500 ng/mL to about 700 ng/mL, about 500 ng/mL to about 650 ng/mL, about 500 ng/mL to about 600 ng/mL, about 500 ng/mL to about 550 ng/mL, about 550 ng/mL to about 1 μg/mL, about 550 ng/mL to about 950 ng/mL, about 550 ng/mL to about 900 ng/mL, about 550 ng/mL to about 850 ng/mL, about 550 ng/mL to about 800 ng/mL, about 550 ng/mL to about 750 ng/mL, about 550 ng/mL to about 700 ng/mL, about 550 ng/mL to about 650 ng/mL, about 550 ng/mL to about 600 ng/mL, about 600 ng/mL to about 1 μg/mL, about 600 ng/mL to about 950 ng/mL, about 600 ng/mL to about 900 ng/mL, about 600 ng/mL to about 850 ng/mL, about 600 ng/mL to about 800 ng/mL, about 600 ng/mL to about 750 ng/mL, about 600 ng/mL to about 700 ng/mL, about 600 ng/mL to about 650 ng/mL, about 650 ng/mL to about 1 μg/mL, about 650 ng/mL to about 950 ng/mL, about 650 ng/mL to about 900 ng/mL, about 650 ng/mL to about 850 ng/mL, about 650 ng/mL to about 800 ng/mL, about 650 ng/mL to about 750 ng/mL, about 650 ng/mL to about 700 ng/mL, about 700 ng/mL to about 1 μg/mL, about 700 ng/mL to about 950 ng/mL, about 700 ng/mL to about 900 ng/mL, about 700 ng/mL to about 850 ng/mL, about 700 ng/mL to about 800 ng/mL, about 700 ng/mL to about 750 ng/mL, about 750 ng/mL to about 1 μg/mL, about 750 ng/mL to about 950 ng/mL, about 750 ng/mL to about 900 ng/mL, about 750 ng/mL to about 850 ng/mL, about 750 ng/mL to about 800 ng/mL, about 800 ng/mL to about 1 μg/mL, about 800 ng/mL to about 950 ng/mL, about 800 ng/mL to about 900 ng/mL, about 800 ng/mL to about 850 ng/mL, about 850 ng/mL to about 1 μg/mL, about 850 ng/mL to about 950 ng/mL, about 850 ng/mL to about 900 ng/mL, about 900 ng/mL to about 1 μg/mL, about 900 ng/mL to about 950 ng/mL, about 950 ng/mL to about 1 μg/mL, of SCF (e.g., human stem cell factor).

In some embodiments, the second culture medium comprises about 0.1 nM to about 200 nM, about 0.1 nM to about 180 nM, about 0.1 nM to about 160 nM, about 0.1 nM to about 140 nM, about 0.1 nM to about 120 nM, about 0.1 nM to about 100 nM, about 0.1 nM to about 80 nM, about 0.1 nM to about 60 nM, about 0.1 nM to about 50 nM, about 0.1 nM to about 40 nM, about 0.1 nM to about 30 nM, about 0.1 nM to about 25 nM, about 0.1 nM to about 20 nM, about 0.1 nM to about 15 nM, about 0.1 nM to about 10 nM, about 0.1 nM to about 5 nM, about 0.1 nM to about 2 nM, about 0.1 nM to about 1 nM, about 1 nM to about 200 nM, about 1 nM to about 180 nM, about 1 nM to about 160 nM, about 1 nM to about 140 nM, about 1 nM to about 120 nM, about 1 nM to about 100 nM, about 1 nM to about 80 nM, about 1 nM to about 60 nM, about 1 nM to about 50 nM, about 1 nM to about 40 nM, about 1 nM to about 30 nM, about 1 nM to about 25 nM, about 1 nM to about 20 nM, about 1 nM to about 15 nM, about 1 nM to about 10 nM, about 1 nM to about 5 nM, about 1 nM to about 2 nM, about 2 nM to about 200 nM, about 2 nM to about 180 nM, about 2 nM to about 160 nM, about 2 nM to about 140 nM, about 2 nM to about 120 nM, about 2 nM to about 100 nM, about 2 nM to about 80 nM, about 2 nM to about 60 nM, about 2 nM to about 50 nM, about 2 nM to about 40 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 5 nM to about 200 nM, about 5 nM to about 180 nM, about 5 nM to about 160 nM, about 5 nM to about 140 nM, about 5 nM to about 120 nM, about 5 nM to about 100 nM, about 5 nM to about 80 nM, about 5 nM to about 60 nM, about 5 nM to about 50 nM, about 5 nM to about 40 nM, about 5 nM to about 30 nM, about 5 nM to about 25 nM, about 5 nM to about 20 nM, about 5 nM to about 15 nM, about 5 nM to about 10 nM, about 10 nM to about 200 nM, about 10 nM to about 180 nM, about 10 nM to about 160 nM, about 10 nM to about 140 nM, about 10 nM to about 120 nM, about 10 nM to about 100 nM, about 10 nM to about 80 nM, about 10 nM to about 60 nM, about 10 nM to about 50 nM, about 10 nM to about 40 nM, about 10 nM to about 30 nM, about 10 nM to about 25 nM, about 10 nM to about 20 nM, about 10 nM to about 15 nM, about 15 nM to about 200 nM, about 15 nM to about 180 nM, about 15 nM to about 160 nM, about 15 nM to about 140 nM, about 15 nM to about 120 nM, about 15 nM to about 100 nM, about 15 nM to about 80 nM, about 15 nM to about 60 nM, about 15 nM to about 50 nM, about 15 nM to about 40 nM, about 15 nM to about 30 nM, about 15 nM to about 25 nM, about 15 nM to about 20 nM, about 20 nM to about 200 nM, about 20 nM to about 180 nM, about 20 nM to about 160 nM, about 20 nM to about 140 nM, about 20 nM to about 120 nM, about 20 nM to about 100 nM, about 20 nM to about 80 nM, about 20 nM to about 60 nM, about 20 nM to about 50 nM, about 20 nM to about 40 nM, about 20 nM to about 30 nM, about 20 nM to about 25 nM, about 25 nM to about 200 nM, about 25 nM to about 180 nM, about 25 nM to about 160 nM, about 25 nM to about 140 nM, about 25 nM to about 120 nM, about 25 nM to about 100 nM, about 25 nM to about 80 nM, about 25 nM to about 60 nM, about 25 nM to about 50 nM, about 25 nM to about 40 nM, about 25 nM to about 30 nM, about 30 nM to about 200 nM, about 30 nM to about 180 nM, about 30 nM to about 160 nM, about 30 nM to about 140 nM, about 30 nM to about 120 nM, about 30 nM to about 100 nM, about 30 nM to about 80 nM, about 30 nM to about 60 nM, about 30 nM to about 50 nM, about 30 nM to about 40 nM, about 40 nM to about 200 nM, about 40 nM to about 180 nM, about 40 nM to about 160 nM, about 40 nM to about 140 nM, about 40 nM to about 120 nM, about 40 nM to about 100 nM, about 40 nM to about 80 nM, about 40 nM to about 60 nM, about 40 nM to about 50 nM, about 50 nM to about 200 nM, about 50 nM to about 180 nM, about 50 nM to about 160 nM, about 50 nM to about 140 nM, about 50 nM to about 120 nM, about 50 nM to about 100 nM, about 50 nM to about 80 nM, about 50 nM to about 60 nM, about 60 nM to about 200 nM, about 60 nM to about 180 nM, about 60 nM to about 160 nM, about 60 nM to about 140 nM, about 60 nM to about 120 nM, about 60 nM to about 100 nM, about 60 nM to about 80 nM, about 80 nM to about 200 nM, about 80 nM to about 180 nM, about 80 nM to about 160 nM, about 80 nM to about 140 nM, about 80 nM to about 120 nM, about 80 nM to about 100 nM, about 100 nM to about 200 nM, about 100 nM to about 180 nM, about 100 nM to about 160 nM, about 100 nM to about 140 nM, about 100 nM to about 120 nM, about 120 nM to about 200 nM, about 120 nM to about 180 nM, about 120 nM to about 160 nM, about 120 nM to about 140 nM, about 140 nM to about 200 nM, about 140 nM to about 180 nM, about 140 nM to about 160 nM, about 160 nM to about 200 nM, about 160 nM to about 180 nM, or about 180 nM to about 200 nM of dexamethasone.

In some embodiments, the second culture medium comprises about 1 ng/mL to about 500 ng/mL, about 1 ng/mL to about 480 ng/mL, about 1 ng/mL to about 460 ng/mL, about 1 ng/mL to about 440 ng/mL, about 1 ng/mL to about 420 ng/mL, about 1 ng/mL to about 400 ng/mL, about 1 ng/mL to about 380 ng/mL, about 1 ng/mL to about 360 ng/mL, about 1 ng/mL to about 340 ng/mL, about 1 ng/mL to about 320 ng/mL, about 1 ng/mL to about 300 ng/mL, about 1 ng/mL to about 280 ng/mL, about 1 ng/mL to about 260 ng/mL, about 1 ng/mL to about 240 ng/mL, about 1 ng/mL to about 220 ng/mL, about 1 ng/mL to about 200 ng/mL, about 1 ng/mL to about 180 ng/mL, about 1 ng/mL to about 160 ng/mL, about 1 ng/mL to about 140 ng/mL, about 1 ng/mL to about 120 ng/mL, about 1 ng/mL to about 100 ng/mL, about 1 ng/mL to about 80 ng/mL, about 1 ng/mL to about 60 ng/mL, about 1 ng/mL to about 40 ng/mL, about 1 ng/mL to about 30 ng/mL, about 1 ng/mL to about 25 ng/mL, about 1 ng/mL to about 20 ng/mL, about 1 ng/mL to about 15 ng/mL, about 1 ng/mL to about 10 ng/mL, about 1 ng/mL to about 5 ng/mL, 5 ng/mL to about 500 ng/mL, about 5 ng/mL to about 480 ng/mL, about 5 ng/mL to about 460 ng/mL, about 5 ng/mL to about 440 ng/mL, about 5 ng/mL to about 420 ng/mL, about 5 ng/mL to about 400 ng/mL, about 5 ng/mL to about 380 ng/mL, about 5 ng/mL to about 360 ng/mL, about 5 ng/mL to about 340 ng/mL, about 5 ng/mL to about 320 ng/mL, about 5 ng/mL to about 300 ng/mL, about 5 ng/mL to about 280 ng/mL, about 5 ng/mL to about 260 ng/mL, about 5 ng/mL to about 240 ng/mL, about 5 ng/mL to about 220 ng/mL, about 5 ng/mL to about 200 ng/mL, about 5 ng/mL to about 180 ng/mL, about 5 ng/mL to about 160 ng/mL, about 5 ng/mL to about 140 ng/mL, about 5 ng/mL to about 120 ng/mL, about 5 ng/mL to about 100 ng/mL, about 5 ng/mL to about 80 ng/mL, about 5 ng/mL to about 60 ng/mL, about 5 ng/mL to about 40 ng/mL, about 5 ng/mL to about 30 ng/mL, about 5 ng/mL to about 25 ng/mL, about 5 ng/mL to about 20 ng/mL, about 5 ng/mL to about 15 ng/mL, about 5 ng/mL to about 10 ng/mL, 10 ng/mL to about 500 ng/mL, about 10 ng/mL to about 480 ng/mL, about 10 ng/mL to about 460 ng/mL, about 10 ng/mL to about 440 ng/mL, about 10 ng/mL to about 420 ng/mL, about 10 ng/mL to about 400 ng/mL, about 10 ng/mL to about 380 ng/mL, about 10 ng/mL to about 360 ng/mL, about 10 ng/mL to about 340 ng/mL, about 10 ng/mL to about 320 ng/mL, about 10 ng/mL to about 300 ng/mL, about 10 ng/mL to about 280 ng/mL, about 10 ng/mL to about 260 ng/mL, about 10 ng/mL to about 240 ng/mL, about 10 ng/mL to about 220 ng/mL, about 10 ng/mL to about 200 ng/mL, about 10 ng/mL to about 180 ng/mL, about 10 ng/mL to about 160 ng/mL, about 10 ng/mL to about 140 ng/mL, about 10 ng/mL to about 120 ng/mL, about 10 ng/mL to about 100 ng/mL, about 10 ng/mL to about 80 ng/mL, about 10 ng/mL to about 60 ng/mL, about 10 ng/mL to about 40 ng/mL, about 10 ng/mL to about 30 ng/mL, about 10 ng/mL to about 25 ng/mL, about 10 ng/mL to about 20 ng/mL, about 10 ng/mL to about 15 ng/mL, 15 ng/mL to about 500 ng/mL, about 15 ng/mL to about 480 ng/mL, about 15 ng/mL to about 460 ng/mL, about 15 ng/mL to about 440 ng/mL, about 15 ng/mL to about 420 ng/mL, about 15 ng/mL to about 400 ng/mL, about 15 ng/mL to about 380 ng/mL, about 15 ng/mL to about 360 ng/mL, about 15 ng/mL to about 340 ng/mL, about 15 ng/mL to about 320 ng/mL, about 15 ng/mL to about 300 ng/mL, about 15 ng/mL to about 280 ng/mL, about 15 ng/mL to about 260 ng/mL, about 15 ng/mL to about 240 ng/mL, about 15 ng/mL to about 220 ng/mL, about 15 ng/mL to about 200 ng/mL, about 15 ng/mL to about 180 ng/mL, about 15 ng/mL to about 160 ng/mL, about 15 ng/mL to about 140 ng/mL, about 15 ng/mL to about 120 ng/mL, about 15 ng/mL to about 100 ng/mL, about 15 ng/mL to about 80 ng/mL, about 15 ng/mL to about 60 ng/mL, about 15 ng/mL to about 40 ng/mL, about 15 ng/mL to about 30 ng/mL, about 15 ng/mL to about 25 ng/mL, about 15 ng/mL to about 20 ng/mL, 20 ng/mL to about 500 ng/mL, about 20 ng/mL to about 480 ng/mL, about 20 ng/mL to about 460 ng/mL, about 20 ng/mL to about 440 ng/mL, about 20 ng/mL to about 420 ng/mL, about 20 ng/mL to about 400 ng/mL, about 20 ng/mL to about 380 ng/mL, about 20 ng/mL to about 360 ng/mL, about 20 ng/mL to about 340 ng/mL, about 20 ng/mL to about 320 ng/mL, about 20 ng/mL to about 300 ng/mL, about 20 ng/mL to about 280 ng/mL, about 20 ng/mL to about 260 ng/mL, about 20 ng/mL to about 240 ng/mL, about 20 ng/mL to about 220 ng/mL, about 20 ng/mL to about 200 ng/mL, about 20 ng/mL to about 180 ng/mL, about 20 ng/mL to about 160 ng/mL, about 20 ng/mL to about 140 ng/mL, about 20 ng/mL to about 120 ng/mL, about 20 ng/mL to about 100 ng/mL, about 20 ng/mL to about 80 ng/mL, about 20 ng/mL to about 60 ng/mL, about 20 ng/mL to about 40 ng/mL, about 20 ng/mL to about 30 ng/mL, about 20 ng/mL to about 25 ng/mL, 25 ng/mL to about 500 ng/mL, about 25 ng/mL to about 480 ng/mL, about 25 ng/mL to about 460 ng/mL, about 25 ng/mL to about 440 ng/mL, about 25 ng/mL to about 420 ng/mL, about 25 ng/mL to about 400 ng/mL, about 25 ng/mL to about 380 ng/mL, about 25 ng/mL to about 360 ng/mL, about 25 ng/mL to about 340 ng/mL, about 25 ng/mL to about 320 ng/mL, about 25 ng/mL to about 300 ng/mL, about 25 ng/mL to about 280 ng/mL, about 25 ng/mL to about 260 ng/mL, about 25 ng/mL to about 240 ng/mL, about 25 ng/mL to about 220 ng/mL, about 25 ng/mL to about 200 ng/mL, about 25 ng/mL to about 180 ng/mL, about 25 ng/mL to about 160 ng/mL, about 25 ng/mL to about 140 ng/mL, about 25 ng/mL to about 120 ng/mL, about 25 ng/mL to about 100 ng/mL, about 25 ng/mL to about 80 ng/mL, about 25 ng/mL to about 60 ng/mL, about 25 ng/mL to about 40 ng/mL, about 25 ng/mL to about 30 ng/mL, 30 ng/mL to about 500 ng/mL, about 30 ng/mL to about 480 ng/mL, about 30 ng/mL to about 460 ng/mL, about 30 ng/mL to about 440 ng/mL, about 30 ng/mL to about 420 ng/mL, about 30 ng/mL to about 400 ng/mL, about 30 ng/mL to about 380 ng/mL, about 30 ng/mL to about 360 ng/mL, about 30 ng/mL to about 340 ng/mL, about 30 ng/mL to about 320 ng/mL, about 30 ng/mL to about 300 ng/mL, about 30 ng/mL to about 280 ng/mL, about 30 ng/mL to about 260 ng/mL, about 30 ng/mL to about 240 ng/mL, about 30 ng/mL to about 220 ng/mL, about 30 ng/mL to about 200 ng/mL, about 30 ng/mL to about 180 ng/mL, about 30 ng/mL to about 160 ng/mL, about 30 ng/mL to about 140 ng/mL, about 30 ng/mL to about 120 ng/mL, about 30 ng/mL to about 100 ng/mL, about 30 ng/mL to about 80 ng/mL, about 30 ng/mL to about 60 ng/mL, about 30 ng/mL to about 40 ng/mL, 40 ng/mL to about 500 ng/mL, about 40 ng/mL to about 480 ng/mL, about 40 ng/mL to about 460 ng/mL, about 40 ng/mL to about 440 ng/mL, about 40 ng/mL to about 420 ng/mL, about 40 ng/mL to about 400 ng/mL, about 40 ng/mL to about 380 ng/mL, about 40 ng/mL to about 360 ng/mL, about 40 ng/mL to about 340 ng/mL, about 40 ng/mL to about 320 ng/mL, about 40 ng/mL to about 300 ng/mL, about 40 ng/mL to about 280 ng/mL, about 40 ng/mL to about 260 ng/mL, about 40 ng/mL to about 240 ng/mL, about 40 ng/mL to about 220 ng/mL, about 40 ng/mL to about 200 ng/mL, about 40 ng/mL to about 180 ng/mL, about 40 ng/mL to about 160 ng/mL, about 40 ng/mL to about 140 ng/mL, about 40 ng/mL to about 120 ng/mL, about 40 ng/mL to about 100 ng/mL, about 40 ng/mL to about 80 ng/mL, about 40 ng/mL to about 60 ng/mL, 60 ng/mL to about 500 ng/mL, about 60 ng/mL to about 480 ng/mL, about 60 ng/mL to about 460 ng/mL, about 60 ng/mL to about 440 ng/mL, about 60 ng/mL to about 420 ng/mL, about 60 ng/mL to about 400 ng/mL, about 60 ng/mL to about 380 ng/mL, about 60 ng/mL to about 360 ng/mL, about 60 ng/mL to about 340 ng/mL, about 60 ng/mL to about 320 ng/mL, about 60 ng/mL to about 300 ng/mL, about 60 ng/mL to about 280 ng/mL, about 60 ng/mL to about 260 ng/mL, about 60 ng/mL to about 240 ng/mL, about 60 ng/mL to about 220 ng/mL, about 60 ng/mL to about 200 ng/mL, about 60 ng/mL to about 180 ng/mL, about 60 ng/mL to about 160 ng/mL, about 60 ng/mL to about 140 ng/mL, about 60 ng/mL to about 120 ng/mL, about 60 ng/mL to about 100 ng/mL, about 60 ng/mL to about 80 ng/mL, 80 ng/mL to about 500 ng/mL, about 80 ng/mL to about 480 ng/mL, about 80 ng/mL to about 460 ng/mL, about 80 ng/mL to about 440 ng/mL, about 80 ng/mL to about 420 ng/mL, about 80 ng/mL to about 400 ng/mL, about 80 ng/mL to about 380 ng/mL, about 80 ng/mL to about 360 ng/mL, about 80 ng/mL to about 340 ng/mL, about 80 ng/mL to about 320 ng/mL, about 80 ng/mL to about 300 ng/mL, about 80 ng/mL to about 280 ng/mL, about 80 ng/mL to about 260 ng/mL, about 80 ng/mL to about 240 ng/mL, about 80 ng/mL to about 220 ng/mL, about 80 ng/mL to about 200 ng/mL, about 80 ng/mL to about 180 ng/mL, about 80 ng/mL to about 160 ng/mL, about 80 ng/mL to about 140 ng/mL, about 80 ng/mL to about 120 ng/mL, about 80 ng/mL to about 100 ng/mL, 100 ng/mL to about 500 ng/mL, about 100 ng/mL to about 480 ng/mL, about 100 ng/mL to about 460 ng/mL, about 100 ng/mL to about 440 ng/mL, about 100 ng/mL to about 420 ng/mL, about 100 ng/mL to about 400 ng/mL, about 100 ng/mL to about 380 ng/mL, about 100 ng/mL to about 360 ng/mL, about 100 ng/mL to about 340 ng/mL, about 100 ng/mL to about 320 ng/mL, about 100 ng/mL to about 300 ng/mL, about 100 ng/mL to about 280 ng/mL, about 100 ng/mL to about 260 ng/mL, about 100 ng/mL to about 240 ng/mL, about 100 ng/mL to about 220 ng/mL, about 100 ng/mL to about 200 ng/mL, about 100 ng/mL to about 180 ng/mL, about 100 ng/mL to about 160 ng/mL, about 100 ng/mL to about 140 ng/mL, about 100 ng/mL to about 120 ng/mL, 120 ng/mL to about 500 ng/mL, about 120 ng/mL to about 480 ng/mL, about 120 ng/mL to about 460 ng/mL, about 120 ng/mL to about 440 ng/mL, about 120 ng/mL to about 420 ng/mL, about 120 ng/mL to about 400 ng/mL, about 120 ng/mL to about 380 ng/mL, about 120 ng/mL to about 360 ng/mL, about 120 ng/mL to about 340 ng/mL, about 120 ng/mL to about 320 ng/mL, about 120 ng/mL to about 300 ng/mL, about 120 ng/mL to about 280 ng/mL, about 120 ng/mL to about 260 ng/mL, about 120 ng/mL to about 240 ng/mL, about 120 ng/mL to about 220 ng/mL, about 120 ng/mL to about 200 ng/mL, about 120 ng/mL to about 180 ng/mL, about 120 ng/mL to about 160 ng/mL, about 120 ng/mL to about 140 ng/mL, 140 ng/mL to about 500 ng/mL, about 140 ng/mL to about 480 ng/mL, about 140 ng/mL to about 460 ng/mL, about 140 ng/mL to about 440 ng/mL, about 140 ng/mL to about 420 ng/mL, about 140 ng/mL to about 400 ng/mL, about 140 ng/mL to about 380 ng/mL, about 140 ng/mL to about 360 ng/mL, about 140 ng/mL to about 340 ng/mL, about 140 ng/mL to about 320 ng/mL, about 140 ng/mL to about 300 ng/mL, about 140 ng/mL to about 280 ng/mL, about 140 ng/mL to about 260 ng/mL, about 140 ng/mL to about 240 ng/mL, about 140 ng/mL to about 220 ng/mL, about 140 ng/mL to about 200 ng/mL, about 140 ng/mL to about 180 ng/mL, about 140 ng/mL to about 160 ng/mL, 160 ng/mL to about 500 ng/mL, about 160 ng/mL to about 480 ng/mL, about 160 ng/mL to about 460 ng/mL, about 160 ng/mL to about 440 ng/mL, about 160 ng/mL to about 420 ng/mL, about 160 ng/mL to about 400 ng/mL, about 160 ng/mL to about 380 ng/mL, about 160 ng/mL to about 360 ng/mL, about 160 ng/mL to about 340 ng/mL, about 160 ng/mL to about 320 ng/mL, about 160 ng/mL to about 300 ng/mL, about 160 ng/mL to about 280 ng/mL, about 160 ng/mL to about 260 ng/mL, about 160 ng/mL to about 240 ng/mL, about 160 ng/mL to about 220 ng/mL, about 160 ng/mL to about 200 ng/mL, about 160 ng/mL to about 180 ng/mL,180 ng/mL to about 500 ng/mL, about 180 ng/mL to about 480 ng/mL, about 180 ng/mL to about 460 ng/mL, about 180 ng/mL to about 440 ng/mL, about 180 ng/mL to about 420 ng/mL, about 180 ng/mL to about 400 ng/mL, about 180 ng/mL to about 380 ng/mL, about 180 ng/mL to about 360 ng/mL, about 180 ng/mL to about 340 ng/mL, about 180 ng/mL to about 320 ng/mL, about 180 ng/mL to about 300 ng/mL, about 180 ng/mL to about 280 ng/mL, about 180 ng/mL to about 260 ng/mL, about 180 ng/mL to about 240 ng/mL, about 180 ng/mL to about 220 ng/mL, about 180 ng/mL to about 200 ng/mL, 200 ng/mL to about 500 ng/mL, about 200 ng/mL to about 480 ng/mL, about 200 ng/mL to about 460 ng/mL, about 200 ng/mL to about 440 ng/mL, about 200 ng/mL to about 420 ng/mL, about 200 ng/mL to about 400 ng/mL, about 200 ng/mL to about 380 ng/mL, about 200 ng/mL to about 360 ng/mL, about 200 ng/mL to about 340 ng/mL, about 200 ng/mL to about 320 ng/mL, about 200 ng/mL to about 300 ng/mL, about 200 ng/mL to about 280 ng/mL, about 200 ng/mL to about 260 ng/mL, about 200 ng/mL to about 240 ng/mL, about 200 ng/mL to about 220 ng/mL, 220 ng/mL to about 500 ng/mL, about 220 ng/mL to about 480 ng/mL, about 220 ng/mL to about 460 ng/mL, about 220 ng/mL to about 440 ng/mL, about 220 ng/mL to about 420 ng/mL, about 220 ng/mL to about 300 ng/mL, about 220 ng/mL to about 380 ng/mL, about 220 ng/mL to about 360 ng/mL, about 220 ng/mL to about 340 ng/mL, about 220 ng/mL to about 320 ng/mL, about 220 ng/mL to about 400 ng/mL, about 220 ng/mL to about 280 ng/mL, about 220 ng/mL to about 260 ng/mL, about 220 ng/mL to about 240 ng/mL, 240 ng/mL to about 500 ng/mL, about 240 ng/mL to about 480 ng/mL, about 240 ng/mL to about 460 ng/mL, about 240 ng/mL to about 440 ng/mL, about 240 ng/mL to about 420 ng/mL, about 240 ng/mL to about 400 ng/mL, about 240 ng/mL to about 380 ng/mL, about 240 ng/mL to about 360 ng/mL, about 240 ng/mL to about 340 ng/mL, about 240 ng/mL to about 320 ng/mL, about 240 ng/mL to about 300 ng/mL, about 240 ng/mL to about 280 ng/mL, about 240 ng/mL to about 260 ng/mL, 260 ng/mL to about 500 ng/mL, about 260 ng/mL to about 480 ng/mL, about 260 ng/mL to about 460 ng/mL, about 260 ng/mL to about 440 ng/mL, about 260 ng/mL to about 420 ng/mL, about 260 ng/mL to about 400 ng/mL, about 260 ng/mL to about 380 ng/mL, about 260 ng/mL to about 360 ng/mL, about 260 ng/mL to about 340 ng/mL, about 260 ng/mL to about 320 ng/mL, about 260 ng/mL to about 300 ng/mL, about 260 ng/mL to about 280 ng/mL, 280 ng/mL to about 500 ng/mL, about 280 ng/mL to about 480 ng/mL, about 280 ng/mL to about 460 ng/mL, about 280 ng/mL to about 440 ng/mL, about 280 ng/mL to about 420 ng/mL, about 280 ng/mL to about 400 ng/mL, about 280 ng/mL to about 380 ng/mL, about 280 ng/mL to about 360 ng/mL, about 280 ng/mL to about 340 ng/mL, about 280 ng/mL to about 320 ng/mL, about 280 ng/mL to about 300 ng/mL, 300 ng/mL to about 500 ng/mL, about 300 ng/mL to about 480 ng/mL, about 300 ng/mL to about 460 ng/mL, about 300 ng/mL to about 440 ng/mL, about 300 ng/mL to about 420 ng/mL, about 300 ng/mL to about 400 ng/mL, about 300 ng/mL to about 380 ng/mL, about 300 ng/mL to about 360 ng/mL, about 300 ng/mL to about 340 ng/mL, about 300 ng/mL to about 320 ng/mL, 320 ng/mL to about 500 ng/mL, about 320 ng/mL to about 480 ng/mL, about 320 ng/mL to about 460 ng/mL, about 320 ng/mL to about 440 ng/mL, about 320 ng/mL to about 420 ng/mL, about 320 ng/mL to about 400 ng/mL, about 320 ng/mL to about 380 ng/mL, about 320 ng/mL to about 360 ng/mL, about 320 ng/mL to about 340 ng/mL, 340 ng/mL to about 500 ng/mL, about 340 ng/mL to about 480 ng/mL, about 340 ng/mL to about 460 ng/mL, about 340 ng/mL to about 440 ng/mL, about 340 ng/mL to about 420 ng/mL, about 340 ng/mL to about 400 ng/mL, about 340 ng/mL to about 380 ng/mL, about 340 ng/mL to about 360 ng/mL, 360 ng/mL to about 500 ng/mL, about 360 ng/mL to about 480 ng/mL, about 360 ng/mL to about 460 ng/mL, about 360 ng/mL to about 440 ng/mL, about 360 ng/mL to about 420 ng/mL, about 360 ng/mL to about 400 ng/mL, about 360 ng/mL to about 380 ng/mL, 380 ng/mL to about 500 ng/mL, about 380 ng/mL to about 480 ng/mL, about 380 ng/mL to about 460 ng/mL, about 380 ng/mL to about 440 ng/mL, about 380 ng/mL to about 420 ng/mL, about 380 ng/mL to about 400 ng/mL, 400 ng/mL to about 500 ng/mL, about 400 ng/mL to about 480 ng/mL, about 400 ng/mL to about 460 ng/mL, about 400 ng/mL to about 440 ng/mL, about 400 ng/mL to about 420 ng/mL, 420 ng/mL to about 500 ng/mL, about 420 ng/mL to about 480 ng/mL, about 420 ng/mL to about 460 ng/mL, about 420 ng/mL to about 440 ng/mL, 440 ng/mL to about 500 ng/mL, about 440 ng/mL to about 480 ng/mL, about 440 ng/mL to about 460 ng/mL, about 460 ng/mL to about 500 ng/mL, about 460 ng/mL to about 480 ng/mL, or about 480 ng/mL to about 500 ng/mL of EPO (e.g., recombinant human EPO) or an EPO-mimetic peptide. Non-limiting examples of EPO-mimetic peptide include, e.g., EPO Mimetic Peptide (EMP1; GGTYSCHFGPLTWVCKPQGG SEQ ID NO: 1) or its peptide dimers using either defined chemical linkers or larger polymeric PEG linkers (such as those described in Johnson and Jolliffe, Nephrology Dialysis Transplantation 15(9):1274-1277, 2000), ERB1-7 (such as those described in McConnell et al., Biol. Chem. 379(10):1279-86, 1998), EPO-R-derived peptide (ERP) (such as those described in Naranda et al., PNAS 96(13):7569-7574, 1999), EMP20 (YSCHFGPLTWVCK (SEQ ID NO: 2)) described in Johnson et al., Biochemistry 37(11):3699-3710, 1998, AGEM400(hydroxyethyl starch), and pegolsihematide (such as those described in Gupta and Wish, Curr. Opin. Nephrol. Hypertens. 27(5):345-350, 2018). Additional examples of EPO-mimetic peptides are known in the art.

In some embodiments, the second culture medium comprises about 0.1 μg/mL to about 50 μg/mL, about 0.1 μg/mL to about 45 μg/mL, about 0.1 μg/mL to about 40 μmL, about 0.1 μg/mL to about 35 μg/mL, about 0.1 μg/mL to about 30 μg/mL, about 0.1 μg/mL to about 25 μg/mL, about 0.1 μg/mL to about 20 μg/mL, about 0.1 μg/mL about 15 μg/mL, about 0.1 μg/mL to about 10 μg/mL, about 0.1 μg/mL to about 5 μg/mL, about 0.1 μg/mL to about 2 μg/mL, about 0.1 μg/mL to about 1 μg/mL, about 1 μg/mL to about 50 μg/mL, about 1 μg/mL to about 45 μg/mL, about 1 μg/mL to about 40 μg/mL, about 1 μg/mL to about 35 μg/mL, about 1 μg/mL to about 30 μg/mL, about 1 μg/mL to about 25 μg/mL, about 1 μg/mL to about 20 μg/mL, about 1 μg/mL about 15 μg/mL, about 1 μg/mL to about 10 μg/mL, about 1 μg/mL to about 5 μg/mL, about 1 μg/mL to about 2 μg/mL, about 2 μg/mL to about 50 μg/mL, about 2 μg/mL to about 45 μg/mL, about 2 μg/mL to about 40 μg/mL, about 2 μg/mL to about 35 μg/mL, about 2 μg/mL to about 30 μg/mL, about 2 μg/mL to about 25 μg/mL, about 2 μg/mL to about 20 μg/mL, about 2 μg/mL about 15 μg/mL, about 2 μg/mL to about 10 μg/mL, about 2 μg/mL to about 5 μg/mL, about 5 μg/mL to about 50 μg/mL, about 5 μg/mL to about 45 μg/mL, about 5 μg/mL to about 40 μg/mL, about 5 μg/mL to about 35 μg/mL, about 5 μg/mL to about 30 μg/mL, about 5 μg/mL to about 25 μg/mL, about 5 μg/mL to about 20 μg/mL, about 5 μg/mL about 15 μg/mL, about 5 μg/mL to about 10 μg/mL, about 10 μg/mL to about 50 μg/mL, about 10 μg/mL to about 45 μg/mL, about 10 μg/mL to about 40 μg/mL, about 10 μg/mL to about 35 μg/mL, about 10 μg/mL to about 30 μg/mL, about 10 μg/mL to about 25 μg/mL, about 10 μg/mL to about 20 μg/mL, about 10 μg/mL about 15 μg/mL, about 15 μg/mL to about 50 μg/mL, about 15 μg/mL to about 45 μg/mL, about 15 μg/mL to about 40 μg/mL, about 15 μg/mL to about 35 μg/mL, about 15 μg/mL to about 30 μg/mL, about 15 μg/mL to about 25 μg/mL, about 15 μg/mL to about 20 μg/mL, about 20 μg/mL to about 50 μg/mL, about 20 μg/mL to about 45 μg/mL, about 20 μg/mL to about 40 μg/mL, about 20 μg/mL to about 35 μg/mL, about 20 μg/mL to about 30 μg/mL, about 20 μg/mL to about 25 μg/mL, about 25 μg/mL to about 50 μg/mL, about 25 μg/mL to about 45 μg/mL, about 25 μg/mL to about 40 μg/mL, about 25 μg/mL to about 35 μg/mL, about 25 μg/mL to about 30 μg/mL, about 30 μg/mL to about 50 μg/mL, about 30 μg/mL to about 45 μg/mL, about 30 μg/mL to about 40 μg/mL, about 30 μg/mL to about 35 μg/mL, about 35 μg/mL to about 50 μg/mL, about 35 μg/mL to about 45 μg/mL, about 35 μg/mL to about 40 μg/mL, about 40 μg/mL to about 50 μg/mL, about 40 μg/mL to about 45 μg/mL, or about 45 μg/mL to about 50 μg/mL of insulin (e.g., human insulin). In some embodiments of any of the second culture media described herein, the second culture medium includes Iscove's modified Dulbecco's medium (IMDM). In some embodiments of any of the second culture media described herein, the second culture medium includes about 1 μg/mL to about 10 μg/mL (or any of the subranges of this range described herein) lipid (e.g., lipid mixture). In some embodiments of any of the second culture media described herein, the second culture medium includes about 0.1 mM to about 10 mM (or any of the subranges of this range described herein) of L-glutamine, L-alanyl-L-glutamine, L-glycyl-L-glutamine, N-acetyl-L-glutamine, or a combination thereof. In some embodiments of any of the second culture media described herein, the second culture medium includes 0.1% w/v to about 4% w/v (or any of the subranges of this range described herein) human serum albumin. In some embodiments of any of the second culture media described herein, the second culture medium includes about 0.1% w/v to about 3% w/v (or any of the subranges of this range described herein) Poloxamer-188 (P188).

In some examples, the second culture medium can be, e.g., a chemically-defined liquid culture medium, an animal component-free liquid culture medium, or a chemically-defined animal component-free liquid culture medium, and/or a serum-free liquid culture medium.

### Optional Culturing Step Before Step A

Some embodiments of any of the methods described herein can further include, prior to step (a), (i) disposing a plurality of erythroid progenitor cells (e.g., any of the exemplary erythroid progenitor cells described herein or known in the art) in a first culture medium comprised within a vessel (e.g., a bioreactor, a shake tube, or a shake flask, e.g., any of the exemplary bioreactors, shake tubes, or shake flasks described herein) to provide the first cell culture (e.g., having an initial cell density of about of about 0.1×10⁵ cells/mL to about 5×10⁶ cells/mL (e.g., about 0.1×10⁵ cells/mL to about 4×10⁶ cells/mL, about 0.1×10⁵ cells/mL to about 3×10⁶ cells/mL, about 0.1×10⁵ cells/mL to about 2×10⁶ cells/mL, about 0.1×10⁵ cells/mL to about 1×10⁶ cells/mL, about 0.1×10⁵ cells/mL to about 8×10⁵ cells/mL, about 0.1×10⁵ cells/mL to about 6×10⁵ cells/mL, about 0.1×10⁵ cells/mL to about 5×10⁵ cells/mL, about 0.1×10⁵ cells/mL to about 4×10⁵ cells/mL, about 0.1×10⁵ cells/mL to about 3×10⁵ cells/mL, about 0.1×10⁵ cells/mL to about 2×10⁵ cells/mL, 0.1×10⁵ cells/mL to about 1.5×10⁵ cells/mL, about 0.1×10⁵ cells/mL to about 1×10⁵ cells/mL, about 0.1×10⁵ cells/mL to about 0.5×10⁵ cells/mL, about 0.5×10⁵ cells/mL to about 5×10⁶ cells/mL, about 0.5×10⁵ cells/mL to about 4×10⁶ cells/mL, about 0.5×10⁵ cells/mL to about 3×10⁶ cells/mL, about 0.5×10⁵ cells/mL to about 2×10⁶ cells/mL, about 0.5×10⁵ cells/mL to about 1×10⁶ cells/mL, about 0.5×10⁵ cells/mL to about 8×10⁵ cells/mL, about 0.5×10⁵ cells/mL to about 6×10⁵ cells/mL, about 0.5×10⁵ cells/mL to about 5×10⁵ cells/mL, about 0.5×10⁵ cells/mL to about 4×10⁵ cells/mL, about 0.5×10⁵ cells/mL to about 3×10⁵ cells/mL, about 0.5×10⁵ cells/mL to about 2×10⁵ cells/mL, 0.5×10⁵ cells/mL to about 1.5×10⁵ cells/mL, about 0.5×10⁵ cells/mL to about 1×10⁵ cells/mL, about 1×10⁵ cells/mL to about 5×10⁶ cells/mL, about 1×10⁵ cells/mL to about 4×10⁶ cells/mL, about 1×10⁵ cells/mL to about 3×10⁶ cells/mL, about 1×10⁵ cells/mL to about 2×10⁶ cells/mL, about 1×10⁵ cells/mL to about 1×10⁶ cells/mL, about 1×10⁵ cells/mL to about 8×10⁵ cells/mL, about 1×10⁵ cells/mL to about 6×10⁵ cells/mL, about 1×10⁵ cells/mL to about 5×10⁵ cells/mL, about 1×10⁵ cells/mL to about 4×10⁵ cells/mL, about 1×10⁵ cells/mL to about 3×10⁵ cells/mL, about 1×10⁵ cells/mL to about 2×10⁵ cells/mL, 1×10⁵ cells/mL to about 1.5×10⁵ cells/mL, about 1.5×10⁵ cells/mL to about 5×10⁶ cells/mL, about 1.5×10⁵ cells/mL to about 4×10⁶ cells/mL, about 1.5×10⁵ cells/mL to about 3×10⁶ cells/mL, about 1.5×10⁵ cells/mL to about 2×10⁶ cells/mL, about 1.5×10⁵ cells/mL to about 1×10⁶ cells/mL, about 1.5×10⁵ cells/mL to about 8×10⁵ cells/mL, about 1.5×10⁵ cells/mL to about 6×10⁵ cells/mL, about 1.5×10⁵ cells/mL to about 5×10⁵ cells/mL, about 1.5×10⁵ cells/mL to about 4×10⁵ cells/mL, about 1.5×10⁵ cells/mL to about 3×10⁵ cells/mL, about 1.5×10⁵ cells/mL to about 2×10⁵ cells/mL, about 2×10⁵ cells/mL to about 5×10⁶ cells/mL, about 2×10⁵ cells/mL to about 4×10⁶ cells/mL, about 2×10⁵ cells/mL to about 3×10⁶ cells/mL, about 2×10⁵ cells/mL to about 2×10⁶ cells/mL, about 2×10⁵ cells/mL to about 1×10⁶ cells/mL, about 2×10⁵ cells/mL to about 8×10⁵ cells/mL, about 2×10⁵ cells/mL to about 6×10⁵ cells/mL, about 2×10⁵ cells/mL to about 5×10⁵ cells/mL, about 2×10⁵ cells/mL to about 4×10⁵ cells/mL, about 2×10⁵ cells/mL to about 3×10⁵ cells/mL, about 3×10⁵ cells/mL to about 5×10⁶ cells/mL, about 3×10⁵ cells/mL to about 4×10⁶ cells/mL, about 3×10⁵ cells/mL to about 3×10⁶ cells/mL, about 3×10⁵ cells/mL to about 2×10⁶ cells/mL, about 3×10⁵ cells/mL to about 1×10⁶ cells/mL, about 3×10⁵ cells/mL to about 8×10⁵ cells/mL, about 3×10⁵ cells/mL to about 6×10⁵ cells/mL, about 3×10⁵ cells/mL to about 5×10⁵ cells/mL, about 3×10⁵ cells/mL to about 4×10⁵ cells/mL, about 4×10⁵ cells/mL to about 5×10⁶ cells/mL, about 4×10⁵ cells/mL to about 4×10⁶ cells/mL, about 4×10⁵ cells/mL to about 3×10⁶ cells/mL, about 4×10⁵ cells/mL to about 2×10⁶ cells/mL, about 4×10⁵ cells/mL to about 1×10⁶ cells/mL, about 4×10⁵ cells/mL to about 8×10⁵ cells/mL, about 4×10⁵ cells/mL to about 6×10⁵ cells/mL, about 4×10⁵ cells/mL to about 5×10⁵ cells/mL, about 5×10⁵ cells/mL to about 5×10⁶ cells/mL, about 5×10⁵ cells/mL to about 4×10⁶ cells/mL, about 5×10⁵ cells/mL to about 3×10⁶ cells/mL, about 5×10⁵ cells/mL to about 2×10⁶ cells/mL, about 5×10⁵ cells/mL to about 1×10⁶ cells/mL, about 5×10⁵ cells/mL to about 8×10⁵ cells/mL, about 5×10⁵ cells/mL to about 6×10⁵ cells/mL, about 6×10⁵ cells/mL to about 5×10⁶ cells/mL, about 6×10⁵ cells/mL to about 4×10⁶ cells/mL, about 6×10⁵ cells/mL to about 3×10⁶ cells/mL, about 6×10⁵ cells/mL to about 2×10⁶ cells/mL, about 6×10⁵ cells/mL to about 1×10⁶ cells/mL, about 6×10⁵ cells/mL to about 8×10⁵ cells/mL, about 8×10⁵ cells/mL to about 5×10⁶ cells/mL, about 8×10⁵ cells/mL to about 4×10⁶ cells/mL, about 8×10⁵ cells/mL to about 3×10⁶ cells/mL, about 8×10⁵ cells/mL to about 2×10⁶ cells/mL, about 8×10⁵ cells/mL to about 1×10⁶ cells/mL, about 1×10⁶ cells/mL to about 5×10⁶ cells/mL, about 1×10⁶ cells/mL to about 4×10⁶ cells/mL, about 1×10⁶ cells/mL to about 3×10⁶ cells/mL, about 1×10⁶ cells/mL to about 2×10⁶ cells/mL, about 2×10⁶ cells/mL to about 5×10⁶ cells/mL, about 2×10⁶ cells/mL to about 4×10⁶ cells/mL, about 2×10⁶ cells/mL to about 3×10⁶ cells/mL, about 3×10⁶ cells/mL to about 5×10⁶ cells/mL, about 3×10⁶ cells/mL to about 4×10⁶ cells/mL, or about 4×10⁶ cells/mL to about 5×10⁶ cells/mL); and (ii) culturing (e.g., batch, fed-batch, or perfusion culturing) the first cell culture for about 1 day to about 15 days (e.g., about 1 day to about 14 days, about 1 day to about 13 days, about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 days to about 15 days, about 2 days to about 14 days, about 2 days to about 13 days, about 2 days to about 12 days, about 2 days to about 11 days, about 2 days to about 10 days, about 2 days to about 9 days, about 2 day to about 8 days, about 2 days to about 7 days, about 2 days to about 6 days, about 2 days to about 5 days, about 2 days to about 4 days, about 2 days to about 3 days, about 3 days to about 15 days, about 3 days to about 14 days, about 3 days to about 13 days, about 3 days to about 12 days, about 3 days to about 11 days, about 3 days to about 10 days, about 3 days to about 9 days, about 3 days to about 8 days, about 3 days to about 7 days, about 3 days to about 6 days, about 3 days to about 5 days, about 3 days to about 4 days, about 4 days to about 15 days, about 4 days to about 14 days, about 4 days to about 13 days, about 4 days to about 12 days, about 4 days to about 11 days, about 4 days to about 10 days, about 4 days to about 9 days, about 4 days to about 8 days, about 4 days to about 7 days, about 4 days to about 6 days, about 4 days to about 5 days, about 5 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, about 5 days to about 6 days, about 6 days to about 15 days, about 6 days to about 14 days, about 6 days to about 13 days, about 6 days to about 12 days, about 6 days to about 11 days, about 6 days to about 10 days, about 6 days to about 9 days, about 6 days to about 8 days, about 6 days to about 7 days, about 7 days to about 15 days, about 7 days to about 14 days, about 7 days to about 13 days, about 7 days to about 12 days, about 7 days to about 11 days, about 7 days to about 10 days, about 7 days to about 9 days, about 7 days to about 8 days, about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 15 days, about 9 days to about 14 days, about 9 days to about 13 days, about 9 days to about 12 days, about 9 days to about 11 days, about 9 days to about 10 days, about 10 days to about 15 days, about 10 days to about 14 days, about 10 days to about 13 days, about 10 days to about 12 days, about 10 days to about 11 days, about 11 days to about 15 days, about 11 days to about 14 days, about 11 days to about 13 days, about 11 days to about 12 days, about 12 days to about 15 days, about 12 days to about 14 days, about 12 days to about 13 days, about 13 days to about 15 days, about 13 days to about 14 days, or about 14 days to about 15 days).

In some embodiments, step (i) includes disposing a plurality of erythroid progenitor cells (e.g., any of the exemplary erythroid progenitor cells described herein or known in the art) in a first culture medium comprised within shake flask (e.g., any of the exemplary shake flasks described herein having any of the exemplary volumes described herein). In such embodiments, step (ii) includes incubating the first cell culture in the shake flask at 0.1×g to about 50×g (or any of the subranges of this range described herein).

In some embodiments, step (i) includes disposing a plurality of erythroid progenitor cells (e.g., any of the exemplary erythroid progenitor cells described herein or known in the art) in a first culture medium comprised within shake tube (e.g., any of the exemplary shake tubes described herein having any of the exemplary volumes described herein). In such embodiments, step (ii) includes incubating the first cell culture in the shake tube at about 0.1×g to about 50×g (or any of the subranges of this range described herein).

In some embodiments, step (i) includes disposing a plurality of erythroid progenitor cells (e.g., any of the exemplary erythroid progenitor cells described herein or known in the art) in a culture bag described herein having any of the exemplary volumes described herein). In such embodiments, step (ii) includes incubating the first cell culture in the culture bag at rocking rate of about 10 rock cycles per minute to about 50 rock cycles per minute (or any of the subranges of this range described herein).

In some embodiments, step (ii) includes batch culturing the first cell culture.

### Fed Batch Culturing

In some embodiments, step (ii) includes fed batch culturing the first cell culture. In some embodiments, fed batch culturing in step (ii) includes adding an additional volume of the first culture medium (e.g., any of the exemplary first culture media described herein) over time. In some examples, the additional volume of the first culture medium is added continuously to the first cell culture over time. In some examples, the additional volume of the first culture medium is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the first cell culture over time. In some embodiments, about 0.1× to about 10× (e.g., about 0.1× to about 9.5×, about 0.1× to about 9.0×, about 0.1× to about 8.5×, about 0.1× to about 8.0×, about 0.1× to about 7.5×, about 0.1× to about 7.0×, about 0.1× to about 6.5×, about 0.1× to about 6.0×, about 0.1× to about 5.5×, about 0.1× to about 5.0×, about 0.1× to about 4.5×, about 0.1× to about 4.0×, about 0.1× to about 3.5×, about 0.1× to about 3.0×, about 0.1× to about 2.5×, about 0.1× to about 2.0×, about 0.1× to about 1.5×, about 0.1× to about 1.0×, about 0.1× to about 0.5×, about 0.1× to about 0.3×, about 0.1× to about 0.2×, about 0.2× to about 10×, about 0.2× to about 9.5×, about 0.2× to about 9.0×, about 0.2× to about 8.5×, about 0.2× to about 8.0×, about 0.2× to about 7.5×, about 0.2× to about 7.0×, about 0.2× to about 6.5×, about 0.2× to about 6.0×, about 0.2× to about 5.5×, about 0.2× to about 5.0×, about 0.2× to about 4.5×, about 0.2× to about 4.0×, about 0.2× to about 3.5×, about 0.2× to about 3.0×, about 0.2× to about 2.5×, about 0.2× to about 2.0×, about 0.2× to about 1.5×, about 0.2× to about 1.0×, about 0.2× to about 0.5×, about 0.2× to about 0.3×, about 0.3× to about 10×, about 0.3× to about 9.5×, about 0.3× to about 9.0×, about 0.3× to about 8.5×, about 0.3× to about 8.0×, about 0.3× to about 7.5×, about 0.3× to about 7.0×, about 0.3× to about 6.5×, about 0.3× to about 6.0×, about 0.3× to about 5.5×, about 0.3× to about 5.0×, about 0.3× to about 4.5×, about 0.3× to about 4.0×, about 0.3× to about 3.5×, about 0.3× to about 3.0×, about 0.3× to about 2.5×, about 0.3× to about 2.0×, about 0.3× to about 1.5×, about 0.3× to about 1.0×, about 0.3× to about 0.5×, about 0.5× to about 10×, about 0.5× to about 9.5×, about 0.5× to about 9.0×, about 0.5× to about 8.5×, about 0.5× to about 8.0×, about 0.5× to about 7.5×, about 0.5× to about 7.0×, about 0.5× to about 6.5×, about 0.5× to about 6.0×, about 0.5× to about 5.5×, about 0.5× to about 5.0×, about 0.5× to about 4.5×, about 0.5× to about 4.0×, about 0.5× to about 3.5×, about 0.5× to about 3.0×, about 0.5× to about 2.5×, about 0.5× to about 2.0×, about 0.5× to about 1.5×, about 0.5× to about 1.0×, about 1.0× to about 10×, about 1.0× to about 9.5×, about 1.0× to about 9.0×, about 1.0× to about 8.5×, about 1.0× to about 8.0×, about 1.0× to about 7.5×, about 1.0× to about 7.0×, about 1.0× to about 6.5×, about 1.0× to about 6.0×, about 1.0× to about 5.5×, about 1.0× to about 5.0×, about 1.0× to about 4.5×, about 1.0× to about 4.0×, about 1.0× to about 3.5×, about 1.0× to about 3.0×, about 1.0× to about 2.5×, about 1.0× to about 2.0×, about 1.0× to about 1.5×, about 1.5× to about 10×, about 1.5× to about 9.5×, about 1.5× to about 9.0×, about 1.5× to about 8.5×, about 1.5× to about 8.0×, about 1.5× to about 7.5×, about 1.5× to about 7.0×, about 1.5× to about 6.5×, about 1.5× to about 6.0×, about 1.5× to about 5.5×, about 1.5× to about 5.0×, about 1.5× to about 4.5×, about 1.5× to about 4.0×, about 1.5× to about 3.5×, about 1.5× to about 3.0×, about 1.5× to about 2.5×, about 1.5× to about 2.0×, about 2.0× to about 10×, about 2.0× to about 9.5×, about 2.0× to about 9.0×, about 2.0× to about 8.5×, about 2.0× to about 8.0×, about 2.0× to about 7.5×, about 2.0× to about 7.0×, about 2.0× to about 6.5×, about 2.0× to about 6.0×, about 2.0× to about 5.5×, about 2.0× to about 5.0×, about 2.0× to about 4.5×, about 2.0× to about 4.0×, about 2.0× to about 3.5×, about 2.0× to about 3.0×, about 2.0× to about 2.5×, about 2.5× to about 10×, about 2.5× to about 9.5×, about 2.5× to about 9.0×, about 2.5× to about 8.5×, about 2.5× to about 8.0×, about 2.5× to about 7.5×, about 2.5× to about 7.0×, about 2.5× to about 6.5×, about 2.5× to about 6.0×, about 2.5× to about 5.5×, about 2.5× to about 5.0×, about 2.5× to about 4.5×, about 2.5× to about 4.0×, about 2.5× to about 3.5×, about 2.5× to about 3.0×, about 3.0× to about 10×, about 3.0× to about 9.5×, about 3.0× to about 9.0×, about 3.0× to about 8.5×, about 3.0× to about 8.0×, about 3.0× to about 7.5×, about 3.0× to about 7.0×, about 3.0× to about 6.5×, about 3.0× to about 6.0×, about 3.0× to about 5.5×, about 3.0× to about 5.0×, about 3.0× to about 4.5×, about 3.0× to about 4.0×, about 3.0× to about 3.5×, about 3.5× to about 10×, about 3.5× to about 9.5×, about 3.5× to about 9.0×, about 3.5× to about 8.5×, about 3.5× to about 8.0×, about 3.5× to about 7.5×, about 3.5× to about 7.0×, about 3.5× to about 6.5×, about 3.5× to about 6.0×, about 3.5× to about 5.5×, about 3.5× to about 5.0×, about 3.5× to about 4.5×, about 3.5× to about 4.0×, about 4.0× to about 10×, about 4.0× to about 9.5×, about 4.0× to about 9.0×, about 4.0× to about 8.5×, about 4.0× to about 8.0×, about 4.0× to about 7.5×, about 4.0× to about 7.0×, about 4.0× to about 6.5×, about 4.0× to about 6.0×, about 4.0× to about 5.5×, about 4.0× to about 5.0×, about 4.0× to about 4.5×, about 4.5× to about 10×, about 4.5× to about 9.5×, about 4.5× to about 9.0×, about 4.5× to about 8.5×, about 4.5× to about 8.0×, about 4.5× to about 7.5×, about 4.5× to about 7.0×, about 4.5× to about 6.5×, about 4.5× to about 6.0×, about 4.5× to about 5.5×, about 4.5× to about 5.0×, about 5.0× to about 10×, about 5.0× to about 9.5×, about 5.0× to about 9.0×, about 5.0× to about 8.5×, about 5.0× to about 8.0×, about 5.0× to about 7.5×, about 5.0× to about 7.0×, about 5.0× to about 6.5×, about 5.0× to about 6.0×, about 5.0× to about 5.5×, about 5.5× to about 10×, about 5.5× to about 9.5×, about 5.5× to about 9.0×, about 5.5× to about 8.5×, about 5.5× to about 8.0×, about 5.5× to about 7.5×, about 5.5× to about 7.0×, about 5.5× to about 6.5×, about 5.5× to about 6.0×, about 6.0× to about 10×, about 6.0× to about 9.5×, about 6.0× to about 9.0×, about 6.0× to about 8.5×, about 6.0× to about 8.0×, about 6.0× to about 7.5×, about 6.0× to about 7.0×, about 6.0× to about 6.5×, about 6.5× to about 10×, about 6.5× to about 9.5×, about 6.5× to about 9.0×, about 6.5× to about 8.5×, about 6.5× to about 8.0×, about 6.5× to about 7.5×, about 6.5× to about 7.0×, about 7.0× to about 10×, about 7.0× to about 9.5×, about 7.0× to about 9.0×, about 7.0× to about 8.5×, about 7.0× to about 8.0×, about 7.0× to about 7.5×, about 7.5× to about 10×, about 7.5× to about 9.5×, about 7.5× to about 9.0×, about 7.5× to about 8.5×, about 7.5× to about 8.0×, about 8.0× to about 10×, about 8.0× to about 9.5×, about 8.0× to about 9.0×, about 8.0× to about 8.5×, about 8.5× to about 10×, about 8.5× to about 9.5×, about 8.5× to about 9.0×, about 9.0× to about 10×, about 9.0× to about 9.5×, or about 9.5× to about 10×, of the volume of the first cell culture immediately after step (i) is added in each 24-hour increment. In some embodiments, the addition of additional volumes of the first culture medium to the first cell culture begins once the first cell culture reaches a target specific cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

### Perfusion Culturing

In some embodiments, the step (ii) includes perfusion culturing the first cell culture. In some embodiments, step (ii) includes agitating the first cell culture (e.g., in any of the bioreactors described herein having any of the exemplary volumes described herein) with a P/V value of about 10 W/m³ to about 200 W/m³ (e.g., about 10 W/m³ to about 180 W/m³, about 10 W/m³ to about 160 W/m³, about 10 W/m³ to about 140 W/m³, about 10 W/m³ to about 120 W/m³, about 10 W/m³ to about 100 W/m³, about 10 W/m³ to about 80 W/m³, about 10 W/m³ to about 60 W/m³, about 10 W/m³ to about 40 W/m³, about 10 W/m³ to about 35 W/m³, about 10 W/m³ to about 30 W/m³, about 10 W/m³ to about 25 W/m³, about 10 W/m³ to about 20 W/m³, about 10 W/m³ to about 15 W/m³, about 15 W/m³ to about 200 W/m³, about 15 W/m³ to about 180 W/m³, about 15 W/m³ to about 160 W/m³, about 15 W/m³ to about 140 W/m³, about 15 W/m³ to about 120 W/m³, about 15 W/m³ to about 100 W/m³, about 15 W/m³ to about 80 W/m³, about 15 W/m³ to about 60 W/m³, about 15 W/m³ to about 40 W/m³, about 15 W/m³ to about 35 W/m³, about 15 W/m³ to about 30 W/m³, about 15 W/m³ to about 25 W/m³, about 15 W/m³ to about 20 W/m³, about 20 W/m³ to about 200 W/m³, about 20 W/m³ to about 180 W/m³, about 20 W/m³ to about 160 W/m³, about 20 W/m³ to about 140 W/m³, about 20 W/m³ to about 120 W/m³, about 20 W/m³ to about 100 W/m³, about 20 W/m³ to about 80 W/m³, about 20 W/m³ to about 60 W/m³, about 20 W/m³ to about 40 W/m³, about 20 W/m³ to about 35 W/m³, about 20 W/m³ to about 30 W/m³, about 20 W/m³ to about 25 W/m³, about 25 W/m³ to about 200 W/m³, about 25 W/m³ to about 180 W/m³, about 25 W/m³ to about 160 W/m³, about 25 W/m³ to about 140 W/m³, about 25 W/m³ to about 120 W/m³, about 25 W/m³ to about 100 W/m³, about 25 W/m³ to about 80 W/m³, about 25 W/m³ to about 60 W/m³, about 25 W/m³ to about 40 W/m³, about 25 W/m³ to about 35 W/m³, about 25 W/m³ to about 30 W/m³, about 30 W/m³ to about 200 W/m³, about 30 W/m³ to about 180 W/m³, about 30 W/m³ to about 160 W/m³, about 30 W/m³ to about 140 W/m³, about 30 W/m³ to about 120 W/m³, about 30 W/m³ to about 100 W/m³, about 30 W/m³ to about 80 W/m³, about 30 W/m³ to about 60 W/m³, about 30 W/m³ to about 40 W/m³, about 30 W/m³ to about 35 W/m³, about 35 W/m³ to about 200 W/m³, about 35 W/m³ to about 180 W/m³, about 35 W/m³ to about 160 W/m³, about 35 W/m³ to about 140 W/m³, about 35 W/m³ to about 120 W/m³, about 35 W/m³ to about 100 W/m³, about 35 W/m³ to about 80 W/m³, about 35 W/m³ to about 60 W/m³, about 35 W/m³ to about 40 W/m³, about 40 W/m³ to about 200 W/m³, about 40 W/m³ to about 180 W/m³, about 40 W/m³ to about 160 W/m³, about 40 W/m³ to about 140 W/m³, about 40 W/m³ to about 120 W/m³, about 40 W/m³ to about 100 W/m³, about 40 W/m³ to about 80 W/m³, about 40 W/m³ to about 60 W/m³, about 60 W/m³ to about 200 W/m³, about 60 W/m³ to about 180 W/m³, about 60 W/m³ to about 160 W/m³, about 60 W/m³ to about 140 W/m³, about 60 W/m³ to about 120 W/m³, about 60 W/m³ to about 100 W/m³, about 60 W/m³ to about 80 W/m³, about 80 W/m³ to about 200 W/m³, about 80 W/m³ to about 180 W/m³, about 80 W/m³ to about 160 W/m³, about 80 W/m³ to about 140 W/m³, about 80 W/m³ to about 120 W/m³, about 80 W/m³ to about 100 W/m³, about 100 W/m³ to about 200 W/m³, about 100 W/m³ to about 180 W/m³, about 100 W/m³ to about 160 W/m³, about 100 W/m³ to about 140 W/m³, about 100 W/m³ to about 120 W/m³, about 120 W/m³ to about 200 W/m³, about 120 W/m³ to about 180 W/m³, about 120 W/m³ to about 160 W/m³, about 120 W/m³ to about 140 W/m³, about 140 W/m³ to about 200 W/m³, about 140 W/m³ to about 180 W/m³, about 140 W/m³ to about 160 W/m³, about 160 W/m³ to about 200 W/m³, about 160 W/m³ to about 180 W/m³, and about 180 W/m³ to about 200 W/m³).

In some embodiments, the perfusion culturing in step (ii) can be performed using a perfusion rate of about 0.04 nL/cell/day to about 60 nL/cell/day, about 0.04 nL/cell/day to about 55 nL/cell/day, about 0.04 nL/cell/day to about 50 nL/cell/day, about 0.04 nL/cell/day to about 45 nL/cell/day, about 0.04 nL/cell/day to about 40 nL/cell/day, about 0.04 nL/cell/day to about 35 nL/cell/day, about 0.04 nL/cell/day to about 30 nL/cell/day, about 0.04 nL/cell/day to about 25 nL/cell/day, about 0.04 nL/cell/day to about 20 nL/cell/day, about 0.04 nL/cell/day to about 15 nL/cell/day, about 0.04 nL/cell/day to about 10 nL/cell/day, about 0.04 nL/cell/day to about 8 nL/cell/day, about 0.04 nL/cell/day to about 6 nL/cell/day, about 0.04 nL/cell/day to about 5 nL/cell/day, about 0.04 nL/cell/day to about 4 nL/cell/day, about 0.04 nL/cell/day to about 2 nL/cell/day, about 0.04 nL/cell/day to about 1 nL/cell/day, about 0.04 nL/cell/day to about 0.5 nL/cell/day, about 0.04 nL/cell/day to about 0.1 nL/cell/day, about 0.1 nL/cell/day to about 60 nL/cell/day, about 0.1 nL/cell/day to about 55 nL/cell/day, about 0.1 nL/cell/day to about 50 nL/cell/day, about 0.1 nL/cell/day to about 45 nL/cell/day, about 0.1 nL/cell/day to about 40 nL/cell/day, about 0.1 nL/cell/day to about 35 nL/cell/day, about 0.1 nL/cell/day to about 30 nL/cell/day, about 0.1 nL/cell/day to about 25 nL/cell/day, about 0.1 nL/cell/day to about 20 nL/cell/day, about 0.1 nL/cell/day to about 15 nL/cell/day, about 0.1 nL/cell/day to about 10 nL/cell/day, about 0.1 nL/cell/day to about 8 nL/cell/day, about 0.1 nL/cell/day to about 6 nL/cell/day, about 0.1 nL/cell/day to about 5 nL/cell/day, about 0.1 nL/cell/day to about 4 nL/cell/day, about 0.1 nL/cell/day to about 2 nL/cell/day, about 0.1 nL/cell/day to about 1 nL/cell/day, about 0.1 nL/cell/day to about 0.5 nL/cell/day, about 0.5 nL/cell/day to about 60 nL/cell/day, about 0.5 nL/cell/day to about 55 nL/cell/day, about 0.5 nL/cell/day to about 50 nL/cell/day, about 0.5 nL/cell/day to about 45 nL/cell/day, about 0.5 nL/cell/day to about 40 nL/cell/day, about 0.5 nL/cell/day to about 35 nL/cell/day, about 0.5 nL/cell/day to about 30 nL/cell/day, about 0.5 nL/cell/day to about 25 nL/cell/day, about 0.5 nL/cell/day to about 20 nL/cell/day, about 0.5 nL/cell/day to about 15 nL/cell/day, about 0.5 nL/cell/day to about 10 nL/cell/day, about 0.5 nL/cell/day to about 8 nL/cell/day, about 0.5 nL/cell/day to about 6 nL/cell/day, about 0.5 nL/cell/day to about 5 nL/cell/day, about 0.5 nL/cell/day to about 4 nL/cell/day, about 0.5 nL/cell/day to about 2 nL/cell/day, about 0.5 nL/cell/day to about 1 nL/cell/day, about 1 nL/cell/day to about 60 nL/cell/day, about 1 nL/cell/day to about 55 nL/cell/day, about 1 nL/cell/day to about 50 nL/cell/day, about 1 nL/cell/day to about 45 nL/cell/day, about 1 nL/cell/day to about 40 nL/cell/day, about 1 nL/cell/day to about 35 nL/cell/day, about 1 nL/cell/day to about 30 nL/cell/day, about 1 nL/cell/day to about 25 nL/cell/day, about 1 nL/cell/day to about 20 nL/cell/day, about 1 nL/cell/day to about 15 nL/cell/day, about 1 nL/cell/day to about 10 nL/cell/day, about 1 nL/cell/day to about 8 nL/cell/day, about 1 nL/cell/day to about 6 nL/cell/day, about 1 nL/cell/day to about 5 nL/cell/day, about 1 nL/cell/day to about 4 nL/cell/day, about 1 nL/cell/day to about 2 nL/cell/day, about 2 nL/cell/day to about 60 nL/cell/day, about 2 nL/cell/day to about 55 nL/cell/day, about 2 nL/cell/day to about 50 nL/cell/day, about 2 nL/cell/day to about 45 nL/cell/day, about 2 nL/cell/day to about 40 nL/cell/day, about 2 nL/cell/day to about 35 nL/cell/day, about 2 nL/cell/day to about 30 nL/cell/day, about 2 nL/cell/day to about 25 nL/cell/day, about 2 nL/cell/day to about 20 nL/cell/day, about 2 nL/cell/day to about 15 nL/cell/day, about 2 nL/cell/day to about 10 nL/cell/day, about 2 nL/cell/day to about 8 nL/cell/day, about 2 nL/cell/day to about 6 nL/cell/day, about 2 nL/cell/day to about 5 nL/cell/day, about 2 nL/cell/day to about 4 nL/cell/day, about 4 nL/cell/day to about 60 nL/cell/day, about 4 nL/cell/day to about 55 nL/cell/day, about 4 nL/cell/day to about 50 nL/cell/day, about 4 nL/cell/day to about 45 nL/cell/day, about 4 nL/cell/day to about 40 nL/cell/day, about 4 nL/cell/day to about 35 nL/cell/day, about 4 nL/cell/day to about 30 nL/cell/day, about 4 nL/cell/day to about 25 nL/cell/day, about 4 nL/cell/day to about 20 nL/cell/day, about 4 nL/cell/day to about 15 nL/cell/day, about 4 nL/cell/day to about 10 nL/cell/day, about 4 nL/cell/day to about 8 nL/cell/day, about 4 nL/cell/day to about 6 nL/cell/day, about 4 nL/cell/day to about 5 nL/cell/day, about 5 nL/cell/day to about 60 nL/cell/day, about 5 nL/cell/day to about 55 nL/cell/day, about 5 nL/cell/day to about 50 nL/cell/day, about 5 nL/cell/day to about 45 nL/cell/day, about 5 nL/cell/day to about 40 nL/cell/day, about 5 nL/cell/day to about 35 nL/cell/day, about 5 nL/cell/day to about 30 nL/cell/day, about 5 nL/cell/day to about 25 nL/cell/day, about 5 nL/cell/day to about 20 nL/cell/day, about 5 nL/cell/day to about 15 nL/cell/day, about 5 nL/cell/day to about 10 nL/cell/day, about 5 nL/cell/day to about 8 nL/cell/day, about 5 nL/cell/day to about 6 nL/cell/day, about 6 nL/cell/day to about 60 nL/cell/day, about 6 nL/cell/day to about 55 nL/cell/day, about 6 nL/cell/day to about 50 nL/cell/day, about 6 nL/cell/day to about 45 nL/cell/day, about 6 nL/cell/day to about 40 nL/cell/day, about 6 nL/cell/day to about 35 nL/cell/day, about 6 nL/cell/day to about 30 nL/cell/day, about 6 nL/cell/day to about 25 nL/cell/day, about 6 nL/cell/day to about 20 nL/cell/day, about 6 nL/cell/day to about 15 nL/cell/day, about 6 nL/cell/day to about 10 nL/cell/day, about 6 nL/cell/day to about 8 nL/cell/day, about 8 nL/cell/day to about 60 nL/cell/day, about 8 nL/cell/day to about 55 nL/cell/day, about 8 nL/cell/day to about 50 nL/cell/day, about 8 nL/cell/day to about 45 nL/cell/day, about 8 nL/cell/day to about 40 nL/cell/day, about 8 nL/cell/day to about 35 nL/cell/day, about 8 nL/cell/day to about 30 nL/cell/day, about 8 nL/cell/day to about 25 nL/cell/day, about 8 nL/cell/day to about 20 nL/cell/day, about 8 nL/cell/day to about 15 nL/cell/day, about 8 nL/cell/day to about 10 nL/cell/day, about 10 nL/cell/day to about 60 nL/cell/day, about 10 nL/cell/day to about 55 nL/cell/day, about 10 nL/cell/day to about 50 nL/cell/day, about 10 nL/cell/day to about 45 nL/cell/day, about 10 nL/cell/day to about 40 nL/cell/day, about 10 nL/cell/day to about 35 nL/cell/day, about 10 nL/cell/day to about 30 nL/cell/day, about 10 nL/cell/day to about 25 nL/cell/day, about 10 nL/cell/day to about 20 nL/cell/day, about 10 nL/cell/day to about 15 nL/cell/day, about 15 nL/cell/day to about 60 nL/cell/day, about 15 nL/cell/day to about 55 nL/cell/day, about 15 nL/cell/day to about 50 nL/cell/day, about 15 nL/cell/day to about 45 nL/cell/day, about 15 nL/cell/day to about 40 nL/cell/day, about 15 nL/cell/day to about 35 nL/cell/day, about 15 nL/cell/day to about 30 nL/cell/day, about 15 nL/cell/day to about 25 nL/cell/day, about 15 nL/cell/day to about 20 nL/cell/day, about 20 nL/cell/day to about 60 nL/cell/day, about 20 nL/cell/day to about 55 nL/cell/day, about 20 nL/cell/day to about 50 nL/cell/day, about 20 nL/cell/day to about 45 nL/cell/day, about 20 nL/cell/day to about 40 nL/cell/day, about 20 nL/cell/day to about 35 nL/cell/day, about 20 nL/cell/day to about 30 nL/cell/day, about 20 nL/cell/day to about 25 nL/cell/day, about 25 nL/cell/day to about 60 nL/cell/day, about 25 nL/cell/day to about 55 nL/cell/day, about 25 nL/cell/day to about 50 nL/cell/day, about 25 nL/cell/day to about 45 nL/cell/day, about 25 nL/cell/day to about 40 nL/cell/day, about 25 nL/cell/day to about 35 nL/cell/day, about 25 nL/cell/day to about 30 nL/cell/day, about 30 nL/cell/day to about 60 nL/cell/day, about 30 nL/cell/day to about 55 nL/cell/day, about 30 nL/cell/day to about 50 nL/cell/day, about 30 nL/cell/day to about 45 nL/cell/day, about 30 nL/cell/day to about 40 nL/cell/day, about 30 nL/cell/day to about 35 nL/cell/day, about 35 nL/cell/day to about 60 nL/cell/day, about 35 nL/cell/day to about 55 nL/cell/day, about 35 nL/cell/day to about 50 nL/cell/day, about 35 nL/cell/day to about 45 nL/cell/day, about 35 nL/cell/day to about 40 nL/cell/day, about 40 nL/cell/day to about 60 nL/cell/day, about 40 nL/cell/day to about 55 nL/cell/day, about 40 nL/cell/day to about 50 nL/cell/day, about 40 nL/cell/day to about 45 nL/cell/day, about 45 nL/cell/day to about 60 nL/cell/day, about 45 nL/cell/day to about 55 nL/cell/day, about 45 nL/cell/day to about 50 nL/cell/day, about 50 nL/cell/day to about 60 nL/cell/day, about 50 nL/cell/day to about 55 nL/cell/day, or about 55 nL/cell/day to about 60 nL/cell/day. In some embodiments, the perfusion rate is increased over time.

In some embodiments, the perfusion culturing in step (ii) can be performed using a perfusion rate of about 0.1 vessel volume per day (VVD) to about 3 VVD (e.g., about 0.1 VVD to about 2.8 VVD, about 0.1 VVD to about 2.6 VVD, about 0.1 VVD to about 2.4 VVD, about 0.1 VVD to about 2.2 VVD, about 0.1 VVD to about 2.0 VVD, about 0.1 VVD to about 1.8 VVD, about 0.1 VVD to about 1.6 VVD, about 0.1 VVD to about 1.4 VVD, about 0.1 VVD to about 1.2 VVD, about 0.1 VVD to about 1.0 VVD, about 0.1 VVD to about 0.8 VVD, about 0.1 VVD to about 0.6 VVD, about 0.1 VVD to about 0.4 VVD, about 0.1 VVD to about 0.2 VVD, about 0.2 VVD to about 3 VVD, about 0.2 VVD to about 2.8 VVD, about 0.2 VVD to about 2.6 VVD, about 0.2 VVD to about 2.4 VVD, about 0.2 VVD to about 2.2 VVD, about 0.2 VVD to about 2.0 VVD, about 0.2 VVD to about 1.8 VVD, about 0.2 VVD to about 1.6 VVD, about 0.2 VVD to about 1.4 VVD, about 0.2 VVD to about 1.2 VVD, about 0.2 VVD to about 1.0 VVD, about 0.2 VVD to about 0.8 VVD, about 0.2 VVD to about 0.6 VVD, about 0.2 VVD to about 0.4 VVD, about 0.4 VVD to about 3 VVD, about 0.4 VVD to about 2.8 VVD, about 0.4 VVD to about 2.6 VVD, about 0.4 VVD to about 2.4 VVD, about 0.4 VVD to about 2.2 VVD, about 0.4 VVD to about 2.0 VVD, about 0.4 VVD to about 1.8 VVD, about 0.4 VVD to about 1.6 VVD, about 0.4 VVD to about 1.4 VVD, about 0.4 VVD to about 1.2 VVD, about 0.4 VVD to about 1.0 VVD, about 0.4 VVD to about 0.8 VVD, about 0.4 VVD to about 0.6 VVD, about 0.6 VVD to about 3 VVD, about 0.6 VVD to about 2.8 VVD, about 0.6 VVD to about 2.6 VVD, about 0.6 VVD to about 2.4 VVD, about 0.6 VVD to about 2.2 VVD, about 0.6 VVD to about 2.0 VVD, about 0.6 VVD to about 1.8 VVD, about 0.6 VVD to about 1.6 VVD, about 0.6 VVD to about 1.4 VVD, about 0.6 VVD to about 1.2 VVD, about 0.6 VVD to about 1.0 VVD, about 0.6 VVD to about 0.8 VVD, about 0.8 VVD to about 3 VVD, about 0.8 VVD to about 2.8 VVD, about 0.8 VVD to about 2.6 VVD, about 0.8 VVD to about 2.4 VVD, about 0.8 VVD to about 2.2 VVD, about 0.8 VVD to about 2.0 VVD, about 0.8 VVD to about 1.8 VVD, about 0.8 VVD to about 1.6 VVD, about 0.8 VVD to about 1.4 VVD, about 0.8 VVD to about 1.2 VVD, about 0.8 VVD to about 1.0 VVD, about 1.0 VVD to about 3 VVD, about 1.0 VVD to about 2.8 VVD, about 1.0 VVD to about 2.6 VVD, about 1.0 VVD to about 2.4 VVD, about 1.0 VVD to about 2.2 VVD, about 1.0 VVD to about 2.0 VVD, about 1.0 VVD to about 1.8 VVD, about 1.0 VVD to about 1.6 VVD, about 1.0 VVD to about 1.4 VVD, about 1.0 VVD to about 1.2 VVD, about 1.2 VVD to about 3 VVD, about 1.2 VVD to about 2.8 VVD, about 1.2 VVD to about 2.6 VVD, about 1.2 VVD to about 2.4 VVD, about 1.2 VVD to about 2.2 VVD, about 1.2 VVD to about 2.0 VVD, about 1.2 VVD to about 1.8 VVD, about 1.2 VVD to about 1.6 VVD, about 1.2 VVD to about 1.4 VVD, about 1.4 VVD to about 3 VVD, about 1.4 VVD to about 2.8 VVD, about 1.4 VVD to about 2.6 VVD, about 1.4 VVD to about 2.4 VVD, about 1.4 VVD to about 2.2 VVD, about 1.4 VVD to about 2.0 VVD, about 1.4 VVD to about 1.8 VVD, about 1.4 VVD to about 1.6 VVD, about 1.6 VVD to about 3 VVD, about 1.6 VVD to about 2.8 VVD, about 1.6 VVD to about 2.6 VVD, about 1.6 VVD to about 2.4 VVD, about 1.6 VVD to about 2.2 VVD, about 1.6 VVD to about 2.0 VVD, about 1.6 VVD to about 1.8 VVD, about 1.8 VVD to about 3 VVD, about 1.8 VVD to about 2.8 VVD, about 1.8 VVD to about 2.6 VVD, about 1.8 VVD to about 2.4 VVD, about 1.8 VVD to about 2.2 VVD, about 1.8 VVD to about 2.0 VVD, about 2.0 VVD to about 3 VVD, about 2.0 VVD to about 2.8 VVD, about 2.0 VVD to about 2.6 VVD, about 2.0 VVD to about 2.4 VVD, about 2.0 VVD to about 2.2 VVD, about 2.2 VVD to about 3 VVD, about 2.2 VVD to about 2.8 VVD, about 2.2 VVD to about 2.6 VVD, about 2.2 VVD to about 2.4 VVD, about 2.4 VVD to about 3 VVD, about 2.4 VVD to about 2.8 VVD, about 2.4 VVD to about 2.6 VVD, about 2.6 VVD to about 3 VVD, about 2.6 VVD to about 2.8 VVD, or about 2.8 VVD to about 3 VVD).

In some examples, the perfusion culturing in step (ii) includes, at least in part, adding an additional volume of culture medium (e.g., the first culture medium (e.g., any of the exemplary first culture media described herein)) to the first cell culture over time.

In some examples, the additional volume of culture medium (e.g., the first culture medium (e.g., any of the exemplary first culture media described herein)) is added continuously to the first cell culture over time. In some embodiments, the additional volume of culture medium (e.g., the first culture medium (e.g., any of the exemplary first culture media described herein)) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the first cell culture over time. The addition of culture medium (e.g., any of the exemplary first culture media described herein) can be performed mechanically, e.g., using a peristaltic pump or a perfusion pump, or manually (e.g., by sterile pipetting).

In some examples, the perfusion culturing in step (ii) includes, at least in part, removing a volume of the culture medium (e.g., substantially cell-free culture medium) over time. In some examples, the culture medium (e.g., substantially cell-free culture medium) is removed continuously over time. In some embodiments, the culture medium is removed periodically (e.g., once every three days, once every two days, once a day, twice a day, three times a day, four times a day, five times a day, six times a day, seven times a day, eight times a day, nine times a day, ten times a day, eleven times a day, or twelve times a day) over time. The removal of culture medium (e.g., substantially cell-free culture medium) can be performed mechanically, e.g., using a tangential flow filtration (TFF) or alternating flow filtration (ATF), or manually (e.g., by sterile pipetting). Additional non-limiting aspects of tangential flow filtration are described herein. In some embodiments, the perfusion culturing includes the use of tangential filtration (e.g., tangential flow filtration (TFF) or alternating tangential filtration (ATF)). In some embodiments, the tangential filtration (e.g., tangential flow filtration or alternating tangential filtration) includes the use of one or more filters that have an average pore size of about 10 nm to about 6.0 μm, about 10 nm to about 5.5 μm, about 10 nm to about 5.0 μm, about 10 nm to about 4.5 μm, about 10 nm to about 4.0 μm, about 10 nm to about 3.5 μm, about 10 nm to about 3.0 μm, about 10 nm to about 2.5 μm, about 10 nm to about 2.0 μm, about 10 nm to about 1.5 μm, about 10 nm to about 1.0 μm, about 10 nm to about 0.5 μm, about 10 nm to about 0.2 μm, 10 nm to about 0.1 μm, about 10 nm to about 50 nm, about 50 nm to about 6.0 μm, about 50 nm to about 5.5 μm, about 50 nm to about 5.0 μm, about 50 nm to about 4.5 μm, about 50 nm to about 4.0 μm, about 50 nm to about 3.5 μm, about 50 nm to about 3.0 μm, about 50 nm to about 2.5 μm, about 50 nm to about 2.0 μm, about 50 nm to about 1.5 μm, about 50 nm to about 1.0 μm, about 50 nm to about 0.5 μm, about 50 nm to about 0.2 μm, about 50 nm to about 0.1 μm, about 0.1 μm to about 6.0 μm, about 0.1 μm to about 5.5 μm, about 0.1 μm to about 5.0 μm, about 0.1 μm to about 4.5 μm, about 0.1 μm to about 4.0 μm, about 0.1 μm to about 3.5 μm, about 0.1 μm to about 3.0 μm, about 0.1 μm to about 2.5 μm, about 0.1 μm to about 2.0 μm, about 0.1 μm to about 1.5 μm, about 0.1 μm to about 1.0 μm, about 0.1 μm to about 0.5 μm, about 0.1 μm to about 0.2 μm, about 0.2 μm to about 6.0 μm, about 0.2 μm to about 5.5 μm, about 0.2 μm to about 5.0 μm, about 0.2 μm to about 4.5 μm, about 0.2 μm to about 4.0 μm, about 0.2 μm to about 3.5 μm, about 0.2 μm to about 3.0 μm, about 0.2 μm to about 2.5 μm, about 0.2 μm to about 2.0 μm, about 0.2 μm to about 1.5 μm, about 0.2 μm to about 1.0 μm, about 0.2 μm to about 0.5 μm, about 0.5 μm to about 6.0 μm, about 0.5 μm to about 5.5 μm, about 0.5 μm to about 5.0 μm, about 0.5 μm to about 4.5 μm, about 0.5 μm to about 4.0 μm, about 0.5 μm to about 3.5 μm, about 0.5 μm to about 3.0 μm, about 0.5 μm to about 2.5 μm, about 0.5 μm to about 2.0 μm, about 0.5 μm to about 1.5 μm, about 0.5 μm to about 1.0 μm, about 1.0 μm to about 6.0 μm, about 1.0 μm to about 5.5 μm, about 1.0 μm to about 5.0 μm, about 1.0 μm to about 4.5 μm, about 1.0 μm to about 4.0 μm, about 1.0 μm to about 3.5 μm, about 1.0 μm to about 3.0 μm, about 1.0 μm to about 2.5 μm, about 1.0 μm to about 2.0 μm, about 1.0 μm to about 1.5 μm, about 1.5 μm to about 6.0 μm, about 1.5 μm to about 5.5 μm, about 1.5 μm to about 5.0 μm, about 1.5 μm to about 4.5 μm, about 1.5 μm to about 4.0 μm, about 1.5 μm to about 3.5 μm, about 1.5 μm to about 3.0 μm, about 1.5 μm to about 2.5 μm, about 1.5 μm to about 2.0 μm, about 2.0 μm to about 6.0 μm, about 2.0 μm to about 5.5 μm, about 2.0 μm to about 5.0 μm, about 2.0 μm to about 4.5 μm, about 2.0 μm to about 4.0 μm, about 2.0 μm to about 3.5 μm, about 2.0 μm to about 3.0 μm, about 2.0 μm to about 2.5 μm, about 2.5 μm to about 6.0 μm, about 2.5 μm to about 5.5 μm, about 2.5 μm to about 5.0 μm, about 2.5 μm to about 4.5 μm, about 2.5 μm to about 4.0 μm, about 2.5 μm to about 3.5 μm, about 2.5 μm to about 3.0 μm, about 3.0 μm to about 6.0 μm, about 3.0 μm to about 5.5 μm, about 3.0 μm to about 5.0 μm, about 3.0 μm to about 4.5 μm, about 3.0 μm to about 4.0 μm, about 3.0 μm to about 3.5 μm, about 3.5 μm to about 6.0 μm, about 3.5 μm to about 5.5 μm, about 3.5 μm to about 5.0 μm, about 3.5 μm to about 4.5 μm, about 3.5 μm to about 4.0 μm, about 4.0 μm to about 6.0 μm, about 4.0 μm to about 5.5 μm, about 4.0 μm to about 5.0 μm, about 4.0 μm to about 4.5 μm, about 4.5 μm to about 6.0 about 4.5 μm to about 5.5 μm, about 4.5 μm to about 5.0 μm, about 5.0 μm to about 6.0 μm, about 5.0 μm to about 5.5 μm, or about 5.5 μm to about 6.0 μm.

In some embodiments, the perfusion of the first cell culture begins once the first cell culture reaches a specific target cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

In some embodiments, the first culture medium includes one or more (e.g., one, two, three, or four) of Flt-3 ligand (e.g., 0.1 ng/mL to about 200 ng/mL Flt-3 ligand or any of the subranges of this range described herein), SCF (e.g., about 1 ng/mL to about 1 μg/mL SCF or any of the subranges of this range described herein), IL-3 (e.g., about 0.1 ng/mL to about 200 ng/mL IL-3 or any of the subranges of this range described herein), and IL-6 (e.g., about 0.1 ng/mL to about 200 ng/mL IL-6 or any of the subranges of this range described herein). In some embodiments of any of the first culture media described herein, the first culture medium includes CellGenix SCGM media. In some embodiments of any of the first culture media, the first culture medium includes about 0.1 mM to about 10 mM (e.g., about 0.1 mM to about 9.5 mM, about 0.1 mM to about 9.0 mM, about 0.1 mM to about 8.5 mM, about 0.1 mM to about 8.0 mM, about 0.1 mM to about 7.5 mM, about 0.1 mM to about 7.0 mM, about 0.1 mM to about 6.5 mM, about 0.1 mM to about 6.0 mM, about 0.1 mM to about 5.5 mM, about 0.1 mM to about 5 mM, about 0.1 mM to about 4.5 mM, about 0.1 mM to about 4.0 mM, about 0.1 mM to about 3.5 mM, about 0.1 mM to about 3.0 mM, about 0.1 mM to about 2.5 mM, about 0.1 mM to about 2.0 mM, about 0.1 mM to about 1.5 mM, about 0.1 mM to about 1.0 mM, about 0.1 mM to about 0.5 mM, about 0.5 mM to about 10 mM, about 0.5 mM to about 9.5 mM, about 0.5 mM to about 9.0 mM, about 0.5 mM to about 8.5 mM, about 0.5 mM to about 8.0 mM, about 0.5 mM to about 7.5 mM, about 0.5 mM to about 7.0 mM, about 0.5 mM to about 6.5 mM, about 0.5 mM to about 6.0 mM, about 0.5 mM to about 5.5 mM, about 0.5 mM to about 5 mM, about 0.5 mM to about 4.5 mM, about 0.5 mM to about 4.0 mM, about 0.5 mM to about 3.5 mM, about 0.5 mM to about 3.0 mM, about 0.5 mM to about 2.5 mM, about 0.5 mM to about 2.0 mM, about 0.5 mM to about 1.5 mM, about 0.5 mM to about 1.0 mM, about 1.0 mM to about 10 mM, about 1.0 mM to about 9.5 mM, about 1.0 mM to about 9.0 mM, about 1.0 mM to about 8.5 mM, about 1.0 mM to about 8.0 mM, about 1.0 mM to about 7.5 mM, about 1.0 mM to about 7.0 mM, about 1.0 mM to about 6.5 mM, about 1.0 mM to about 6.0 mM, about 1.0 mM to about 5.5 mM, about 1.0 mM to about 5 mM, about 1.0 mM to about 4.5 mM, about 1.0 mM to about 4.0 mM, about 1.0 mM to about 3.5 mM, about 1.0 mM to about 3.0 mM, about 1.0 mM to about 2.5 mM, about 1.0 mM to about 2.0 mM, about 1.0 mM to about 1.5 mM, about 1.5 mM to about 10 mM, about 1.5 mM to about 9.5 mM, about 1.5 mM to about 9.0 mM, about 1.5 mM to about 8.5 mM, about 1.5 mM to about 8.0 mM, about 1.5 mM to about 7.5 mM, about 1.5 mM to about 7.0 mM, about 1.5 mM to about 6.5 mM, about 1.5 mM to about 6.0 mM, about 1.5 mM to about 5.5 mM, about 1.5 mM to about 5 mM, about 1.5 mM to about 4.5 mM, about 1.5 mM to about 4.0 mM, about 1.5 mM to about 3.5 mM, about 1.5 mM to about 3.0 mM, about 1.5 mM to about 2.5 mM, about 1.5 mM to about 2.0 mM, about 2.0 mM to about 10 mM, about 2.0 mM to about 9.5 mM, about 2.0 mM to about 9.0 mM, about 2.0 mM to about 8.5 mM, about 2.0 mM to about 8.0 mM, about 2.0 mM to about 7.5 mM, about 2.0 mM to about 7.0 mM, about 2.0 mM to about 6.5 mM, about 2.0 mM to about 6.0 mM, about 2.0 mM to about 5.5 mM, about 2.0 mM to about 5 mM, about 2.0 mM to about 4.5 mM, about 2.0 mM to about 4.0 mM, about 2.0 mM to about 3.5 mM, about 2.0 mM to about 3.0 mM, about 2.0 mM to about 2.5 mM, about 2.5 mM to about 10 mM, about 2.5 mM to about 9.5 mM, about 2.5 mM to about 9.0 mM, about 2.5 mM to about 8.5 mM, about 2.5 mM to about 8.0 mM, about 2.5 mM to about 7.5 mM, about 2.5 mM to about 7.0 mM, about 2.5 mM to about 6.5 mM, about 2.5 mM to about 6.0 mM, about 2.5 mM to about 5.5 mM, about 2.5 mM to about 5 mM, about 2.5 mM to about 4.5 mM, about 2.5 mM to about 4.0 mM, about 2.5 mM to about 3.5 mM, about 2.5 mM to about 3.0 mM, about 3.0 mM to about 10 mM, about 3.0 mM to about 9.5 mM, about 3.0 mM to about 9.0 mM, about 3.0 mM to about 8.5 mM, about 3.0 mM to about 8.0 mM, about 3.0 mM to about 7.5 mM, about 3.0 mM to about 7.0 mM, about 3.0 mM to about 6.5 mM, about 3.0 mM to about 6.0 mM, about 3.0 mM to about 5.5 mM, about 3.0 mM to about 5 mM, about 3.0 mM to about 4.5 mM, about 3.0 mM to about 4.0 mM, about 3.0 mM to about 3.5 mM, about 3.5 mM to about 10 mM, about 3.5 mM to about 9.5 mM, about 3.5 mM to about 9.0 mM, about 3.5 mM to about 8.5 mM, about 3.5 mM to about 8.0 mM, about 3.5 mM to about 7.5 mM, about 3.5 mM to about 7.0 mM, about 3.5 mM to about 6.5 mM, about 3.5 mM to about 6.0 mM, about 3.5 mM to about 5.5 mM, about 3.5 mM to about 5 mM, about 3.5 mM to about 4.5 mM, about 3.5 mM to about 4.0 mM, about 4.0 mM to about 10 mM, about 4.0 mM to about 9.5 mM, about 4.0 mM to about 9.0 mM, about 4.0 mM to about 8.5 mM, about 4.0 mM to about 8.0 mM, about 4.0 mM to about 7.5 mM, about 4.0 mM to about 7.0 mM, about 4.0 mM to about 6.5 mM, about 4.0 mM to about 6.0 mM, about 4.0 mM to about 5.5 mM, about 4.0 mM to about 5 mM, about 4.0 mM to about 4.5 mM, about 4.5 mM to about 10 mM, about 4.5 mM to about 9.5 mM, about 4.5 mM to about 9.0 mM, about 4.5 mM to about 8.5 mM, about 4.5 mM to about 8.0 mM, about 4.5 mM to about 7.5 mM, about 4.5 mM to about 7.0 mM, about 4.5 mM to about 6.5 mM, about 4.5 mM to about 6.0 mM, about 4.5 mM to about 5.5 mM, about 4.5 mM to about 5.0 mM, about 5.0 mM to about 10 mM, about 5.0 mM to about 9.5 mM, about 5.0 mM to about 9.0 mM, about 5.0 mM to about 8.5 mM, about 5.0 mM to about 8.0 mM, about 5.0 mM to about 7.5 mM, about 5.0 mM to about 7.0 mM, about 5.0 mM to about 6.5 mM, about 5.0 mM to about 6.0 mM, about 5.0 mM to about 5.5 mM, about 5.5 mM to about 10 mM, about 5.5 mM to about 9.5 mM, about 5.5 mM to about 9.0 mM, about 5.5 mM to about 8.5 mM, about 5.5 mM to about 8.0 mM, about 5.5 mM to about 7.5 mM, about 5.5 mM to about 7.0 mM, about 5.5 mM to about 6.5 mM, about 5.5 mM to about 6.0 mM, about 6.0 mM to about 10 mM, about 6.0 mM to about 9.5 mM, about 6.0 mM to about 9.0 mM, about 6.0 mM to about 8.5 mM, about 6.0 mM to about 8.0 mM, about 6.0 mM to about 7.5 mM, about 6.0 mM to about 7.0 mM, about 6.0 mM to about 6.5 mM, about 6.5 mM to about 10 mM, about 6.5 mM to about 9.5 mM, about 6.5 mM to about 9.0 mM, about 6.5 mM to about 8.5 mM, about 6.5 mM to about 8.0 mM, about 6.5 mM to about 7.5 mM, about 6.5 mM to about 7.0 mM, about 7.0 mM to about 10 mM, about 7.0 mM to about 9.5 mM, about 7.0 mM to about 9.0 mM, about 7.0 mM to about 8.5 mM, about 7.0 mM to about 8.0 mM, about 7.0 mM to about 7.5 mM, about 7.5 mM to about 10 mM, about 7.5 mM to about 9.5 mM, about 7.5 mM to about 9.0 mM, about 7.5 mM to about 8.5 mM, about 7.5 mM to about 8.0 mM, about 8.0 mM to about 10 mM, about 8.0 mM to about 9.5 mM, about 8.0 mM to about 9.0 mM, about 8.0 mM to about 8.5 mM, about 8.5 mM to about 10 mM, about 8.5 mM to about 9.5 mM, about 8.5 mM to about 9.0 mM, about 9.0 mM to about 10 mM, about 9.0 mM to about 9.5 mM, or about 9.5 mM to about 10 mM) of L-glutamine, L-alanyl-L-glutamine, L-glycyl-L-glutamine, N-acetyl-L-glutamine, or a combination thereof. In some embodiments of any of the first culture media described herein, the first culture medium includes about 1 μg/mL to about 20 μg/mL (e.g., about 1 μg/mL to about 19 μg/mL, about 1 μg/mL to about 18 μg/mL, about 1 μg/mL to about 17 μg/mL, about 1 μg/mL to about 16 μg/mL, about 1 μg/mL to about 15 μg/mL, about 1 μg/mL to about 14 μg/mL, about 1 μg/mL to about 13 μg/mL, about 1 μg/mL to about 12 μg/mL, about 1 μg/mL to about 11 μg/mL, about 1 μg/mL to about 10 μg/mL, about 1 μg/mL to about 9 μg/mL, about 1 μg/mL to about 8 μg/mL, about 1 μg/mL to about 7 μg/mL, about 1 μg/mL to about 6 μg/mL, about 1 μg/mL to about 5 μg/mL, about 1 μg/mL to about 4 μg/mL, about 1 μg/mL to about 3 μg/mL, about 1 μg/mL to about 2 μg/mL, about 2 μg/mL to about 20 μg/mL, about 2 μg/mL to about 19 μg/mL, about 2 μg/mL to about 18 μg/mL, about 2 μg/mL to about 17 μg/mL, about 2 μg/mL to about 16 μg/mL, about 2 μg/mL to about 15 μg/mL, about 2 μg/mL to about 14 μg/mL, about 2 μg/mL to about 13 μg/mL, about 2 μg/mL to about 12 μg/mL, about 2 μg/mL to about 11 μg/mL, about 2 μg/mL to about 10 μg/mL, about 2 μg/mL to about 9 μg/mL, about 2 μg/mL to about 8 μg/mL, about 2 μg/mL to about 7 μg/mL, about 2 μg/mL to about 6 μg/mL, about 2 μg/mL to about 5 μg/mL, about 2 μg/mL to about 4 μg/mL, about 2 μg/mL to about 3 μg/mL, about 3 μg/mL to about 20 μg/mL, about 3 μg/mL to about 19 μg/mL, about 3 μg/mL to about 18 μg/mL, about 3 μg/mL to about 17 μg/mL, about 3 μg/mL to about 16 μg/mL, about 3 μg/mL to about 15 μg/mL, about 3 μg/mL to about 14 μg/mL, about 3 μg/mL to about 13 μg/mL, about 3 μg/mL to about 12 μg/mL, about 3 μg/mL to about 11 μg/mL, about 3 μg/mL to about 10 μg/mL, about 3 μg/mL to about 9 μg/mL, about 3 μg/mL to about 8 μg/mL, about 3 μg/mL to about 7 μg/mL, about 3 μg/mL to about 6 μg/mL, about 3 μg/mL to about 5 μg/mL, about 3 μg/mL to about 4 μg/mL, about 4 μg/mL to about 20 μg/mL, about 4 μg/mL to about 19 μg/mL, about 4 μg/mL to about 18 μg/mL, about 4 μg/mL to about 17 μg/mL, about 4 μg/mL to about 16 μg/mL, about 4 μg/mL to about 15 μg/mL, about 4 μg/mL to about 14 μg/mL, about 4 μg/mL to about 13 μg/mL, about 4 μg/mL to about 12 μg/mL, about 4 μg/mL to about 11 μg/mL, about 4 μg/mL to about 10 μg/mL, about 4 μg/mL to about 9 μg/mL, about 4 μg/mL to about 8 μg/mL, about 4 μg/mL to about 7 μg/mL, about 4 μg/mL to about 6 μg/mL, about 4 μg/mL to about 5 μg/mL, about 5 μg/mL to about 20 μg/mL, about 5 μg/mL to about 19 μg/mL, about 5 μg/mL to about 18 μg/mL, about 5 μg/mL to about 17 μg/mL, about 5 μg/mL to about 16 μg/mL, about 5 μg/mL to about 15 μg/mL, about 5 μg/mL to about 14 μg/mL, about 5 μg/mL to about 13 μg/mL, about 5 μg/mL to about 12 μg/mL, about 5 μg/mL to about 11 μg/mL, about 5 μg/mL to about 10 μg/mL, about 5 μg/mL to about 9 μg/mL, about 5 μg/mL to about 8 μg/mL, about 5 μg/mL to about 7 μg/mL, about 5 μg/mL to about 6 μg/mL, about 6 μg/mL to about 20 μg/mL, about 6 μg/mL to about 19 μg/mL, about 6 μg/mL to about 18 μg/mL, about 6 μg/mL to about 17 μg/mL, about 6 μg/mL to about 16 μg/mL, about 6 μg/mL to about 15 μg/mL, about 6 μg/mL to about 14 μg/mL, about 6 μg/mL to about 13 μg/mL, about 6 μg/mL to about 12 μg/mL, about 6 μg/mL to about 11 μg/mL, about 6 μg/mL to about 10 μg/mL, about 6 μg/mL to about 9 μg/mL, about 6 μg/mL to about 8 μg/mL, about 6 μg/mL to about 7 μg/mL, about 7 μg/mL to about 20 μg/mL, about 7 μg/mL to about 19 μg/mL, about 7 μg/mL to about 18 μg/mL, about 7 μg/mL to about 17 μg/mL, about 7 μg/mL to about 16 μg/mL, about 7 μg/mL to about 15 μg/mL, about 7 μg/mL to about 14 μg/mL, about 7 μg/mL to about 13 μg/mL, about 7 μg/mL to about 12 μg/mL, about 7 μg/mL to about 11 μg/mL, about 7 μg/mL to about 10 μg/mL, about 7 μg/mL to about 9 μg/mL, about 7 μg/mL to about 8 μg/mL, about 8 μg/mL to about 20 μg/mL, about 8 μg/mL to about 19 μg/mL, about 8 μg/mL to about 18 μg/mL, about 8 μg/mL to about 17 μg/mL, about 8 μg/mL to about 16 μg/mL, about 8 μg/mL to about 15 μg/mL, about 8 μg/mL to about 14 μg/mL, about 8 μg/mL to about 13 μg/mL, about 8 μg/mL to about 12 μg/mL, about 8 μg/mL to about 11 μg/mL, about 8 μg/mL to about 10 μg/mL, about 8 μg/mL to about 9 μg/mL, about 9 μg/mL to about 20 μg/mL, about 9 μg/mL to about 19 μg/mL, about 9 μg/mL to about 18 μg/mL, about 9 μg/mL to about 17 μg/mL, about 9 μg/mL to about 16 μg/mL, about 9 μg/mL to about 15 μg/mL, about 9 μg/mL to about 14 μg/mL, about 9 μg/mL to about 13 μg/mL, about 9 μg/mL to about 12 μg/mL, about 9 μg/mL to about 11 μg/mL, about 9 μg/mL to about 10 μg/mL, about 10 μg/mL to about 20 μg/mL, about 10 μg/mL to about 19 μg/mL, about 10 μg/mL to about 18 μg/mL, about 10 μg/mL to about 17 μg/mL, about 10 μg/mL to about 16 μg/mL, about 10 μg/mL to about 15 μg/mL, about 10 μg/mL to about 14 μg/mL, about 10 μg/mL to about 13 μg/mL, about 10 μg/mL to about 12 μg/mL, about 10 μg/mL to about 11 μg/mL, about 11 μg/mL to about 20 μg/mL, about 11 μg/mL to about 19 μg/mL, about 11 μg/mL to about 18 μg/mL, about 11 μg/mL to about 17 μg/mL, about 11 μg/mL to about 16 μg/mL, about 11 μg/mL to about 15 μg/mL, about 11 μg/mL to about 14 μg/mL, about 11 μg/mL to about 13 μg/mL, about 11 μg/mL to about 12 μg/mL, 12 μg/mL to about 20 μg/mL, about 12 μg/mL to about 19 μg/mL, about 12 μg/mL to about 18 μg/mL, about 12 μg/mL to about 17 μg/mL, about 12 μg/mL to about 16 μg/mL, about 12 μg/mL to about 15 μg/mL, about 12 μg/mL to about 14 μg/mL, about 12 μg/mL to about 13 μg/mL, 13 μg/mL to about 20 μg/mL, about 13 μg/mL to about 19 μg/mL, about 13 μg/mL to about 18 μg/mL, about 13 μg/mL to about 17 μg/mL, about 13 μg/mL to about 16 μg/mL, about 13 μg/mL to about 15 μg/mL, about 13 μg/mL to about 14 μg/mL, 14 μg/mL to about 20 μg/mL, about 14 μg/mL to about 19 μg/mL, about 14 μg/mL to about 18 μg/mL, about 14 μg/mL to about 17 μg/mL, about 14 μg/mL to about 16 μg/mL, about 14 μg/mL to about 15 μg/mL, 15 μg/mL to about 20 μg/mL, about 15 μg/mL to about 19 μg/mL, about 15 μg/mL to about 18 μg/mL, about 15 μg/mL to about 17 μg/mL, about 15 μg/mL to about 16 μg/mL, 16 μg/mL to about 20 μg/mL, about 16 μg/mL to about 19 μg/mL, about 16 μg/mL to about 18 μg/mL, about 16 μg/mL to about 17 μg/mL, 17 μg/mL to about 20 μg/mL, about 17 μg/mL to about 19 μg/mL, about 17 μg/mL to about 18 μg/mL, about 18 μg/mL to about 20 μg/mL, about 18 μg/mL to about 19 μg/mL, or about 19 μg/mL to about 20 μg/mL) insulin (e.g., human insulin).

In some embodiments of any of the first culture media described herein, the first culture medium includes lipid (e.g., a lipid mixture). In some embodiments of any of the first culture media described herein, the first culture medium includes transferrin (e.g., apotransferrin, holo transferrin, or a combination thereof) (e.g., about 50 μg/mL to about 400 μg/mL, about 50 μg/mL to about 350 μg/mL, about 50 μg/mL to about 300 μg/mL, about 50 μg/mL to about 250 μg/mL, about 50 μg/mL to about 200 μg/mL, about 50 μg/mL to about 150 μg/mL, about 50 μg/mL to about 100 μg/mL, about 100 μg/mL to about 400 μg/mL, about 100 μg/mL to about 350 μg/mL, about 100 μg/mL to about 300 μg/mL, about 100 μg/mL to about 250 μg/mL, about 100 μg/mL to about 200 μg/mL, about 100 μg/mL to about 150 μg/mL, about 150 μg/mL to about 400 μg/mL, about 150 μg/mL to about 350 μg/mL, about 150 μg/mL to about 300 μg/mL, about 150 μg/mL to about 250 μg/mL, about 150 μg/mL to about 200 μg/mL, about 200 μg/mL to about 400 μg/mL, about 200 μg/mL to about 350 μg/mL, about 200 μg/mL to about 300 μg/mL, about 200 μg/mL to about 250 μg/mL, about 250 μg/mL to about 400 μg/mL, about 250 μg/mL to about 350 μg/mL, about 250 μg/mL to about 300 μg/mL, about 300 μg/mL to about 400 μg/mL, about 300 μg/mL to about 350 μg/mL, or about 350 to about 400 μg/mL of transferrin (e.g., apotransferrin, holo transferrin, or a combination thereof).

In some embodiments, the first culture medium comprises about 0.1 ng/mL to about 200 ng/mL, about 0.1 ng/mL to about 190 ng/mL, about 0.1 ng/mL to about 180 ng/mL, about 0.1 ng/mL to about 170 ng/mL, about 0.1 ng/mL to about 160 ng/mL, about 0.1 ng/mL to about 150 ng/mL, about 0.1 ng/mL to about 140 ng/mL, about 0.1 ng/mL to about 130 ng/mL, about 0.1 ng/mL to about 120 ng/mL, about 0.1 ng/mL to about 110 ng/mL, about 0.1 ng/mL to about 100 ng/mL, about 0.1 ng/mL to about 90 ng/mL, about 0.1 ng/mL to about 80 ng/mL, about 0.1 ng/mL to about 70 ng/mL, about 0.1 ng/mL to about 60 ng/mL, about 0.1 ng/mL to about 50 ng/mL, about 0.1 ng/mL to about 40 ng/mL, about 0.1 ng/mL to about 30 ng/mL, about 0.1 ng/mL to about 20 ng/mL, about 0.1 ng/mL to about 10 ng/mL, about 0.1 ng/mL to about 5 ng/mL, about 5 ng/mL to about 200 ng/mL, about 5 ng/mL to about 190 ng/mL, about 5 ng/mL to about 180 ng/mL, about 5 ng/mL to about 170 ng/mL, about 5 ng/mL to about 160 ng/mL, about 5 ng/mL to about 150 ng/mL, about 5 ng/mL to about 140 ng/mL, about 5 ng/mL to about 130 ng/mL, about 5 ng/mL to about 120 ng/mL, about 5 ng/mL to about 110 ng/mL, about 5 ng/mL to about 100 ng/mL, about 5 ng/mL to about 90 ng/mL, about 5 ng/mL to about 80 ng/mL, about 5 ng/mL to about 70 ng/mL, about 5 ng/mL to about 60 ng/mL, about 5 ng/mL to about 50 ng/mL, about 5 ng/mL to about 40 ng/mL, about 5 ng/mL to about 30 ng/mL, about 5 ng/mL to about 20 ng/mL, about 5 ng/mL to about 10 ng/mL, about 10 ng/mL to about 200 ng/mL, about 10 ng/mL to about 190 ng/mL, about 10 ng/mL to about 180 ng/mL, about 10 ng/mL to about 170 ng/mL, about 10 ng/mL to about 160 ng/mL, about 10 ng/mL to about 150 ng/mL, about 10 ng/mL to about 140 ng/mL, about 10 ng/mL to about 130 ng/mL, about 10 ng/mL to about 120 ng/mL, about 10 ng/mL to about 110 ng/mL, about 10 ng/mL to about 100 ng/mL, about 10 ng/mL to about 90 ng/mL, about 10 ng/mL to about 80 ng/mL, about 10 ng/mL to about 70 ng/mL, about 10 ng/mL to about 60 ng/mL, about 10 ng/mL to about 50 ng/mL, about 10 ng/mL to about 40 ng/mL, about 10 ng/mL to about 30 ng/mL, about 10 ng/mL to about 20 ng/mL, about 20 ng/mL to about 200 ng/mL, about 20 ng/mL to about 190 ng/mL, about 20 ng/mL to about 180 ng/mL, about 20 ng/mL to about 170 ng/mL, about 20 ng/mL to about 160 ng/mL, about 20 ng/mL to about 150 ng/mL, about 20 ng/mL to about 140 ng/mL, about 20 ng/mL to about 130 ng/mL, about 20 ng/mL to about 120 ng/mL, about 20 ng/mL to about 110 ng/mL, about 20 ng/mL to about 100 ng/mL, about 20 ng/mL to about 90 ng/mL, about 20 ng/mL to about 80 ng/mL, about 20 ng/mL to about 70 ng/mL, about 20 ng/mL to about 60 ng/mL, about 20 ng/mL to about 50 ng/mL, about 20 ng/mL to about 40 ng/mL, about 20 ng/mL to about 30 ng/mL, about 30 ng/mL to about 200 ng/mL, about 30 ng/mL to about 190 ng/mL, about 30 ng/mL to about 180 ng/mL, about 30 ng/mL to about 170 ng/mL, about 30 ng/mL to about 160 ng/mL, about 30 ng/mL to about 150 ng/mL, about 30 ng/mL to about 140 ng/mL, about 30 ng/mL to about 130 ng/mL, about 30 ng/mL to about 120 ng/mL, about 30 ng/mL to about 110 ng/mL, about 30 ng/mL to about 100 ng/mL, about 30 ng/mL to about 90 ng/mL, about 30 ng/mL to about 80 ng/mL, about 30 ng/mL to about 70 ng/mL, about 30 ng/mL to about 60 ng/mL, about 30 ng/mL to about 50 ng/mL, about 30 ng/mL to about 40 ng/mL, about 40 ng/mL to about 200 ng/mL, about 40 ng/mL to about 190 ng/mL, about 40 ng/mL to about 180 ng/mL, about 40 ng/mL to about 170 ng/mL, about 40 ng/mL to about 160 ng/mL, about 40 ng/mL to about 150 ng/mL, about 40 ng/mL to about 140 ng/mL, about 40 ng/mL to about 130 ng/mL, about 40 ng/mL to about 120 ng/mL, about 40 ng/mL to about 110 ng/mL, about 40 ng/mL to about 100 ng/mL, about 40 ng/mL to about 90 ng/mL, about 40 ng/mL to about 80 ng/mL, about 40 ng/mL to about 70 ng/mL, about 40 ng/mL to about 60 ng/mL, about 40 ng/mL to about 50 ng/mL, about 50 ng/mL to about 200 ng/mL, about 50 ng/mL to about 190 ng/mL, about 50 ng/mL to about 180 ng/mL, about 50 ng/mL to about 170 ng/mL, about 50 ng/mL to about 160 ng/mL, about 50 ng/mL to about 150 ng/mL, about 50 ng/mL to about 140 ng/mL, about 50 ng/mL to about 130 ng/mL, about 50 ng/mL to about 120 ng/mL, about 50 ng/mL to about 110 ng/mL, about 50 ng/mL to about 100 ng/mL, about 50 ng/mL to about 90 ng/mL, about 50 ng/mL to about 80 ng/mL, about 50 ng/mL to about 70 ng/mL, about 50 ng/mL to about 60 ng/mL, about 60 ng/mL to about 200 ng/mL, about 60 ng/mL to about 190 ng/mL, about 60 ng/mL to about 180 ng/mL, about 60 ng/mL to about 170 ng/mL, about 60 ng/mL to about 160 ng/mL, about 60 ng/mL to about 150 ng/mL, about 60 ng/mL to about 140 ng/mL, about 60 ng/mL to about 130 ng/mL, about 60 ng/mL to about 120 ng/mL, about 60 ng/mL to about 110 ng/mL, about 60 ng/mL to about 100 ng/mL, about 60 ng/mL to about 90 ng/mL, about 60 ng/mL to about 80 ng/mL, about 60 ng/mL to about 70 ng/mL, about 70 ng/mL to about 200 ng/mL, about 70 ng/mL to about 190 ng/mL, about 70 ng/mL to about 180 ng/mL, about 70 ng/mL to about 170 ng/mL, about 70 ng/mL to about 160 ng/mL, about 70 ng/mL to about 150 ng/mL, about 70 ng/mL to about 140 ng/mL, about 70 ng/mL to about 130 ng/mL, about 70 ng/mL to about 120 ng/mL, about 70 ng/mL to about 110 ng/mL, about 70 ng/mL to about 100 ng/mL, about 70 ng/mL to about 90 ng/mL, about 70 ng/mL to about 80 ng/mL, about 80 ng/mL to about 200 ng/mL, about 80 ng/mL to about 190 ng/mL, about 80 ng/mL to about 180 ng/mL, about 80 ng/mL to about 170 ng/mL, about 80 ng/mL to about 160 ng/mL, about 80 ng/mL to about 150 ng/mL, about 80 ng/mL to about 140 ng/mL, about 80 ng/mL to about 130 ng/mL, about 80 ng/mL to about 120 ng/mL, about 80 ng/mL to about 110 ng/mL, about 80 ng/mL to about 100 ng/mL, about 80 ng/mL to about 90 ng/mL, about 90 ng/mL to about 200 ng/mL, about 90 ng/mL to about 190 ng/mL, about 90 ng/mL to about 180 ng/mL, about 90 ng/mL to about 170 ng/mL, about 90 ng/mL to about 160 ng/mL, about 90 ng/mL to about 150 ng/mL, about 90 ng/mL to about 140 ng/mL, about 90 ng/mL to about 130 ng/mL, about 90 ng/mL to about 120 ng/mL, about 90 ng/mL to about 110 ng/mL, about 90 ng/mL to about 100 ng/mL, about 100 ng/mL to about 200 ng/mL, about 100 ng/mL to about 190 ng/mL, about 100 ng/mL to about 180 ng/mL, about 100 ng/mL to about 170 ng/mL, about 100 ng/mL to about 160 ng/mL, about 100 ng/mL to about 150 ng/mL, about 100 ng/mL to about 140 ng/mL, about 100 ng/mL to about 130 ng/mL, about 100 ng/mL to about 120 ng/mL, about 100 ng/mL to about 110 ng/mL, about 110 ng/mL to about 200 ng/mL, about 110 ng/mL to about 190 ng/mL, about 110 ng/mL to about 180 ng/mL, about 110 ng/mL to about 170 ng/mL, about 110 ng/mL to about 160 ng/mL, about 110 ng/mL to about 150 ng/mL, about 110 ng/mL to about 140 ng/mL, about 110 ng/mL to about 130 ng/mL, about 110 ng/mL to about 120 ng/mL, about 120 ng/mL to about 200 ng/mL, about 120 ng/mL to about 190 ng/mL, about 120 ng/mL to about 180 ng/mL, about 120 ng/mL to about 170 ng/mL, about 120 ng/mL to about 160 ng/mL, about 120 ng/mL to about 150 ng/mL, about 120 ng/mL to about 140 ng/mL, about 120 ng/mL to about 130 ng/mL, about 130 ng/mL to about 200 ng/mL, about 130 ng/mL to about 190 ng/mL, about 130 ng/mL to about 180 ng/mL, about 130 ng/mL to about 170 ng/mL, about 130 ng/mL to about 160 ng/mL, about 130 ng/mL to about 150 ng/mL, about 130 ng/mL to about 140 ng/mL, about 140 ng/mL to about 200 ng/mL, about 140 ng/mL to about 190 ng/mL, about 140 ng/mL to about 180 ng/mL, about 140 ng/mL to about 170 ng/mL, about 140 ng/mL to about 160 ng/mL, about 140 ng/mL to about 150 ng/mL, about 150 ng/mL to about 200 ng/mL, about 150 ng/mL to about 190 ng/mL, about 150 ng/mL to about 180 ng/mL, about 150 ng/mL to about 170 ng/mL, about 150 ng/mL to about 160 ng/mL, about 160 ng/mL to about 200 ng/mL, about 160 ng/mL to about 190 ng/mL, about 160 ng/mL to about 180 ng/mL, about 160 ng/mL to about 170 ng/mL, about 170 ng/mL to about 200 ng/mL, about 170 ng/mL to about 190 ng/mL, about 170 ng/mL to about 180 ng/mL, about 180 ng/mL to about 200 ng/mL, about 180 ng/mL to about 190 ng/mL, or about 190 ng/mL to about 200 ng/mL, of Flt-3 ligand (e.g., recombinant human Flt-3 ligand).

In some embodiments, the first culture medium includes about 1 ng/mL to about 1 μg/mL, about 1 ng/mL to about 950 ng/mL, about 1 ng/mL to about 900 ng/mL, about 1 ng/mL to about 850 ng/mL, about 1 ng/mL to about 800 ng/mL, about 1 ng/mL to about 750 ng/mL, about 1 ng/mL to about 700 ng/mL, about 1 ng/mL to about 650 ng/mL, about 1 ng/mL to about 600 ng/mL, about 1 ng/mL to about 550 ng/mL, about 1 ng/mL to about 500 ng/mL, about 1 ng/mL to about 450 ng/mL, about 1 ng/mL to about 400 ng/mL, about 1 ng/mL to about 350 ng/mL, about 1 ng/mL to about 300 ng/mL, about 1 ng/mL to about 250 ng/mL, about 1 ng/mL to about 200 ng/mL, about 1 ng/mL to about 150 ng/mL, about 1 ng/mL to about 100 ng/mL, about 1 ng/mL to about 50 ng/mL, about 1 ng/mL to about 25 ng/mL, about 1 ng/mL to about 20 ng/mL, about 1 ng/mL to about 15 ng/mL, about 1 ng/mL to about 10 ng/mL, about 1 ng/mL to about 5 ng/mL, about 5 ng/mL to about 1 μg/mL, about 5 ng/mL to about 950 ng/mL, about 5 ng/mL to about 900 ng/mL, about 5 ng/mL to about 850 ng/mL, about 5 ng/mL to about 800 ng/mL, about 5 ng/mL to about 750 ng/mL, about 5 ng/mL to about 700 ng/mL, about 5 ng/mL to about 650 ng/mL, about 5 ng/mL to about 600 ng/mL, about 5 ng/mL to about 550 ng/mL, about 5 ng/mL to about 500 ng/mL, about 5 ng/mL to about 450 ng/mL, about 5 ng/mL to about 400 ng/mL, about 5 ng/mL to about 350 ng/mL, about 5 ng/mL to about 300 ng/mL, about 5 ng/mL to about 250 ng/mL, about 5 ng/mL to about 200 ng/mL, about 5 ng/mL to about 150 ng/mL, about 5 ng/mL to about 100 ng/mL, about 5 ng/mL to about 50 ng/mL, about 5 ng/mL to about 25 ng/mL, about 5 ng/mL to about 20 ng/mL, about 5 ng/mL to about 15 ng/mL, about 5 ng/mL to about 10 ng/mL, about10 ng/mL to about 1 μg/mL, about 10 ng/mL to about 950 ng/mL, about 10 ng/mL to about 900 ng/mL, about 10 ng/mL to about 850 ng/mL, about 10 ng/mL to about 800 ng/mL, about 10 ng/mL to about 750 ng/mL, about 10 ng/mL to about 700 ng/mL, about 10 ng/mL to about 650 ng/mL, about 10 ng/mL to about 600 ng/mL, about 10 ng/mL to about 550 ng/mL, about 10 ng/mL to about 500 ng/mL, about 10 ng/mL to about 450 ng/mL, about 10 ng/mL to about 400 ng/mL, about 10 ng/mL to about 350 ng/mL, about 10 ng/mL to about 300 ng/mL, about 10 ng/mL to about 250 ng/mL, about 10 ng/mL to about 200 ng/mL, about 10 ng/mL to about 150 ng/mL, about 10 ng/mL to about 100 ng/mL, about 10 ng/mL to about 50 ng/mL, about 10 ng/mL to about 25 ng/mL, about 10 ng/mL to about 20 ng/mL, about 10 ng/mL to about 15 ng/mL, about 15 ng/mL to about 1 μg/mL, about 15 ng/mL to about 950 ng/mL, about 15 ng/mL to about 900 ng/mL, about 15 ng/mL to about 850 ng/mL, about 15 ng/mL to about 800 ng/mL, about 15 ng/mL to about 750 ng/mL, about 15 ng/mL to about 700 ng/mL, about 15 ng/mL to about 650 ng/mL, about 15 ng/mL to about 600 ng/mL, about 15 ng/mL to about 550 ng/mL, about 15 ng/mL to about 500 ng/mL, about 15 ng/mL to about 450 ng/mL, about 15 ng/mL to about 400 ng/mL, about 15 ng/mL to about 350 ng/mL, about 15 ng/mL to about 300 ng/mL, about 15 ng/mL to about 250 ng/mL, about 15 ng/mL to about 200 ng/mL, about 15 ng/mL to about 150 ng/mL, about 15 ng/mL to about 100 ng/mL, about 15 ng/mL to about 50 ng/mL, about 15 ng/mL to about 25 ng/mL, about 15 ng/mL to about 20 ng/mL, about 20 ng/mL to about 1 μg/mL, about 20 ng/mL to about 950 ng/mL, about 20 ng/mL to about 900 ng/mL, about 20 ng/mL to about 850 ng/mL, about 20 ng/mL to about 800 ng/mL, about 20 ng/mL to about 750 ng/mL, about 20 ng/mL to about 700 ng/mL, about 20 ng/mL to about 650 ng/mL, about 20 ng/mL to about 600 ng/mL, about 20 ng/mL to about 550 ng/mL, about 20 ng/mL to about 500 ng/mL, about 20 ng/mL to about 450 ng/mL, about 20 ng/mL to about 400 ng/mL, about 20 ng/mL to about 350 ng/mL, about 20 ng/mL to about 300 ng/mL, about 20 ng/mL to about 250 ng/mL, about 20 ng/mL to about 200 ng/mL, about 20 ng/mL to about 150 ng/mL, about 20 ng/mL to about 100 ng/mL, about 20 ng/mL to about 50 ng/mL, about 20 ng/mL to about 25 ng/mL, about 25 ng/mL to about 1 μg/mL, about 25 ng/mL to about 950 ng/mL, about 25 ng/mL to about 900 ng/mL, about 25 ng/mL to about 850 ng/mL, about 25 ng/mL to about 800 ng/mL, about 25 ng/mL to about 750 ng/mL, about 25 ng/mL to about 700 ng/mL, about 25 ng/mL to about 650 ng/mL, about 25 ng/mL to about 600 ng/mL, about 25 ng/mL to about 550 ng/mL, about 25 ng/mL to about 500 ng/mL, about 25 ng/mL to about 450 ng/mL, about 25 ng/mL to about 400 ng/mL, about 25 ng/mL to about 350 ng/mL, about 25 ng/mL to about 300 ng/mL, about 25 ng/mL to about 250 ng/mL, about 25 ng/mL to about 200 ng/mL, about 25 ng/mL to about 150 ng/mL, about 25 ng/mL to about 100 ng/mL, about 25 ng/mL to about 50 ng/mL, about 50 ng/mL to about 1 μg/mL, about 50 ng/mL to about 950 ng/mL, about 50 ng/mL to about 900 ng/mL, about 50 ng/mL to about 850 ng/mL, about 50 ng/mL to about 800 ng/mL, about 50 ng/mL to about 750 ng/mL, about 50 ng/mL to about 700 ng/mL, about 50 ng/mL to about 650 ng/mL, about 50 ng/mL to about 600 ng/mL, about 50 ng/mL to about 550 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 50 ng/mL to about 250 ng/mL, about 50 ng/mL to about 200 ng/mL, about 50 ng/mL to about 150 ng/mL, about 50 ng/mL to about 100 ng/mL, about 100 ng/mL to about 1 μg/mL, about 100 ng/mL to about 950 ng/mL, about 100 ng/mL to about 900 ng/mL, about 100 ng/mL to about 850 ng/mL, about 100 ng/mL to about 800 ng/mL, about 100 ng/mL to about 750 ng/mL, about 100 ng/mL to about 700 ng/mL, about 100 ng/mL to about 650 ng/mL, about 100 ng/mL to about 600 ng/mL, about 100 ng/mL to about 550 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 100 ng/mL to about 250 ng/mL, about 100 ng/mL to about 200 ng/mL, about 100 ng/mL to about 150 ng/mL, about 150 ng/mL to about 1 μg/mL, about 150 ng/mL to about 950 ng/mL, about 150 ng/mL to about 900 ng/mL, about 150 ng/mL to about 850 ng/mL, about 150 ng/mL to about 800 ng/mL, about 150 ng/mL to about 750 ng/mL, about 150 ng/mL to about 700 ng/mL, about 150 ng/mL to about 650 ng/mL, about 150 ng/mL to about 600 ng/mL, about 150 ng/mL to about 550 ng/mL, about 150 ng/mL to about 500 ng/mL, about 150 ng/mL to about 450 ng/mL, about 150 ng/mL to about 400 ng/mL, about 150 ng/mL to about 350 ng/mL, about 150 ng/mL to about 300 ng/mL, about 150 ng/mL to about 250 ng/mL, about 150 ng/mL to about 200 ng/mL, about 200 ng/mL to about 1 μg/mL, about 200 ng/mL to about 950 ng/mL, about 200 ng/mL to about 900 ng/mL, about 200 ng/mL to about 850 ng/mL, about 200 ng/mL to about 800 ng/mL, about 200 ng/mL to about 750 ng/mL, about 200 ng/mL to about 700 ng/mL, about 200 ng/mL to about 650 ng/mL, about 200 ng/mL to about 600 ng/mL, about 200 ng/mL to about 550 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 200 ng/mL to about 250 ng/mL, about 250 ng/mL to about 1 μg/mL, about 250 ng/mL to about 950 ng/mL, about 250 ng/mL to about 900 ng/mL, about 250 ng/mL to about 850 ng/mL, about 250 ng/mL to about 800 ng/mL, about 250 ng/mL to about 750 ng/mL, about 250 ng/mL to about 700 ng/mL, about 250 ng/mL to about 650 ng/mL, about 250 ng/mL to about 600 ng/mL, about 250 ng/mL to about 550 ng/mL, about 250 ng/mL to about 500 ng/mL, about 250 ng/mL to about 450 ng/mL, about 250 ng/mL to about 400 ng/mL, about 250 ng/mL to about 350 ng/mL, about 250 ng/mL to about 300 ng/mL, about 300 ng/mL to about 1 μg/mL, about 300 ng/mL to about 950 ng/mL, about 300 ng/mL to about 900 ng/mL, about 300 ng/mL to about 850 ng/mL, about 300 ng/mL to about 800 ng/mL, about 300 ng/mL to about 750 ng/mL, about 300 ng/mL to about 700 ng/mL, about 300 ng/mL to about 650 ng/mL, about 300 ng/mL to about 600 ng/mL, about 300 ng/mL to about 550 ng/mL, about 300 ng/mL to about 500 ng/mL, about 300 ng/mL to about 450 ng/mL, about 300 ng/mL to about 400 ng/mL, about 300 ng/mL to about 350 ng/mL, about 350 ng/mL to about 1 μg/mL, about 350 ng/mL to about 950 ng/mL, about 350 ng/mL to about 900 ng/mL, about 350 ng/mL to about 850 ng/mL, about 350 ng/mL to about 800 ng/mL, about 350 ng/mL to about 750 ng/mL, about 350 ng/mL to about 700 ng/mL, about 350 ng/mL to about 650 ng/mL, about 350 ng/mL to about 600 ng/mL, about 350 ng/mL to about 550 ng/mL, about 350 ng/mL to about 500 ng/mL, about 350 ng/mL to about 450 ng/mL, about 350 ng/mL to about 400 ng/mL, about 400 ng/mL to about 1 μg/mL, about 400 ng/mL to about 950 ng/mL, about 400 ng/mL to about 900 ng/mL, about 400 ng/mL to about 850 ng/mL, about 400 ng/mL to about 800 ng/mL, about 400 ng/mL to about 750 ng/mL, about 400 ng/mL to about 700 ng/mL, about 400 ng/mL to about 650 ng/mL, about 400 ng/mL to about 600 ng/mL, about 400 ng/mL to about 550 ng/mL, about 400 ng/mL to about 500 ng/mL, about 400 ng/mL to about 450 ng/mL, about 450 ng/mL to about 1 μg/mL, about 450 ng/mL to about 950 ng/mL, about 450 ng/mL to about 900 ng/mL, about 450 ng/mL to about 850 ng/mL, about 450 ng/mL to about 800 ng/mL, about 450 ng/mL to about 750 ng/mL, about 450 ng/mL to about 700 ng/mL, about 450 ng/mL to about 650 ng/mL, about 450 ng/mL to about 600 ng/mL, about 450 ng/mL to about 550 ng/mL, about 450 ng/mL to about 500 ng/mL, about 500 ng/mL to about 1 μg/mL, about 500 ng/mL to about 950 ng/mL, about 500 ng/mL to about 900 ng/mL, about 500 ng/mL to about 850 ng/mL, about 500 ng/mL to about 800 ng/mL, about 500 ng/mL to about 750 ng/mL, about 500 ng/mL to about 700 ng/mL, about 500 ng/mL to about 650 ng/mL, about 500 ng/mL to about 600 ng/mL, about 500 ng/mL to about 550 ng/mL, about 550 ng/mL to about 1 μg/mL, about 550 ng/mL to about 950 ng/mL, about 550 ng/mL to about 900 ng/mL, about 550 ng/mL to about 850 ng/mL, about 550 ng/mL to about 800 ng/mL, about 550 ng/mL to about 750 ng/mL, about 550 ng/mL to about 700 ng/mL, about 550 ng/mL to about 650 ng/mL, about 550 ng/mL to about 600 ng/mL, about 600 ng/mL to about 1 μg/mL, about 600 ng/mL to about 950 ng/mL, about 600 ng/mL to about 900 ng/mL, about 600 ng/mL to about 850 ng/mL, about 600 ng/mL to about 800 ng/mL, about 600 ng/mL to about 750 ng/mL, about 600 ng/mL to about 700 ng/mL, about 600 ng/mL to about 650 ng/mL, about 650 ng/mL to about 1 μg/mL, about 650 ng/mL to about 950 ng/mL, about 650 ng/mL to about 900 ng/mL, about 650 ng/mL to about 850 ng/mL, about 650 ng/mL to about 800 ng/mL, about 650 ng/mL to about 750 ng/mL, about 650 ng/mL to about 700 ng/mL, about 700 ng/mL to about 1 μg/mL, about 700 ng/mL to about 950 ng/mL, about 700 ng/mL to about 900 ng/mL, about 700 ng/mL to about 850 ng/mL, about 700 ng/mL to about 800 ng/mL, about 700 ng/mL to about 750 ng/mL, about 750 ng/mL to about 1 μg/mL, about 750 ng/mL to about 950 ng/mL, about 750 ng/mL to about 900 ng/mL, about 750 ng/mL to about 850 ng/mL, about 750 ng/mL to about 800 ng/mL, about 800 ng/mL to about 1 μg/mL, about 800 ng/mL to about 950 ng/mL, about 800 ng/mL to about 900 ng/mL, about 800 ng/mL to about 850 ng/mL, about 850 ng/mL to about 1 μg/mL, about 850 ng/mL to about 950 ng/mL, about 850 ng/mL to about 900 ng/mL, about 900 ng/mL to about 1 μg/mL, about 900 ng/mL to about 950 ng/mL, or about 950 ng/mL to about 1 μg/mL, of SCF (e.g., recombinant human stem cell factor).

In some embodiments, the first culture medium comprises about 0.1 ng/mL to about 200 ng/mL, about 0.1 ng/mL to about 190 ng/mL, about 0.1 ng/mL to about 180 ng/mL, about 0.1 ng/mL to about 170 ng/mL, about 0.1 ng/mL to about 160 ng/mL, about 0.1 ng/mL to about 150 ng/mL, about 0.1 ng/mL to about 140 ng/mL, about 0.1 ng/mL to about 130 ng/mL, about 0.1 ng/mL to about 120 ng/mL, about 0.1 ng/mL to about 110 ng/mL, about 0.1 ng/mL to about 100 ng/mL, about 0.1 ng/mL to about 90 ng/mL, about 0.1 ng/mL to about 80 ng/mL, about 0.1 ng/mL to about 70 ng/mL, about 0.1 ng/mL to about 60 ng/mL, about 0.1 ng/mL to about 50 ng/mL, about 0.1 ng/mL to about 40 ng/mL, about 0.1 ng/mL to about 30 ng/mL, about 0.1 ng/mL to about 20 ng/mL, about 0.1 ng/mL to about 10 ng/mL, about 0.1 ng/mL to about 5 ng/mL, about 0.1 ng/mL to about 2 ng/mL, 2 ng/mL to about 200 ng/mL, about 2 ng/mL to about 190 ng/mL, about 2 ng/mL to about 180 ng/mL, about 2 ng/mL to about 170 ng/mL, about 2 ng/mL to about 160 ng/mL, about 2 ng/mL to about 150 ng/mL, about 2 ng/mL to about 140 ng/mL, about 2 ng/mL to about 130 ng/mL, about 2 ng/mL to about 120 ng/mL, about 2 ng/mL to about 110 ng/mL, about 2 ng/mL to about 100 ng/mL, about 2 ng/mL to about 90 ng/mL, about 2 ng/mL to about 80 ng/mL, about 2 ng/mL to about 70 ng/mL, about 2 ng/mL to about 60 ng/mL, about 2 ng/mL to about 50 ng/mL, about 2 ng/mL to about 40 ng/mL, about 2 ng/mL to about 30 ng/mL, about 2 ng/mL to about 20 ng/mL, about 2 ng/mL to about 10 ng/mL, about 2 ng/mL to about 5 ng/mL, about 5 ng/mL to about 200 ng/mL, about 5 ng/mL to about 190 ng/mL, about 5 ng/mL to about 180 ng/mL, about 5 ng/mL to about 170 ng/mL, about 5 ng/mL to about 160 ng/mL, about 5 ng/mL to about 150 ng/mL, about 5 ng/mL to about 140 ng/mL, about 5 ng/mL to about 130 ng/mL, about 5 ng/mL to about 120 ng/mL, about 5 ng/mL to about 110 ng/mL, about 5 ng/mL to about 100 ng/mL, about 5 ng/mL to about 90 ng/mL, about 5 ng/mL to about 80 ng/mL, about 5 ng/mL to about 70 ng/mL, about 5 ng/mL to about 60 ng/mL, about 5 ng/mL to about 50 ng/mL, about 5 ng/mL to about 40 ng/mL, about 5 ng/mL to about 30 ng/mL, about 5 ng/mL to about 20 ng/mL, about 5 ng/mL to about 10 ng/mL, about 10 ng/mL to about 200 ng/mL, about 10 ng/mL to about 190 ng/mL, about 10 ng/mL to about 180 ng/mL, about 10 ng/mL to about 170 ng/mL, about 10 ng/mL to about 160 ng/mL, about 10 ng/mL to about 150 ng/mL, about 10 ng/mL to about 140 ng/mL, about 10 ng/mL to about 130 ng/mL, about 10 ng/mL to about 120 ng/mL, about 10 ng/mL to about 110 ng/mL, about 10 ng/mL to about 100 ng/mL, about 10 ng/mL to about 90 ng/mL, about 10 ng/mL to about 80 ng/mL, about 10 ng/mL to about 70 ng/mL, about 10 ng/mL to about 60 ng/mL, about 10 ng/mL to about 50 ng/mL, about 10 ng/mL to about 40 ng/mL, about 10 ng/mL to about 30 ng/mL, about 10 ng/mL to about 20 ng/mL, about 20 ng/mL to about 200 ng/mL, about 20 ng/mL to about 190 ng/mL, about 20 ng/mL to about 180 ng/mL, about 20 ng/mL to about 170 ng/mL, about 20 ng/mL to about 160 ng/mL, about 20 ng/mL to about 150 ng/mL, about 20 ng/mL to about 140 ng/mL, about 20 ng/mL to about 130 ng/mL, about 20 ng/mL to about 120 ng/mL, about 20 ng/mL to about 110 ng/mL, about 20 ng/mL to about 100 ng/mL, about 20 ng/mL to about 90 ng/mL, about 20 ng/mL to about 80 ng/mL, about 20 ng/mL to about 70 ng/mL, about 20 ng/mL to about 60 ng/mL, about 20 ng/mL to about 50 ng/mL, about 20 ng/mL to about 40 ng/mL, about 20 ng/mL to about 30 ng/mL, about 30 ng/mL to about 200 ng/mL, about 30 ng/mL to about 190 ng/mL, about 30 ng/mL to about 180 ng/mL, about 30 ng/mL to about 170 ng/mL, about 30 ng/mL to about 160 ng/mL, about 30 ng/mL to about 150 ng/mL, about 30 ng/mL to about 140 ng/mL, about 30 ng/mL to about 130 ng/mL, about 30 ng/mL to about 120 ng/mL, about 30 ng/mL to about 110 ng/mL, about 30 ng/mL to about 100 ng/mL, about 30 ng/mL to about 90 ng/mL, about 30 ng/mL to about 80 ng/mL, about 30 ng/mL to about 70 ng/mL, about 30 ng/mL to about 60 ng/mL, about 30 ng/mL to about 50 ng/mL, about 30 ng/mL to about 40 ng/mL, about 40 ng/mL to about 200 ng/mL, about 40 ng/mL to about 190 ng/mL, about 40 ng/mL to about 180 ng/mL, about 40 ng/mL to about 170 ng/mL, about 40 ng/mL to about 160 ng/mL, about 40 ng/mL to about 150 ng/mL, about 40 ng/mL to about 140 ng/mL, about 40 ng/mL to about 130 ng/mL, about 40 ng/mL to about 120 ng/mL, about 40 ng/mL to about 110 ng/mL, about 40 ng/mL to about 100 ng/mL, about 40 ng/mL to about 90 ng/mL, about 40 ng/mL to about 80 ng/mL, about 40 ng/mL to about 70 ng/mL, about 40 ng/mL to about 60 ng/mL, about 40 ng/mL to about 50 ng/mL, about 50 ng/mL to about 200 ng/mL, about 50 ng/mL to about 190 ng/mL, about 50 ng/mL to about 180 ng/mL, about 50 ng/mL to about 170 ng/mL, about 50 ng/mL to about 160 ng/mL, about 50 ng/mL to about 150 ng/mL, about 50 ng/mL to about 140 ng/mL, about 50 ng/mL to about 130 ng/mL, about 50 ng/mL to about 120 ng/mL, about 50 ng/mL to about 110 ng/mL, about 50 ng/mL to about 100 ng/mL, about 50 ng/mL to about 90 ng/mL, about 50 ng/mL to about 80 ng/mL, about 50 ng/mL to about 70 ng/mL, about 50 ng/mL to about 60 ng/mL, about 60 ng/mL to about 200 ng/mL, about 60 ng/mL to about 190 ng/mL, about 60 ng/mL to about 180 ng/mL, about 60 ng/mL to about 170 ng/mL, about 60 ng/mL to about 160 ng/mL, about 60 ng/mL to about 150 ng/mL, about 60 ng/mL to about 140 ng/mL, about 60 ng/mL to about 130 ng/mL, about 60 ng/mL to about 120 ng/mL, about 60 ng/mL to about 110 ng/mL, about 60 ng/mL to about 100 ng/mL, about 60 ng/mL to about 90 ng/mL, about 60 ng/mL to about 80 ng/mL, about 60 ng/mL to about 70 ng/mL, about 70 ng/mL to about 200 ng/mL, about 70 ng/mL to about 190 ng/mL, about 70 ng/mL to about 180 ng/mL, about 70 ng/mL to about 170 ng/mL, about 70 ng/mL to about 160 ng/mL, about 70 ng/mL to about 150 ng/mL, about 70 ng/mL to about 140 ng/mL, about 70 ng/mL to about 130 ng/mL, about 70 ng/mL to about 120 ng/mL, about 70 ng/mL to about 110 ng/mL, about 70 ng/mL to about 100 ng/mL, about 70 ng/mL to about 90 ng/mL, about 70 ng/mL to about 80 ng/mL, about 80 ng/mL to about 200 ng/mL, about 80 ng/mL to about 190 ng/mL, about 80 ng/mL to about 180 ng/mL, about 80 ng/mL to about 170 ng/mL, about 80 ng/mL to about 160 ng/mL, about 80 ng/mL to about 150 ng/mL, about 80 ng/mL to about 140 ng/mL, about 80 ng/mL to about 130 ng/mL, about 80 ng/mL to about 120 ng/mL, about 80 ng/mL to about 110 ng/mL, about 80 ng/mL to about 100 ng/mL, about 80 ng/mL to about 90 ng/mL, about 90 ng/mL to about 200 ng/mL, about 90 ng/mL to about 190 ng/mL, about 90 ng/mL to about 180 ng/mL, about 90 ng/mL to about 170 ng/mL, about 90 ng/mL to about 160 ng/mL, about 90 ng/mL to about 150 ng/mL, about 90 ng/mL to about 140 ng/mL, about 90 ng/mL to about 130 ng/mL, about 90 ng/mL to about 120 ng/mL, about 90 ng/mL to about 110 ng/mL, about 90 ng/mL to about 100 ng/mL, about 100 ng/mL to about 200 ng/mL, about 100 ng/mL to about 190 ng/mL, about 100 ng/mL to about 180 ng/mL, about 100 ng/mL to about 170 ng/mL, about 100 ng/mL to about 160 ng/mL, about 100 ng/mL to about 150 ng/mL, about 100 ng/mL to about 140 ng/mL, about 100 ng/mL to about 130 ng/mL, about 100 ng/mL to about 120 ng/mL, about 100 ng/mL to about 110 ng/mL, about 110 ng/mL to about 200 ng/mL, about 110 ng/mL to about 190 ng/mL, about 110 ng/mL to about 180 ng/mL, about 110 ng/mL to about 170 ng/mL, about 110 ng/mL to about 160 ng/mL, about 110 ng/mL to about 150 ng/mL, about 110 ng/mL to about 140 ng/mL, about 110 ng/mL to about 130 ng/mL, about 110 ng/mL to about 120 ng/mL, about 120 ng/mL to about 200 ng/mL, about 120 ng/mL to about 190 ng/mL, about 120 ng/mL to about 180 ng/mL, about 120 ng/mL to about 170 ng/mL, about 120 ng/mL to about 160 ng/mL, about 120 ng/mL to about 150 ng/mL, about 120 ng/mL to about 140 ng/mL, about 120 ng/mL to about 130 ng/mL, about 130 ng/mL to about 200 ng/mL, about 130 ng/mL to about 190 ng/mL, about 130 ng/mL to about 180 ng/mL, about 130 ng/mL to about 170 ng/mL, about 130 ng/mL to about 160 ng/mL, about 130 ng/mL to about 150 ng/mL, about 130 ng/mL to about 140 ng/mL, about 140 ng/mL to about 200 ng/mL, about 140 ng/mL to about 190 ng/mL, about 140 ng/mL to about 180 ng/mL, about 140 ng/mL to about 170 ng/mL, about 140 ng/mL to about 160 ng/mL, about 140 ng/mL to about 150 ng/mL, about 150 ng/mL to about 200 ng/mL, about 150 ng/mL to about 190 ng/mL, about 150 ng/mL to about 180 ng/mL, about 150 ng/mL to about 170 ng/mL, about 150 ng/mL to about 160 ng/mL, about 160 ng/mL to about 200 ng/mL, about 160 ng/mL to about 190 ng/mL, about 160 ng/mL to about 180 ng/mL, about 160 ng/mL to about 170 ng/mL, about 170 ng/mL to about 200 ng/mL, about 170 ng/mL to about 190 ng/mL, about 170 ng/mL to about 180 ng/mL, about 180 ng/mL to about 200 ng/mL, about 180 ng/mL to about 190 ng/mL, or about 190 ng/mL to about 200 ng/mL, of IL-3 (e.g., recombinant human IL-3).

In some embodiments, the first culture medium comprises about 0.1 ng/mL to about 200 ng/mL, about 0.1 ng/mL to about 190 ng/mL, about 0.1 ng/mL to about 180 ng/mL, about 0.1 ng/mL to about 170 ng/mL, about 0.1 ng/mL to about 160 ng/mL, about 0.1 ng/mL to about 150 ng/mL, about 0.1 ng/mL to about 140 ng/mL, about 0.1 ng/mL to about 130 ng/mL, about 0.1 ng/mL to about 120 ng/mL, about 0.1 ng/mL to about 110 ng/mL, about 0.1 ng/mL to about 100 ng/mL, about 0.1 ng/mL to about 90 ng/mL, about 0.1 ng/mL to about 80 ng/mL, about 0.1 ng/mL to about 70 ng/mL, about 0.1 ng/mL to about 60 ng/mL, about 0.1 ng/mL to about 50 ng/mL, about 0.1 ng/mL to about 40 ng/mL, about 0.1 ng/mL to about 30 ng/mL, about 0.1 ng/mL to about 20 ng/mL, about 0.1 ng/mL to about 10 ng/mL, about 0.1 ng/mL to about 5 ng/mL, about 0.1 ng/mL to about 2 ng/mL, 2 ng/mL to about 200 ng/mL, about 2 ng/mL to about 190 ng/mL, about 2 ng/mL to about 180 ng/mL, about 2 ng/mL to about 170 ng/mL, about 2 ng/mL to about 160 ng/mL, about 2 ng/mL to about 150 ng/mL, about 2 ng/mL to about 140 ng/mL, about 2 ng/mL to about 130 ng/mL, about 2 ng/mL to about 120 ng/mL, about 2 ng/mL to about 110 ng/mL, about 2 ng/mL to about 100 ng/mL, about 2 ng/mL to about 90 ng/mL, about 2 ng/mL to about 80 ng/mL, about 2 ng/mL to about 70 ng/mL, about 2 ng/mL to about 60 ng/mL, about 2 ng/mL to about 50 ng/mL, about 2 ng/mL to about 40 ng/mL, about 2 ng/mL to about 30 ng/mL, about 2 ng/mL to about 20 ng/mL, about 2 ng/mL to about 10 ng/mL, about 2 ng/mL to about 5 ng/mL, about 5 ng/mL to about 200 ng/mL, about 5 ng/mL to about 190 ng/mL, about 5 ng/mL to about 180 ng/mL, about 5 ng/mL to about 170 ng/mL, about 5 ng/mL to about 160 ng/mL, about 5 ng/mL to about 150 ng/mL, about 5 ng/mL to about 140 ng/mL, about 5 ng/mL to about 130 ng/mL, about 5 ng/mL to about 120 ng/mL, about 5 ng/mL to about 110 ng/mL, about 5 ng/mL to about 100 ng/mL, about 5 ng/mL to about 90 ng/mL, about 5 ng/mL to about 80 ng/mL, about 5 ng/mL to about 70 ng/mL, about 5 ng/mL to about 60 ng/mL, about 5 ng/mL to about 50 ng/mL, about 5 ng/mL to about 40 ng/mL, about 5 ng/mL to about 30 ng/mL, about 5 ng/mL to about 20 ng/mL, about 5 ng/mL to about 10 ng/mL, about 10 ng/mL to about 200 ng/mL, about 10 ng/mL to about 190 ng/mL, about 10 ng/mL to about 180 ng/mL, about 10 ng/mL to about 170 ng/mL, about 10 ng/mL to about 160 ng/mL, about 10 ng/mL to about 150 ng/mL, about 10 ng/mL to about 140 ng/mL, about 10 ng/mL to about 130 ng/mL, about 10 ng/mL to about 120 ng/mL, about 10 ng/mL to about 110 ng/mL, about 10 ng/mL to about 100 ng/mL, about 10 ng/mL to about 90 ng/mL, about 10 ng/mL to about 80 ng/mL, about 10 ng/mL to about 70 ng/mL, about 10 ng/mL to about 60 ng/mL, about 10 ng/mL to about 50 ng/mL, about 10 ng/mL to about 40 ng/mL, about 10 ng/mL to about 30 ng/mL, about 10 ng/mL to about 20 ng/mL, about 20 ng/mL to about 200 ng/mL, about 20 ng/mL to about 190 ng/mL, about 20 ng/mL to about 180 ng/mL, about 20 ng/mL to about 170 ng/mL, about 20 ng/mL to about 160 ng/mL, about 20 ng/mL to about 150 ng/mL, about 20 ng/mL to about 140 ng/mL, about 20 ng/mL to about 130 ng/mL, about 20 ng/mL to about 120 ng/mL, about 20 ng/mL to about 110 ng/mL, about 20 ng/mL to about 100 ng/mL, about 20 ng/mL to about 90 ng/mL, about 20 ng/mL to about 80 ng/mL, about 20 ng/mL to about 70 ng/mL, about 20 ng/mL to about 60 ng/mL, about 20 ng/mL to about 50 ng/mL, about 20 ng/mL to about 40 ng/mL, about 20 ng/mL to about 30 ng/mL, about 30 ng/mL to about 200 ng/mL, about 30 ng/mL to about 190 ng/mL, about 30 ng/mL to about 180 ng/mL, about 30 ng/mL to about 170 ng/mL, about 30 ng/mL to about 160 ng/mL, about 30 ng/mL to about 150 ng/mL, about 30 ng/mL to about 140 ng/mL, about 30 ng/mL to about 130 ng/mL, about 30 ng/mL to about 120 ng/mL, about 30 ng/mL to about 110 ng/mL, about 30 ng/mL to about 100 ng/mL, about 30 ng/mL to about 90 ng/mL, about 30 ng/mL to about 80 ng/mL, about 30 ng/mL to about 70 ng/mL, about 30 ng/mL to about 60 ng/mL, about 30 ng/mL to about 50 ng/mL, about 30 ng/mL to about 40 ng/mL, about 40 ng/mL to about 200 ng/mL, about 40 ng/mL to about 190 ng/mL, about 40 ng/mL to about 180 ng/mL, about 40 ng/mL to about 170 ng/mL, about 40 ng/mL to about 160 ng/mL, about 40 ng/mL to about 150 ng/mL, about 40 ng/mL to about 140 ng/mL, about 40 ng/mL to about 130 ng/mL, about 40 ng/mL to about 120 ng/mL, about 40 ng/mL to about 110 ng/mL, about 40 ng/mL to about 100 ng/mL, about 40 ng/mL to about 90 ng/mL, about 40 ng/mL to about 80 ng/mL, about 40 ng/mL to about 70 ng/mL, about 40 ng/mL to about 60 ng/mL, about 40 ng/mL to about 50 ng/mL, about 50 ng/mL to about 200 ng/mL, about 50 ng/mL to about 190 ng/mL, about 50 ng/mL to about 180 ng/mL, about 50 ng/mL to about 170 ng/mL, about 50 ng/mL to about 160 ng/mL, about 50 ng/mL to about 150 ng/mL, about 50 ng/mL to about 140 ng/mL, about 50 ng/mL to about 130 ng/mL, about 50 ng/mL to about 120 ng/mL, about 50 ng/mL to about 110 ng/mL, about 50 ng/mL to about 100 ng/mL, about 50 ng/mL to about 90 ng/mL, about 50 ng/mL to about 80 ng/mL, about 50 ng/mL to about 70 ng/mL, about 50 ng/mL to about 60 ng/mL, about 60 ng/mL to about 200 ng/mL, about 60 ng/mL to about 190 ng/mL, about 60 ng/mL to about 180 ng/mL, about 60 ng/mL to about 170 ng/mL, about 60 ng/mL to about 160 ng/mL, about 60 ng/mL to about 150 ng/mL, about 60 ng/mL to about 140 ng/mL, about 60 ng/mL to about 130 ng/mL, about 60 ng/mL to about 120 ng/mL, about 60 ng/mL to about 110 ng/mL, about 60 ng/mL to about 100 ng/mL, about 60 ng/mL to about 90 ng/mL, about 60 ng/mL to about 80 ng/mL, about 60 ng/mL to about 70 ng/mL, about 70 ng/mL to about 200 ng/mL, about 70 ng/mL to about 190 ng/mL, about 70 ng/mL to about 180 ng/mL, about 70 ng/mL to about 170 ng/mL, about 70 ng/mL to about 160 ng/mL, about 70 ng/mL to about 150 ng/mL, about 70 ng/mL to about 140 ng/mL, about 70 ng/mL to about 130 ng/mL, about 70 ng/mL to about 120 ng/mL, about 70 ng/mL to about 110 ng/mL, about 70 ng/mL to about 100 ng/mL, about 70 ng/mL to about 90 ng/mL, about 70 ng/mL to about 80 ng/mL, about 80 ng/mL to about 200 ng/mL, about 80 ng/mL to about 190 ng/mL, about 80 ng/mL to about 180 ng/mL, about 80 ng/mL to about 170 ng/mL, about 80 ng/mL to about 160 ng/mL, about 80 ng/mL to about 150 ng/mL, about 80 ng/mL to about 140 ng/mL, about 80 ng/mL to about 130 ng/mL, about 80 ng/mL to about 120 ng/mL, about 80 ng/mL to about 110 ng/mL, about 80 ng/mL to about 100 ng/mL, about 80 ng/mL to about 90 ng/mL, about 90 ng/mL to about 200 ng/mL, about 90 ng/mL to about 190 ng/mL, about 90 ng/mL to about 180 ng/mL, about 90 ng/mL to about 170 ng/mL, about 90 ng/mL to about 160 ng/mL, about 90 ng/mL to about 150 ng/mL, about 90 ng/mL to about 140 ng/mL, about 90 ng/mL to about 130 ng/mL, about 90 ng/mL to about 120 ng/mL, about 90 ng/mL to about 110 ng/mL, about 90 ng/mL to about 100 ng/mL, about 100 ng/mL to about 200 ng/mL, about 100 ng/mL to about 190 ng/mL, about 100 ng/mL to about 180 ng/mL, about 100 ng/mL to about 170 ng/mL, about 100 ng/mL to about 160 ng/mL, about 100 ng/mL to about 150 ng/mL, about 100 ng/mL to about 140 ng/mL, about 100 ng/mL to about 130 ng/mL, about 100 ng/mL to about 120 ng/mL, about 100 ng/mL to about 110 ng/mL, about 110 ng/mL to about 200 ng/mL, about 110 ng/mL to about 190 ng/mL, about 110 ng/mL to about 180 ng/mL, about 110 ng/mL to about 170 ng/mL, about 110 ng/mL to about 160 ng/mL, about 110 ng/mL to about 150 ng/mL, about 110 ng/mL to about 140 ng/mL, about 110 ng/mL to about 130 ng/mL, about 110 ng/mL to about 120 ng/mL, about 120 ng/mL to about 200 ng/mL, about 120 ng/mL to about 190 ng/mL, about 120 ng/mL to about 180 ng/mL, about 120 ng/mL to about 170 ng/mL, about 120 ng/mL to about 160 ng/mL, about 120 ng/mL to about 150 ng/mL, about 120 ng/mL to about 140 ng/mL, about 120 ng/mL to about 130 ng/mL, about 130 ng/mL to about 200 ng/mL, about 130 ng/mL to about 190 ng/mL, about 130 ng/mL to about 180 ng/mL, about 130 ng/mL to about 170 ng/mL, about 130 ng/mL to about 160 ng/mL, about 130 ng/mL to about 150 ng/mL, about 130 ng/mL to about 140 ng/mL, about 140 ng/mL to about 200 ng/mL, about 140 ng/mL to about 190 ng/mL, about 140 ng/mL to about 180 ng/mL, about 140 ng/mL to about 170 ng/mL, about 140 ng/mL to about 160 ng/mL, about 140 ng/mL to about 150 ng/mL, about 150 ng/mL to about 200 ng/mL, about 150 ng/mL to about 190 ng/mL, about 150 ng/mL to about 180 ng/mL, about 150 ng/mL to about 170 ng/mL, about 150 ng/mL to about 160 ng/mL, about 160 ng/mL to about 200 ng/mL, about 160 ng/mL to about 190 ng/mL, about 160 ng/mL to about 180 ng/mL, about 160 ng/mL to about 170 ng/mL, about 170 ng/mL to about 200 ng/mL, about 170 ng/mL to about 190 ng/mL, about 170 ng/mL to about 180 ng/mL, about 180 ng/mL to about 200 ng/mL, about 180 ng/mL to about 190 ng/mL, or about 190 ng/mL to about 200 ng/mL, of IL-6 (e.g., recombinant human IL-6).

In some examples, the first culture medium can be, e.g., a chemically-defined liquid culture medium, an animal component-free liquid culture medium, or a chemically-defined animal component-free liquid culture medium, and/or a serum-free liquid culture medium.

### Culturing Step B

Perfusion Culturing

In some embodiments of any of the methods described herein, step (b) includes perfusion culturing the second cell culture (e.g., any of the second cell cultures in any of the exemplary bioreactors having any of the exemplary volumes described herein) for about 2 days to about 15 days (e.g., about 2 days to about 14 days, about 2 days to about 13 days, about 2 days to about 12 days, about 2 days to about 11 days, about 2 days to about 10 days, about 2 days to about 9 days, about 2 day to about 8 days, about 2 days to about 7 days, about 2 days to about 6 days, about 2 days to about 5 days, about 2 days to about 4 days, about 2 days to about 3 days, about 3 days to about 15 days, about 3 days to about 14 days, about 3 days to about 13 days, about 3 days to about 12 days, about 3 days to about 11 days, about 3 days to about 10 days, about 3 days to about 9 days, about 3 days to about 8 days, about 3 days to about 7 days, about 3 days to about 6 days, about 3 days to about 5 days, about 3 days to about 4 days, about 4 days to about 15 days, about 4 days to about 14 days, about 4 days to about 13 days, about 4 days to about 12 days, about 4 days to about 11 days, about 4 days to about 10 days, about 4 days to about 9 days, about 4 days to about 8 days, about 4 days to about 7 days, about 4 days to about 6 days, about 4 days to about 5 days, about 5 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, about 5 days to about 6 days, about 6 days to about 15 days, about 6 days to about 14 days, about 6 days to about 13 days, about 6 days to about 12 days, about 6 days to about 11 days, about 6 days to about 10 days, about 6 days to about 9 days, about 6 days to about 8 days, about 6 days to about 7 days, about 7 days to about 15 days, about 7 days to about 14 days, about 7 days to about 13 days, about 7 days to about 12 days, about 7 days to about 11 days, about 7 days to about 10 days, about 7 days to about 9 days, about 7 days to about 8 days, about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 15 days, about 9 days to about 14 days, about 9 days to about 13 days, about 9 days to about 12 days, about 9 days to about 11 days, about 9 days to about 10 days, about 10 days to about 15 days, about 10 days to about 14 days, about 10 days to about 13 days, about 10 days to about 12 days, about 10 days to about 11 days, about 11 days to about 15 days, about 11 days to about 14 days, about 11 days to about 13 days, about 11 days to about 12 days, about 12 days to about 15 days, about 12 days to about 14 days, about 12 days to about 13 days, about 13 days to about 15 days, about 13 days to about 14 days, or about 14 days to about 15 days). In some embodiments, step (b) includes agitating the second cell culture (e.g., in any of the bioreactors described herein having any of the exemplary volumes described herein) with a P/V value of about 10 W/m³ to about 200 W/m³ (e.g., about 10 W/m³ to about 180 W/m³, about 10 W/m³ to about 160 W/m³, about 10 W/m³ to about 140 W/m³, about 10 W/m³ to about 120 W/m³, about 10 W/m³ to about 100 W/m³, about 10 W/m³ to about 80 W/m³, about 10 W/m³ to about 60 W/m³, about 10 W/m³ to about 40 W/m³, about 10 W/m³ to about 35 W/m³, about 10 W/m³ to about 30 W/m³, about 10 W/m³ to about 25 W/m³, about 10 W/m³ to about 20 W/m³, about 10 W/m³ to about 15 W/m³, about 15 W/m³ to about 200 W/m³, about 15 W/m³ to about 180 W/m³, about 15 W/m³ to about 160 W/m³, about 15 W/m³ to about 140 W/m³, about 15 W/m³ to about 120 W/m³, about 15 W/m³ to about 100 W/m³, about 15 W/m³ to about 80 W/m³, about 15 W/m³ to about 60 W/m³, about 15 W/m³ to about 40 W/m³, about 15 W/m³ to about 35 W/m³, about 15 W/m³ to about 30 W/m³, about 15 W/m³ to about 25 W/m³, about 15 W/m³ to about 20 W/m³, about 20 W/m³ to about 200 W/m³, about 20 W/m³ to about 180 W/m³, about 20 W/m³ to about 160 W/m³, about 20 W/m³ to about 140 W/m³, about 20 W/m³ to about 120 W/m³, about 20 W/m³ to about 100 W/m³, about 20 W/m³ to about 80 W/m³, about 20 W/m³ to about 60 W/m³, about 20 W/m³ to about 40 W/m³, about 20 W/m³ to about 35 W/m³, about 20 W/m³ to about 30 W/m³, about 20 W/m³ to about 25 W/m³, about 25 W/m³ to about 200 W/m³, about 25 W/m³ to about 180 W/m³, about 25 W/m³ to about 160 W/m³, about 25 W/m³ to about 140 W/m³, about 25 W/m³ to about 120 W/m³, about 25 W/m³ to about 100 W/m³, about 25 W/m³ to about 80 W/m³, about 25 W/m³ to about 60 W/m³, about 25 W/m³ to about 40 W/m³, about 25 W/m³ to about 35 W/m³, about 25 W/m³ to about 30 W/m³, about 30 W/m³ to about 200 W/m³, about 30 W/m³ to about 180 W/m³, about 30 W/m³ to about 160 W/m³, about 30 W/m³ to about 140 W/m³, about 30 W/m³ to about 120 W/m³, about 30 W/m³ to about 100 W/m³, about 30 W/m³ to about 80 W/m³, about 30 W/m³ to about 60 W/m³, about 30 W/m³ to about 40 W/m³, about 30 W/m³ to about 35 W/m³, about 35 W/m³ to about 200 W/m³, about 35 W/m³ to about 180 W/m³, about 35 W/m³ to about 160 W/m³, about 35 W/m³ to about 140 W/m³, about 35 W/m³ to about 120 W/m³, about 35 W/m³ to about 100 W/m³, about 35 W/m³ to about 80 W/m³, about 35 W/m³ to about 60 W/m³, about 35 W/m³ to about 40 W/m³, about 40 W/m³ to about 200 W/m³, about 40 W/m³ to about 180 W/m³, about 40 W/m³ to about 160 W/m³, about 40 W/m³ to about 140 W/m³, about 40 W/m³ to about 120 W/m³, about 40 W/m³ to about 100 W/m³, about 40 W/m³ to about 80 W/m³, about 40 W/m³ to about 60 W/m³, about 60 W/m³ to about 200 W/m³, about 60 W/m³ to about 180 W/m³, about 60 W/m³ to about 160 W/m³, about 60 W/m³ to about 140 W/m³, about 60 W/m³ to about 120 W/m³, about 60 W/m³ to about 100 W/m³, about 60 W/m³ to about 80 W/m³, about 80 W/m³ to about 200 W/m³, about 80 W/m³ to about 180 W/m³, about 80 W/m³ to about 160 W/m³, about 80 W/m³ to about 140 W/m³, about 80 W/m³ to about 120 W/m³, about 80 W/m³ to about 100 W/m³, about 100 W/m³ to about 200 W/m³, about 100 W/m³ to about 180 W/m³, about 100 W/m³ to about 160 W/m³, about 100 W/m³ to about 140 W/m³, about 100 W/m³ to about 120 W/m³, about 120 W/m³ to about 200 W/m³, about 120 W/m³ to about 180 W/m³, about 120 W/m³ to about 160 W/m³, about 120 W/m³ to about 140 W/m³, about 140 W/m³ to about 200 W/m³, about 140 W/m³ to about 180 W/m³, about 140 W/m³ to about 160 W/m³, about 160 W/m³ to about 200 W/m³, about 160 W/m³ to about 180 W/m³, and about 180 W/m³ to about 200 W/m³).

In some embodiments, the perfusion culturing in step (b) can be performed using a perfusion rate of about 0.1 nL/cell/day to about 60 nL/cell/day, about 0.1 nL/cell/day to about 55 nL/cell/day, about 0.1 nL/cell/day to about 50 nL/cell/day, about 0.1 nL/cell/day to about 45 nL/cell/day, about 0.1 nL/cell/day to about 40 nL/cell/day, about 0.1 nL/cell/day to about 35 nL/cell/day, about 0.1 nL/cell/day to about 30 nL/cell/day, about 0.1 nL/cell/day to about 25 nL/cell/day, about 0.1 nL/cell/day to about 20 nL/cell/day, about 0.1 nL/cell/day to about 15 nL/cell/day, about 0.1 nL/cell/day to about 10 nL/cell/day, about 0.1 nL/cell/day to about 8 nL/cell/day, about 0.1 nL/cell/day to about 6 nL/cell/day, about 0.1 nL/cell/day to about 5 nL/cell/day, about 0.1 nL/cell/day to about 4 nL/cell/day, about 0.1 nL/cell/day to about 2 nL/cell/day, about 0.1 nL/cell/day to about 1 nL/cell/day, about 0.1 nL/cell/day to about 0.5 nL/cell/day, about 0.5 nL/cell/day to about 60 nL/cell/day, about 0.5 nL/cell/day to about 55 nL/cell/day, about 0.5 nL/cell/day to about 50 nL/cell/day, about 0.5 nL/cell/day to about 45 nL/cell/day, about 0.5 nL/cell/day to about 40 nL/cell/day, about 0.5 nL/cell/day to about 35 nL/cell/day, about 0.5 nL/cell/day to about 30 nL/cell/day, about 0.5 nL/cell/day to about 25 nL/cell/day, about 0.5 nL/cell/day to about 20 nL/cell/day, about 0.5 nL/cell/day to about 15 nL/cell/day, about 0.5 nL/cell/day to about 10 nL/cell/day, about 0.5 nL/cell/day to about 8 nL/cell/day, about 0.5 nL/cell/day to about 6 nL/cell/day, about 0.5 nL/cell/day to about 5 nL/cell/day, about 0.5 nL/cell/day to about 4 nL/cell/day, about 0.5 nL/cell/day to about 2 nL/cell/day, about 0.5 nL/cell/day to about 1 nL/cell/day, about 1 nL/cell/day to about 60 nL/cell/day, about 1 nL/cell/day to about 55 nL/cell/day, about 1 nL/cell/day to about 50 nL/cell/day, about 1 nL/cell/day to about 45 nL/cell/day, about 1 nL/cell/day to about 40 nL/cell/day, about 1 nL/cell/day to about 35 nL/cell/day, about 1 nL/cell/day to about 30 nL/cell/day, about 1 nL/cell/day to about 25 nL/cell/day, about 1 nL/cell/day to about 20 nL/cell/day, about 1 nL/cell/day to about 15 nL/cell/day, about 1 nL/cell/day to about 10 nL/cell/day, about 1 nL/cell/day to about 8 nL/cell/day, about 1 nL/cell/day to about 6 nL/cell/day, about 1 nL/cell/day to about 5 nL/cell/day, about 1 nL/cell/day to about 4 nL/cell/day, about 1 nL/cell/day to about 2 nL/cell/day, about 2 nL/cell/day to about 60 nL/cell/day, about 2 nL/cell/day to about 55 nL/cell/day, about 2 nL/cell/day to about 50 nL/cell/day, about 2 nL/cell/day to about 45 nL/cell/day, about 2 nL/cell/day to about 40 nL/cell/day, about 2 nL/cell/day to about 35 nL/cell/day, about 2 nL/cell/day to about 30 nL/cell/day, about 2 nL/cell/day to about 25 nL/cell/day, about 2 nL/cell/day to about 20 nL/cell/day, about 2 nL/cell/day to about 15 nL/cell/day, about 2 nL/cell/day to about 10 nL/cell/day, about 2 nL/cell/day to about 8 nL/cell/day, about 2 nL/cell/day to about 6 nL/cell/day, about 2 nL/cell/day to about 5 nL/cell/day, about 2 nL/cell/day to about 4 nL/cell/day, about 4 nL/cell/day to about 60 nL/cell/day, about 4 nL/cell/day to about 55 nL/cell/day, about 4 nL/cell/day to about 50 nL/cell/day, about 4 nL/cell/day to about 45 nL/cell/day, about 4 nL/cell/day to about 40 nL/cell/day, about 4 nL/cell/day to about 35 nL/cell/day, about 4 nL/cell/day to about 30 nL/cell/day, about 4 nL/cell/day to about 25 nL/cell/day, about 4 nL/cell/day to about 20 nL/cell/day, about 4 nL/cell/day to about 15 nL/cell/day, about 4 nL/cell/day to about 10 nL/cell/day, about 4 nL/cell/day to about 8 nL/cell/day, about 4 nL/cell/day to about 6 nL/cell/day, about 4 nL/cell/day to about 5 nL/cell/day, about 5 nL/cell/day to about 60 nL/cell/day, about 5 nL/cell/day to about 55 nL/cell/day, about 5 nL/cell/day to about 50 nL/cell/day, about 5 nL/cell/day to about 45 nL/cell/day, about 5 nL/cell/day to about 40 nL/cell/day, about 5 nL/cell/day to about 35 nL/cell/day, about 5 nL/cell/day to about 30 nL/cell/day, about 5 nL/cell/day to about 25 nL/cell/day, about 5 nL/cell/day to about 20 nL/cell/day, about 5 nL/cell/day to about 15 nL/cell/day, about 5 nL/cell/day to about 10 nL/cell/day, about 5 nL/cell/day to about 8 nL/cell/day, about 5 nL/cell/day to about 6 nL/cell/day, about 6 nL/cell/day to about 60 nL/cell/day, about 6 nL/cell/day to about 55 nL/cell/day, about 6 nL/cell/day to about 50 nL/cell/day, about 6 nL/cell/day to about 45 nL/cell/day, about 6 nL/cell/day to about 40 nL/cell/day, about 6 nL/cell/day to about 35 nL/cell/day, about 6 nL/cell/day to about 30 nL/cell/day, about 6 nL/cell/day to about 25 nL/cell/day, about 6 nL/cell/day to about 20 nL/cell/day, about 6 nL/cell/day to about 15 nL/cell/day, about 6 nL/cell/day to about 10 nL/cell/day, about 6 nL/cell/day to about 8 nL/cell/day, about 8 nL/cell/day to about 60 nL/cell/day, about 8 nL/cell/day to about 55 nL/cell/day, about 8 nL/cell/day to about 50 nL/cell/day, about 8 nL/cell/day to about 45 nL/cell/day, about 8 nL/cell/day to about 40 nL/cell/day, about 8 nL/cell/day to about 35 nL/cell/day, about 8 nL/cell/day to about 30 nL/cell/day, about 8 nL/cell/day to about 25 nL/cell/day, about 8 nL/cell/day to about 20 nL/cell/day, about 8 nL/cell/day to about 15 nL/cell/day, about 8 nL/cell/day to about 10 nL/cell/day, about 10 nL/cell/day to about 60 nL/cell/day, about 10 nL/cell/day to about 55 nL/cell/day, about 10 nL/cell/day to about 50 nL/cell/day, about 10 nL/cell/day to about 45 nL/cell/day, about 10 nL/cell/day to about 40 nL/cell/day, about 10 nL/cell/day to about 35 nL/cell/day, about 10 nL/cell/day to about 30 nL/cell/day, about 10 nL/cell/day to about 25 nL/cell/day, about 10 nL/cell/day to about 20 nL/cell/day, about 10 nL/cell/day to about 15 nL/cell/day, about 15 nL/cell/day to about 60 nL/cell/day, about 15 nL/cell/day to about 55 nL/cell/day, about 15 nL/cell/day to about 50 nL/cell/day, about 15 nL/cell/day to about 45 nL/cell/day, about 15 nL/cell/day to about 40 nL/cell/day, about 15 nL/cell/day to about 35 nL/cell/day, about 15 nL/cell/day to about 30 nL/cell/day, about 15 nL/cell/day to about 25 nL/cell/day, about 15 nL/cell/day to about 20 nL/cell/day, about 20 nL/cell/day to about 60 nL/cell/day, about 20 nL/cell/day to about 55 nL/cell/day, about 20 nL/cell/day to about 50 nL/cell/day, about 20 nL/cell/day to about 45 nL/cell/day, about 20 nL/cell/day to about 40 nL/cell/day, about 20 nL/cell/day to about 35 nL/cell/day, about 20 nL/cell/day to about 30 nL/cell/day, about 20 nL/cell/day to about 25 nL/cell/day, about 25 nL/cell/day to about 60 nL/cell/day, about 25 nL/cell/day to about 55 nL/cell/day, about 25 nL/cell/day to about 50 nL/cell/day, about 25 nL/cell/day to about 45 nL/cell/day, about 25 nL/cell/day to about 40 nL/cell/day, about 25 nL/cell/day to about 35 nL/cell/day, about 25 nL/cell/day to about 30 nL/cell/day, about 30 nL/cell/day to about 60 nL/cell/day, about 30 nL/cell/day to about 55 nL/cell/day, about 30 nL/cell/day to about 50 nL/cell/day, about 30 nL/cell/day to about 45 nL/cell/day, about 30 nL/cell/day to about 40 nL/cell/day, about 30 nL/cell/day to about 35 nL/cell/day, about 35 nL/cell/day to about 60 nL/cell/day, about 35 nL/cell/day to about 55 nL/cell/day, about 35 nL/cell/day to about 50 nL/cell/day, about 35 nL/cell/day to about 45 nL/cell/day, about 35 nL/cell/day to about 40 nL/cell/day, about 40 nL/cell/day to about 60 nL/cell/day, about 40 nL/cell/day to about 55 nL/cell/day, about 40 nL/cell/day to about 50 nL/cell/day, about 40 nL/cell/day to about 45 nL/cell/day, about 45 nL/cell/day to about 60 nL/cell/day, about 45 nL/cell/day to about 55 nL/cell/day, about 45 nL/cell/day to about 50 nL/cell/day, about 50 nL/cell/day to about 60 nL/cell/day, about 50 nL/cell/day to about 55 nL/cell/day, or about 55 nL/cell/day to about 60 nL/cell/day. In some embodiments, the perfusion rate is increased over time. In some embodiments, the perfusion culturing in step (b) can be performed using a perfusion rate of about 0.1 vessel volume per day (VVD) to about 3 VVD (e.g., about 0.1 VVD to about 2.8 VVD, about 0.1 VVD to about 2.6 VVD, about 0.1 VVD to about 2.4 VVD, about 0.1 VVD to about 2.2 VVD, about 0.1 VVD to about 2.0 VVD, about 0.1 VVD to about 1.8 VVD, about 0.1 VVD to about 1.6 VVD, about 0.1 VVD to about 1.4 VVD, about 0.1 VVD to about 1.2 VVD, about 0.1 VVD to about 1.0 VVD, about 0.1 VVD to about 0.8 VVD, about 0.1 VVD to about 0.6 VVD, about 0.1 VVD to about 0.4 VVD, about 0.1 VVD to about 0.2 VVD, about 0.2 VVD to about 3 VVD, about 0.2 VVD to about 2.8 VVD, about 0.2 VVD to about 2.6 VVD, about 0.2 VVD to about 2.4 VVD, about 0.2 VVD to about 2.2 VVD, about 0.2 VVD to about 2.0 VVD, about 0.2 VVD to about 1.8 VVD, about 0.2 VVD to about 1.6 VVD, about 0.2 VVD to about 1.4 VVD, about 0.2 VVD to about 1.2 VVD, about 0.2 VVD to about 1.0 VVD, about 0.2 VVD to about 0.8 VVD, about 0.2 VVD to about 0.6 VVD, about 0.2 VVD to about 0.4 VVD, about 0.4 VVD to about 3 VVD, about 0.4 VVD to about 2.8 VVD, about 0.4 VVD to about 2.6 VVD, about 0.4 VVD to about 2.4 VVD, about 0.4 VVD to about 2.2 VVD, about 0.4 VVD to about 2.0 VVD, about 0.4 VVD to about 1.8 VVD, about 0.4 VVD to about 1.6 VVD, about 0.4 VVD to about 1.4 VVD, about 0.4 VVD to about 1.2 VVD, about 0.4 VVD to about 1.0 VVD, about 0.4 VVD to about 0.8 VVD, about 0.4 VVD to about 0.6 VVD, about 0.6 VVD to about 3 VVD, about 0.6 VVD to about 2.8 VVD, about 0.6 VVD to about 2.6 VVD, about 0.6 VVD to about 2.4 VVD, about 0.6 VVD to about 2.2 VVD, about 0.6 VVD to about 2.0 VVD, about 0.6 VVD to about 1.8 VVD, about 0.6 VVD to about 1.6 VVD, about 0.6 VVD to about 1.4 VVD, about 0.6 VVD to about 1.2 VVD, about 0.6 VVD to about 1.0 VVD, about 0.6 VVD to about 0.8 VVD, about 0.8 VVD to about 3 VVD, about 0.8 VVD to about 2.8 VVD, about 0.8 VVD to about 2.6 VVD, about 0.8 VVD to about 2.4 VVD, about 0.8 VVD to about 2.2 VVD, about 0.8 VVD to about 2.0 VVD, about 0.8 VVD to about 1.8 VVD, about 0.8 VVD to about 1.6 VVD, about 0.8 VVD to about 1.4 VVD, about 0.8 VVD to about 1.2 VVD, about 0.8 VVD to about 1.0 VVD, about 1.0 VVD to about 3 VVD, about 1.0 VVD to about 2.8 VVD, about 1.0 VVD to about 2.6 VVD, about 1.0 VVD to about 2.4 VVD, about 1.0 VVD to about 2.2 VVD, about 1.0 VVD to about 2.0 VVD, about 1.0 VVD to about 1.8 VVD, about 1.0 VVD to about 1.6 VVD, about 1.0 VVD to about 1.4 VVD, about 1.0 VVD to about 1.2 VVD, about 1.2 VVD to about 3 VVD, about 1.2 VVD to about 2.8 VVD, about 1.2 VVD to about 2.6 VVD, about 1.2 VVD to about 2.4 VVD, about 1.2 VVD to about 2.2 VVD, about 1.2 VVD to about 2.0 VVD, about 1.2 VVD to about 1.8 VVD, about 1.2 VVD to about 1.6 VVD, about 1.2 VVD to about 1.4 VVD, about 1.4 VVD to about 3 VVD, about 1.4 VVD to about 2.8 VVD, about 1.4 VVD to about 2.6 VVD, about 1.4 VVD to about 2.4 VVD, about 1.4 VVD to about 2.2 VVD, about 1.4 VVD to about 2.0 VVD, about 1.4 VVD to about 1.8 VVD, about 1.4 VVD to about 1.6 VVD, about 1.6 VVD to about 3 VVD, about 1.6 VVD to about 2.8 VVD, about 1.6 VVD to about 2.6 VVD, about 1.6 VVD to about 2.4 VVD, about 1.6 VVD to about 2.2 VVD, about 1.6 VVD to about 2.0 VVD, about 1.6 VVD to about 1.8 VVD, about 1.8 VVD to about 3 VVD, about 1.8 VVD to about 2.8 VVD, about 1.8 VVD to about 2.6 VVD, about 1.8 VVD to about 2.4 VVD, about 1.8 VVD to about 2.2 VVD, about 1.8 VVD to about 2.0 VVD, about 2.0 VVD to about 3 VVD, about 2.0 VVD to about 2.8 VVD, about 2.0 VVD to about 2.6 VVD, about 2.0 VVD to about 2.4 VVD, about 2.0 VVD to about 2.2 VVD, about 2.2 VVD to about 3 VVD, about 2.2 VVD to about 2.8 VVD, about 2.2 VVD to about 2.6 VVD, about 2.2 VVD to about 2.4 VVD, about 2.4 VVD to about 3 VVD, about 2.4 VVD to about 2.8 VVD, about 2.4 VVD to about 2.6 VVD, about 2.6 VVD to about 3 VVD, about 2.6 VVD to about 2.8 VVD, or about 2.8 VVD to about 3 VVD).

In some examples, the perfusion culturing in step (b) includes, at least in part, adding an additional volume of culture medium (e.g., the second culture medium (e.g., any of the exemplary second culture media described herein)) to the second cell culture over time. In some examples, the additional volume of culture medium (e.g., the second culture medium (e.g., any of the exemplary second culture media described herein)) is added continuously to the second cell culture over time. In some embodiments, the additional volume of culture medium (e.g., the second culture medium (e.g., any of the exemplary second culture media described herein)) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the second cell culture over time. The addition of culture medium (e.g., any of the exemplary second culture media described herein) can be performed mechanically, e.g., using a peristaltic pump or a perfusion pump, or manually (e.g., by sterile pipetting).

In some examples, the perfusion culturing in step (b) includes, at least in part, removing a volume of the culture medium (e.g., substantially cell-free culture medium) over time. In some examples, the culture medium (e.g., substantially cell-free culture medium) is removed continuously over time. In some embodiments, the culture medium is removed periodically (e.g., once every three days, once every two days, once a day, twice a day, three times a day, four times a day, five times a day, six times a day, seven times a day, eight times a day, nine times a day, ten times a day, eleven times a day, or twelve times a day) over time. The removal of culture medium (e.g., substantially cell-free culture medium) can be performed mechanically, e.g., using a tangential flow filtration (TFF) or alternating flow filtration (ATF), or manually (e.g., by sterile pipetting). Additional non-limiting aspects of tangential flow filtration are described herein.

In some examples of these methods, the second culture medium includes one or more (e.g., one, two, three, four, five, or six) of: transferrin (e.g., about 1 μg/mL to about 500 μg/mL transferrin (e.g., human apotransferrin, human holo transferrin, or a combination thereof) or any of the subranges of this range described herein), IL-3 (e.g., about 0.1 ng/mL to about 200 ng/mL IL-3 (e.g., recombinant human IL-3) or any of the subranges of this range described herein), SCF (e.g., about 1 ng/mL to about 1 μg/mL SCF (e.g., recombinant human SCF) or any of the subranges of this range described herein), dexamethasone (e.g., about 0.1 nM to about 200 nM dexamethasone or any of the subranges of this range described herein), erythropoietin (EPO) or an EPO-mimetic peptide (e.g., about 1 ng/mL to about 500 ng/mL of EPO (e.g., recombinant human EPO) or an EPO-mimetic peptide or any of the subranges of this range described herein), and insulin (e.g., about 0.1 μg/mL to about 50 μg/mL insulin or any of the subranges of this range described herein). In some examples of these methods, the second culture medium includes lipid (e.g., lipid mixture). In some examples of these methods, the second culture medium includes about 0.1 mM to about 10 mM (or any of the subranges of this range described herein) of L-glutamine, L-alanyl-L-glutamine, L-glycyl-L-glutamine, N-acetyl-L-glutamine, or a combination thereof.

In some embodiments, the perfusion culturing includes the use of tangential filtration (e.g., tangential flow filtration (TFF) or alternating tangential filtration (ATF)). In some embodiments, the tangential filtration (e.g., tangential flow filtration or alternating tangential filtration) includes the use of one or more filters that have an average pore size of about 10 nm to about 6.0 μm, about 10 nm to about 5.5 μm, about 10 nm to about 5.0 μam, about 10 nm to about 4.5 μm, about 10 nm to about 4.0 μm, about 10 nm to about 3.5 μm, about 10 nm to about 3.0 μm, about 10 nm to about 2.5 μm, about 10 nm to about 2.0 μm, about 10 nm to about 1.5 μm, about 10 nm to about 1.0 μm, about 10 nm to about 0.5 μm, about 10 nm to about 0.2 μm, 10 nm to about 0.1 μm, about 10 nm to about 50 nm, about 50 nm to about 6.0 μm, about 50 nm to about 5.5 μm, about 50 nm to about 5.0 μm, about 50 nm to about 4.5 μm, about 50 nm to about 4.0 μm, about 50 nm to about 3.5 μm, about 50 nm to about 3.0 μm, about 50 nm to about 2.5 μm, about 50 nm to about 2.0 μm, about 50 nm to about 1.5 μm, about 50 nm to about 1.0 μm, about 50 nm to about 0.5 μm, about 50 nm to about 0.2 μm, about 50 nm to about 0.1 μm, about 0.1 μm to about 6.0 μm, about 0.1 μm to about 5.5 μm, about 0.1 μm to about 5.0 μm, about 0.1 μm to about 4.5 μm, about 0.1 μm to about 4.0 μm, about 0.1 μm to about 3.5 μm, about 0.1 μm to about 3.0 μm, about 0.1 μm to about 2.5 μm, about 0.1 μm to about 2.0 μm, about 0.1 μm to about 1.5 μm, about 0.1 μm to about 1.0 μm, about 0.1 μm to about 0.5 μm, about 0.1 μm to about 0.2 μm, about 0.2 μm to about 6.0 μm, about 0.2 μm to about 5.5 μm, about 0.2 μm to about 5.0 μm, about 0.2 μm to about 4.5 μm, about 0.2 μm to about 4.0 μm, about 0.2 μm to about 3.5 μm, about 0.2 μm to about 3.0 μm, about 0.2 μm to about 2.5 μm, about 0.2 μm to about 2.0 μm, about 0.2 μm to about 1.5 μm, about 0.2 μm to about 1.0 μm, about 0.2 μm to about 0.5 μm, about 0.5 μm to about 6.0 μm, about 0.5 μm to about 5.5 μm, about 0.5 μm to about 5.0 μm, about 0.5 μm to about 4.5 μm, about 0.5 μm to about 4.0 μm, about 0.5 μm to about 3.5 μm, about 0.5 μm to about 3.0 μm, about 0.5 μm to about 2.5 μm, about 0.5 μm to about 2.0 μm, about 0.5 μm to about 1.5 μm, about 0.5 μm to about 1.0 μm, about 1.0 μm to about 6.0 μm, about 1.0 μm to about 5.5 μm, about 1.0 μm to about 5.0 μm, about 1.0 μm to about 4.5 μm, about 1.0 μm to about 4.0 μm, about 1.0 μm to about 3.5 μm, about 1.0 μm to about 3.0 μm, about 1.0 μm to about 2.5 μm, about 1.0 μm to about 2.0 μm, about 1.0 μm to about 1.5 μm, about 1.5 μm to about 6.0 μm, about 1.5 μm to about 5.5 μm, about 1.5 μm to about 5.0 μm, about 1.5 μm to about 4.5 μm, about 1.5 μm to about 4.0 μm, about 1.5 μm to about 3.5 μm, about 1.5 μm to about 3.0 μm, about 1.5 μm to about 2.5 μm, about 1.5 μm to about 2.0 μm, about 2.0 μm to about 6.0 μm, about 2.0 μm to about 5.5 μm, about 2.0 μm to about 5.0 μm, about 2.0 μm to about 4.5 μm, about 2.0 μm to about 4.0 μm, about 2.0 μm to about 3.5 μm, about 2.0 μm to about 3.0 μm, about 2.0 μm to about 2.5 μm, about 2.5 μm to about 6.0 μm, about 2.5 μm to about 5.5 μm, about 2.5 μm to about 5.0 μm, about 2.5 μm to about 4.5 μm, about 2.5 μm to about 4.0 μm, about 2.5 μm to about 3.5 μm, about 2.5 μm to about 3.0 μm, about 3.0 μm to about 6.0 μm, about 3.0 μm to about 5.5 μm, about 3.0 μm to about 5.0 μm, about 3.0 μm to about 4.5 μm, about 3.0 μm to about 4.0 μm, about 3.0 μm to about 3.5 μm, about 3.5 μm to about 6.0 μm, about 3.5 μm to about 5.5 μm, about 3.5 μm to about 5.0 μm, about 3.5 μm to about 4.5 μm, about 3.5 μm to about 4.0 μm, about 4.0 μm to about 6.0 μm, about 4.0 μm to about 5.5 μm, about 4.0 μm to about 5.0 μm, about 4.0 μm to about 4.5 μm, about 4.5 μm to about 6.0 μM, about 4.5 μm to about 5.5 μm, about 4.5 μm to about 5.0 μm, about 5.0 μm to about 6.0 μm, about 5.0 μm to about 5.5 μm, or about 5.5 μm to about 6.0 μm.

In some embodiments, the perfusion of the second cell culture begins once the first cell culture reaches a specific target cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

Batch or Fed Batch Culturing

In some embodiments of any of the methods described herein, step (b) includes batch or fed batch culturing the second cell culture (e.g., any of the second cell cultures in any of the exemplary shake flasks, shake tubes, culture bags, or bioreactors described herein having any of the exemplary volumes described herein) for about 2 days to about 15 days (e.g., about 2 days to about 14 days, about 2 days to about 13 days, about 2 days to about 12 days, about 2 days to about 11 days, about 2 days to about 10 days, about 2 days to about 9 days, about 2 day to about 8 days, about 2 days to about 7 days, about 2 days to about 6 days, about 2 days to about 5 days, about 2 days to about 4 days, about 2 days to about 3 days, about 3 days to about 15 days, about 3 days to about 14 days, about 3 days to about 13 days, about 3 days to about 12 days, about 3 days to about 11 days, about 3 days to about 10 days, about 3 days to about 9 days, about 3 days to about 8 days, about 3 days to about 7 days, about 3 days to about 6 days, about 3 days to about 5 days, about 3 days to about 4 days, about 4 days to about 15 days, about 4 days to about 14 days, about 4 days to about 13 days, about 4 days to about 12 days, about 4 days to about 11 days, about 4 days to about 10 days, about 4 days to about 9 days, about 4 days to about 8 days, about 4 days to about 7 days, about 4 days to about 6 days, about 4 days to about 5 days, about 5 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, about 5 days to about 6 days, about 6 days to about 15 days, about 6 days to about 14 days, about 6 days to about 13 days, about 6 days to about 12 days, about 6 days to about 11 days, about 6 days to about 10 days, about 6 days to about 9 days, about 6 days to about 8 days, about 6 days to about 7 days, about 7 days to about 15 days, about 7 days to about 14 days, about 7 days to about 13 days, about 7 days to about 12 days, about 7 days to about 11 days, about 7 days to about 10 days, about 7 days to about 9 days, about 7 days to about 8 days, about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 15 days, about 9 days to about 14 days, about 9 days to about 13 days, about 9 days to about 12 days, about 9 days to about 11 days, about 9 days to about 10 days, about 10 days to about 15 days, about 10 days to about 14 days, about 10 days to about 13 days, about 10 days to about 12 days, about 10 days to about 11 days, about 11 days to about 15 days, about 11 days to about 14 days, about 11 days to about 13 days, about 11 days to about 12 days, about 12 days to about 15 days, about 12 days to about 14 days, about 12 days to about 13 days, about 13 days to about 15 days, about 13 days to about 14 days, or about 14 days to about 15 days).

In some embodiments, step (b) includes batch or fed batch culturing the second cell culture disposed in a bioreactor (e.g., any of the exemplary bioreactors described herein having any of the exemplary volumes described herein). In such embodiments, step (b) includes incubating the second cell culture in the bioreactor with a P/V value of about 10 W/m³ to about 200 W/m³ (or any of the subranges of this range described herein).

In some embodiments, step (b) includes batch or fed batch culturing the second cell culture disposed in a shake flask (e.g., any of the exemplary shake flasks described herein having any of the exemplary volumes described herein). In such embodiments, step (b) includes incubating the second cell culture in the shake flask at about 0.1×g to about 50×g (or any of the subranges of this range described herein).

In some embodiments, step (b) includes batch or fed batch culturing the second cell culture disposed in a shake tube (e.g., any of the exemplary shake tubes described herein (e.g., a conical container) having any of the exemplary volumes described herein). In such embodiments, step (b) includes incubating the second cell culture in the shake tube at about 0.1×g to about 50×g (or any of the subranges of this range described herein).

In some embodiments, step (b) includes batch or fed batch culturing the second cell culture disposed in a culture bag (e.g., any of the exemplary culture bags described herein having any of the exemplary volumes described herein). In such embodiments, step (b) includes incubating the second cell culture in the culture bag at rocking rate of 10 rock cycles per minute to about 50 rock cycles per minute (or any of the subranges of this range described herein).

In some embodiments, step (b) includes batch culturing the second cell culture.

In some embodiments, step (b) includes fed batch culturing the second cell culture. In some embodiments, fed batch culturing in step (b) includes adding an additional volume of the second culture medium (e.g., any of the exemplary second culture media described herein) over time. In some examples, the additional volume of culture medium (e.g., the second culture medium (e.g., any of the exemplary second culture media described herein)) is added continuously to the second cell culture over time. In some examples, the additional volume of culture medium (e.g., the second culture medium (e.g., any of the exemplary second culture media described herein)) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the second cell culture over time. In some embodiments, about 0.1× to about 10× (e.g., about 0.1× to about 9.5×, about 0.1× to about 9.0×, about 0.1× to about 8.5×, about 0.1× to about 8.0×, about 0.1× to about 7.5×, about 0.1× to about 7.0×, about 0.1× to about 6.5×, about 0.1× to about 6.0×, about 0.1× to about 5.5×, about 0.1× to about 5.0×, about 0.1× to about 4.5×, about 0.1× to about 4.0×, about 0.1× to about 3.5×, about 0.1× to about 3.0×, about 0.1× to about 2.5×, about 0.1× to about 2.0×, about 0.1× to about 1.5×, about 0.1× to about 1.0×, about 0.1× to about 0.5×, about 0.1× to about 0.3×, about 0.1× to about 0.2×, about 0.2× to about 10×, about 0.2× to about 9.5×, about 0.2× to about 9.0×, about 0.2× to about 8.5×, about 0.2× to about 8.0×, about 0.2× to about 7.5×, about 0.2× to about 7.0×, about 0.2× to about 6.5×, about 0.2× to about 6.0×, about 0.2× to about 5.5×, about 0.2× to about 5.0×, about 0.2× to about 4.5×, about 0.2× to about 4.0×, about 0.2× to about 3.5×, about 0.2× to about 3.0×, about 0.2× to about 2.5×, about 0.2× to about 2.0×, about 0.2× to about 1.5×, about 0.2× to about 1.0×, about 0.2× to about 0.5×, about 0.2× to about 0.3×, about 0.3× to about 10×, about 0.3× to about 9.5×, about 0.3× to about 9.0×, about 0.3× to about 8.5×, about 0.3× to about 8.0×, about 0.3× to about 7.5×, about 0.3× to about 7.0×, about 0.3× to about 6.5×, about 0.3× to about 6.0×, about 0.3× to about 5.5×, about 0.3× to about 5.0×, about 0.3× to about 4.5×, about 0.3× to about 4.0×, about 0.3× to about 3.5×, about 0.3× to about 3.0×, about 0.3× to about 2.5×, about 0.3× to about 2.0×, about 0.3× to about 1.5×, about 0.3× to about 1.0×, about 0.3× to about 0.5×, about 0.5× to about 10×, about 0.5× to about 9.5×, about 0.5× to about 9.0×, about 0.5× to about 8.5×, about 0.5× to about 8.0×, about 0.5× to about 7.5×, about 0.5× to about 7.0×, about 0.5× to about 6.5×, about 0.5× to about 6.0×, about 0.5× to about 5.5×, about 0.5× to about 5.0×, about 0.5× to about 4.5×, about 0.5× to about 4.0×, about 0.5× to about 3.5×, about 0.5× to about 3.0×, about 0.5× to about 2.5×, about 0.5× to about 2.0×, about 0.5× to about 1.5×, about 0.5× to about 1.0×, about 1.0× to about 10×, about 1.0× to about 9.5×, about 1.0× to about 9.0×, about 1.0× to about 8.5×, about 1.0× to about 8.0×, about 1.0× to about 7.5×, about 1.0× to about 7.0×, about 1.0× to about 6.5×, about 1.0× to about 6.0×, about 1.0× to about 5.5×, about 1.0× to about 5.0×, about 1.0× to about 4.5×, about 1.0× to about 4.0×, about 1.0× to about 3.5×, about 1.0× to about 3.0×, about 1.0× to about 2.5×, about 1.0× to about 2.0×, about 1.0× to about 1.5×, about 1.5× to about 10×, about 1.5× to about 9.5×, about 1.5× to about 9.0×, about 1.5× to about 8.5×, about 1.5× to about 8.0×, about 1.5× to about 7.5×, about 1.5× to about 7.0×, about 1.5× to about 6.5×, about 1.5× to about 6.0×, about 1.5× to about 5.5×, about 1.5× to about 5.0×, about 1.5× to about 4.5×, about 1.5× to about 4.0×, about 1.5× to about 3.5×, about 1.5× to about 3.0×, about 1.5× to about 2.5×, about 1.5× to about 2.0×, about 2.0× to about 10×, about 2.0× to about 9.5×, about 2.0× to about 9.0×, about 2.0× to about 8.5×, about 2.0× to about 8.0×, about 2.0× to about 7.5×, about 2.0× to about 7.0×, about 2.0× to about 6.5×, about 2.0× to about 6.0×, about 2.0× to about 5.5×, about 2.0× to about 5.0×, about 2.0× to about 4.5×, about 2.0× to about 4.0×, about 2.0× to about 3.5×, about 2.0× to about 3.0×, about 2.0× to about 2.5×, about 2.5× to about 10×, about 2.5× to about 9.5×, about 2.5× to about 9.0×, about 2.5× to about 8.5×, about 2.5× to about 8.0×, about 2.5× to about 7.5×, about 2.5× to about 7.0×, about 2.5× to about 6.5×, about 2.5× to about 6.0×, about 2.5× to about 5.5×, about 2.5× to about 5.0×, about 2.5× to about 4.5×, about 2.5× to about 4.0×, about 2.5× to about 3.5×, about 2.5× to about 3.0×, about 3.0× to about 10×, about 3.0× to about 9.5×, about 3.0× to about 9.0×, about 3.0× to about 8.5×, about 3.0× to about 8.0×, about 3.0× to about 7.5×, about 3.0× to about 7.0×, about 3.0× to about 6.5×, about 3.0× to about 6.0×, about 3.0× to about 5.5×, about 3.0× to about 5.0×, about 3.0× to about 4.5×, about 3.0× to about 4.0×, about 3.0× to about 3.5×, about 3.5× to about 10×, about 3.5× to about 9.5×, about 3.5× to about 9.0×, about 3.5× to about 8.5×, about 3.5× to about 8.0×, about 3.5× to about 7.5×, about 3.5× to about 7.0×, about 3.5× to about 6.5×, about 3.5× to about 6.0×, about 3.5× to about 5.5×, about 3.5× to about 5.0×, about 3.5× to about 4.5×, about 3.5× to about 4.0×, about 4.0× to about 10×, about 4.0× to about 9.5×, about 4.0× to about 9.0×, about 4.0× to about 8.5×, about 4.0× to about 8.0×, about 4.0× to about 7.5×, about 4.0× to about 7.0×, about 4.0× to about 6.5×, about 4.0× to about 6.0×, about 4.0× to about 5.5×, about 4.0× to about 5.0×, about 4.0× to about 4.5×, about 4.5× to about 10×, about 4.5× to about 9.5×, about 4.5× to about 9.0×, about 4.5× to about 8.5×, about 4.5× to about 8.0×, about 4.5× to about 7.5×, about 4.5× to about 7.0×, about 4.5× to about 6.5×, about 4.5× to about 6.0×, about 4.5× to about 5.5×, about 4.5× to about 5.0×, about 5.0× to about 10×, about 5.0× to about 9.5×, about 5.0× to about 9.0×, about 5.0× to about 8.5×, about 5.0× to about 8.0×, about 5.0× to about 7.5×, about 5.0× to about 7.0×, about 5.0× to about 6.5×, about 5.0× to about 6.0×, about 5.0× to about 5.5×, about 5.5× to about 10×, about 5.5× to about 9.5×, about 5.5× to about 9.0×, about 5.5× to about 8.5×, about 5.5× to about 8.0×, about 5.5× to about 7.5×, about 5.5× to about 7.0×, about 5.5× to about 6.5×, about 5.5× to about 6.0×, about 6.0× to about 10×, about 6.0× to about 9.5×, about 6.0× to about 9.0×, about 6.0× to about 8.5×, about 6.0× to about 8.0×, about 6.0× to about 7.5×, about 6.0× to about 7.0×, about 6.0× to about 6.5×, about 6.5× to about 10×, about 6.5× to about 9.5×, about 6.5× to about 9.0×, about 6.5× to about 8.5×, about 6.5× to about 8.0×, about 6.5× to about 7.5×, about 6.5× to about 7.0×, about 7.0× to about 10×, about 7.0× to about 9.5×, about 7.0× to about 9.0×, about 7.0× to about 8.5×, about 7.0× to about 8.0×, about 7.0× to about 7.5×, about 7.5× to about 10×, about 7.5× to about 9.5×, about 7.5× to about 9.0×, about 7.5× to about 8.5×, about 7.5× to about 8.0×, about 8.0× to about 10×, about 8.0× to about 9.5×, about 8.0× to about 9.0×, about 8.0× to about 8.5×, about 8.5× to about 10×, about 8.5× to about 9.5×, about 8.5× to about 9.0×, about 9.0× to about 10×, about 9.0× to about 9.5×, or about 9.5× to about 10×) of the volume of the second cell culture immediately after step (a) is added in each 24-hour increment. In some embodiments, the addition of additional volumes of culture medium (e.g., the second culture medium (e.g., any of the exemplary second culture media described herein) to the second cell culture begins once the first cell culture reaches a specific target cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

The addition of culture medium (e.g., any of the exemplary second culture media described herein) can be performed mechanically, e.g., using a peristaltic pump, or manually (e.g., by sterile pipetting).

In some examples of these methods, the second culture medium includes one or more (e.g., one, two, three, four, five, or six) of: transferrin (e.g., about 1 μg/mL to about 500 μg/mL transferrin (e.g., (e.g., human apotransferrin, human holo transferrin, or a combination thereof)) or any of the subranges of this range described herein), IL-3 (e.g., about 0.1 ng/mL to about 200 ng/mL IL-3 (e.g., recombinant human IL-3) or any of the subranges of this range described herein), SCF (e.g., about 1 ng/mL to about 1 μg/mL SCF (e.g., recombinant human SCF) or any of the subranges of this range described herein), dexamethasone (e.g., about 0.1 nM to about 200 nM dexamethasone or any of the subranges of this range described herein), erythropoietin (EPO) or an EPO-mimetic peptide (e.g., about 1 ng/mL to about 500 ng/mL of EPO (e.g., recombinant human EPO) or an EPO-mimetic peptide (e.g., any of the EPO-mimetic peptides described herein) or any of the subranges of this range described herein), and insulin (e.g., about 0.1 μg/mL to about 50 μg/mL insulin or any of the subranges of this range described herein). In some examples of these methods, the second culture medium includes lipid (e.g., lipid mixture). In some examples of these methods, the second culture medium includes about 0.1 mM to about 10 mM (or any of the subranges of this range described herein) of L-glutamine, L-alanyl-L-glutamine, L-glycyl-L-glutamine, N-acetyl-L-glutamine, or a combination thereof

### Providing a Third Cell Culture (Step C)

The methods described herein include a step (step (c)) of disposing a volume of the second cell culture (e.g., any of the exemplary second cell cultures described herein) of step (b) into a third culture medium (e.g., any of the exemplary third culture media described herein) comprised within a vessel (e.g., a bioreactor (e.g., a perfusion bioreactor), a shake tube, a shake flask, or a culture bag) to provide a third cell culture with an initial cell density of about 0.5×10⁵ cells/mL to about 1×10⁷ cells/mL, about 0.5×10⁵ cells/mL to about 0.5×10⁷ cells/mL, about 0.5×10⁵ cells/mL to about 1×10⁶ cells/mL, about 0.5×10⁵ cells/mL to about 0.5×10⁶ cells/mL, about 0.5×10⁵ cells/mL to about 0.1×10⁶ cells/mL, about 0.5×10⁵ cells/mL to about 8×10⁵ cells/mL, about 0.5×10⁵ cells/mL to about 6×10⁵ cells/mL, about 0.5×10⁵ cells/mL to about 4×10⁵ cells/mL, about 0.5×10⁵ cells/mL to about 2×10⁵ cells/mL, about 0.5×10⁵ cells/mL to about 1×10⁵ cells/mL, about 1×10⁵ cells/mL to about 1×10⁷ cells/mL, about 1×10⁵ cells/mL to about 0.5×10⁷ cells/mL, about 1×10⁵ cells/mL to about 1×10⁶ cells/mL, about 1×10⁵ cells/mL to about 0.5×10⁶ cells/mL, about 1×10⁵ cells/mL to about 0.1×10⁶ cells/mL, about 1×10⁵ cells/mL to about 8×10⁵ cells/mL, about 1×10⁵ cells/mL to about 6×10⁵ cells/mL, about 1×10⁵ cells/mL to about 4×10⁵ cells/mL, about 1×10⁵ cells/mL to about 2×10⁵ cells/mL, about 2×10⁵ cells/mL to about 1×10⁷ cells/mL, about 2×10⁵ cells/mL to about 0.5×10⁷ cells/mL, about 2×10⁵ cells/mL to about 1×10⁶ cells/mL, about 2×10⁵ cells/mL to about 0.5×10⁶ cells/mL, about 2×10⁵ cells/mL to about 0.1×10⁶ cells/mL, about 2×10⁵ cells/mL to about 8×10⁵ cells/mL, about 2×10⁵ cells/mL to about 6×10⁵ cells/mL, about 2×10⁵ cells/mL to about 4×10⁵ cells/mL, about 4×10⁵ cells/mL to about 1×10⁷ cells/mL, about 4×10⁵ cells/mL to about 0.5×10⁷ cells/mL, about 4×10⁵ cells/mL to about 1×10⁶ cells/mL, about 4×10⁵ cells/mL to about 0.5×10⁶ cells/mL, about 4×10⁵ cells/mL to about 0.1×10⁶ cells/mL, about 4×10⁵ cells/mL to about 8×10⁵ cells/mL, about 4×10⁵ cells/mL to about 6×10⁵ cells/mL, about 6×10⁵ cells/mL to about 1×10⁷ cells/mL, about 6×10⁵ cells/mL to about 0.5×10⁷ cells/mL, about 6×10⁵ cells/mL to about 1×10⁶ cells/mL, about 6×10⁵ cells/mL to about 0.5×10⁶ cells/mL, about 6×10⁵ cells/mL to about 0.1×10⁶ cells/mL, about 6×10⁵ cells/mL to about 8×10⁵ cells/mL, about 8×10⁵ cells/mL to about 1×10⁷ cells/mL, about 8×10⁵ cells/mL to about 0.5×10⁷ cells/mL, about 8×10⁵ cells/mL to about 1×10⁶ cells/mL, about 8×10⁵ cells/mL to about 0.5×10⁶ cells/mL, about 8×10⁵ cells/mL to about 0.1×10⁶ cells/mL, about 0.1×10⁶ cells/mL to about 1×10⁷ cells/mL, about 0.1×10⁶ cells/mL to about 0.5×10⁷ cells/mL, about 0.1×10⁶ cells/mL to about 1×10⁶ cells/mL, about 0.1×10⁶ cells/mL to about 0.5×10⁶ cells/mL, about 0.5×10⁶ cells/mL to about 1×10⁷ cells/mL, about 0.5×10⁶ cells/mL to about 0.5×10⁷ cells/mL, about 0.5×10⁶ cells/mL to about 1×10⁶ cells/mL, about 1×10⁶ cells/mL to about 1×10⁷ cells/mL, about 1×10⁶ cells/mL to about 0.5×10⁷ cells/mL, or about 0.5×10⁷ cells/mL to about 1×10⁷ cells/mL.

In some embodiments, step (c) includes disposing a volume of the second cell culture (e.g., any of the exemplary second cell cultures described herein) of step (b) into a third culture medium comprised within a bioreactor (e.g., any of the bioreactors (e.g., perfusion bioreactors) having any of the exemplary volumes described herein) to provide a third cell culture with an initial cell density of about 0.5×10⁵ cells/mL to about 1×10⁷ cells/mL (or any of the subranges of this range described herein).

In some embodiments, step (c) includes disposing a volume of the second cell culture (e.g., any of the exemplary second cell cultures described herein) of step (b) into a third culture medium comprised within a shake flask (e.g., any of the exemplary shake flasks described herein having any of the exemplary volumes described herein) to provide a third cell culture with an initial cell density of about 0.5×10⁵ cells/mL to about 1×10⁷ cells/mL (or any of the subranges of this range described herein).

In some embodiments, step (c) includes disposing a volume of the second cell culture (e.g., any of the exemplary second cell cultures described herein) of step (b) into a third culture medium comprised within a shake tube (e.g., any of the exemplary shake tubes described herein (e.g., a conical container) having any of the exemplary volumes described herein) to provide a third cell culture with an initial cell density of about 0.5×10⁵ cells/mL to about 1×10⁷ cells/mL (or any of the subranges of this range described herein).

In some embodiments, step (c) includes disposing a volume of the second cell culture (e.g., any of the exemplary second cell cultures described herein) of step (b) into a third culture medium comprised within a culture bag (e.g., any of the exemplary culture bags described herein having any of the exemplary volumes described herein) to provide a third cell culture with an initial cell density of about 0.5×10⁵ cells/mL to about 1×10⁷ cells/mL (or any of the subranges of this range described herein).

In some examples, the third culture medium includes one or more (e.g., two, three, or four) transferrin (e.g., about 1 μg/mL to about 500 transferrin (e.g., apotransferrin, holo transferrin, or a combination thereof) or any of the subranges of this range described herein), insulin (e.g., about 0.1 μg/mL to about 50 μg/mL insulin (e.g., recombinant human insulin) or any of the subranges of this range described herein), SCF (e.g., about 1 ng/mL to about 500 ng/mL SCF (e.g., recombinant human SCF) or any of the subranges of this range described herein), and EPO or an EPO mimetic peptide (e.g., about 1 ng/mL to about 500 ng/mL EPO (e.g., recombinant human EPO) or an EPO mimetic peptide (e.g., any of the exemplary EPO mimetic peptides described herein or known in the art), or any of the subranges of this range described herein). In some embodiments of any of the third culture media described herein, the third culture medium includes Iscove's modified Dulbecco's medium (IMDM). In some embodiments of any of the third culture media described herein, the third culture medium includes about 0.1% v/v to about 10% v/v (e.g., about 0.1% v/v to about 9.5% v/v, about 0.1% v/v to about 9.0% v/v, about 0.1% v/v to about 8.5% v/v, about 0.1% v/v to about 8.0% v/v, about 0.1% v/v to about 7.5% v/v, about 0.1% v/v to about 7.0% v/v, about 0.1% v/v to about 6.5% v/v, about 0.1% v/v to about 6.0% v/v, about 0.1% v/v to about 5.5% v/v, about 0.1% v/v to about 5.0% v/v, about 0.1% v/v to about 4.5% v/v, about 0.1% v/v to about 4.0% v/v, about 0.1% v/v to about 3.5% v/v, about 0.1% v/v to about 3.0% v/v, about 0.1% v/v to about 2.5% v/v, about 0.1% v/v to about 2.0% v/v, about 0.1% v/v to about 1.5% v/v, about 0.1% v/v to about 1.0% v/v, about 0.5% v/v to about 1.0% v/v, about 0.5% v/v to about 10% v/v, about 0.5% v/v to about 9.5% v/v, about 0.5% v/v to about 9.0% v/v, about 0.5% v/v to about 8.5% v/v, about 0.5% v/v to about 8.0% v/v, about 0.5% v/v to about 7.5% v/v, about 0.5% v/v to about 7.0% v/v, about 0.5% v/v to about 6.5% v/v, about 0.5% v/v to about 6.0% v/v, about 0.5% v/v to about 5.5% v/v, about 0.5% v/v to about 5% v/v, about 0.5% v/v to about 4.5% v/v, about 0.5% v/v to about 4.0% v/v, about 0.5% v/v to about 3.5% v/v, about 0.5% v/v to about 3.0% v/v, about 0.5% v/v to about 2.5% v/v, about 0.5% v/v to about 2.0% v/v, about 0.5% v/v to about 1.5% v/v, about 0.5% v/v to about 1.0% v/v, about 1.0% v/v to about 10% v/v, about 1.0% v/v to about 9.5% v/v, about 1.0% v/v to about 9.0% v/v, about 1.0% v/v to about 8.5% v/v, about 1.0% v/v to about 8.0% v/v, about 1.0% v/v to about 7.5% v/v, about 1.0% v/v to about 7.0% v/v, about 1.0% v/v to about 6.5% v/v, about 1.0% v/v to about 6.0% v/v, about 1.0% v/v to about 5.5% v/v, about 1.0% v/v to about 5% v/v, about 1.0% v/v to about 4.5% v/v, about 1.0% v/v to about 4.0% v/v, about 1.0% v/v to about 3.5% v/v, about 1.0% v/v to about 3.0% v/v, about 1.0% v/v to about 2.5% v/v, about 1.0% v/v to about 2.0% v/v, about 1.0% v/v to about 1.5% v/v, about 1.5% v/v to about 10% v/v, about 1.5% v/v to about 9.5% v/v, about 1.5% v/v to about 9.0% v/v, about 1.5% v/v to about 8.5% v/v, about 1.5% v/v to about 8.0% v/v, about 1.5% v/v to about 7.5% v/v, about 1.5% v/v to about 7.0% v/v, about 1.5% v/v to about 6.5% v/v, about 1.5% v/v to about 6.0% v/v, about 1.5% v/v to about 5.5% v/v, about 1.5% v/v to about 5% v/v, about 1.5% v/v to about 4.5% v/v, about 1.5% v/v to about 4.0% v/v, about 1.5% v/v to about 3.5% v/v, about 1.5% v/v to about 3.0% v/v, about 1.5% v/v to about 2.5% v/v, about 1.5% v/v to about 2.0% v/v, about 2.0% v/v to about 10% v/v, about 2.0% v/v to about 9.5% v/v, about 2.0% v/v to about 9.0% v/v, about 2.0% v/v to about 8.5% v/v, about 2.0% v/v to about 8.000% v/v, about 2.0% v/v to about 7.5% v/v, about 2.0% v/v to about 7.0% v/v, about 2.0% v/v to about 6.5% v/v, about 2.0% v/v to about 6.0% v/v, about 2.0% v/v to about 5.5% v/v, about 2.0% v/v to about 5% v/v, about 2.0% v/v to about 4.5% v/v, about 2.0% v/v to about 4.0% v/v, about 2.0% v/v to about 3.5% v/v, about 2.0% v/v to about 3.0% v/v, about 2.0% v/v to about 2.5% v/v, about 2.5% v/v to about 10% v/v, about 2.5% v/v to about 9.5% v/v, about 2.5% v/v to about 9.0% v/v, about 2.5% v/v to about 8.5% v/v, about 2.5% v/v to about 8.0% v/v, about 2.5% v/v to about 7.5% v/v, about 2.5% v/v to about 7.0% v/v, about 2.5% v/v to about 6.5% v/v, about 2.5% v/v to about 6.0% v/v, about 2.5% v/v to about 5.5% v/v, about 2.5% v/v to about 5% v/v, about 2.5% v/v to about 4.5% v/v, about 2.5% v/v to about 4.0% v/v, about 2.5% v/v to about 3.5% v/v, about 2.5% v/v to about 3.0% v/v, about 3.0% v/v to about 10% v/v, about 3.0% v/v to about 9.5% v/v, about 3.0% v/v to about 9.0% v/v, about 3.0% v/v to about 8.5% v/v, about 3.0% v/v to about 8.0% v/v, about 3.0% v/v to about 7.5% v/v, about 3.0% v/v to about 7.0% v/v, about 3.0% v/v to about 6.5% v/v, about 3.0% v/v to about 6.0% v/v, about 3.0% v/v to about 5.5% v/v, about 3.0% v/v to about 5% v/v, about 3.0% v/v to about 4.5% v/v, about 3.0% v/v to about 4.0% v/v, about 3.0% v/v to about 3.5% v/v, about 3.5% v/v to about 10% v/v, about 3.5% v/v to about 9.5% v/v, about 3.5% v/v to about 9.0% v/v, about 3.5% v/v to about 8.5% v/v, about 3.5% v/v to about 8.0% v/v, about 3.5% v/v to about 7.5% v/v, about 3.5% v/v to about 7.0% v/v, about 3.5% v/v to about 6.5% v/v, about 3.5% v/v to about 6.0% v/v, about 3.5% v/v to about 5.5% v/v, about 3.5% v/v to about 5% v/v, about 3.5% v/v to about 4.5% v/v, about 3.5% v/v to about 4.0% v/v, about 4.0% v/v to about 10% v/v, about 4.0% v/v to about 9.5% v/v, about 4.0% v/v to about 9.0% v/v, about 4.0% v/v to about 8.5% v/v, about 4.0% v/v to about 8.0% v/v, about 4.0% v/v to about 7.5% v/v, about 4.0% v/v to about 7.0% v/v, about 4.0% v/v to about 6.5% v/v, about 4.0% v/v to about 6.0% v/v, about 4.0% v/v to about 5.5% v/v, about 4.0% v/v to about 5% v/v, about 4.0% v/v to about 4.5% v/v, about 4.5% v/v to about 10% v/v, about 4.5% v/v to about 9.5% v/v, about 4.5% v/v to about 9.0% v/v, about 4.5% v/v to about 8.5% v/v, about 4.5% v/v to about 8.0% v/v, about 4.5% v/v to about 7.5% v/v, about 4.5% v/v to about 7.0% v/v, about 4.5% v/v to about 6.5% v/v, about 4.5% v/v to about 6.0% v/v, about 4.5% v/v to about 5.5% v/v, about 4.5% v/v to about 5.0% v/v, about 5.0% v/v to about 10% v/v, about 5.0% v/v to about 9.5% v/v, about 5.0% v/v to about 9.0% v/v, about 5.0% v/v to about 8.5% v/v, about 5.0% v/v to about 8.0% v/v, about 5.0% v/v to about 7.5% v/v, about 5.0% v/v to about 7.0% v/v, about 5.0% v/v to about 6.5% v/v, about 5.0% v/v to about 6.0% v/v, about 5.0% v/v to about 5.5% v/v, about 5.5% v/v to about 10% v/v, about 5.5% v/v to about 9.5% v/v, about 5.5% v/v to about 9.0% v/v, about 5.5% v/v to about 8.5% v/v, about 5.5% v/v to about 8.0% v/v, about 5.5% v/v to about 7.5% v/v, about 5.5% v/v to about 7.0% v/v, about 5.5% v/v to about 6.5% v/v, about 5.5% v/v to about 6.0% v/v, about 6.0% v/v to about 10% v/v, about 6.0% v/v to about 9.5% v/v, about 6.0% v/v to about 9.0% v/v, about 6.0% v/v to about 8.5% v/v, about 6.0% v/v to about 8.0% v/v, about 6.0% v/v to about 7.5% v/v, about 6.0% v/v to about 7.000% v/v, about 6.0% v/v to about 6.5% v/v, about 6.5% v/v to about 10% v/v, about 6.5% v/v to about 9.5% v/v, about 6.5% v/v to about 9.0% v/v, about 6.5% v/v to about 8.5% v/v, about 6.5% v/v to about 8.0% v/v, about 6.5% v/v to about 7.5% v/v, about 6.5% v/v to about 7.0% v/v, about 7.0% v/v to about 10% v/v, about 7.0% v/v to about 9.5% v/v, about 7.0% v/v to about 9.0% v/v, about 7.0% v/v to about 8.5% v/v, about 7.0% v/v to about 8.0% v/v, about 7.0% v/v to about 7.5% v/v, about 7.5% v/v to about 10% v/v, about 7.5% v/v to about 9.5% v/v, about 7.5% v/v to about 9.0% v/v, about 7.5% v/v to about 8.5% v/v, about 7.5% v/v to about 8.0% v/v, about 8.0% v/v to about 10% v/v, about 8.0% v/v to about 9.5% v/v, about 8.0% v/v to about 9.0% v/v, about 8.000% v/v to about 8.5% v/v, about 8.5% v/v to about 10% v/v, about 8.5% v/v to about 9.5% v/v, about 8.5% v/v to about 9.0% v/v, about 9.0% v/v to about 10% v/v, about 9.0% v/v to about 9.5% v/v, or about 9.5% v/v to about 10% v/v) serum (e.g., human AB serum). In some embodiments, the third culture media includes 0.1% v/v to about 5% v/v (e.g., about 0.1% v/v to about 4.5% v/v, about 0.1% v/v to about 4.0% v/v, about 0.1% v/v to about 3.5% v/v, about 0.1% v/v to about 3.0% v/v, about 0.1% v/v to about 2.5% v/v, about 0.1% v/v to about 2.0% v/v, about 0.1% v/v to about 1.5% v/v, about 0.1% v/v to about 1.0% v/v, about 0.1% v/v to about 0.5% v/v, about 0.5% v/v to about 5% v/v, about 0.5% v/v to about 4.5% v/v, about 0.5% v/v to about 4.0% v/v, about 0.5% v/v to about 3.5% v/v, about 0.5% v/v to about 3.0% v/v, about 0.5% v/v to about 2.5% v/v, about 0.5% v/v to about 2.0% v/v, about 0.5% v/v to about 1.5% v/v, about 0.5% v/v to about 1.0% v/v, about 1.0% v/v to about 5% v/v, about 1.0% v/v to about 4.5% v/v, about 1.0% v/v to about 4.0% v/v, about 1.0% v/v to about 3.5% v/v, about 1.0% v/v to about 3.0% v/v, about 1.0% v/v to about 2.5% v/v, about 1.0% v/v to about 2.0% v/v, about 1.0% v/v to about 1.5% v/v, about 1.5% v/v to about 5% v/v, about 1.5% v/v to about 4.5% v/v, about 1.5% v/v to about 4.0% v/v, about 1.5% v/v to about 3.5% v/v, about 1.5% v/v to about 3.0% v/v, about 1.5% v/v to about 2.5% v/v, about 1.5% v/v to about 2.0% v/v, about 2.0% v/v to about 5% v/v, about 2.0% v/v to about 4.5% v/v, about 2.0% v/v to about 4.0% v/v, about 2.0% v/v to about 3.5% v/v, about 2.0% v/v to about 3.0% v/v, about 2.0% v/v to about 2.5% v/v, about 2.5% v/v to about 5% v/v, about 2.5% v/v to about 4.5% v/v, about 2.5% v/v to about 4.0% v/v, about 2.5% v/v to about 3.5% v/v, about 2.5% v/v to about 3.0% v/v, about 3.0% v/v to about 5% v/v, about 3.0% v/v to about 4.5% v/v, about 3.0% v/v to about 4.0% v/v, about 3.0% v/v to about 3.5% v/v, about 3.5% v/v to about 5% v/v, about 3.5% v/v to about 4.5% v/v, about 3.5% v/v to about 4.0% v/v, about 4.0% v/v to about 5% v/v, about 4.0% v/v to about 4.5% v/v, or about 4.5% v/v to about 5% v/v) human plasma. In some embodiments, the third culture medium includes about 1 U/mL to about 5 U/mL (e.g., about 1 U/mL to about 4 U/mL, about 1 U/mL to about 3 U/mL, about 1 U/mL to about 2 U/mL, about 2 U/mL to about 5 U/mL, about 2 U/mL to about 4 U/mL, about 2 U/mL to about 3 U/mL, about 3 U/mL to about 5 U/mL, about 3 U/mL to about 4 U/mL, or about 4 U/mL to about 5 U/mL) heparin. In some examples, the third culture medium includes about 0.1% w/v to about 3% w/v (e.g., about 0.1% w/v to about 2.5% w/v, about 0.1% w/v to about 2.0% w/v, about 0.1% w/v to about 1.5% w/v, about 0.1% w/v to about 1.0% w/v, about 0.1% w/v to about 0.5% w/v, about 0.5% w/v to about 3% w/v, about 0.5% w/v to about 2.5% w/v, about 0.5% w/v to about 2.0% w/v, about 0.5% w/v to about 1.5% w/v, about 0.5% w/v to about 1.0% w/v, about 1.0% w/v to about 3% w/v, about 1.0% w/v to about 2.5% w/v, about 1.0% w/v to about 2.0% w/v, about 1.0% w/v to about 1.5% w/v, about 1.5% w/v to about 3% w/v, about 1.5% w/v to about 2.5% w/v, about 1.5% w/v to about 2.0% w/v, about 2.0% w/v to about 3% w/v, about 2.0% w/v to about 2.5% w/v, or about 2.5% w/v to about 3% w/v) Poloxamer-188 (P188).). In some embodiments of any of the third culture media described herein, the third culture medium includes about 1 mM to about 8 mM (e.g., about 1 mM to about 7 mM, about 1 mM to about 6 mM, about 1 mM to about 5 mM, about 1 mM to about 4 mM, about 1 mM to about 3 mM, about 1 mM to about 2 mM, about 2 mM to about 8 mM, about 2 mM to about 7 mM, about 2 mM to about 6 mM, about 2 mM to about 5 mM, about 2 mM to about 4 mM, about 2 mM to about 3 mM, about 3 mM to about 8 mM, about 3 mM to about 7 mM, about 3 mM to about 6 mM, about 3 mM to about 5 mM, about 3 mM to about 4 mM, about 4 mM to about 8 mM, about 4 mM to about 7 mM, about 4 mM to about 6 mM, about 4 mM to about 5 mM, about 5 mM to about 8 mM, about 5 mM to about 7 mM, about 5 mM to about 6 mM, about 6 mM to about 8 mM, about 6 mM to about 7 mM, or about 7 mM to about 8 mM) of L-glutamine, L-alanyl-L-glutamine, L-glycyl-L-glutamine, N-acetyl-L-glutamine, or a combination thereof.

In some examples, the third culture medium includes about 1 μg/mL to about 500 μg/mL, about 1 μg/mL to about 450 μg/mL, about 1 μg/mL to about 400 μg/mL, about 1 μg/mL to about 350 μg/mL, about 1 μg/mL to about 300 μg/mL, about 1 μg/mL to about 250 μg/mL, about 1 μg/mL to about 200 μg/mL, about 1 μg/mL to about 180 μg/mL, about 1 μg/mL to about 160 μg/mL, about 1 μg/mL to about 140 μg/mL, about 1 μg/mL to about 120 μg/mL, about 1 μg/mL to about 100 μg/mL, about 1 μg/mL to about 80 μg/mL, about 1 μg/mL to about 60 μg/mL, about 1 μg/mL to about 40 μg/mL, about 1 μg/mL to about 20 μg/mL, about 1 μg/mL to about 10 μg/mL, about 1 μg/mL to about 5 μg/mL, 5 μg/mL to about 500 μg/mL, about 5 μg/mL to about 450 μg/mL, about 5 μg/mL to about 400 μg/mL, about 5 μg/mL to about 350 μg/mL, about 5 μg/mL to about 300 μg/mL, about 5 μg/mL to about 250 μg/mL, about 5 μg/mL to about 200 μg/mL, about 5 μg/mL to about 180 μg/mL, about 5 μg/mL to about 160 μg/mL, about 5 μg/mL to about 140 μg/mL, about 5 μg/mL to about 120 μg/mL, about 5 μg/mL to about 100 μg/mL, about 5 μg/mL to about 80 μg/mL, about 5 μg/mL to about 60 μg/mL, about 5 μg/mL to about 40 μg/mL, about 5 μg/mL to about 20 μg/mL, about 5 μg/mL to about 10 μg/mL, 10 μg/mL to about 500 μg/mL, about 10 μg/mL to about 450 μg/mL, about 10 μg/mL to about 400 μg/mL, about 10 μg/mL to about 350 μg/mL, about 10 μg/mL to about 300 μg/mL, about 10 μg/mL to about 250 μg/mL, about 10 μg/mL to about 200 μg/mL, about 10 μg/mL to about 180 μg/mL, about 10 μg/mL to about 160 μg/mL, about 10 μg/mL to about 140 μg/mL, about 10 μg/mL to about 120 μg/mL, about 10 μg/mL to about 100 μg/mL, about 10 μg/mL to about 80 μg/mL, about 10 μg/mL to about 60 μg/mL, about 10 μg/mL to about 40 μg/mL, about 10 μg/mL to about 20 μg/mL, 20 μg/mL to about 500 μg/mL, about 20 μg/mL to about 450 μg/mL, about 20 μg/mL to about 400 μg/mL, about 20 μg/mL to about 350 μg/mL, about 20 μg/mL to about 300 μg/mL, about 20 μg/mL to about 250 μg/mL, about 20 μg/mL to about 200 μg/mL, about 20 μg/mL to about 180 μg/mL, about 20 μg/mL to about 160 μg/mL, about 20 μg/mL to about 140 μg/mL, about 20 μg/mL to about 120 μg/mL, about 20 μg/mL to about 100 μg/mL, about 20 μg/mL to about 80 μg/mL, about 20 μg/mL to about 60 μg/mL, about 20 μg/mL to about 40 μg/mL, 40 μg/mL to about 500 μg/mL, about 40 μg/mL to about 450 μg/mL, about 40 μg/mL to about 400 μg/mL, about 40 μg/mL to about 350 μg/mL, about 40 μg/mL to about 300 μg/mL, about 40 μg/mL to about 250 μg/mL, about 40 μg/mL to about 200 μg/mL, about 40 μg/mL to about 180 μg/mL, about 40 μg/mL to about 160 μg/mL, about 40 μg/mL to about 140 μg/mL, about 40 μg/mL to about 120 μg/mL, about 40 μg/mL to about 100 μg/mL, about 40 μg/mL to about 80 μg/mL, about 40 μg/mL to about 60 μg/mL, 60 μg/mL to about 500 μg/mL, about 60 μg/mL to about 450 μg/mL, about 60 μg/mL to about 400 μg/mL, about 60 μg/mL to about 350 μg/mL, about 60 μg/mL to about 300 μg/mL, about 60 μg/mL to about 250 μg/mL, about 60 μg/mL to about 200 μg/mL, about 60 μg/mL to about 180 μg/mL, about 60 μg/mL to about 160 μg/mL, about 60 μg/mL to about 140 μg/mL, about 60 μg/mL to about 120 μg/mL, about 60 μg/mL to about 100 μg/mL, about 60 μg/mL to about 80 μg/mL, 80 μg/mL to about 500 μg/mL, about 80 μg/mL to about 450 μg/mL, about 80 μg/mL to about 400 μg/mL, about 80 μg/mL to about 350 μg/mL, about 80 μg/mL to about 300 μg/mL, about 80 μg/mL to about 250 μg/mL, about 80 μg/mL to about 200 μg/mL, about 80 μg/mL to about 180 μg/mL, about 80 μg/mL to about 160 μg/mL, about 80 μg/mL to about 140 μg/mL, about 80 μg/mL to about 120 μg/mL, about 80 μg/mL to about 100 μg/mL, 100 μg/mL to about 500 μg/mL, about 100 μg/mL to about 450 μg/mL, about 100 μg/mL to about 400 μg/mL, about 100 μg/mL to about 350 μg/mL, about 100 μg/mL to about 300 μg/mL, about 100 μg/mL to about 250 μg/mL, about 100 μg/mL to about 200 μg/mL, about 100 μg/mL to about 180 μg/mL, about 100 μg/mL to about 160 μg/mL, about 100 μg/mL to about 140 μg/mL, about 100 μg/mL to about 120 μg/mL, 120 μg/mL to about 500 μg/mL, about 120 μg/mL to about 450 μg/mL, about 120 μg/mL to about 400 μg/mL, about 120 μg/mL to about 350 μg/mL, about 120 μg/mL to about 300 μg/mL, about 120 μg/mL to about 250 μg/mL, about 120 μg/mL to about 200 μg/mL, about 120 μg/mL to about 180 μg/mL, about 120 μg/mL to about 160 μg/mL, about 120 μg/mL to about 140 μg/mL, 140 μg/mL to about 500 μg/mL, about 140 μg/mL to about 450 μg/mL, about 140 μg/mL to about 400 μg/mL, about 140 μg/mL to about 350 μg/mL, about 140 μg/mL to about 300 μg/mL, about 140 μg/mL to about 250 μg/mL, about 140 μg/mL to about 200 μg/mL, about 140 μg/mL to about 180 μg/mL, about 140 μg/mL to about 160 μg/mL, 160 μg/mL to about 500 μg/mL, about 160 μg/mL to about 450 μg/mL, about 160 μg/mL to about 400 μg/mL, about 160 μg/mL to about 350 μg/mL, about 160 μg/mL to about 300 μg/mL, about 160 μg/mL to about 250 μg/mL, about 160 μg/mL to about 200 μg/mL, about 160 μg/mL to about 180 μg/mL, 180 μg/mL to about 500 μg/mL, about 180 μg/mL to about 450 μg/mL, about 180 μg/mL to about 400 μg/mL, about 180 μg/mL to about 350 μg/mL, about 180 μg/mL to about 300 μg/mL, about 180 μg/mL to about 250 μg/mL, about 180 μg/mL to about 200 μg/mL, 200 μg/mL to about 500 μg/mL, about 200 μg/mL to about 450 μg/mL, about 200 μg/mL to about 400 μg/mL, about 200 μg/mL to about 350 μg/mL, about 200 μg/mL to about 300 μg/mL, about 200 μg/mL to about 250 μg/mL, 250 μg/mL to about 500 μg/mL, about 250 μg/mL to about 450 μg/mL, about 250 μg/mL to about 400 μg/mL, about 250 μg/mL to about 350 μg/mL, about 250 μg/mL to about 300 μg/mL, 300 μg/mL to about 500 μg/mL, about 300 μg/mL to about 450 μg/mL, about 300 μg/mL to about 400 μg/mL, about 300 μg/mL to about 350 μg/mL, 350 μg/mL to about 500 μg/mL, about 350 μg/mL to about 450 μg/mL, about 350 μg/mL to about 400 μg/mL, 400 μg/mL to about 500 μg/mL, about 400 μg/mL to about 450 μg/mL, or 450 μg/mL to about 500 μg/mL, of transferrin (e.g., apotransferrin, holo transferrin, or a combination thereof).

In some examples, the third culture medium includes about 1 ng/mL to about 1 μg/mL, about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 450 ng/mL, about 1 ng/mL to about 400 ng/mL, about 1 ng/mL to about 350 ng/mL, about 1 ng/mL to about 300 ng/mL, about 1 ng/mL to about 250 ng/mL, about 1 ng/mL to about 200 ng/mL, about 1 ng/mL to about 150 ng/mL, about 1 ng/mL to about 100 ng/mL, about 1 ng/mL to about 50 ng/mL, about 1 ng/mL to about 20 ng/mL, about 1 ng/mL to about 5 ng/mL, about 5 ng/mL to about 1 μg/mL, about 5 ng/mL to about 900 ng/mL, about 5 ng/mL to about 800 ng/mL, about 5 ng/mL to about 700 ng/mL, about 5 ng/mL to about 600 ng/mL, about 5 ng/mL to about 500 ng/mL, about 5 ng/mL to about 450 ng/mL, about 5 ng/mL to about 400 ng/mL, about 5 ng/mL to about 350 ng/mL, about 5 ng/mL to about 300 ng/mL, about 5 ng/mL to about 250 ng/mL, about 5 ng/mL to about 200 ng/mL, about 5 ng/mL to about 150 ng/mL, about 5 ng/mL to about 100 ng/mL, about 5 ng/mL to about 50 ng/mL, about 5 ng/mL to about 20 ng/mL, about 20 ng/mL to about 1 μg/mL, about 20 ng/mL to about 900 ng/mL, about 20 ng/mL to about 800 ng/mL, about 20 ng/mL to about 700 ng/mL, about 20 ng/mL to about 600 ng/mL, about 20 ng/mL to about 500 ng/mL, about 20 ng/mL to about 450 ng/mL, about 20 ng/mL to about 400 ng/mL, about 20 ng/mL to about 350 ng/mL, about 20 ng/mL to about 300 ng/mL, about 20 ng/mL to about 250 ng/mL, about 20 ng/mL to about 200 ng/mL, about 20 ng/mL to about 150 ng/mL, about 20 ng/mL to about 100 ng/mL, about 20 ng/mL to about 50 ng/mL, about 50 ng/mL to about 1 μg/mL, about 50 ng/mL to about 900 ng/mL, about 50 ng/mL to about 800 ng/mL, about 50 ng/mL to about 700 ng/mL, about 50 ng/mL to about 600 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 50 ng/mL to about 250 ng/mL, about 50 ng/mL to about 200 ng/mL, about 50 ng/mL to about 150 ng/mL, about 50 ng/mL to about 100 ng/mL, about 100 ng/mL to about 1 μg/mL, about 100 ng/mL to about 900 ng/mL, about 100 ng/mL to about 800 ng/mL, about 100 ng/mL to about 700 ng/mL, about 100 ng/mL to about 600 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 100 ng/mL to about 250 ng/mL, about 100 ng/mL to about 200 ng/mL, about 100 ng/mL to about 150 ng/mL, about 150 ng/mL to about 1 μg/mL, about 150 ng/mL to about 900 ng/mL, about 150 ng/mL to about 800 ng/mL, about 150 ng/mL to about 700 ng/mL, about 150 ng/mL to about 600 ng/mL, about 150 ng/mL to about 500 ng/mL, about 150 ng/mL to about 450 ng/mL, about 150 ng/mL to about 400 ng/mL, about 150 ng/mL to about 350 ng/mL, about 150 ng/mL to about 300 ng/mL, about 150 ng/mL to about 250 ng/mL, about 150 ng/mL to about 200 ng/mL, about 200 ng/mL to about 1 μg/mL, about 200 ng/mL to about 900 ng/mL, about 200 ng/mL to about 800 ng/mL, about 200 ng/mL to about 700 ng/mL, about 200 ng/mL to about 600 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 200 ng/mL to about 250 ng/mL, about 250 ng/mL to about 1 μg/mL, about 250 ng/mL to about 900 ng/mL, about 250 ng/mL to about 800 ng/mL, about 250 ng/mL to about 700 ng/mL, about 250 ng/mL to about 600 ng/mL, about 250 ng/mL to about 500 ng/mL, about 250 ng/mL to about 450 ng/mL, about 250 ng/mL to about 400 ng/mL, about 250 ng/mL to about 350 ng/mL, about 250 ng/mL to about 300 ng/mL, about 300 ng/mL to about 1 μg/mL, about 300 ng/mL to about 900 ng/mL, about 300 ng/mL to about 800 ng/mL, about 300 ng/mL to about 700 ng/mL, about 300 ng/mL to about 600 ng/mL, about 300 ng/mL to about 500 ng/mL, about 300 ng/mL to about 450 ng/mL, about 300 ng/mL to about 400 ng/mL, about 300 ng/mL to about 350 ng/mL, about 350 ng/mL to about 1 μg/mL, about 350 ng/mL to about 900 ng/mL, about 350 ng/mL to about 800 ng/mL, about 350 ng/mL to about 700 ng/mL, about 350 ng/mL to about 600 ng/mL, about 350 ng/mL to about 500 ng/mL, about 350 ng/mL to about 450 ng/mL, about 350 ng/mL to about 400 ng/mL, about 400 ng/mL to about 1 μg/mL, about 400 ng/mL to about 900 ng/mL, about 400 ng/mL to about 800 ng/mL, about 400 ng/mL to about 700 ng/mL, about 400 ng/mL to about 600 ng/mL, about 400 ng/mL to about 500 ng/mL, about 400 ng/mL to about 450 ng/mL, about 450 ng/mL to about 1 μg/mL, about 450 ng/mL to about 900 ng/mL, about 450 ng/mL to about 800 ng/mL, about 450 ng/mL to about 700 ng/mL, about 450 ng/mL to about 600 ng/mL, about 450 ng/mL to about 500 ng/mL, about 500 ng/mL to about 1 μg/mL, about 500 ng/mL to about 900 ng/mL, about 500 ng/mL to about 800 ng/mL, about 500 ng/mL to about 700 ng/mL, about 500 ng/mL to about 600 ng/mL, about 600 ng/mL to about 1 μg/mL, about 600 ng/mL to about 900 ng/mL, about 600 ng/mL to about 800 ng/mL, about 600 ng/mL to about 700 ng/mL, about 700 ng/mL to about 1 μg/mL, about 700 ng/mL to about 900 ng/mL, about 700 ng/mL to about 800 ng/mL, about 800 ng/mL to about 1 μg/mL, about 800 ng/mL to about 900 ng/mL, about 900 ng/mL to about 1 μg/mL, of SCF (e.g., recombinant human SCF).

In some embodiments, the third culture medium comprises about 1 ng/mL to about 500 ng/mL, about 1 ng/mL to about 480 ng/mL, about 1 ng/mL to about 460 ng/mL, about 1 ng/mL to about 440 ng/mL, about 1 ng/mL to about 420 ng/mL, about 1 ng/mL to about 400 ng/mL, about 1 ng/mL to about 380 ng/mL, about 1 ng/mL to about 360 ng/mL, about 1 ng/mL to about 340 ng/mL, about 1 ng/mL to about 320 ng/mL, about 1 ng/mL to about 300 ng/mL, about 1 ng/mL to about 280 ng/mL, about 1 ng/mL to about 260 ng/mL, about 1 ng/mL to about 240 ng/mL, about 1 ng/mL to about 220 ng/mL, about 1 ng/mL to about 200 ng/mL, about 1 ng/mL to about 180 ng/mL, about 1 ng/mL to about 160 ng/mL, about 1 ng/mL to about 140 ng/mL, about 1 ng/mL to about 120 ng/mL, about 1 ng/mL to about 100 ng/mL, about 1 ng/mL to about 80 ng/mL, about 1 ng/mL to about 60 ng/mL, about 1 ng/mL to about 40 ng/mL, about 1 ng/mL to about 30 ng/mL, about 1 ng/mL to about 25 ng/mL, about 1 ng/mL to about 20 ng/mL, about 1 ng/mL to about 15 ng/mL, about 1 ng/mL to about 10 ng/mL, about 1 ng/mL to about 5 ng/mL, 5 ng/mL to about 500 ng/mL, about 5 ng/mL to about 480 ng/mL, about 5 ng/mL to about 460 ng/mL, about 5 ng/mL to about 440 ng/mL, about 5 ng/mL to about 420 ng/mL, about 5 ng/mL to about 400 ng/mL, about 5 ng/mL to about 380 ng/mL, about 5 ng/mL to about 360 ng/mL, about 5 ng/mL to about 340 ng/mL, about 5 ng/mL to about 320 ng/mL, about 5 ng/mL to about 300 ng/mL, about 5 ng/mL to about 280 ng/mL, about 5 ng/mL to about 260 ng/mL, about 5 ng/mL to about 240 ng/mL, about 5 ng/mL to about 220 ng/mL, about 5 ng/mL to about 200 ng/mL, about 5 ng/mL to about 180 ng/mL, about 5 ng/mL to about 160 ng/mL, about 5 ng/mL to about 140 ng/mL, about 5 ng/mL to about 120 ng/mL, about 5 ng/mL to about 100 ng/mL, about 5 ng/mL to about 80 ng/mL, about 5 ng/mL to about 60 ng/mL, about 5 ng/mL to about 40 ng/mL, about 5 ng/mL to about 30 ng/mL, about 5 ng/mL to about 25 ng/mL, about 5 ng/mL to about 20 ng/mL, about 5 ng/mL to about 15 ng/mL, about 5 ng/mL to about 10 ng/mL, 10 ng/mL to about 500 ng/mL, about 10 ng/mL to about 480 ng/mL, about 10 ng/mL to about 460 ng/mL, about 10 ng/mL to about 440 ng/mL, about 10 ng/mL to about 420 ng/mL, about 10 ng/mL to about 400 ng/mL, about 10 ng/mL to about 380 ng/mL, about 10 ng/mL to about 360 ng/mL, about 10 ng/mL to about 340 ng/mL, about 10 ng/mL to about 320 ng/mL, about 10 ng/mL to about 300 ng/mL, about 10 ng/mL to about 280 ng/mL, about 10 ng/mL to about 260 ng/mL, about 10 ng/mL to about 240 ng/mL, about 10 ng/mL to about 220 ng/mL, about 10 ng/mL to about 200 ng/mL, about 10 ng/mL to about 180 ng/mL, about 10 ng/mL to about 160 ng/mL, about 10 ng/mL to about 140 ng/mL, about 10 ng/mL to about 120 ng/mL, about 10 ng/mL to about 100 ng/mL, about 10 ng/mL to about 80 ng/mL, about 10 ng/mL to about 60 ng/mL, about 10 ng/mL to about 40 ng/mL, about 10 ng/mL to about 30 ng/mL, about 10 ng/mL to about 25 ng/mL, about 10 ng/mL to about 20 ng/mL, about 10 ng/mL to about 15 ng/mL, 15 ng/mL to about 500 ng/mL, about 15 ng/mL to about 480 ng/mL, about 15 ng/mL to about 460 ng/mL, about 15 ng/mL to about 440 ng/mL, about 15 ng/mL to about 420 ng/mL, about 15 ng/mL to about 400 ng/mL, about 15 ng/mL to about 380 ng/mL, about 15 ng/mL to about 360 ng/mL, about 15 ng/mL to about 340 ng/mL, about 15 ng/mL to about 320 ng/mL, about 15 ng/mL to about 300 ng/mL, about 15 ng/mL to about 280 ng/mL, about 15 ng/mL to about 260 ng/mL, about 15 ng/mL to about 240 ng/mL, about 15 ng/mL to about 220 ng/mL, about 15 ng/mL to about 200 ng/mL, about 15 ng/mL to about 180 ng/mL, about 15 ng/mL to about 160 ng/mL, about 15 ng/mL to about 140 ng/mL, about 15 ng/mL to about 120 ng/mL, about 15 ng/mL to about 100 ng/mL, about 15 ng/mL to about 80 ng/mL, about 15 ng/mL to about 60 ng/mL, about 15 ng/mL to about 40 ng/mL, about 15 ng/mL to about 30 ng/mL, about 15 ng/mL to about 25 ng/mL, about 15 ng/mL to about 20 ng/mL, 20 ng/mL to about 500 ng/mL, about 20 ng/mL to about 480 ng/mL, about 20 ng/mL to about 460 ng/mL, about 20 ng/mL to about 440 ng/mL, about 20 ng/mL to about 420 ng/mL, about 20 ng/mL to about 400 ng/mL, about 20 ng/mL to about 380 ng/mL, about 20 ng/mL to about 360 ng/mL, about 20 ng/mL to about 340 ng/mL, about 20 ng/mL to about 320 ng/mL, about 20 ng/mL to about 300 ng/mL, about 20 ng/mL to about 280 ng/mL, about 20 ng/mL to about 260 ng/mL, about 20 ng/mL to about 240 ng/mL, about 20 ng/mL to about 220 ng/mL, about 20 ng/mL to about 200 ng/mL, about 20 ng/mL to about 180 ng/mL, about 20 ng/mL to about 160 ng/mL, about 20 ng/mL to about 140 ng/mL, about 20 ng/mL to about 120 ng/mL, about 20 ng/mL to about 100 ng/mL, about 20 ng/mL to about 80 ng/mL, about 20 ng/mL to about 60 ng/mL, about 20 ng/mL to about 40 ng/mL, about 20 ng/mL to about 30 ng/mL, about 20 ng/mL to about 25 ng/mL, 25 ng/mL to about 500 ng/mL, about 25 ng/mL to about 480 ng/mL, about 25 ng/mL to about 460 ng/mL, about 25 ng/mL to about 440 ng/mL, about 25 ng/mL to about 420 ng/mL, about 25 ng/mL to about 400 ng/mL, about 25 ng/mL to about 380 ng/mL, about 25 ng/mL to about 360 ng/mL, about 25 ng/mL to about 340 ng/mL, about 25 ng/mL to about 320 ng/mL, about 25 ng/mL to about 300 ng/mL, about 25 ng/mL to about 280 ng/mL, about 25 ng/mL to about 260 ng/mL, about 25 ng/mL to about 240 ng/mL, about 25 ng/mL to about 220 ng/mL, about 25 ng/mL to about 200 ng/mL, about 25 ng/mL to about 180 ng/mL, about 25 ng/mL to about 160 ng/mL, about 25 ng/mL to about 140 ng/mL, about 25 ng/mL to about 120 ng/mL, about 25 ng/mL to about 100 ng/mL, about 25 ng/mL to about 80 ng/mL, about 25 ng/mL to about 60 ng/mL, about 25 ng/mL to about 40 ng/mL, about 25 ng/mL to about 30 ng/mL, 30 ng/mL to about 500 ng/mL, about 30 ng/mL to about 480 ng/mL, about 30 ng/mL to about 460 ng/mL, about 30 ng/mL to about 440 ng/mL, about 30 ng/mL to about 420 ng/mL, about 30 ng/mL to about 400 ng/mL, about 30 ng/mL to about 380 ng/mL, about 30 ng/mL to about 360 ng/mL, about 30 ng/mL to about 340 ng/mL, about 30 ng/mL to about 320 ng/mL, about 30 ng/mL to about 300 ng/mL, about 30 ng/mL to about 280 ng/mL, about 30 ng/mL to about 260 ng/mL, about 30 ng/mL to about 240 ng/mL, about 30 ng/mL to about 220 ng/mL, about 30 ng/mL to about 200 ng/mL, about 30 ng/mL to about 180 ng/mL, about 30 ng/mL to about 160 ng/mL, about 30 ng/mL to about 140 ng/mL, about 30 ng/mL to about 120 ng/mL, about 30 ng/mL to about 100 ng/mL, about 30 ng/mL to about 80 ng/mL, about 30 ng/mL to about 60 ng/mL, about 30 ng/mL to about 40 ng/mL, 40 ng/mL to about 500 ng/mL, about 40 ng/mL to about 480 ng/mL, about 40 ng/mL to about 460 ng/mL, about 40 ng/mL to about 440 ng/mL, about 40 ng/mL to about 420 ng/mL, about 40 ng/mL to about 400 ng/mL, about 40 ng/mL to about 380 ng/mL, about 40 ng/mL to about 360 ng/mL, about 40 ng/mL to about 340 ng/mL, about 40 ng/mL to about 320 ng/mL, about 40 ng/mL to about 300 ng/mL, about 40 ng/mL to about 280 ng/mL, about 40 ng/mL to about 260 ng/mL, about 40 ng/mL to about 240 ng/mL, about 40 ng/mL to about 220 ng/mL, about 40 ng/mL to about 200 ng/mL, about 40 ng/mL to about 180 ng/mL, about 40 ng/mL to about 160 ng/mL, about 40 ng/mL to about 140 ng/mL, about 40 ng/mL to about 120 ng/mL, about 40 ng/mL to about 100 ng/mL, about 40 ng/mL to about 80 ng/mL, about 40 ng/mL to about 60 ng/mL, 60 ng/mL to about 500 ng/mL, about 60 ng/mL to about 480 ng/mL, about 60 ng/mL to about 460 ng/mL, about 60 ng/mL to about 440 ng/mL, about 60 ng/mL to about 420 ng/mL, about 60 ng/mL to about 400 ng/mL, about 60 ng/mL to about 380 ng/mL, about 60 ng/mL to about 360 ng/mL, about 60 ng/mL to about 340 ng/mL, about 60 ng/mL to about 320 ng/mL, about 60 ng/mL to about 300 ng/mL, about 60 ng/mL to about 280 ng/mL, about 60 ng/mL to about 260 ng/mL, about 60 ng/mL to about 240 ng/mL, about 60 ng/mL to about 220 ng/mL, about 60 ng/mL to about 200 ng/mL, about 60 ng/mL to about 180 ng/mL, about 60 ng/mL to about 160 ng/mL, about 60 ng/mL to about 140 ng/mL, about 60 ng/mL to about 120 ng/mL, about 60 ng/mL to about 100 ng/mL, about 60 ng/mL to about 80 ng/mL, 80 ng/mL to about 500 ng/mL, about 80 ng/mL to about 480 ng/mL, about 80 ng/mL to about 460 ng/mL, about 80 ng/mL to about 440 ng/mL, about 80 ng/mL to about 420 ng/mL, about 80 ng/mL to about 400 ng/mL, about 80 ng/mL to about 380 ng/mL, about 80 ng/mL to about 360 ng/mL, about 80 ng/mL to about 340 ng/mL, about 80 ng/mL to about 320 ng/mL, about 80 ng/mL to about 300 ng/mL, about 80 ng/mL to about 280 ng/mL, about 80 ng/mL to about 260 ng/mL, about 80 ng/mL to about 240 ng/mL, about 80 ng/mL to about 220 ng/mL, about 80 ng/mL to about 200 ng/mL, about 80 ng/mL to about 180 ng/mL, about 80 ng/mL to about 160 ng/mL, about 80 ng/mL to about 140 ng/mL, about 80 ng/mL to about 120 ng/mL, about 80 ng/mL to about 100 ng/mL, 100 ng/mL to about 500 ng/mL, about 100 ng/mL to about 480 ng/mL, about 100 ng/mL to about 460 ng/mL, about 100 ng/mL to about 440 ng/mL, about 100 ng/mL to about 420 ng/mL, about 100 ng/mL to about 400 ng/mL, about 100 ng/mL to about 380 ng/mL, about 100 ng/mL to about 360 ng/mL, about 100 ng/mL to about 340 ng/mL, about 100 ng/mL to about 320 ng/mL, about 100 ng/mL to about 300 ng/mL, about 100 ng/mL to about 280 ng/mL, about 100 ng/mL to about 260 ng/mL, about 100 ng/mL to about 240 ng/mL, about 100 ng/mL to about 220 ng/mL, about 100 ng/mL to about 200 ng/mL, about 100 ng/mL to about 180 ng/mL, about 100 ng/mL to about 160 ng/mL, about 100 ng/mL to about 140 ng/mL, about 100 ng/mL to about 120 ng/mL, 120 ng/mL to about 500 ng/mL, about 120 ng/mL to about 480 ng/mL, about 120 ng/mL to about 460 ng/mL, about 120 ng/mL to about 440 ng/mL, about 120 ng/mL to about 420 ng/mL, about 120 ng/mL to about 400 ng/mL, about 120 ng/mL to about 380 ng/mL, about 120 ng/mL to about 360 ng/mL, about 120 ng/mL to about 340 ng/mL, about 120 ng/mL to about 320 ng/mL, about 120 ng/mL to about 300 ng/mL, about 120 ng/mL to about 280 ng/mL, about 120 ng/mL to about 260 ng/mL, about 120 ng/mL to about 240 ng/mL, about 120 ng/mL to about 220 ng/mL, about 120 ng/mL to about 200 ng/mL, about 120 ng/mL to about 180 ng/mL, about 120 ng/mL to about 160 ng/mL, about 120 ng/mL to about 140 ng/mL, 140 ng/mL to about 500 ng/mL, about 140 ng/mL to about 480 ng/mL, about 140 ng/mL to about 460 ng/mL, about 140 ng/mL to about 440 ng/mL, about 140 ng/mL to about 420 ng/mL, about 140 ng/mL to about 400 ng/mL, about 140 ng/mL to about 380 ng/mL, about 140 ng/mL to about 360 ng/mL, about 140 ng/mL to about 340 ng/mL, about 140 ng/mL to about 320 ng/mL, about 140 ng/mL to about 300 ng/mL, about 140 ng/mL to about 280 ng/mL, about 140 ng/mL to about 260 ng/mL, about 140 ng/mL to about 240 ng/mL, about 140 ng/mL to about 220 ng/mL, about 140 ng/mL to about 200 ng/mL, about 140 ng/mL to about 180 ng/mL, about 140 ng/mL to about 160 ng/mL, 160 ng/mL to about 500 ng/mL, about 160 ng/mL to about 480 ng/mL, about 160 ng/mL to about 460 ng/mL, about 160 ng/mL to about 440 ng/mL, about 160 ng/mL to about 420 ng/mL, about 160 ng/mL to about 400 ng/mL, about 160 ng/mL to about 380 ng/mL, about 160 ng/mL to about 360 ng/mL, about 160 ng/mL to about 340 ng/mL, about 160 ng/mL to about 320 ng/mL, about 160 ng/mL to about 300 ng/mL, about 160 ng/mL to about 280 ng/mL, about 160 ng/mL to about 260 ng/mL, about 160 ng/mL to about 240 ng/mL, about 160 ng/mL to about 220 ng/mL, about 160 ng/mL to about 200 ng/mL, about 160 ng/mL to about 180 ng/mL,180 ng/mL to about 500 ng/mL, about 180 ng/mL to about 480 ng/mL, about 180 ng/mL to about 460 ng/mL, about 180 ng/mL to about 440 ng/mL, about 180 ng/mL to about 420 ng/mL, about 180 ng/mL to about 400 ng/mL, about 180 ng/mL to about 380 ng/mL, about 180 ng/mL to about 360 ng/mL, about 180 ng/mL to about 340 ng/mL, about 180 ng/mL to about 320 ng/mL, about 180 ng/mL to about 300 ng/mL, about 180 ng/mL to about 280 ng/mL, about 180 ng/mL to about 260 ng/mL, about 180 ng/mL to about 240 ng/mL, about 180 ng/mL to about 220 ng/mL, about 180 ng/mL to about 200 ng/mL, 200 ng/mL to about 500 ng/mL, about 200 ng/mL to about 480 ng/mL, about 200 ng/mL to about 460 ng/mL, about 200 ng/mL to about 440 ng/mL, about 200 ng/mL to about 420 ng/mL, about 200 ng/mL to about 400 ng/mL, about 200 ng/mL to about 380 ng/mL, about 200 ng/mL to about 360 ng/mL, about 200 ng/mL to about 340 ng/mL, about 200 ng/mL to about 320 ng/mL, about 200 ng/mL to about 300 ng/mL, about 200 ng/mL to about 280 ng/mL, about 200 ng/mL to about 260 ng/mL, about 200 ng/mL to about 240 ng/mL, about 200 ng/mL to about 220 ng/mL, 220 ng/mL to about 500 ng/mL, about 220 ng/mL to about 480 ng/mL, about 220 ng/mL to about 460 ng/mL, about 220 ng/mL to about 440 ng/mL, about 220 ng/mL to about 420 ng/mL, about 220 ng/mL to about 300 ng/mL, about 220 ng/mL to about 380 ng/mL, about 220 ng/mL to about 360 ng/mL, about 220 ng/mL to about 340 ng/mL, about 220 ng/mL to about 320 ng/mL, about 220 ng/mL to about 400 ng/mL, about 220 ng/mL to about 280 ng/mL, about 220 ng/mL to about 260 ng/mL, about 220 ng/mL to about 240 ng/mL, 240 ng/mL to about 500 ng/mL, about 240 ng/mL to about 480 ng/mL, about 240 ng/mL to about 460 ng/mL, about 240 ng/mL to about 440 ng/mL, about 240 ng/mL to about 420 ng/mL, about 240 ng/mL to about 400 ng/mL, about 240 ng/mL to about 380 ng/mL, about 240 ng/mL to about 360 ng/mL, about 240 ng/mL to about 340 ng/mL, about 240 ng/mL to about 320 ng/mL, about 240 ng/mL to about 300 ng/mL, about 240 ng/mL to about 280 ng/mL, about 240 ng/mL to about 260 ng/mL, 260 ng/mL to about 500 ng/mL, about 260 ng/mL to about 480 ng/mL, about 260 ng/mL to about 460 ng/mL, about 260 ng/mL to about 440 ng/mL, about 260 ng/mL to about 420 ng/mL, about 260 ng/mL to about 400 ng/mL, about 260 ng/mL to about 380 ng/mL, about 260 ng/mL to about 360 ng/mL, about 260 ng/mL to about 340 ng/mL, about 260 ng/mL to about 320 ng/mL, about 260 ng/mL to about 300 ng/mL, about 260 ng/mL to about 280 ng/mL, 280 ng/mL to about 500 ng/mL, about 280 ng/mL to about 480 ng/mL, about 280 ng/mL to about 460 ng/mL, about 280 ng/mL to about 440 ng/mL, about 280 ng/mL to about 420 ng/mL, about 280 ng/mL to about 400 ng/mL, about 280 ng/mL to about 380 ng/mL, about 280 ng/mL to about 360 ng/mL, about 280 ng/mL to about 340 ng/mL, about 280 ng/mL to about 320 ng/mL, about 280 ng/mL to about 300 ng/mL, 300 ng/mL to about 500 ng/mL, about 300 ng/mL to about 480 ng/mL, about 300 ng/mL to about 460 ng/mL, about 300 ng/mL to about 440 ng/mL, about 300 ng/mL to about 420 ng/mL, about 300 ng/mL to about 400 ng/mL, about 300 ng/mL to about 380 ng/mL, about 300 ng/mL to about 360 ng/mL, about 300 ng/mL to about 340 ng/mL, about 300 ng/mL to about 320 ng/mL, 320 ng/mL to about 500 ng/mL, about 320 ng/mL to about 480 ng/mL, about 320 ng/mL to about 460 ng/mL, about 320 ng/mL to about 440 ng/mL, about 320 ng/mL to about 420 ng/mL, about 320 ng/mL to about 400 ng/mL, about 320 ng/mL to about 380 ng/mL, about 320 ng/mL to about 360 ng/mL, about 320 ng/mL to about 340 ng/mL, 340 ng/mL to about 500 ng/mL, about 340 ng/mL to about 480 ng/mL, about 340 ng/mL to about 460 ng/mL, about 340 ng/mL to about 440 ng/mL, about 340 ng/mL to about 420 ng/mL, about 340 ng/mL to about 400 ng/mL, about 340 ng/mL to about 380 ng/mL, about 340 ng/mL to about 360 ng/mL, 360 ng/mL to about 500 ng/mL, about 360 ng/mL to about 480 ng/mL, about 360 ng/mL to about 460 ng/mL, about 360 ng/mL to about 440 ng/mL, about 360 ng/mL to about 420 ng/mL, about 360 ng/mL to about 400 ng/mL, about 360 ng/mL to about 380 ng/mL, 380 ng/mL to about 500 ng/mL, about 380 ng/mL to about 480 ng/mL, about 380 ng/mL to about 460 ng/mL, about 380 ng/mL to about 440 ng/mL, about 380 ng/mL to about 420 ng/mL, about 380 ng/mL to about 400 ng/mL, 400 ng/mL to about 500 ng/mL, about 400 ng/mL to about 480 ng/mL, about 400 ng/mL to about 460 ng/mL, about 400 ng/mL to about 440 ng/mL, about 400 ng/mL to about 420 ng/mL, 420 ng/mL to about 500 ng/mL, about 420 ng/mL to about 480 ng/mL, about 420 ng/mL to about 460 ng/mL, about 420 ng/mL to about 440 ng/mL, 440 ng/mL to about 500 ng/mL, about 440 ng/mL to about 480 ng/mL, about 440 ng/mL to about 460 ng/mL, about 460 ng/mL to about 500 ng/mL, about 460 ng/mL to about 480 ng/mL, or about 480 ng/mL to about 500 ng/mL of EPO (e.g., recombinant human EPO) or an EPO-mimetic peptide. Non-limiting examples of EPO-mimetic peptide are described herein. Additional examples of EPO-mimetic peptides are known in the art.

In some embodiments, the third culture medium comprises about 0.1 μg/mL to about 50 μg/mL, about 0.1 μg/mL to about 45 μg/mL, about 0.1 μg/mL to about 40 μg/mL, about 0.1 μg/mL to about 35 μg/mL, about 0.1 μg/mL to about 30 μg/mL, about 0.1 μg/mL to about 25 μg/mL, about 0.1 μg/mL to about 20 μg/mL, about 0.1 μg/mL about 15 μg/mL, about 0.1 μg/mL to about 10 μg/mL, about 0.1 μg/mL to about 5 μg/mL, about 0.1 μg/mL to about 2 μg/mL, about 0.1 μg/mL to about 1 μg/mL, about 1 μg/mL to about 50 μg/mL, about 1 μg/mL to about 45 μg/mL, about 1 μg/mL to about 40 μg/mL, about 1 μg/mL to about 35 μg/mL, about 1 μg/mL to about 30 μg/mL, about 1 μg/mL to about 25 μg/mL, about 1 μg/mL to about 20 μg/mL, about 1 μg/mL about 15 μg/mL, about 1 μg/mL to about 10 μg/mL, about 1 μg/mL to about 5 μg/mL, about 1 μg/mL to about 2 μg/mL, about 2 μg/mL to about 50 μg/mL, about 2 μg/mL to about 45 μg/mL, about 2 μg/mL to about 40 μg/mL, about 2 μg/mL to about 35 μg/mL, about 2 μg/mL to about 30 μg/mL, about 2 μg/mL to about 25 μg/mL, about 2 μg/mL to about 20 μg/mL, about 2 μg/mL about 15 μg/mL, about 2 μg/mL to about 10 μg/mL, about 2 μg/mL to about 5 μg/mL, about 5 μg/mL to about 50 μg/mL, about 5 μg/mL to about 45 μg/mL, about 5 μg/mL to about 40 μg/mL, about 5 μg/mL to about 35 μg/mL, about 5 μg/mL to about 30 μg/mL, about 5 μg/mL to about 25 μg/mL, about 5 μg/mL to about 20 μg/mL, about 5 μg/mL about 15 μg/mL, about 5 μg/mL to about 10 μg/mL, about 10 μg/mL to about 50 μg/mL, about 10 μg/mL to about 45 μg/mL, about 10 μg/mL to about 40 μg/mL, about 10 μg/mL to about 35 μg/mL, about 10 μg/mL to about 30 μg/mL, about 10 μg/mL to about 25 μg/mL, about 10 μg/mL to about 20 μg/mL, about 10 μg/mL about 15 μg/mL, about 15 μg/mL to about 50 μg/mL, about 15 μg/mL to about 45 μg/mL, about 15 μg/mL to about 40 μg/mL, about 15 μg/mL to about 35 μg/mL, about 15 μg/mL to about 30 μg/mL, about 15 μg/mL to about 25 μg/mL, about 15 μg/mL to about 20 μg/mL, about 20 μg/mL to about 50 μg/mL, about 20 μg/mL to about 45 μg/mL, about 20 μg/mL to about 40 μg/mL, about 20 μg/mL to about 35 μg/mL, about 20 μg/mL to about 30 μg/mL, about 20 μg/mL to about 25 μg/mL, about 25 μg/mL to about 50 μg/mL, about 25 μg/mL to about 45 μg/mL, about 25 μg/mL to about 40 μg/mL, about 25 μg/mL to about 35 μg/mL, about 25 μg/mL to about 30 μg/mL, about 30 μg/mL to about 50 μg/mL, about 30 μg/mL to about 45 μg/mL, about 30 μg/mL to about 40 μg/mL, about 30 μg/mL to about 35 μg/mL, about 35 μg/mL to about 50 μg/mL, about 35 μg/mL to about 45 μg/mL, about 35 μg/mL to about 40 μg/mL, about 40 μg/mL to about 50 μg/mL, about 40 μg/mL to about 45 μg/mL, or about 45 μg/mL to about 50 μg/mL, of insulin (e.g., human insulin).

In some examples, the third culture medium can be, e.g., a chemically-defined liquid culture medium, an animal component-free liquid culture medium, or a chemically-defined animal component-free liquid culture medium, and/or a serum-free liquid culture medium.

### Culturing Step D

Perfusion Culturing

In some embodiments of any of the methods described herein, step (d) includes perfusion culturing the third cell culture (e.g., any of the third cell cultures described herein in any of the exemplary bioreactors (e.g., perfusion bioreactors) described herein having any of the exemplary volumes described herein) for about 5 days to about 20 days (e.g., about 5 days to about 19 days, about 5 days to about 18 days, about 5 days to about 17 days, about 5 days to about 16 days, about 5 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, about 5 days to about 6 days, about 6 days to about 20 days, about 6 days to about 19 days, about 6 days to about 18 days, about 6 days to about 17 days, about 6 days to about 16 days, about 6 days to about 15 days, about 6 days to about 14 days, about 6 days to about 13 days, about 6 days to about 12 days, about 6 days to about 11 days, about 6 days to about 10 days, about 6 days to about 9 days, about 6 days to about 8 days, about 6 days to about 7 days, about 7 days to about 20 days, about 7 days to about 19 days, about 7 days to about 18 days, about 7 days to about 17 days, about 7 days to about 16 days, about 7 days to about 15 days, about 7 days to about 14 days, about 7 days to about 13 days, about 7 days to about 12 days, about 7 days to about 11 days, about 7 days to about 10 days, about 7 days to about 9 days, about 7 days to about 8 days, about 8 days to about 20 days, about 8 days to about 19 days, about 8 days to about 18 days, about 8 days to about 17 days, about 8 days to about 16 days, about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 20 days, about 9 days to about 19 days, about 9 days to about 18 days, about 9 days to about 17 days, about 9 days to about 16 days, about 9 days to about 15 days, about 9 days to about 14 days, about 9 days to about 13 days, about 9 days to about 12 days, about 9 days to about 11 days, about 9 days to about 10 days, about 10 days to about 20 days, about 10 days to about 19 days, about 10 days to about 18 days, about 10 days to about 17 days, about 10 days to about 16 days, about 10 days to about 15 days, about 10 days to about 14 days, about 10 days to about 13 days, about 10 days to about 12 days, about 10 days to about 11 days, about 11 days to about 20 days, about 11 days to about 19 days, about 11 days to about 18 days, about 11 days to about 17 days, about 11 days to about 16 days, about 11 days to about 15 days, about 11 days to about 14 days, about 11 days to about 13 days, about 11 days to about 12 days, about 12 days to about 20 days, about 12 days to about 19 days, about 12 days to about 18 days, about 12 days to about 17 days, about 12 days to about 16 days, about 12 days to about 15 days, about 12 days to about 14 days, about 12 days to about 13 days, about 13 days to about 20 days, about 13 days to about 19 days, about 13 days to about 18 days, about 13 days to about 17 days, about 13 days to about 16 days, about 13 days to about 15 days, about 13 days to about 14 days, about 14 days to about 20 days, about 14 days to about 19 days, about 14 days to about 18 days, about 14 days to about 17 days, about 14 days to about 16 days, about 14 days to about 15 days, about 15 days to about 20 days, about 15 days to about 19 days, about 15 days to about 18 days, about 15 days to about 17 days, about 15 days to about 16 days, about 16 days to about 20 days, about 16 days to about 19 days, about 16 days to about 18 days, about 16 days to about 17 days, about 17 days to about 20 days, about 17 days to about 19 days, about 17 days to about 18 days, about 18 days to about 20 days, about 18 days to about 19 days, or about 19 days to about 20 days), wherein after step (d) the third culture medium includes a population of enucleated erythroid cells (e.g., any of the exemplary enucleated erythroid cells described herein). In some embodiments, step (d) includes agitating the third cell culture (e.g., in any of the bioreactors described herein having any of the exemplary volumes described herein) with a P/V value of about 10 W/m³ to about 200 W/m³ (e.g., any of the subranges of any of the ranges described herein).

In some embodiments, the perfusion culturing in step (d) can be performed using a perfusion rate of about 0.1 nL/cell/day to about 60 nL/cell/day, about 0.1 nL/cell/day to about 55 nL/cell/day, about 0.1 nL/cell/day to about 50 nL/cell/day, about 0.1 nL/cell/day to about 45 nL/cell/day, about 0.1 nL/cell/day to about 40 nL/cell/day, about 0.1 nL/cell/day to about 35 nL/cell/day, about 0.1 nL/cell/day to about 30 nL/cell/day, about 0.1 nL/cell/day to about 25 nL/cell/day, about 0.1 nL/cell/day to about 20 nL/cell/day, about 0.1 nL/cell/day to about 15 nL/cell/day, about 0.1 nL/cell/day to about 10 nL/cell/day, about 0.1 nL/cell/day to about 8 nL/cell/day, about 0.1 nL/cell/day to about 6 nL/cell/day, about 0.1 nL/cell/day to about 5 nL/cell/day, about 0.1 nL/cell/day to about 4 nL/cell/day, about 0.1 nL/cell/day to about 2 nL/cell/day, about 0.1 nL/cell/day to about 1 nL/cell/day, about 0.1 nL/cell/day to about 0.5 nL/cell/day, about 0.5 nL/cell/day to about 60 nL/cell/day, about 0.5 nL/cell/day to about 55 nL/cell/day, about 0.5 nL/cell/day to about 50 nL/cell/day, about 0.5 nL/cell/day to about 45 nL/cell/day, about 0.5 nL/cell/day to about 40 nL/cell/day, about 0.5 nL/cell/day to about 35 nL/cell/day, about 0.5 nL/cell/day to about 30 nL/cell/day, about 0.5 nL/cell/day to about 25 nL/cell/day, about 0.5 nL/cell/day to about 20 nL/cell/day, about 0.5 nL/cell/day to about 15 nL/cell/day, about 0.5 nL/cell/day to about 10 nL/cell/day, about 0.5 nL/cell/day to about 8 nL/cell/day, about 0.5 nL/cell/day to about 6 nL/cell/day, about 0.5 nL/cell/day to about 5 nL/cell/day, about 0.5 nL/cell/day to about 4 nL/cell/day, about 0.5 nL/cell/day to about 2 nL/cell/day, about 0.5 nL/cell/day to about 1 nL/cell/day, about 1 nL/cell/day to about 60 nL/cell/day, about 1 nL/cell/day to about 55 nL/cell/day, about 1 nL/cell/day to about 50 nL/cell/day, about 1 nL/cell/day to about 45 nL/cell/day, about 1 nL/cell/day to about 40 nL/cell/day, about 1 nL/cell/day to about 35 nL/cell/day, about 1 nL/cell/day to about 30 nL/cell/day, about 1 nL/cell/day to about 25 nL/cell/day, about 1 nL/cell/day to about 20 nL/cell/day, about 1 nL/cell/day to about 15 nL/cell/day, about 1 nL/cell/day to about 10 nL/cell/day, about 1 nL/cell/day to about 8 nL/cell/day, about 1 nL/cell/day to about 6 nL/cell/day, about 1 nL/cell/day to about 5 nL/cell/day, about 1 nL/cell/day to about 4 nL/cell/day, about 1 nL/cell/day to about 2 nL/cell/day, about 2 nL/cell/day to about 60 nL/cell/day, about 2 nL/cell/day to about 55 nL/cell/day, about 2 nL/cell/day to about 50 nL/cell/day, about 2 nL/cell/day to about 45 nL/cell/day, about 2 nL/cell/day to about 40 nL/cell/day, about 2 nL/cell/day to about 35 nL/cell/day, about 2 nL/cell/day to about 30 nL/cell/day, about 2 nL/cell/day to about 25 nL/cell/day, about 2 nL/cell/day to about 20 nL/cell/day, about 2 nL/cell/day to about 15 nL/cell/day, about 2 nL/cell/day to about 10 nL/cell/day, about 2 nL/cell/day to about 8 nL/cell/day, about 2 nL/cell/day to about 6 nL/cell/day, about 2 nL/cell/day to about 5 nL/cell/day, about 2 nL/cell/day to about 4 nL/cell/day, about 4 nL/cell/day to about 60 nL/cell/day, about 4 nL/cell/day to about 55 nL/cell/day, about 4 nL/cell/day to about 50 nL/cell/day, about 4 nL/cell/day to about 45 nL/cell/day, about 4 nL/cell/day to about 40 nL/cell/day, about 4 nL/cell/day to about 35 nL/cell/day, about 4 nL/cell/day to about 30 nL/cell/day, about 4 nL/cell/day to about 25 nL/cell/day, about 4 nL/cell/day to about 20 nL/cell/day, about 4 nL/cell/day to about 15 nL/cell/day, about 4 nL/cell/day to about 10 nL/cell/day, about 4 nL/cell/day to about 8 nL/cell/day, about 4 nL/cell/day to about 6 nL/cell/day, about 4 nL/cell/day to about 5 nL/cell/day, about 5 nL/cell/day to about 60 nL/cell/day, about 5 nL/cell/day to about 55 nL/cell/day, about 5 nL/cell/day to about 50 nL/cell/day, about 5 nL/cell/day to about 45 nL/cell/day, about 5 nL/cell/day to about 40 nL/cell/day, about 5 nL/cell/day to about 35 nL/cell/day, about 5 nL/cell/day to about 30 nL/cell/day, about 5 nL/cell/day to about 25 nL/cell/day, about 5 nL/cell/day to about 20 nL/cell/day, about 5 nL/cell/day to about 15 nL/cell/day, about 5 nL/cell/day to about 10 nL/cell/day, about 5 nL/cell/day to about 8 nL/cell/day, about 5 nL/cell/day to about 6 nL/cell/day, about 6 nL/cell/day to about 60 nL/cell/day, about 6 nL/cell/day to about 55 nL/cell/day, about 6 nL/cell/day to about 50 nL/cell/day, about 6 nL/cell/day to about 45 nL/cell/day, about 6 nL/cell/day to about 40 nL/cell/day, about 6 nL/cell/day to about 35 nL/cell/day, about 6 nL/cell/day to about 30 nL/cell/day, about 6 nL/cell/day to about 25 nL/cell/day, about 6 nL/cell/day to about 20 nL/cell/day, about 6 nL/cell/day to about 15 nL/cell/day, about 6 nL/cell/day to about 10 nL/cell/day, about 6 nL/cell/day to about 8 nL/cell/day, about 8 nL/cell/day to about 60 nL/cell/day, about 8 nL/cell/day to about 55 nL/cell/day, about 8 nL/cell/day to about 50 nL/cell/day, about 8 nL/cell/day to about 45 nL/cell/day, about 8 nL/cell/day to about 40 nL/cell/day, about 8 nL/cell/day to about 35 nL/cell/day, about 8 nL/cell/day to about 30 nL/cell/day, about 8 nL/cell/day to about 25 nL/cell/day, about 8 nL/cell/day to about 20 nL/cell/day, about 8 nL/cell/day to about 15 nL/cell/day, about 8 nL/cell/day to about 10 nL/cell/day, about 10 nL/cell/day to about 60 nL/cell/day, about 10 nL/cell/day to about 55 nL/cell/day, about 10 nL/cell/day to about 50 nL/cell/day, about 10 nL/cell/day to about 45 nL/cell/day, about 10 nL/cell/day to about 40 nL/cell/day, about 10 nL/cell/day to about 35 nL/cell/day, about 10 nL/cell/day to about 30 nL/cell/day, about 10 nL/cell/day to about 25 nL/cell/day, about 10 nL/cell/day to about 20 nL/cell/day, about 10 nL/cell/day to about 15 nL/cell/day, about 15 nL/cell/day to about 60 nL/cell/day, about 15 nL/cell/day to about 55 nL/cell/day, about 15 nL/cell/day to about 50 nL/cell/day, about 15 nL/cell/day to about 45 nL/cell/day, about 15 nL/cell/day to about 40 nL/cell/day, about 15 nL/cell/day to about 35 nL/cell/day, about 15 nL/cell/day to about 30 nL/cell/day, about 15 nL/cell/day to about 25 nL/cell/day, about 15 nL/cell/day to about 20 nL/cell/day, about 20 nL/cell/day to about 60 nL/cell/day, about 20 nL/cell/day to about 55 nL/cell/day, about 20 nL/cell/day to about 50 nL/cell/day, about 20 nL/cell/day to about 45 nL/cell/day, about 20 nL/cell/day to about 40 nL/cell/day, about 20 nL/cell/day to about 35 nL/cell/day, about 20 nL/cell/day to about 30 nL/cell/day, about 20 nL/cell/day to about 25 nL/cell/day, about 25 nL/cell/day to about 60 nL/cell/day, about 25 nL/cell/day to about 55 nL/cell/day, about 25 nL/cell/day to about 50 nL/cell/day, about 25 nL/cell/day to about 45 nL/cell/day, about 25 nL/cell/day to about 40 nL/cell/day, about 25 nL/cell/day to about 35 nL/cell/day, about 25 nL/cell/day to about 30 nL/cell/day, about 30 nL/cell/day to about 60 nL/cell/day, about 30 nL/cell/day to about 55 nL/cell/day, about 30 nL/cell/day to about 50 nL/cell/day, about 30 nL/cell/day to about 45 nL/cell/day, about 30 nL/cell/day to about 40 nL/cell/day, about 30 nL/cell/day to about 35 nL/cell/day, about 35 nL/cell/day to about 60 nL/cell/day, about 35 nL/cell/day to about 55 nL/cell/day, about 35 nL/cell/day to about 50 nL/cell/day, about 35 nL/cell/day to about 45 nL/cell/day, about 35 nL/cell/day to about 40 nL/cell/day, about 40 nL/cell/day to about 60 nL/cell/day, about 40 nL/cell/day to about 55 nL/cell/day, about 40 nL/cell/day to about 50 nL/cell/day, about 40 nL/cell/day to about 45 nL/cell/day, about 45 nL/cell/day to about 60 nL/cell/day, about 45 nL/cell/day to about 55 nL/cell/day, about 45 nL/cell/day to about 50 nL/cell/day, about 50 nL/cell/day to about 60 nL/cell/day, about 50 nL/cell/day to about 55 nL/cell/day, or about 55 nL/cell/day to about 60 nL/cell/day. In some embodiments, the perfusion rate is increased over time.

In some embodiments, the perfusion culturing in step (d) can be performed using a perfusion rate of about 0.1 VVD to about 3 VVD (or any of the subranges of this range described herein).

In some examples, the perfusion culturing in step (d) includes: (i) adding an additional volume of the a third culture medium (e.g., any of the exemplary third culture media described herein) to the third cell culture (e.g., any of the exemplary third cell cultures described herein) for a first period of time; and (ii) adding an additional volume of a fourth culture medium (e.g., any of the exemplary fourth culture media described herein) to the third cell culture (e.g., any of the exemplary third cell cultures described herein) for a second period of time.

In some examples, the additional volume of the third culture medium (e.g., any of the exemplary third culture media described herein) in (i) is added continuously to the third cell culture (e.g., any of the exemplary third cell cultures described herein) for the first period of time; and/or the additional volume of the fourth culture medium (e.g., any of the exemplary fourth culture media described herein) in (ii) is added continuously to the third cell culture (e.g., any of the exemplary third cell cultures described herein) for the second period of time.

In some examples, the additional volume of the third culture medium (e.g., any of the exemplary third culture media described herein) in (i) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the third cell culture (e.g., any of the exemplary third cell cultures described herein) for the first period of time; and/or (ii) the additional volume of the fourth culture medium (e.g., any of the exemplary fourth culture media described herein) in (ii) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the third cell culture (e.g., any of the exemplary third cell cultures described herein) for the second period of time.

The addition of culture medium (e.g., any of the exemplary third or fourth culture media described herein) can be performed mechanically, e.g., using a peristaltic pump, or manually (e.g., by sterile pipetting).

The removal of culture medium (e.g., substantially cell-free culture medium) can be performed mechanically, e.g., using a tangential flow filtration (TFF) or alternating flow filtration (ATF), or manually (e.g., by sterile pipetting). Additional non-limiting aspects of tangential flow filtration are described herein. In some examples, the first period of time in (i) is about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 day to about 12 days, about 2 day to about 11 days, about 2 day to about 10 days, about 2 day to about 9 days, about 2 day to about 8 days, about 2 day to about 7 days, about 2 day to about 6 days, about 2 day to about 5 days, about 2 day to about 4 days, about 2 day to about 3 days, about 3 day to about 12 days, about 3 day to about 11 days, about 3 day to about 10 days, about 3 day to about 9 days, about 3 day to about 8 days, about 3 day to about 7 days, about 3 day to about 6 days, about 3 day to about 5 days, about 3 day to about 4 days, about 4 day to about 12 days, about 4 day to about 11 days, about 4 day to about 10 days, about 4 day to about 9 days, about 4 day to about 8 days, about 4 day to about 7 days, about 4 day to about 6 days, about 4 day to about 5 days, about 5 day to about 12 days, about 5 day to about 11 days, about 5 day to about 10 days, about 5 day to about 9 days, about 5 day to about 8 days, about 5 day to about 7 days, about 5 day to about 6 days, about 6 day to about 12 days, about 6 day to about 11 days, about 6 day to about 10 days, about 6 day to about 9 days, about 6 day to about 8 days, about 6 day to about 7 days, about 7 day to about 12 days, about 7 day to about 11 days, about 7 day to about 10 days, about 7 day to about 9 days, about 7 day to about 8 days, about 8 day to about 12 days, about 8 day to about 11 days, about 8 day to about 10 days, about 8 day to about 9 days, about 9 day to about 12 days, about 9 day to about 11 days, about 9 day to about 10 days, about 10 day to about 12 days, about 10 day to about 11 days, or about 11 day to about 12 days.

In some examples, the second period of time in (ii) is about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 day to about 12 days, about 2 day to about 11 days, about 2 day to about 10 days, about 2 day to about 9 days, about 2 day to about 8 days, about 2 day to about 7 days, about 2 day to about 6 days, about 2 day to about 5 days, about 2 day to about 4 days, about 2 day to about 3 days, about 3 day to about 12 days, about 3 day to about 11 days, about 3 day to about 10 days, about 3 day to about 9 days, about 3 day to about 8 days, about 3 day to about 7 days, about 3 day to about 6 days, about 3 day to about 5 days, about 3 day to about 4 days, about 4 day to about 12 days, about 4 day to about 11 days, about 4 day to about 10 days, about 4 day to about 9 days, about 4 day to about 8 days, about 4 day to about 7 days, about 4 day to about 6 days, about 4 day to about 5 days, about 5 day to about 12 days, about 5 day to about 11 days, about 5 day to about 10 days, about 5 day to about 9 days, about 5 day to about 8 days, about 5 day to about 7 days, about 5 day to about 6 days, about 6 day to about 12 days, about 6 day to about 11 days, about 6 day to about 10 days, about 6 day to about 9 days, about 6 day to about 8 days, about 6 day to about 7 days, about 7 day to about 12 days, about 7 day to about 11 days, about 7 day to about 10 days, about 7 day to about 9 days, about 7 day to about 8 days, about 8 day to about 12 days, about 8 day to about 11 days, about 8 day to about 10 days, about 8 day to about 9 days, about 9 day to about 12 days, about 9 day to about 11 days, about 9 day to about 10 days, about 10 day to about 12 days, about 10 day to about 11 days, or about 11 day to about 12 days.

In some examples of these methods, the fourth culture medium includes one or more (e.g., one, two, or three) of: transferrin (e.g., about 100 μg/mL to about 2 mg/mL transferrin (e.g., human apotransferrin, human holo transferrin, or a combination thereof) or any of the subranges of this range described herein), erythropoietin (EPO) or an EPO-mimetic peptide (e.g., about 1 ng/mL to about 500 ng/mL of EPO (e.g., recombinant human EPO) or an EPO-mimetic peptide or any of the subranges of this range described herein, and insulin (e.g., about 0.1 μg/mL to about 50 μg/mL insulin or any of the subranges of this range described herein).

In some embodiments of any of the fourth culture media described herein, the fourth culture medium includes Iscove's modified Dulbecco's medium (IMDM). In some embodiments of any of the fourth culture media described herein, the fourth culture medium includes about 0.1% v/v to about 10% v/v (e.g., about 0.1% v/v to about 9.5% v/v, about 0.1% v/v to about 9.0% v/v, about 0.1% v/v to about 8.5% v/v, about 0.1% v/v to about 8.0% v/v, about 0.1% v/v to about 7.5% v/v, about 0.1% v/v to about 7.0% v/v, about 0.1% v/v to about 6.5% v/v, about 0.1% v/v to about 6.0% v/v, about 0.1% v/v to about 5.5% v/v, about 0.1% v/v to about 5.0% v/v, about 0.1% v/v to about 4.5% v/v, about 0.1% v/v to about 4.0% v/v, about 0.1% v/v to about 3.5% v/v, about 0.1% v/v to about 3.0% v/v, about 0.1% v/v to about 2.5% v/v, about 0.1% v/v to about 2.0% v/v, about 0.1% v/v to about 1.5% v/v, about 0.1% v/v to about 1.0% v/v, about 0.5% v/v to about 1.0% v/v, about 0.5% v/v to about 10% v/v, about 0.5% v/v to about 9.5% v/v, about 0.5% v/v to about 9.0% v/v, about 0.5% v/v to about 8.5% v/v, about 0.5% v/v to about 8.0% v/v, about 0.5% v/v to about 7.5% v/v, about 0.5% v/v to about 7.0% v/v, about 0.5% v/v to about 6.5% v/v, about 0.5% v/v to about 6.0% v/v, about 0.5% v/v to about 5.5% v/v, about 0.5% v/v to about 5% v/v, about 0.5% v/v to about 4.5% v/v, about 0.5% v/v to about 4.0% v/v, about 0.5% v/v to about 3.5% v/v, about 0.5% v/v to about 3.0% v/v, about 0.5% v/v to about 2.5% v/v, about 0.5% v/v to about 2.0% v/v, about 0.5% v/v to about 1.5% v/v, about 0.5% v/v to about 1.0% v/v, about 1.0% v/v to about 10% v/v, about 1.0% v/v to about 9.5% v/v, about 1.0% v/v to about 9.0% v/v, about 1.0% v/v to about 8.5% v/v, about 1.0% v/v to about 8.0% v/v, about 1.0% v/v to about 7.5% v/v, about 1.0% v/v to about 7.0% v/v, about 1.0% v/v to about 6.5% v/v, about 1.0% v/v to about 6.0% v/v, about 1.0% v/v to about 5.5% v/v, about 1.0% v/v to about 5% v/v, about 1.0% v/v to about 4.5% v/v, about 1.0% v/v to about 4.0% v/v, about 1.0% v/v to about 3.5% v/v, about 1.0% v/v to about 3.0% v/v, about 1.0% v/v to about 2.5% v/v, about 1.0% v/v to about 2.0% v/v, about 1.0% v/v to about 1.5% v/v, about 1.5% v/v to about 10% v/v, about 1.5% v/v to about 9.5% v/v, about 1.5% v/v to about 9.0% v/v, about 1.5% v/v to about 8.5% v/v, about 1.5% v/v to about 8.0% v/v, about 1.5% v/v to about 7.5% v/v, about 1.5% v/v to about 7.0% v/v, about 1.5% v/v to about 6.5% v/v, about 1.5% v/v to about 6.0% v/v, about 1.5% v/v to about 5.5% v/v, about 1.5% v/v to about 5% v/v, about 1.5% v/v to about 4.5% v/v, about 1.5% v/v to about 4.0% v/v, about 1.5% v/v to about 3.5% v/v, about 1.5% v/v to about 3.0% v/v, about 1.5% v/v to about 2.5% v/v, about 1.5% v/v to about 2.0% v/v, about 2.0% v/v to about 10% v/v, about 2.0% v/v to about 9.5% v/v, about 2.0% v/v to about 9.0% v/v, about 2.0% v/v to about 8.5% v/v, about 2.0% v/v to about 8.000% v/v, about 2.0% v/v to about 7.5% v/v, about 2.0% v/v to about 7.0% v/v, about 2.0% v/v to about 6.5% v/v, about 2.0% v/v to about 6.0% v/v, about 2.0% v/v to about 5.5% v/v, about 2.0% v/v to about 5% v/v, about 2.0% v/v to about 4.5% v/v, about 2.0% v/v to about 4.0% v/v, about 2.0% v/v to about 3.5% v/v, about 2.0% v/v to about 3.0% v/v, about 2.0% v/v to about 2.5% v/v, about 2.5% v/v to about 10% v/v, about 2.5% v/v to about 9.5% v/v, about 2.5% v/v to about 9.0% v/v, about 2.5% v/v to about 8.5% v/v, about 2.5% v/v to about 8.0% v/v, about 2.5% v/v to about 7.5% v/v, about 2.5% v/v to about 7.0% v/v, about 2.5% v/v to about 6.5% v/v, about 2.5% v/v to about 6.0% v/v, about 2.5% v/v to about 5.5% v/v, about 2.5% v/v to about 5% v/v, about 2.5% v/v to about 4.5% v/v, about 2.5% v/v to about 4.0% v/v, about 2.5% v/v to about 3.5% v/v, about 2.5% v/v to about 3.0% v/v, about 3.0% v/v to about 10% v/v, about 3.0% v/v to about 9.5% v/v, about 3.0% v/v to about 9.0% v/v, about 3.0% v/v to about 8.5% v/v, about 3.0% v/v to about 8.0% v/v, about 3.0% v/v to about 7.5% v/v, about 3.0% v/v to about 7.0% v/v, about 3.0% v/v to about 6.5% v/v, about 3.0% v/v to about 6.0% v/v, about 3.0% v/v to about 5.5% v/v, about 3.0% v/v to about 5% v/v, about 3.0% v/v to about 4.5% v/v, about 3.0% v/v to about 4.0% v/v, about 3.0% v/v to about 3.5% v/v, about 3.5% v/v to about 10% v/v, about 3.5% v/v to about 9.5% v/v, about 3.5% v/v to about 9.0% v/v, about 3.5% v/v to about 8.5% v/v, about 3.5% v/v to about 8.0% v/v, about 3.5% v/v to about 7.5% v/v, about 3.5% v/v to about 7.0% v/v, about 3.5% v/v to about 6.5% v/v, about 3.5% v/v to about 6.0% v/v, about 3.5% v/v to about 5.5% v/v, about 3.5% v/v to about 5% v/v, about 3.5% v/v to about 4.5% v/v, about 3.5% v/v to about 4.0% v/v, about 4.0% v/v to about 10% v/v, about 4.0% v/v to about 9.5% v/v, about 4.0% v/v to about 9.0% v/v, about 4.0% v/v to about 8.5% v/v, about 4.0% v/v to about 8.0% v/v, about 4.0% v/v to about 7.5% v/v, about 4.0% v/v to about 7.0% v/v, about 4.0% v/v to about 6.5% v/v, about 4.0% v/v to about 6.0% v/v, about 4.0% v/v to about 5.5% v/v, about 4.0% v/v to about 5% v/v, about 4.0% v/v to about 4.5% v/v, about 4.5% v/v to about 10% v/v, about 4.5% v/v to about 9.5% v/v, about 4.5% v/v to about 9.0% v/v, about 4.5% v/v to about 8.5% v/v, about 4.5% v/v to about 8.0% v/v, about 4.5% v/v to about 7.5% v/v, about 4.5% v/v to about 7.0% v/v, about 4.5% v/v to about 6.5% v/v, about 4.5% v/v to about 6.0% v/v, about 4.5% v/v to about 5.5% v/v, about 4.5% v/v to about 5.0% v/v, about 5.0% v/v to about 10% v/v, about 5.0% v/v to about 9.5% v/v, about 5.0% v/v to about 9.0% v/v, about 5.0% v/v to about 8.5% v/v, about 5.0% v/v to about 8.0% v/v, about 5.0% v/v to about 7.5% v/v, about 5.0% v/v to about 7.0% v/v, about 5.0% v/v to about 6.5% v/v, about 5.0% v/v to about 6.0% v/v, about 5.0% v/v to about 5.5% v/v, about 5.5% v/v to about 10% v/v, about 5.5% v/v to about 9.5% v/v, about 5.5% v/v to about 9.0% v/v, about 5.5% v/v to about 8.5% v/v, about 5.5% v/v to about 8.0% v/v, about 5.5% v/v to about 7.5% v/v, about 5.5% v/v to about 7.0% v/v, about 5.5% v/v to about 6.5% v/v, about 5.5% v/v to about 6.0% v/v, about 6.0% v/v to about 10% v/v, about 6.0% v/v to about 9.5% v/v, about 6.0% v/v to about 9.0% v/v, about 6.0% v/v to about 8.5% v/v, about 6.0% v/v to about 8.0% v/v, about 6.0% v/v to about 7.5% v/v, about 6.0% v/v to about 7.0% v/v, about 6.0% v/v to about 6.5% v/v, about 6.5% v/v to about 10% v/v, about 6.5% v/v to about 9.5% v/v, about 6.5% v/v to about 9.0% v/v, about 6.5% v/v to about 8.5% v/v, about 6.5% v/v to about 8.0% v/v, about 6.5% v/v to about 7.5% v/v, about 6.5% v/v to about 7.0% v/v, about 7.0% v/v to about 10% v/v, about 7.0% v/v to about 9.5% v/v, about 7.0% v/v to about 9.0% v/v, about 7.0% v/v to about 8.5% v/v, about 7.0% v/v to about 8.0% v/v, about 7.0% v/v to about 7.5% v/v, about 7.5% v/v to about 10% v/v, about 7.5% v/v to about 9.5% v/v, about 7.5% v/v to about 9.0% v/v, about 7.5% v/v to about 8.5% v/v, about 7.5% v/v to about 8.0% v/v, about 8.0% v/v to about 10% v/v, about 8.0% v/v to about 9.5% v/v, about 8.0% v/v to about 9.0% v/v, about 8.0% v/v to about 8.5% v/v, about 8.5% v/v to about 10% v/v, about 8.5% v/v to about 9.5% v/v, about 8.5% v/v to about 9.0% v/v, about 9.0% v/v to about 10% v/v, about 9.0% v/v to about 9.5% v/v, or about 9.5% v/v to about 10% v/v) serum (e.g., human AB serum). In some embodiments, the fourth culture medium includes 0.1% v/v to about 5% v/v (e.g., about 0.1% v/v to about 4.5% v/v, about 0.1% v/v to about 4.0% v/v, about 0.1% v/v to about 3.5% v/v, about 0.1% v/v to about 3.0% v/v, about 0.1% v/v to about 2.5% v/v, about 0.1% v/v to about 2.0% v/v, about 0.1% v/v to about 1.5% v/v, about 0.1% v/v to about 1.0% v/v, about 0.1% v/v to about 0.5% v/v, about 0.5% v/v to about 5% v/v, about 0.5% v/v to about 4.5% v/v, about 0.5% v/v to about 4.0% v/v, about 0.5% v/v to about 3.5% v/v, about 0.5% v/v to about 3.0% v/v, about 0.5% v/v to about 2.5% v/v, about 0.5% v/v to about 2.0% v/v, about 0.5% v/v to about 1.5% v/v, about 0.5% v/v to about 1.0% v/v, about 1.0% v/v to about 5% v/v, about 1.0% v/v to about 4.5% v/v, about 1.0% v/v to about 4.0% v/v, about 1.0% v/v to about 3.5% v/v, about 1.0% v/v to about 3.0% v/v, about 1.0% v/v to about 2.5% v/v, about 1.0% v/v to about 2.0% v/v, about 1.0% v/v to about 1.5% v/v, about 1.5% v/v to about 5% v/v, about 1.5% v/v to about 4.5% v/v, about 1.5% v/v to about 4.0% v/v, about 1.5% v/v to about 3.5% v/v, about 1.5% v/v to about 3.0% v/v, about 1.5% v/v to about 2.5% v/v, about 1.5% v/v to about 2.0% v/v, about 2.0% v/v to about 5% v/v, about 2.0% v/v to about 4.5% v/v, about 2.0% v/v to about 4.0% v/v, about 2.0% v/v to about 3.5% v/v, about 2.0% v/v to about 3.0% v/v, about 2.0% v/v to about 2.5% v/v, about 2.5% v/v to about 5% v/v, about 2.5% v/v to about 4.5% v/v, about 2.5% v/v to about 4.0% v/v, about 2.5% v/v to about 3.5% v/v, about 2.5% v/v to about 3.0% v/v, about 3.0% v/v to about 5% v/v, about 3.0% v/v to about 4.5% v/v, about 3.0% v/v to about 4.0% v/v, about 3.0% v/v to about 3.5% v/v, about 3.5% v/v to about 5% v/v, about 3.5% v/v to about 4.5% v/v, about 3.5% v/v to about 4.0% v/v, about 4.0% v/v to about 5% v/v, about 4.0% v/v to about 4.5% v/v, or about 4.5% v/v to about 5% v/v) human plasma. In some embodiments, the fourth culture medium includes about 1 U/mL to about 5 U/mL (e.g., about 1 U/mL to about 4 U/mL, about 1 U/mL to about 3 U/mL, about 1 U/mL to about 2 U/mL, about 2 U/mL to about 5 U/mL, about 2 U/mL to about 4 U/mL, about 2 U/mL to about 3 U/mL, about 3 U/mL to about 5 U/mL, about 3 U/mL to about 4 U/mL, or about 4 U/mL to about 5 U/mL) heparin. In some examples, the fourth culture medium includes about 0.1% w/v to about 3% w/v (e.g., about 0.1% w/v to about 2.5% w/v, about 0.1% w/v to about 2.0% w/v, about 0.1% w/v to about 1.5% w/v, about 0.1% w/v to about 1.0% w/v, about 0.1% w/v to about 0.5% w/v, about 0.5% w/v to about 3% w/v, about 0.5% w/v to about 2.5% w/v, about 0.5% w/v to about 2.0% w/v, about 0.5% w/v to about 1.5% w/v, about 0.5% w/v to about 1.0% w/v, about 1.0% w/v to about 3% w/v, about 1.0% w/v to about 2.5% w/v, about 1.0% w/v to about 2.0% w/v, about 1.0% w/v to about 1.5% w/v, about 1.5% w/v to about 3% w/v, about 1.5% w/v to about 2.5% w/v, about 1.5% w/v to about 2.0% w/v, about 2.0% w/v to about 3% w/v, about 2.0% w/v to about 2.5% w/v, or about 2.5% w/v to about 3% w/v) Poloxamer-188 (P188). In some embodiments, the fourth culture medium includes about 1 mM to about 8 mM (e.g. any of the subranges of this range described herein) of L-glutamine, L-alanyl-L-glutamine, L-glycyl-L-glutamine, N-acetyl-L-glutamine, or a combination thereof.

In some examples, the fourth culture medium includes about 100 μg/mL to about 2 mg/mL, about 100 μg/mL to about 1.8 mg/mL, about 100 μg/mL to about 1.6 mg/mL, about 100 μg/mL to about 1.5 mg/mL, about 100 μg/mL to about 1.4 mg/mL, about 100 μg/mL to about 1.3 mg/mL, about100 μg/mL to about 1.2 mg/mL, about 100 μg/mL to about 1.1 mg/mL, about 100 μg/mL to about 1.0 mg/mL, about 100 μg/mL to about 900 μg/mL, about 100 μg/mL to about 800 μg/mL, about 100 μg/mL to about 700 μg/mL, about 100 μg/mL to about 600 μg/mL, about 100 μg/mL to about 500 μg/mL, about 100 μg/mL to about 400 μg/mL, about 100 μg/mL to about 300 μg/mL, about 100 μg/mL to about 200 μg/mL, about 200 μg/mL to about 2 mg/mL, about 200 μg/mL to about 1.8 mg/mL, about 200 μg/mL to about 1.6 mg/mL, about 200 μg/mL to about 1.5 mg/mL, about 200 μg/mL to about 1.4 mg/mL, about 200 μg/mL to about 1.3 mg/mL, about 200 μg/mL to about 1.2 mg/mL, about 200 μg/mL to about 1.1 mg/mL, about 200 μg/mL to about 1.0 mg/mL, about 200 μg/mL to about 900 μg/mL, about 200 μg/mL to about 800 μg/mL, about 200 μg/mL to about 700 μg/mL, about 200 μg/mL to about 600 μg/mL, about 200 μg/mL to about 500 μg/mL, about 200 μg/mL to about 400 μg/mL, about 200 μg/mL to about 300 μg/mL, about 300 μg/mL to about 2 mg/mL, about 300 μg/mL to about 1.8 mg/mL, about 300 μg/mL to about 1.6 mg/mL, about 300 μg/mL to about 1.5 mg/mL, about 300 μg/mL to about 1.4 mg/mL, about 300 μg/mL to about 1.3 mg/mL, about 300 μg/mL to about 1.2 mg/mL, about 300 μg/mL to about 1.1 mg/mL, about 300 μg/mL to about 1.0 mg/mL, about 300 μg/mL to about 900 μg/mL, about 300 μg/mL to about 800 μg/mL, about 300 μg/mL to about 700 μg/mL, about 300 μg/mL to about 600 μg/mL, about 300 μg/mL to about 500 μg/mL, about 300 μg/mL to about 400 μg/mL, about 400 μg/mL to about 2 mg/mL, about 400 μg/mL to about 1.8 mg/mL, about 400 μg/mL to about 1.6 mg/mL, about 400 μg/mL to about 1.5 mg/mL, about 400 μg/mL to about 1.4 mg/mL, about 400 μg/mL to about 1.3 mg/mL, about 400 μg/mL to about 1.2 mg/mL, about 400 μg/mL to about 1.1 mg/mL, about 400 μg/mL to about 1.0 mg/mL, about 400 μg/mL to about 900 μg/mL, about 400 μg/mL to about 800 μg/mL, about 400 μg/mL to about 700 μg/mL, about 400 μg/mL to about 600 μg/mL, about 400 μg/mL to about 500 μg/mL, about 500 μg/mL to about 2 mg/mL, about 500 μg/mL to about 1.8 mg/mL, about 500 μg/mL to about 1.6 mg/mL, 500 μg/mL to about 1.5 mg/mL, about 500 μg/mL to about 1.4 mg/mL, about 500 μg/mL to about 1.3 mg/mL, about 500 μg/mL to about 1.2 mg/mL, about 500 μg/mL to about 1.1 mg/mL, about 500 μg/mL to about 1.0 mg/mL, about 500 μg/mL to about 900 μg/mL, about 500 μg/mL to about 800 μg/mL, about 500 μg/mL to about 700 μg/mL, about 500 μg/mL to about 600 μg/mL, about 600 μg/mL to about 2 mg/mL, about 600 μg/mL to about 1.8 mg/mL, about 600 μg/mL to about 1.6 mg/mL, about 600 μg/mL to about 1.5 mg/mL, about 600 μg/mL to about 1.4 mg/mL, about 600 μg/mL to about 1.3 mg/mL, about 600 μg/mL to about 1.2 mg/mL, about 600 μg/mL to about 1.1 mg/mL, about 600 μg/mL to about 1.0 mg/mL, about 600 μg/mL to about 900 μg/mL, about 600 μg/mL to about 800 μg/mL, about 600 μg/mL to about 700 μg/mL, about 700 μg/mL to about 2 mg/mL, about 700 μg/mL to about 1.8 mg/mL, about 700 μg/mL to about 1.6 mg/mL, about 700 μg/mL to about 1.5 mg/mL, about 700 μg/mL to about 1.4 mg/mL, about 700 μg/mL to about 1.3 mg/mL, about 700 μg/mL to about 1.2 mg/mL, about 700 μg/mL to about 1.1 mg/mL, about 700 μg/mL to about 1.0 mg/mL, about 700 μg/mL to about 900 μg/mL, about 700 μg/mL to about 800 μg/mL, about 800 μg/mL to about 2 mg/mL, about 800 μg/mL to about 1.8 mg/mL, about 800 μg/mL to about 1.6 mg/mL, about 800 μg/mL to about 1.5 mg/mL, about 800 μg/mL to about 1.4 mg/mL, about 800 μg/mL to about 1.3 mg/mL, about 800 μg/mL to about 1.2 mg/mL, about 800 μg/mL to about 1.1 mg/mL, about 800 μg/mL to about 1.0 mg/mL, about 800 μg/mL to about 900 μg/mL, about 900 μg/mL to about 2 mg/mL, about 900 μg/mL to about 1.8 mg/mL, about 900 μg/mL to about 1.6 mg/mL, about 900 μg/mL to about 1.5 mg/mL, about 900 μg/mL to about 1.4 mg/mL, about 900 μg/mL to about 1.3 mg/mL, about 900 μg/mL to about 1.2 mg/mL, about 900 μg/mL to about 1.1 mg/mL, about 900 μg/mL to about 1.0 mg/mL, about 1.0 mg/mL to about 2 mg/mL, about 1.0 mg/mL to about 1.8 mg/mL, about 1.0 mg/mL to about 1.6 mg/mL, about 1.0 mg/mL to about 1.5 mg/mL, about 1.0 mg/mL to about 1.4 mg/mL, about 1.0 mg/mL to about 1.3 mg/mL, about 1.0 mg/mL to about 1.2 mg/mL, about 1.0 mg/mL to about 1.1 mg/mL, about 1.1 mg/mL to about 2 mg/mL, about 1.1 mg/mL to about 1.8 mg/mL, about 1.1 mg/mL to about 1.6 mg/mL, about 1.1 mg/mL to about 1.5 mg/mL, about 1.1 mg/mL to about 1.4 mg/mL, about 1.1 mg/mL to about 1.3 mg/mL, about 1.1 mg/mL to about 1.2 mg/mL, about 1.2 mg/mL to about 2 mg/mL, about 1.2 mg/mL to about 1.8 mg/mL, about 1.2 mg/mL to about 1.6 mg/mL, about 1.2 mg/mL to about 1.5 mg/mL, about 1.2 mg/mL to about 1.4 mg/mL, about 1.2 mg/mL to about 1.3 mg/mL, about 1.3 mg/mL to about 2 mg/mL, about 1.3 mg/mL to about 1.8 mg/mL, about 1.3 mg/mL to about 1.6 mg/mL, about 1.3 mg/mL to about 1.5 mg/mL, about 1.3 mg/mL to about 1.4 mg/mL, about 1.4 mg/mL to about 2 mg/mL, about 1.4 mg/mL to about 1.8 mg/mL, about 1.4 mg/mL to about 1.6 mg/mL, about 1.4 mg/mL to about 1.5 mg/mL, about 1.5 mg/mL to about 2 mg/mL, about 1.5 mg/mL to about 1.8 mg/mL, about 1.5 mg/mL to about 1.6 mg/mL, about 1.6 mg/mL to about 2 mg/mL, about 1.6 mg/mL to about 1.8 mg/mL, or about 1.8 mg/mL to about 2 mg/mL of transferrin (e.g., apotransferrin, holo transferrin, or a combination thereof).

In some examples, the fourth culture medium includes about 1 ng/mL to about 500 ng/mL, about 1 ng/mL to about 480 ng/mL, about 1 ng/mL to about 460 ng/mL, about 1 ng/mL to about 440 ng/mL, about 1 ng/mL to about 420 ng/mL, about 1 ng/mL to about 400 ng/mL, about 1 ng/mL to about 380 ng/mL, about 1 ng/mL to about 360 ng/mL, about 1 ng/mL to about 340 ng/mL, about 1 ng/mL to about 320 ng/mL, about 1 ng/mL to about 300 ng/mL, about 1 ng/mL to about 280 ng/mL, about 1 ng/mL to about 260 ng/mL, about 1 ng/mL to about 240 ng/mL, about 1 ng/mL to about 220 ng/mL, about 1 ng/mL to about 200 ng/mL, about 1 ng/mL to about 180 ng/mL, about 1 ng/mL to about 160 ng/mL, about 1 ng/mL to about 140 ng/mL, about 1 ng/mL to about 120 ng/mL, about 1 ng/mL to about 100 ng/mL, about 1 ng/mL to about 80 ng/mL, about 1 ng/mL to about 60 ng/mL, about 1 ng/mL to about 40 ng/mL, about 1 ng/mL to about 30 ng/mL, about 1 ng/mL to about 25 ng/mL, about 1 ng/mL to about 20 ng/mL, about 1 ng/mL to about 15 ng/mL, about 1 ng/mL to about 10 ng/mL, about 1 ng/mL to about 5 ng/mL, 5 ng/mL to about 500 ng/mL, about 5 ng/mL to about 480 ng/mL, about 5 ng/mL to about 460 ng/mL, about 5 ng/mL to about 440 ng/mL, about 5 ng/mL to about 420 ng/mL, about 5 ng/mL to about 400 ng/mL, about 5 ng/mL to about 380 ng/mL, about 5 ng/mL to about 360 ng/mL, about 5 ng/mL to about 340 ng/mL, about 5 ng/mL to about 320 ng/mL, about 5 ng/mL to about 300 ng/mL, about 5 ng/mL to about 280 ng/mL, about 5 ng/mL to about 260 ng/mL, about 5 ng/mL to about 240 ng/mL, about 5 ng/mL to about 220 ng/mL, about 5 ng/mL to about 200 ng/mL, about 5 ng/mL to about 180 ng/mL, about 5 ng/mL to about 160 ng/mL, about 5 ng/mL to about 140 ng/mL, about 5 ng/mL to about 120 ng/mL, about 5 ng/mL to about 100 ng/mL, about 5 ng/mL to about 80 ng/mL, about 5 ng/mL to about 60 ng/mL, about 5 ng/mL to about 40 ng/mL, about 5 ng/mL to about 30 ng/mL, about 5 ng/mL to about 25 ng/mL, about 5 ng/mL to about 20 ng/mL, about 5 ng/mL to about 15 ng/mL, about 5 ng/mL to about 10 ng/mL, 10 ng/mL to about 500 ng/mL, about 10 ng/mL to about 480 ng/mL, about 10 ng/mL to about 460 ng/mL, about 10 ng/mL to about 440 ng/mL, about 10 ng/mL to about 420 ng/mL, about 10 ng/mL to about 400 ng/mL, about 10 ng/mL to about 380 ng/mL, about 10 ng/mL to about 360 ng/mL, about 10 ng/mL to about 340 ng/mL, about 10 ng/mL to about 320 ng/mL, about 10 ng/mL to about 300 ng/mL, about 10 ng/mL to about 280 ng/mL, about 10 ng/mL to about 260 ng/mL, about 10 ng/mL to about 240 ng/mL, about 10 ng/mL to about 220 ng/mL, about 10 ng/mL to about 200 ng/mL, about 10 ng/mL to about 180 ng/mL, about 10 ng/mL to about 160 ng/mL, about 10 ng/mL to about 140 ng/mL, about 10 ng/mL to about 120 ng/mL, about 10 ng/mL to about 100 ng/mL, about 10 ng/mL to about 80 ng/mL, about 10 ng/mL to about 60 ng/mL, about 10 ng/mL to about 40 ng/mL, about 10 ng/mL to about 30 ng/mL, about 10 ng/mL to about 25 ng/mL, about 10 ng/mL to about 20 ng/mL, about 10 ng/mL to about 15 ng/mL, 15 ng/mL to about 500 ng/mL, about 15 ng/mL to about 480 ng/mL, about 15 ng/mL to about 460 ng/mL, about 15 ng/mL to about 440 ng/mL, about 15 ng/mL to about 420 ng/mL, about 15 ng/mL to about 400 ng/mL, about 15 ng/mL to about 380 ng/mL, about 15 ng/mL to about 360 ng/mL, about 15 ng/mL to about 340 ng/mL, about 15 ng/mL to about 320 ng/mL, about 15 ng/mL to about 300 ng/mL, about 15 ng/mL to about 280 ng/mL, about 15 ng/mL to about 260 ng/mL, about 15 ng/mL to about 240 ng/mL, about 15 ng/mL to about 220 ng/mL, about 15 ng/mL to about 200 ng/mL, about 15 ng/mL to about 180 ng/mL, about 15 ng/mL to about 160 ng/mL, about 15 ng/mL to about 140 ng/mL, about 15 ng/mL to about 120 ng/mL, about 15 ng/mL to about 100 ng/mL, about 15 ng/mL to about 80 ng/mL, about 15 ng/mL to about 60 ng/mL, about 15 ng/mL to about 40 ng/mL, about 15 ng/mL to about 30 ng/mL, about 15 ng/mL to about 25 ng/mL, about 15 ng/mL to about 20 ng/mL, 20 ng/mL to about 500 ng/mL, about 20 ng/mL to about 480 ng/mL, about 20 ng/mL to about 460 ng/mL, about 20 ng/mL to about 440 ng/mL, about 20 ng/mL to about 420 ng/mL, about 20 ng/mL to about 400 ng/mL, about 20 ng/mL to about 380 ng/mL, about 20 ng/mL to about 360 ng/mL, about 20 ng/mL to about 340 ng/mL, about 20 ng/mL to about 320 ng/mL, about 20 ng/mL to about 300 ng/mL, about 20 ng/mL to about 280 ng/mL, about 20 ng/mL to about 260 ng/mL, about 20 ng/mL to about 240 ng/mL, about 20 ng/mL to about 220 ng/mL, about 20 ng/mL to about 200 ng/mL, about 20 ng/mL to about 180 ng/mL, about 20 ng/mL to about 160 ng/mL, about 20 ng/mL to about 140 ng/mL, about 20 ng/mL to about 120 ng/mL, about 20 ng/mL to about 100 ng/mL, about 20 ng/mL to about 80 ng/mL, about 20 ng/mL to about 60 ng/mL, about 20 ng/mL to about 40 ng/mL, about 20 ng/mL to about 30 ng/mL, about 20 ng/mL to about 25 ng/mL, 25 ng/mL to about 500 ng/mL, about 25 ng/mL to about 480 ng/mL, about 25 ng/mL to about 460 ng/mL, about 25 ng/mL to about 440 ng/mL, about 25 ng/mL to about 420 ng/mL, about 25 ng/mL to about 400 ng/mL, about 25 ng/mL to about 380 ng/mL, about 25 ng/mL to about 360 ng/mL, about 25 ng/mL to about 340 ng/mL, about 25 ng/mL to about 320 ng/mL, about 25 ng/mL to about 300 ng/mL, about 25 ng/mL to about 280 ng/mL, about 25 ng/mL to about 260 ng/mL, about 25 ng/mL to about 240 ng/mL, about 25 ng/mL to about 220 ng/mL, about 25 ng/mL to about 200 ng/mL, about 25 ng/mL to about 180 ng/mL, about 25 ng/mL to about 160 ng/mL, about 25 ng/mL to about 140 ng/mL, about 25 ng/mL to about 120 ng/mL, about 25 ng/mL to about 100 ng/mL, about 25 ng/mL to about 80 ng/mL, about 25 ng/mL to about 60 ng/mL, about 25 ng/mL to about 40 ng/mL, about 25 ng/mL to about 30 ng/mL, 30 ng/mL to about 500 ng/mL, about 30 ng/mL to about 480 ng/mL, about 30 ng/mL to about 460 ng/mL, about 30 ng/mL to about 440 ng/mL, about 30 ng/mL to about 420 ng/mL, about 30 ng/mL to about 400 ng/mL, about 30 ng/mL to about 380 ng/mL, about 30 ng/mL to about 360 ng/mL, about 30 ng/mL to about 340 ng/mL, about 30 ng/mL to about 320 ng/mL, about 30 ng/mL to about 300 ng/mL, about 30 ng/mL to about 280 ng/mL, about 30 ng/mL to about 260 ng/mL, about 30 ng/mL to about 240 ng/mL, about 30 ng/mL to about 220 ng/mL, about 30 ng/mL to about 200 ng/mL, about 30 ng/mL to about 180 ng/mL, about 30 ng/mL to about 160 ng/mL, about 30 ng/mL to about 140 ng/mL, about 30 ng/mL to about 120 ng/mL, about 30 ng/mL to about 100 ng/mL, about 30 ng/mL to about 80 ng/mL, about 30 ng/mL to about 60 ng/mL, about 30 ng/mL to about 40 ng/mL, 40 ng/mL to about 500 ng/mL, about 40 ng/mL to about 480 ng/mL, about 40 ng/mL to about 460 ng/mL, about 40 ng/mL to about 440 ng/mL, about 40 ng/mL to about 420 ng/mL, about 40 ng/mL to about 400 ng/mL, about 40 ng/mL to about 380 ng/mL, about 40 ng/mL to about 360 ng/mL, about 40 ng/mL to about 340 ng/mL, about 40 ng/mL to about 320 ng/mL, about 40 ng/mL to about 300 ng/mL, about 40 ng/mL to about 280 ng/mL, about 40 ng/mL to about 260 ng/mL, about 40 ng/mL to about 240 ng/mL, about 40 ng/mL to about 220 ng/mL, about 40 ng/mL to about 200 ng/mL, about 40 ng/mL to about 180 ng/mL, about 40 ng/mL to about 160 ng/mL, about 40 ng/mL to about 140 ng/mL, about 40 ng/mL to about 120 ng/mL, about 40 ng/mL to about 100 ng/mL, about 40 ng/mL to about 80 ng/mL, about 40 ng/mL to about 60 ng/mL, 60 ng/mL to about 500 ng/mL, about 60 ng/mL to about 480 ng/mL, about 60 ng/mL to about 460 ng/mL, about 60 ng/mL to about 440 ng/mL, about 60 ng/mL to about 420 ng/mL, about 60 ng/mL to about 400 ng/mL, about 60 ng/mL to about 380 ng/mL, about 60 ng/mL to about 360 ng/mL, about 60 ng/mL to about 340 ng/mL, about 60 ng/mL to about 320 ng/mL, about 60 ng/mL to about 300 ng/mL, about 60 ng/mL to about 280 ng/mL, about 60 ng/mL to about 260 ng/mL, about 60 ng/mL to about 240 ng/mL, about 60 ng/mL to about 220 ng/mL, about 60 ng/mL to about 200 ng/mL, about 60 ng/mL to about 180 ng/mL, about 60 ng/mL to about 160 ng/mL, about 60 ng/mL to about 140 ng/mL, about 60 ng/mL to about 120 ng/mL, about 60 ng/mL to about 100 ng/mL, about 60 ng/mL to about 80 ng/mL, 80 ng/mL to about 500 ng/mL, about 80 ng/mL to about 480 ng/mL, about 80 ng/mL to about 460 ng/mL, about 80 ng/mL to about 440 ng/mL, about 80 ng/mL to about 420 ng/mL, about 80 ng/mL to about 400 ng/mL, about 80 ng/mL to about 380 ng/mL, about 80 ng/mL to about 360 ng/mL, about 80 ng/mL to about 340 ng/mL, about 80 ng/mL to about 320 ng/mL, about 80 ng/mL to about 300 ng/mL, about 80 ng/mL to about 280 ng/mL, about 80 ng/mL to about 260 ng/mL, about 80 ng/mL to about 240 ng/mL, about 80 ng/mL to about 220 ng/mL, about 80 ng/mL to about 200 ng/mL, about 80 ng/mL to about 180 ng/mL, about 80 ng/mL to about 160 ng/mL, about 80 ng/mL to about 140 ng/mL, about 80 ng/mL to about 120 ng/mL, about 80 ng/mL to about 100 ng/mL, 100 ng/mL to about 500 ng/mL, about 100 ng/mL to about 480 ng/mL, about 100 ng/mL to about 460 ng/mL, about 100 ng/mL to about 440 ng/mL, about 100 ng/mL to about 420 ng/mL, about 100 ng/mL to about 400 ng/mL, about 100 ng/mL to about 380 ng/mL, about 100 ng/mL to about 360 ng/mL, about 100 ng/mL to about 340 ng/mL, about 100 ng/mL to about 320 ng/mL, about 100 ng/mL to about 300 ng/mL, about 100 ng/mL to about 280 ng/mL, about 100 ng/mL to about 260 ng/mL, about 100 ng/mL to about 240 ng/mL, about 100 ng/mL to about 220 ng/mL, about 100 ng/mL to about 200 ng/mL, about 100 ng/mL to about 180 ng/mL, about 100 ng/mL to about 160 ng/mL, about 100 ng/mL to about 140 ng/mL, about 100 ng/mL to about 120 ng/mL, 120 ng/mL to about 500 ng/mL, about 120 ng/mL to about 480 ng/mL, about 120 ng/mL to about 460 ng/mL, about 120 ng/mL to about 440 ng/mL, about 120 ng/mL to about 420 ng/mL, about 120 ng/mL to about 400 ng/mL, about 120 ng/mL to about 380 ng/mL, about 120 ng/mL to about 360 ng/mL, about 120 ng/mL to about 340 ng/mL, about 120 ng/mL to about 320 ng/mL, about 120 ng/mL to about 300 ng/mL, about 120 ng/mL to about 280 ng/mL, about 120 ng/mL to about 260 ng/mL, about 120 ng/mL to about 240 ng/mL, about 120 ng/mL to about 220 ng/mL, about 120 ng/mL to about 200 ng/mL, about 120 ng/mL to about 180 ng/mL, about 120 ng/mL to about 160 ng/mL, about 120 ng/mL to about 140 ng/mL, 140 ng/mL to about 500 ng/mL, about 140 ng/mL to about 480 ng/mL, about 140 ng/mL to about 460 ng/mL, about 140 ng/mL to about 440 ng/mL, about 140 ng/mL to about 420 ng/mL, about 140 ng/mL to about 400 ng/mL, about 140 ng/mL to about 380 ng/mL, about 140 ng/mL to about 360 ng/mL, about 140 ng/mL to about 340 ng/mL, about 140 ng/mL to about 320 ng/mL, about 140 ng/mL to about 300 ng/mL, about 140 ng/mL to about 280 ng/mL, about 140 ng/mL to about 260 ng/mL, about 140 ng/mL to about 240 ng/mL, about 140 ng/mL to about 220 ng/mL, about 140 ng/mL to about 200 ng/mL, about 140 ng/mL to about 180 ng/mL, about 140 ng/mL to about 160 ng/mL, 160 ng/mL to about 500 ng/mL, about 160 ng/mL to about 480 ng/mL, about 160 ng/mL to about 460 ng/mL, about 160 ng/mL to about 440 ng/mL, about 160 ng/mL to about 420 ng/mL, about 160 ng/mL to about 400 ng/mL, about 160 ng/mL to about 380 ng/mL, about 160 ng/mL to about 360 ng/mL, about 160 ng/mL to about 340 ng/mL, about 160 ng/mL to about 320 ng/mL, about 160 ng/mL to about 300 ng/mL, about 160 ng/mL to about 280 ng/mL, about 160 ng/mL to about 260 ng/mL, about 160 ng/mL to about 240 ng/mL, about 160 ng/mL to about 220 ng/mL, about 160 ng/mL to about 200 ng/mL, about 160 ng/mL to about 180 ng/mL,180 ng/mL to about 500 ng/mL, about 180 ng/mL to about 480 ng/mL, about 180 ng/mL to about 460 ng/mL, about 180 ng/mL to about 440 ng/mL, about 180 ng/mL to about 420 ng/mL, about 180 ng/mL to about 400 ng/mL, about 180 ng/mL to about 380 ng/mL, about 180 ng/mL to about 360 ng/mL, about 180 ng/mL to about 340 ng/mL, about 180 ng/mL to about 320 ng/mL, about 180 ng/mL to about 300 ng/mL, about 180 ng/mL to about 280 ng/mL, about 180 ng/mL to about 260 ng/mL, about 180 ng/mL to about 240 ng/mL, about 180 ng/mL to about 220 ng/mL, about 180 ng/mL to about 200 ng/mL, 200 ng/mL to about 500 ng/mL, about 200 ng/mL to about 480 ng/mL, about 200 ng/mL to about 460 ng/mL, about 200 ng/mL to about 440 ng/mL, about 200 ng/mL to about 420 ng/mL, about 200 ng/mL to about 400 ng/mL, about 200 ng/mL to about 380 ng/mL, about 200 ng/mL to about 360 ng/mL, about 200 ng/mL to about 340 ng/mL, about 200 ng/mL to about 320 ng/mL, about 200 ng/mL to about 300 ng/mL, about 200 ng/mL to about 280 ng/mL, about 200 ng/mL to about 260 ng/mL, about 200 ng/mL to about 240 ng/mL, about 200 ng/mL to about 220 ng/mL, 220 ng/mL to about 500 ng/mL, about 220 ng/mL to about 480 ng/mL, about 220 ng/mL to about 460 ng/mL, about 220 ng/mL to about 440 ng/mL, about 220 ng/mL to about 420 ng/mL, about 220 ng/mL to about 300 ng/mL, about 220 ng/mL to about 380 ng/mL, about 220 ng/mL to about 360 ng/mL, about 220 ng/mL to about 340 ng/mL, about 220 ng/mL to about 320 ng/mL, about 220 ng/mL to about 400 ng/mL, about 220 ng/mL to about 280 ng/mL, about 220 ng/mL to about 260 ng/mL, about 220 ng/mL to about 240 ng/mL, 240 ng/mL to about 500 ng/mL, about 240 ng/mL to about 480 ng/mL, about 240 ng/mL to about 460 ng/mL, about 240 ng/mL to about 440 ng/mL, about 240 ng/mL to about 420 ng/mL, about 240 ng/mL to about 400 ng/mL, about 240 ng/mL to about 380 ng/mL, about 240 ng/mL to about 360 ng/mL, about 240 ng/mL to about 340 ng/mL, about 240 ng/mL to about 320 ng/mL, about 240 ng/mL to about 300 ng/mL, about 240 ng/mL to about 280 ng/mL, about 240 ng/mL to about 260 ng/mL, 260 ng/mL to about 500 ng/mL, about 260 ng/mL to about 480 ng/mL, about 260 ng/mL to about 460 ng/mL, about 260 ng/mL to about 440 ng/mL, about 260 ng/mL to about 420 ng/mL, about 260 ng/mL to about 400 ng/mL, about 260 ng/mL to about 380 ng/mL, about 260 ng/mL to about 360 ng/mL, about 260 ng/mL to about 340 ng/mL, about 260 ng/mL to about 320 ng/mL, about 260 ng/mL to about 300 ng/mL, about 260 ng/mL to about 280 ng/mL, 280 ng/mL to about 500 ng/mL, about 280 ng/mL to about 480 ng/mL, about 280 ng/mL to about 460 ng/mL, about 280 ng/mL to about 440 ng/mL, about 280 ng/mL to about 420 ng/mL, about 280 ng/mL to about 400 ng/mL, about 280 ng/mL to about 380 ng/mL, about 280 ng/mL to about 360 ng/mL, about 280 ng/mL to about 340 ng/mL, about 280 ng/mL to about 320 ng/mL, about 280 ng/mL to about 300 ng/mL, 300 ng/mL to about 500 ng/mL, about 300 ng/mL to about 480 ng/mL, about 300 ng/mL to about 460 ng/mL, about 300 ng/mL to about 440 ng/mL, about 300 ng/mL to about 420 ng/mL, about 300 ng/mL to about 400 ng/mL, about 300 ng/mL to about 380 ng/mL, about 300 ng/mL to about 360 ng/mL, about 300 ng/mL to about 340 ng/mL, about 300 ng/mL to about 320 ng/mL, 320 ng/mL to about 500 ng/mL, about 320 ng/mL to about 480 ng/mL, about 320 ng/mL to about 460 ng/mL, about 320 ng/mL to about 440 ng/mL, about 320 ng/mL to about 420 ng/mL, about 320 ng/mL to about 400 ng/mL, about 320 ng/mL to about 380 ng/mL, about 320 ng/mL to about 360 ng/mL, about 320 ng/mL to about 340 ng/mL, 340 ng/mL to about 500 ng/mL, about 340 ng/mL to about 480 ng/mL, about 340 ng/mL to about 460 ng/mL, about 340 ng/mL to about 440 ng/mL, about 340 ng/mL to about 420 ng/mL, about 340 ng/mL to about 400 ng/mL, about 340 ng/mL to about 380 ng/mL, about 340 ng/mL to about 360 ng/mL, 360 ng/mL to about 500 ng/mL, about 360 ng/mL to about 480 ng/mL, about 360 ng/mL to about 460 ng/mL, about 360 ng/mL to about 440 ng/mL, about 360 ng/mL to about 420 ng/mL, about 360 ng/mL to about 400 ng/mL, about 360 ng/mL to about 380 ng/mL, 380 ng/mL to about 500 ng/mL, about 380 ng/mL to about 480 ng/mL, about 380 ng/mL to about 460 ng/mL, about 380 ng/mL to about 440 ng/mL, about 380 ng/mL to about 420 ng/mL, about 380 ng/mL to about 400 ng/mL, 400 ng/mL to about 500 ng/mL, about 400 ng/mL to about 480 ng/mL, about 400 ng/mL to about 460 ng/mL, about 400 ng/mL to about 440 ng/mL, about 400 ng/mL to about 420 ng/mL, 420 ng/mL to about 500 ng/mL, about 420 ng/mL to about 480 ng/mL, about 420 ng/mL to about 460 ng/mL, about 420 ng/mL to about 440 ng/mL, 440 ng/mL to about 500 ng/mL, about 440 ng/mL to about 480 ng/mL, about 440 ng/mL to about 460 ng/mL, about 460 ng/mL to about 500 ng/mL, about 460 ng/mL to about 480 ng/mL, or about 480 ng/mL to about 500 ng/mL of EPO (e.g., recombinant human EPO) or an EPO-mimetic peptide. Non-limiting examples of EPO-mimetic peptides are described herein. Additional examples of EPO-mimetic peptides are known in the art.

In some embodiments, the fourth culture medium includes about 0.1 μg/mL to about 50 μg/mL, about 0.1 μg/mL to about 45 μg/mL, about 0.1 μg/mL to about 40 μg/mL, about 0.1 μg/mL to about 35 μg/mL, about 0.1 μg/mL to about 30 μg/mL, about 0.1 μg/mL to about 25 μg/mL, about 0.1 μg/mL to about 20 μg/mL, about 0.1 μg/mL about 15 μg/mL, about 0.1 μg/mL to about 10 μg/mL, about 0.1 μg/mL to about 5 μg/mL, about 0.1 μg/mL to about 2 μg/mL, about 0.1 μg/mL to about 1 μg/mL, about 1 μg/mL to about 50 μg/mL, about 1 μg/mL to about 45 μg/mL, about 1 μg/mL to about 40 μg/mL, about 1 μg/mL to about 35 μg/mL, about 1 μg/mL to about 30 μg/mL, about 1 μg/mL to about 25 μg/mL, about 1 μg/mL to about 20 μg/mL, about 1 μg/mL about 15 μg/mL, about 1 μg/mL to about 10 μg/mL, about 1 μg/mL to about 5 μg/mL, about 1 μg/mL to about 2 μg/mL, about 2 μg/mL to about 50 μg/mL, about 2 μg/mL to about 45 μg/mL, about 2 μg/mL to about 40 μg/mL, about 2 μg/mL to about 35 μg/mL, about 2 μg/mL to about 30 μg/mL, about 2 μg/mL to about 25 μg/mL, about 2 μg/mL to about 20 μg/mL, about 2 μg/mL about 15 μg/mL, about 2 μg/mL to about 10 μg/mL, about 2 μg/mL to about 5 μg/mL, about 5 μg/mL to about 50 μg/mL, about 5 μg/mL to about 45 μg/mL, about 5 μg/mL to about 40 μg/mL, about 5 μg/mL to about 35 μg/mL, about 5 μg/mL to about 30 μg/mL, about 5 μg/mL to about 25 μg/mL, about 5 μg/mL to about 20 μg/mL, about 5 μg/mL about 15 μg/mL, about 5 μg/mL to about 10 μg/mL, about 10 μg/mL to about 50 μg/mL, about 10 μg/mL to about 45 μg/mL, about 10 μg/mL to about 40 μg/mL, about 10 μg/mL to about 35 μg/mL, about 10 μg/mL to about 30 μg/mL, about 10 μg/mL to about 25 μg/mL, about 10 μg/mL to about 20 μg/mL, about 10 μg/mL about 15 μg/mL, about 15 μg/mL to about 50 μg/mL, about 15 μg/mL to about 45 μg/mL, about 15 μg/mL to about 40 μg/mL, about 15 μg/mL to about 35 μg/mL, about 15 μg/mL to about 30 μg/mL, about 15 μg/mL to about 25 μg/mL, about 15 μg/mL to about 20 μg/mL, about 20 μg/mL to about 50 μg/mL, about 20 μg/mL to about 45 μg/mL, about 20 μg/mL to about 40 μg/mL, about 20 μg/mL to about 35 μg/mL, about 20 μg/mL to about 30 μg/mL, about 20 μg/mL to about 25 μg/mL, about 25 μg/mL to about 50 μg/mL, about 25 μg/mL to about 45 μg/mL, about 25 μg/mL to about 40 μg/mL, about 25 μg/mL to about 35 μg/mL, about 25 μg/mL to about 30 μg/mL, about 30 μg/mL to about 50 μg/mL, about 30 μg/mL to about 45 μg/mL, about 30 μg/mL to about 40 μg/mL, about 30 μg/mL to about 35 μg/mL, about 35 μg/mL to about 50 μg/mL, about 35 μg/mL to about 45 μg/mL, about 35 μg/mL to about 40 μg/mL, about 40 μg/mL to about 50 μg/mL, about 40 μg/mL to about 45 μg/mL, or about 45 μg/mL to about 50 μg/mL, of insulin (e.g., human insulin).

In some embodiments, the perfusion culturing includes the use of tangential filtration (e.g., tangential flow filtration (TFF) or alternating tangential filtration (ATF)). In some embodiments, the tangential filtration (e.g., TFF or ATF) includes the use of one or more filters that have an average pore size of about 10 nm to about 6.0 μM (or any of the subranges of this range described herein).

In some examples, the fourth culture medium can be, e.g., a chemically-defined liquid culture medium, an animal component-free liquid culture medium, or a chemically-defined animal component-free liquid culture medium, and/or a serum-free liquid culture medium.

In some embodiments, the perfusion of the third cell culture begins once the first cell culture reaches a specific target cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

In some embodiments, the perfusion culturing of step (d) is performed for about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 15 days, about 9 days to about 14 days, about 9 days to about 13 days, about 9 days to about 12 days, about 9 days to about 11 days, about 9 days to about 10 days, about 10 days to about 15 days, about 10 days to about 14 days, about 10 days to about 13 days, about 10 days to about 12 days, about 10 days to about 11 days, about 11 days to about 15 days, about 11 days to about 14 days, about 11 days to about 13 days, about 11 days to about 12 days, about 12 days to about 15 days, about 12 days to about 14 days, about 12 days to about 13 days, about 13 days to about 15 days, about 13 days to about 14 days, or about 14 days to about 15 days.

In some embodiments, step (d) results in a cell density of about 5×10⁶ enucleated erythroid cells/mL to about 2×10⁸ enucleated erythroid cells/mL, about 5×10⁶ enucleated erythroid cells/mL to about 1×10⁸ enucleated erythoid cells/mL, about 5×10⁶ enucleated erythroid cells/mL to about 8×10⁷ enucleated erythroid cells/mL, about 5×10⁶ enucleated erythroid cells/mL to about 6×10⁷ enucleated erythroid cells/mL, about 5×10⁶ enucleated erythroid cells/mL to about 4×10⁷ enucleated erythroid cells/mL, about 5×10⁶ enucleated erythroid cells/mL to about 2×10⁷ enucleated erythroid cells/mL, about 5×10⁶ enucleated erythroid cells/mL to about 1×10⁷ enucleated erythroid cells/mL, about 5×10⁶ enucleated erythroid cells/mL to about 8×10⁶ enucleated erythroid cells/mL, about 5×10⁶ enucleated erythroid cells/mL to about 6×10⁶ enucleated erythroid cells/mL, about 6×10⁶ enucleated erythroid cells/mL to about 1×10⁸ enucleated erythoid cells/mL, about 6×10⁶ enucleated erythroid cells/mL to about 8×10⁷ enucleated erythroid cells/mL, about 6×10⁶ enucleated erythroid cells/mL to about 6×10⁷ enucleated erythroid cells/mL, about 6×10⁶ enucleated erythroid cells/mL to about 4×10⁷ enucleated erythroid cells/mL, about 6×10⁶ enucleated erythroid cells/mL to about 2×10⁷ enucleated erythroid cells/mL, about 6×10⁶ enucleated erythroid cells/mL to about 1×10⁷ enucleated erythroid cells/mL, about 6×10⁶ enucleated erythroid cells/mL to about 8×10⁶ enucleated erythroid cells/mL, about 8×10⁶ enucleated erythroid cells/mL to about 1×10⁸ enucleated erythoid cells/mL, about 8×10⁶ enucleated erythroid cells/mL to about 8×10⁷ enucleated erythroid cells/mL, about 8×10⁶ enucleated erythroid cells/mL to about 6×10⁷ enucleated erythroid cells/mL, about 8×10⁶ enucleated erythroid cells/mL to about 4×10⁷ enucleated erythroid cells/mL, about 8×10⁶ enucleated erythroid cells/mL to about 2×10⁷ enucleated erythroid cells/mL, about 8×10⁶ enucleated erythroid cells/mL to about 1×10⁷ enucleated erythroid cells/mL, about 1×10⁷ enucleated erythroid cells/mL to about 1×10⁸ enucleated erythoid cells/mL, about 1×10⁷ enucleated erythroid cells/mL to about 8×10⁷ enucleated erythroid cells/mL, about 1×10⁷ enucleated erythroid cells/mL to about 6×10⁷ enucleated erythroid cells/mL, about 1×10⁷ enucleated erythroid cells/mL to about 4×10⁷ enucleated erythroid cells/mL, about 1×10⁷ enucleated erythroid cells/mL to about 2×10⁷ enucleated erythroid cells/mL, about 2×10⁷ enucleated erythroid cells/mL to about 1×10⁸ enucleated erythoid cells/mL, about 2×10⁷ enucleated erythroid cells/mL to about 8×10⁷ enucleated erythroid cells/mL, about 2×10⁷ enucleated erythroid cells/mL to about 6×10⁷ enucleated erythroid cells/mL, about 2×10⁷ enucleated erythroid cells/mL to about 4×10⁷ enucleated erythroid cells/mL, about 4×10⁷ enucleated erythroid cells/mL to about 1×10⁸ enucleated erythoid cells/mL, about 4×10⁷ enucleated erythroid cells/mL to about 8×10⁷ enucleated erythroid cells/mL, about 4×10⁷ enucleated erythroid cells/mL to about 6×10⁷ enucleated erythroid cells/mL, about 6×10⁷ enucleated erythroid cells/mL to about 1×10⁸ enucleated erythoid cells/mL, about 6×10⁷ enucleated erythroid cells/mL to about 8×10⁷ enucleated erythroid cells/mL, about 8×10⁷ enucleated erythroid cells/mL to about 1×10⁸ enucleated erythoid cells/mL.

Batch or Fed Batch Culturing

In some embodiments of any of the methods described herein, step (d) includes batch or fed batch culturing the third cell culture (e.g., any of the exemplary third cell cultures described herein in any of the exemplary shake flasks, shake tubes, culture bags, or bioreactors described herein having any of the exemplary volumes described herein) for about 5 days to about 20 days (e.g., about 5 days to about 19 days, about 5 days to about 18 days, about 5 days to about 17 days, about 5 days to about 16 days, about 5 days to about 15 days, about 5 days to about 14 days, about 5 days to about 13 days, about 5 days to about 12 days, about 5 days to about 11 days, about 5 days to about 10 days, about 5 days to about 9 days, about 5 days to about 8 days, about 5 days to about 7 days, about 5 days to about 6 days, about 6 days to about 20 days, about 6 days to about 19 days, about 6 days to about 18 days, about 6 days to about 17 days, about 6 days to about 16 days, about 6 days to about 15 days, about 6 days to about 14 days, about 6 days to about 13 days, about 6 days to about 12 days, about 6 days to about 11 days, about 6 days to about 10 days, about 6 days to about 9 days, about 6 days to about 8 days, about 6 days to about 7 days, about 7 days to about 20 days, about 7 days to about 19 days, about 7 days to about 18 days, about 7 days to about 17 days, about 7 days to about 16 days, about 7 days to about 15 days, about 7 days to about 14 days, about 7 days to about 13 days, about 7 days to about 12 days, about 7 days to about 11 days, about 7 days to about 10 days, about 7 days to about 9 days, about 7 days to about 8 days, about 8 days to about 20 days, about 8 days to about 19 days, about 8 days to about 18 days, about 8 days to about 17 days, about 8 days to about 16 days, about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 20 days, about 9 days to about 19 days, about 9 days to about 18 days, about 9 days to about 17 days, about 9 days to about 16 days, about 9 days to about 15 days, about 9 days to about 14 days, about 9 days to about 13 days, about 9 days to about 12 days, about 9 days to about 11 days, about 9 days to about 10 days, about 10 days to about 20 days, about 10 days to about 19 days, about 10 days to about 18 days, about 10 days to about 17 days, about 10 days to about 16 days, about 10 days to about 15 days, about 10 days to about 14 days, about 10 days to about 13 days, about 10 days to about 12 days, about 10 days to about 11 days, about 11 days to about 20 days, about 11 days to about 19 days, about 11 days to about 18 days, about 11 days to about 17 days, about 11 days to about 16 days, about 11 days to about 15 days, about 11 days to about 14 days, about 11 days to about 13 days, about 11 days to about 12 days, about 12 days to about 20 days, about 12 days to about 19 days, about 12 days to about 18 days, about 12 days to about 17 days, about 12 days to about 16 days, about 12 days to about 15 days, about 12 days to about 14 days, about 12 days to about 13 days, about 13 days to about 20 days, about 13 days to about 19 days, about 13 days to about 18 days, about 13 days to about 17 days, about 13 days to about 16 days, about 13 days to about 15 days, about 13 days to about 14 days, about 14 days to about 20 days, about 14 days to about 19 days, about 14 days to about 18 days, about 14 days to about 17 days, about 14 days to about 16 days, about 14 days to about 15 days, about 15 days to about 20 days, about 15 days to about 19 days, about 15 days to about 18 days, about 15 days to about 17 days, about 15 days to about 16 days, about 16 days to about 20 days, about 16 days to about 19 days, about 16 days to about 18 days, about 16 days to about 17 days, about 17 days to about 20 days, about 17 days to about 19 days, about 17 days to about 18 days, about 18 days to about 20 days, about 18 days to about 19 days, or about 19 days to about 20 days), wherein after step (d) the third culture medium includes a population of enucleated erythroid cells (e.g., any of the exemplary enucleated erythroid cells described herein).

In some embodiments, step (d) includes batch or fed batch culturing the third cell culture (e.g., any of the exemplary third cell cultures described herein) disposed in a bioreactor (e.g., any of the exemplary bioreactors described herein having any of the exemplary volumes described herein). In such embodiments, step (d) includes incubating the third cell culture (e.g., any of the exemplary third cell cultures described herein) in the bioreactor with agitation at a P/V value of about 10 W/m³ to about 200 W/m³ (or any of the subranges of this range described herein).

In some embodiments, step (d) includes batch or fed batch culturing the third cell culture (e.g., any of the exemplary third cell cultures described herein) disposed in a shake flask (e.g., any of the exemplary shake flasks described herein having any of the exemplary volumes described herein). In such embodiments, step (d) includes incubating the third cell culture (e.g., any of the exemplary third cell cultures described herein) in the shake flask at about 0.1×g to about 50×g (e.g., any of the subranges of this range described herein).

In some embodiments, step (d) includes batch or fed batch culturing the third cell culture (e.g., any of the exemplary third cell cultures described herein) disposed in a shake tube (e.g., any of the exemplary shake tubes described herein (e.g., a conical container) having any of the exemplary volumes described herein). In such embodiments, step (d) includes incubating the third cell culture (e.g., any of the exemplary third cell cultures described herein) in the shake tube at about 0.1×g to about 50×g (e.g., or any of the subranges of this range described herein).

In some embodiments, step (d) includes batch or fed batch culturing the third cell culture (e.g., any of the exemplary third cell cultures described herein) disposed in a culture bag (e.g., any of the exemplary culture bags described herein having any of the exemplary volumes described herein). In such embodiments, step (d) includes incubating the third cell culture (e.g., any of the exemplary third cell cultures described herein) in the culture bag at rocking rate of 10 rock cycles per minute to about 50 rock cycles per minute (or any of the subranges of this range described herein).

In some embodiments, step (d) includes batch culturing the third cell culture (e.g., any of the exemplary third cell cultures described herein).

In some embodiments, step (d) includes fed batch culturing the third cell culture (e.g., any of the exemplary third cell cultures described herein). In some embodiments, fed batch culturing in step (d) includes adding an additional volume of the third culture medium (e.g., any of the exemplary third culture media described herein) and/or the fourth culture medium (e.g., any of the exemplary fourth culture media described herein) to the third cell culture (e.g., any of the exemplary third cell cultures described herein) over time. In some examples, the additional volume of the third culture medium (e.g., any of the exemplary third culture media described herein) and/or the fourth culture medium (e.g., any of the exemplary fourth culture media described herein) is added continuously to the third cell culture (e.g., any of the exemplary third cell cultures described herein) over time. In some examples, the additional volume of the third culture medium (e.g., any of the exemplary third cell cultures described herein) and/or the fourth culture media (e.g., any of the exemplary fourth culture media described herein) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the third cell culture (e.g., any of the exemplary third cell cultures described herein) over time. In some embodiments, about 0.1× to about 10× (e.g., about 0.1× to about 9.5×, about 0.1× to about 9.0×, about 0.1× to about 8.5×, about 0.1× to about 8.0×, about 0.1× to about 7.5×, about 0.1× to about 7.0×, about 0.1× to about 6.5×, about 0.1× to about 6.0×, about 0.1× to about 5.5×, about 0.1× to about 5.0×, about 0.1× to about 4.5×, about 0.1× to about 4.0×, about 0.1× to about 3.5×, about 0.1× to about 3.0×, about 0.1× to about 2.5×, about 0.1× to about 2.0×, about 0.1× to about 1.5×, about 0.1× to about 1.0×, about 0.1× to about 0.5×, about 0.1× to about 0.3×, about 0.1× to about 0.2×, about 0.2× to about 10×, about 0.2× to about 9.5×, about 0.2× to about 9.0×, about 0.2× to about 8.5×, about 0.2× to about 8.0×, about 0.2× to about 7.5×, about 0.2× to about 7.0×, about 0.2× to about 6.5×, about 0.2× to about 6.0×, about 0.2× to about 5.5×, about 0.2× to about 5.0×, about 0.2× to about 4.5×, about 0.2× to about 4.0×, about 0.2× to about 3.5×, about 0.2× to about 3.0×, about 0.2× to about 2.5×, about 0.2× to about 2.0×, about 0.2× to about 1.5×, about 0.2× to about 1.0×, about 0.2× to about 0.5×, about 0.2× to about 0.3×, about 0.3× to about 10×, about 0.3× to about 9.5×, about 0.3× to about 9.0×, about 0.3× to about 8.5×, about 0.3× to about 8.0×, about 0.3× to about 7.5×, about 0.3× to about 7.0×, about 0.3× to about 6.5×, about 0.3× to about 6.0×, about 0.3× to about 5.5×, about 0.3× to about 5.0×, about 0.3× to about 4.5×, about 0.3× to about 4.0×, about 0.3× to about 3.5×, about 0.3× to about 3.0×, about 0.3× to about 2.5×, about 0.3× to about 2.0×, about 0.3× to about 1.5×, about 0.3× to about 1.0×, about 0.3× to about 0.5×, about 0.5× to about 10×, about 0.5× to about 9.5×, about 0.5× to about 9.0×, about 0.5× to about 8.5×, about 0.5× to about 8.0×, about 0.5× to about 7.5×, about 0.5× to about 7.0×, about 0.5× to about 6.5×, about 0.5× to about 6.0×, about 0.5× to about 5.5×, about 0.5× to about 5.0×, about 0.5× to about 4.5×, about 0.5× to about 4.0×, about 0.5× to about 3.5×, about 0.5× to about 3.0×, about 0.5× to about 2.5×, about 0.5× to about 2.0×, about 0.5× to about 1.5×, about 0.5× to about 1.0×, about 1.0× to about 10×, about 1.0× to about 9.5×, about 1.0× to about 9.0×, about 1.0× to about 8.5×, about 1.0× to about 8.0×, about 1.0× to about 7.5×, about 1.0× to about 7.0×, about 1.0× to about 6.5×, about 1.0× to about 6.0×, about 1.0× to about 5.5×, about 1.0× to about 5.0×, about 1.0× to about 4.5×, about 1.0× to about 4.0×, about 1.0× to about 3.5×, about 1.0× to about 3.0×, about 1.0× to about 2.5×, about 1.0× to about 2.0×, about 1.0× to about 1.5×, about 1.5× to about 10×, about 1.5× to about 9.5×, about 1.5× to about 9.0×, about 1.5× to about 8.5×, about 1.5× to about 8.0×, about 1.5× to about 7.5×, about 1.5× to about 7.0×, about 1.5× to about 6.5×, about 1.5× to about 6.0×, about 1.5× to about 5.5×, about 1.5× to about 5.0×, about 1.5× to about 4.5×, about 1.5× to about 4.0×, about 1.5× to about 3.5×, about 1.5× to about 3.0×, about 1.5× to about 2.5×, about 1.5× to about 2.0×, about 2.0× to about 10×, about 2.0× to about 9.5×, about 2.0× to about 9.0×, about 2.0× to about 8.5×, about 2.0× to about 8.0×, about 2.0× to about 7.5×, about 2.0× to about 7.0×, about 2.0× to about 6.5×, about 2.0× to about 6.0×, about 2.0× to about 5.5×, about 2.0× to about 5.0×, about 2.0× to about 4.5×, about 2.0× to about 4.0×, about 2.0× to about 3.5×, about 2.0× to about 3.0×, about 2.0× to about 2.5×, about 2.5× to about 10×, about 2.5× to about 9.5×, about 2.5× to about 9.0×, about 2.5× to about 8.5×, about 2.5× to about 8.0×, about 2.5× to about 7.5×, about 2.5× to about 7.0×, about 2.5× to about 6.5×, about 2.5× to about 6.0×, about 2.5× to about 5.5×, about 2.5× to about 5.0×, about 2.5× to about 4.5×, about 2.5× to about 4.0×, about 2.5× to about 3.5×, about 2.5× to about 3.0×, about 3.0× to about 10×, about 3.0× to about 9.5×, about 3.0× to about 9.0×, about 3.0× to about 8.5×, about 3.0× to about 8.0×, about 3.0× to about 7.5×, about 3.0× to about 7.0×, about 3.0× to about 6.5×, about 3.0× to about 6.0×, about 3.0× to about 5.5×, about 3.0× to about 5.0×, about 3.0× to about 4.5×, about 3.0× to about 4.0×, about 3.0× to about 3.5×, about 3.5× to about 10×, about 3.5× to about 9.5×, about 3.5× to about 9.0×, about 3.5× to about 8.5×, about 3.5× to about 8.0×, about 3.5× to about 7.5×, about 3.5× to about 7.0×, about 3.5× to about 6.5×, about 3.5× to about 6.0×, about 3.5× to about 5.5×, about 3.5× to about 5.0×, about 3.5× to about 4.5×, about 3.5× to about 4.0×, about 4.0× to about 10×, about 4.0× to about 9.5×, about 4.0× to about 9.0×, about 4.0× to about 8.5×, about 4.0× to about 8.0×, about 4.0× to about 7.5×, about 4.0× to about 7.0×, about 4.0× to about 6.5×, about 4.0× to about 6.0×, about 4.0× to about 5.5×, about 4.0× to about 5.0×, about 4.0× to about 4.5×, about 4.5× to about 10×, about 4.5× to about 9.5×, about 4.5× to about 9.0×, about 4.5× to about 8.5×, about 4.5× to about 8.0×, about 4.5× to about 7.5×, about 4.5× to about 7.0×, about 4.5× to about 6.5×, about 4.5× to about 6.0×, about 4.5× to about 5.5×, about 4.5× to about 5.0×, about 5.0× to about 10×, about 5.0× to about 9.5×, about 5.0× to about 9.0×, about 5.0× to about 8.5×, about 5.0× to about 8.0×, about 5.0× to about 7.5×, about 5.0× to about 7.0×, about 5.0× to about 6.5×, about 5.0× to about 6.0×, about 5.0× to about 5.5×, about 5.5× to about 10×, about 5.5× to about 9.5×, about 5.5× to about 9.0×, about 5.5× to about 8.5×, about 5.5× to about 8.0×, about 5.5× to about 7.5×, about 5.5× to about 7.0×, about 5.5× to about 6.5×, about 5.5× to about 6.0×, about 6.0× to about 10×, about 6.0× to about 9.5×, about 6.0× to about 9.0×, about 6.0× to about 8.5×, about 6.0× to about 8.0×, about 6.0× to about 7.5×, about 6.0× to about 7.0×, about 6.0× to about 6.5×, about 6.5× to about 10×, about 6.5× to about 9.5×, about 6.5× to about 9.0×, about 6.5× to about 8.5×, about 6.5× to about 8.0×, about 6.5× to about 7.5×, about 6.5× to about 7.0×, about 7.0× to about 10×, about 7.0× to about 9.5×, about 7.0× to about 9.0×, about 7.0× to about 8.5×, about 7.0× to about 8.0×, about 7.0× to about 7.5×, about 7.5× to about 10×, about 7.5× to about 9.5×, about 7.5× to about 9.0×, about 7.5× to about 8.5×, about 7.5× to about 8.0×, about 8.0× to about 10×, about 8.0× to about 9.5×, about 8.0× to about 9.0×, about 8.0× to about 8.5×, about 8.5× to about 10×, about 8.5× to about 9.5×, about 8.5× to about 9.0×, about 9.0× to about 10×, about 9.0× to about 9.5×, or about 9.5× to about 10×) of the volume of the third cell culture immediately after step (c) is added in each 24-hour increment. In some embodiments, the addition of additional volumes of the third culture medium (e.g., any of the exemplary third culture media described herein) and/or the fourth culture medium (e.g., any of the exemplary fourth culture media described herein) to the third cell culture begins once the third cell culture reaches a specific target cell density, e.g., about 1.0×10⁶ cells/mL, about 1.5×10⁶ cells/mL, about 2.0×10⁶ cells/mL, about 2.5×10⁶ cells/mL, about 3.0×10⁶ cells/mL, about 3.5×10⁶ cells/mL, about 4.0×10⁶ cells/mL, about 4.5×10⁶ cells/mL, about 5.0×10⁶ cells/mL, about 5.5×10⁶ cells/mL, about 6.0×10⁶ cells/mL, about 6.5×10⁶ cells/mL, about 7.0×10⁶ cells/mL, about 7.5×10⁶ cells/mL, about 8.0×10⁶ cells/mL, about 8.5×10⁶ cells/mL, about 9.0×10⁶ cells/mL, about 9.5×10⁶ cells/mL, or about 1.0×10⁷ cell/mL.

In some examples, the third culture medium (e.g., any of the exemplary third culture media described herein) includes one or more (e.g., two, three, or four) transferrin (e.g., about 1 μg/mL to about 500 μg/mL transferrin (e.g., human apotransferrin, human holo transferrin, or a combination thereof) or any of the subranges of this range described herein), insulin (e.g., about 0.1 μg/mL to about 50 μg/mL insulin (e.g., recombinant human insulin) or any of the subranges of this range described herein), SCF (e.g., about 1 ng/mL to about 500 ng/mL SCF (e.g., recombinant human SCF) or any of the subranges of this range described herein), and EPO or an EPO mimetic peptide (e.g., about 1 ng/mL to about 500 ng/mL EPO (e.g., recombinant human EPO) or an EPO mimetic peptide (e.g., any of the exemplary EPO mimetic peptides described herein or known in the art), or any of the subranges of this range described herein). In some embodiments of any of the third culture media described herein, the third culture medium includes about 0.1% v/v to about 10% v/v (e.g. any of the subranges of this range described herein) serum (e.g., human AB serum). In some embodiments of any of the third culture media described herein, the third culture medium includes about 1 mM to about 8 mM (e.g. any of the subranges of this range described herein) L-glutamine (e.g., L-alanyl-L-glutamine).

In some examples, the fed batch culturing in step (d) includes: (i) adding an additional volume of the a third culture medium (e.g., any of the exemplary third culture media described herein) to the third cell culture (e.g., any of the exemplary third cell cultures described herein) for a first period of time; and (ii) adding an additional volume of a fourth culture medium (e.g., any of the exemplary fourth culture media described herein) to the third cell culture (e.g., any of the exemplary third cell cultures described herein) for a second period of time.

In some examples, the additional volume of the third culture medium (e.g., any of the exemplary third culture media described herein) in (i) is added continuously to the third cell culture (e.g., any of the exemplary third cell cultures described herein) for the first period of time; and/or the additional volume of the fourth culture medium (e.g., any of the exemplary fourth culture media described herein) in (ii) is added continuously to the third cell culture (e.g., any of the exemplary third cell cultures described herein) for the second period of time.

In some examples, the additional volume of the third culture medium (e.g., any of the exemplary third culture media described herein) in (i) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the third cell culture (e.g., any of the exemplary third cell cultures described herein) for the first period of time; and/or (ii) the additional volume of the fourth culture medium (e.g., any of the exemplary fourth culture media described herein) in (ii) is added periodically (e.g., once every three days, once every two days, once a day, twice a day, three times a day, four times a day, five times a day, six times a day, seven times a day, eight times a day, nine times a day, ten times a day, eleven times a day, or twelve times a day) to the third cell culture (e.g., any of the exemplary third cell cultures described herein) for the second period of time.

The addition of culture medium (e.g., any of the exemplary third culture media described herein and/or any of the exemplary fourth culture media described herein) can be performed mechanically, e.g., using a peristaltic pump, or manually (e.g., by sterile pipetting).

In some examples, the first period of time in (i) is about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 day to about 12 days, about 2 day to about 11 days, about 2 day to about 10 days, about 2 day to about 9 days, about 2 day to about 8 days, about 2 day to about 7 days, about 2 day to about 6 days, about 2 day to about 5 days, about 2 day to about 4 days, about 2 day to about 3 days, about 3 day to about 12 days, about 3 day to about 11 days, about 3 day to about 10 days, about 3 day to about 9 days, about 3 day to about 8 days, about 3 day to about 7 days, about 3 day to about 6 days, about 3 day to about 5 days, about 3 day to about 4 days, about 4 day to about 12 days, about 4 day to about 11 days, about 4 day to about 10 days, about 4 day to about 9 days, about 4 day to about 8 days, about 4 day to about 7 days, about 4 day to about 6 days, about 4 day to about 5 days, about 5 day to about 12 days, about 5 day to about 11 days, about 5 day to about 10 days, about 5 day to about 9 days, about 5 day to about 8 days, about 5 day to about 7 days, about 5 day to about 6 days, about 6 day to about 12 days, about 6 day to about 11 days, about 6 day to about 10 days, about 6 day to about 9 days, about 6 day to about 8 days, about 6 day to about 7 days, about 7 day to about 12 days, about 7 day to about 11 days, about 7 day to about 10 days, about 7 day to about 9 days, about 7 day to about 8 days, about 8 day to about 12 days, about 8 day to about 11 days, about 8 day to about 10 days, about 8 day to about 9 days, about 9 day to about 12 days, about 9 day to about 11 days, about 9 day to about 10 days, about 10 day to about 12 days, about 10 day to about 11 days, or about 11 day to about 12 days.

In some examples, the second period of time in (ii) is about 1 day to about 12 days, about 1 day to about 11 days, about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, about 1 day to about 2 days, about 2 day to about 12 days, about 2 day to about 11 days, about 2 day to about 10 days, about 2 day to about 9 days, about 2 day to about 8 days, about 2 day to about 7 days, about 2 day to about 6 days, about 2 day to about 5 days, about 2 day to about 4 days, about 2 day to about 3 days, about 3 day to about 12 days, about 3 day to about 11 days, about 3 day to about 10 days, about 3 day to about 9 days, about 3 day to about 8 days, about 3 day to about 7 days, about 3 day to about 6 days, about 3 day to about 5 days, about 3 day to about 4 days, about 4 day to about 12 days, about 4 day to about 11 days, about 4 day to about 10 days, about 4 day to about 9 days, about 4 day to about 8 days, about 4 day to about 7 days, about 4 day to about 6 days, about 4 day to about 5 days, about 5 day to about 12 days, about 5 day to about 11 days, about 5 day to about 10 days, about 5 day to about 9 days, about 5 day to about 8 days, about 5 day to about 7 days, about 5 day to about 6 days, about 6 day to about 12 days, about 6 day to about 11 days, about 6 day to about 10 days, about 6 day to about 9 days, about 6 day to about 8 days, about 6 day to about 7 days, about 7 day to about 12 days, about 7 day to about 11 days, about 7 day to about 10 days, about 7 day to about 9 days, about 7 day to about 8 days, about 8 day to about 12 days, about 8 day to about 11 days, about 8 day to about 10 days, about 8 day to about 9 days, about 9 day to about 12 days, about 9 day to about 11 days, about 9 day to about 10 days, about 10 day to about 12 days, about 10 day to about 11 days, or about 11 day to about 12 days.

In some examples of these methods, the fourth culture medium (e.g., any of the exemplary fourth culture media described herein) includes one or more (e.g., one, two, or three) of: transferrin (e.g., about 100 μg/mL to about 2 mg/mL transferrin (e.g., apotransferrin, holotransferrin, or a combination thereof) or any of the subranges of this range described herein), erythropoietin (EPO) or an EPO-mimetic peptide (e.g., any of the exemplary EPO-mimetic peptides described herein) (e.g., about 1 ng/mL to about 500 ng/mL of EPO (e.g., recombinant human EPO) or an EPO-mimetic peptide or any of the subranges of this range described herein), and insulin (e.g., about 0.1 μg/mL to about 50 μg/mL insulin or any of the subranges of this range described herein). In some embodiments of any of the fourth culture media described herein, the fourth culture medium includes about 0.1% v/v to about 10% v/v (e.g., any of the subranges of this range described herein) serum (e.g., human AB serum). In some embodiments of any of the fourth culture media described herein, the fourth culture medium includes about 1 mM to about 8 mM (e.g., any of the subranges of this range described herein) of L-glutamine, L-alanyl-L-glutamine, L-glycyl-L-glutamine, N-acetyl-L-glutamine, or a combination thereof.

In some embodiments, the batch or fed batch culturing of step (d) is performed for about 8 days to about 15 days, about 8 days to about 14 days, about 8 days to about 13 days, about 8 days to about 12 days, about 8 days to about 11 days, about 8 days to about 10 days, about 8 days to about 9 days, about 9 days to about 15 days, about 9 days to about 14 days, about 9 days to about 13 days, about 9 days to about 12 days, about 9 days to about 11 days, about 9 days to about 10 days, about 10 days to about 15 days, about 10 days to about 14 days, about 10 days to about 13 days, about 10 days to about 12 days, about 10 days to about 11 days, about 11 days to about 15 days, about 11 days to about 14 days, about 11 days to about 13 days, about 11 days to about 12 days, about 12 days to about 15 days, about 12 days to about 14 days, about 12 days to about 13 days, about 13 days to about 15 days, about 13 days to about 14 days, or about 14 days to about 15 days.

In some embodiments, step (d) results in a cell density of about 5×10⁶ enucleated erythroid cells/mL to about 2×10⁸ enucleated erythroid cells/mL (or any of the subranges about 5×10⁶ enucleated erythroid cells/mL to about 1×10⁸ enucleated erythoid cells/mL, about 5×10⁶ enucleated erythroid cells/mL to about 8×10⁷ enucleated erythroid cells/mL, about 5×10⁶ enucleated erythroid cells/mL to about 6×10⁷ enucleated erythroid cells/mL, about 5×10⁶ enucleated erythroid cells/mL to about 4×10⁷ enucleated erythroid cells/mL, about 5×10⁶ enucleated erythroid cells/mL to about 2×10⁷ enucleated erythroid cells/mL, about 5×10⁶ enucleated erythroid cells/mL to about 1×10⁷ enucleated erythroid cells/mL, about 5×10⁶ enucleated erythroid cells/mL to about 8×10⁶ enucleated erythroid cells/mL, about 5×10⁶ enucleated erythroid cells/mL to about 6×10⁶ enucleated erythroid cells/mL, about 6×10⁶ enucleated erythroid cells/mL to about 1×10⁸ enucleated erythoid cells/mL, about 6×10⁶ enucleated erythroid cells/mL to about 8×10⁷ enucleated erythroid cells/mL, about 6×10⁶ enucleated erythroid cells/mL to about 6×10⁷ enucleated erythroid cells/mL, about 6×10⁶ enucleated erythroid cells/mL to about 4×10⁷ enucleated erythroid cells/mL, about 6×10⁶ enucleated erythroid cells/mL to about 2×10⁷ enucleated erythroid cells/mL, about 6×10⁶ enucleated erythroid cells/mL to about 1×10⁷ enucleated erythroid cells/mL, about 6×10⁶ enucleated erythroid cells/mL to about 8×10⁶ enucleated erythroid cells/mL, about 8×10⁶ enucleated erythroid cells/mL to about 1×10⁸ enucleated erythoid cells/mL, about 8×10⁶ enucleated erythroid cells/mL to about 8×10⁷ enucleated erythroid cells/mL, about 8×10⁶ enucleated erythroid cells/mL to about 6×10⁷ enucleated erythroid cells/mL, about 8×10⁶ enucleated erythroid cells/mL to about 4×10⁷ enucleated erythroid cells/mL, about 8×10⁶ enucleated erythroid cells/mL to about 2×10⁷ enucleated erythroid cells/mL, about 8×10⁶ enucleated erythroid cells/mL to about 1×10⁷ enucleated erythroid cells/mL, about 1×10⁷ enucleated erythroid cells/mL to about 1×10⁸ enucleated erythoid cells/mL, about 1×10⁷ enucleated erythroid cells/mL to about 8×10⁷ enucleated erythroid cells/mL, about 1×10⁷ enucleated erythroid cells/mL to about 6×10⁷ enucleated erythroid cells/mL, about 1×10⁷ enucleated erythroid cells/mL to about 4×10⁷ enucleated erythroid cells/mL, about 1×10⁷ enucleated erythroid cells/mL to about 2×10⁷ enucleated erythroid cells/mL, about 2×10⁷ enucleated erythroid cells/mL to about 1×10⁸ enucleated erythoid cells/mL, about 2×10⁷ enucleated erythroid cells/mL to about 8×10⁷ enucleated erythroid cells/mL, about 2×10⁷ enucleated erythroid cells/mL to about 6×10⁷ enucleated erythroid cells/mL, about 2×10⁷ enucleated erythroid cells/mL to about 4×10⁷ enucleated erythroid cells/mL, about 4×10⁷ enucleated erythroid cells/mL to about 1×10⁸ enucleated erythoid cells/mL, about 4×10⁷ enucleated erythroid cells/mL to about 8×10⁷ enucleated erythroid cells/mL, about 4×10⁷ enucleated erythroid cells/mL to about 6×10⁷ enucleated erythroid cells/mL, about 6×10⁷ enucleated erythroid cells/mL to about 1×10⁸ enucleated erythoid cells/mL, about 6×10⁷ enucleated erythroid cells/mL to about 8×10⁷ enucleated erythroid cells/mL, about 8×10⁷ enucleated erythroid cells/mL to about 1×10⁸ enucleated erythoid cells/mL.

### Enucleated Erythroid Cells

In some embodiments, the enucleated erythroid cells (e.g., human enucleated erythroid cells) described herein are negative for (i.e., do not include) one or more minor blood group antigens, e.g., Le(a⁻b⁻) (for Lewis antigen system), Fy(a⁻b⁻) (for Duffy system), Jk(a⁻b⁻) (for Kidd system), M⁻N⁻(for MNS system), K⁻k⁻(for Kell system), Lu(a⁻b⁻) (for Lutheran system), and H-antigen negative (Bombay phenotype), or any combination thereof. In some embodiments, the enucleated erythroid cells are also Type O and/or Rh⁻. Minor blood groups are described, e.g., in Agarwal et al., “Blood group phenotype frequencies in blood donors from a tertiary care hospital in north India,” Blood Res. 48(1):51-54, 2013, and Mitra et al., “Blood groups systems,” Indian J. Anaesth. 58(5):524-528, 2014, the description of which is incorporated herein by reference.

In some embodiments, the enucleated erythroid cells (e.g., human enucleated erythroid cells) described herein exhibit substantially the same osmotic membrane fragility as an isolated, uncultured enucleated erythroid cell that does not comprise an exogenous protein (e.g., any of the exogenous proteins described herein or known in the art). In some embodiments, the population of enucleated erythroid cells has an osmotic fragility of less than 50% cell lysis at 0.3%, 0.35%, 0.4%, 0.45%, or 0.5% NaCl. Osmotic fragility is determined, in some embodiments, using the method described in Example 59 of WO 2015/073587 (the description of which is incorporated herein by reference).

In some embodiments, the enucleated erythroid cells (e.g., human enucleated erythroid cells) have approximately the same diameter or volume as a wild-type, untreated enucleated erythroid cell. In some embodiments, the population of enucleated erythroid cells (e.g., human enucleated erythroid cells) have an average diameter of about 4, 5, 6, 7, 8, 9, 10, 11 or 12 microns, or about 4.0 to about 12.0 microns, about 4.0 to about 11.5 microns, about 4.0 to about 11.0 microns, about 4.0 to about 10.5 microns, about 4.0 to about 10 microns, about 4.0 to about 9.5 microns, about 4.0 to about 9.0 microns, about 4.0 to about 8.5 microns, about 4.0 to about 8.0 microns, about 4.0 to about 7.5 microns, about 4.0 to about 7.0 microns, about 4.0 to about 6.5 microns, about 4.0 to about 6.0 microns, about 4.0 to about 5.5 microns, about 4.0 to about 5.0 microns, about 4.0 to about 4.5 microns, about 4.5 to about 12.0 microns, about 4.5 to about 11.5 microns, about 4.5 to about 11.0 microns, about 4.5 to about 10.5 microns, about 4.5 to about 10.0 microns, about 4.5 to about 9.5 microns, about 4.5 to about 9.0 microns, about 4.5 to about 8.5 microns, about 4.5 to about 8.0 microns, about 4.5 to about 7.5 microns, about 4.5 to about 7.0 microns, about 4.5 to about 6.5 microns, about 4.5 to about 6.0 microns, about 4.5 to about 5.5 microns, about 4.5 to about 5.0 microns, about 5.0 to about 12.0 microns, about 5.0 to about 11.5 microns, about 5.0 to about 11.0 microns, about 5.0 to about 10.5 microns, about 5.0 to about 10.0 microns, about 5.0 to about 9.5 microns, about 5.0 to about 9.0 microns, about 5.0 to about 8.5 microns, about 5.0 to about 8.0 microns, about 5.0 to about 7.5 microns, about 5.0 to about 7.0 microns, about 5.0 to about 6.5 microns, about 5.0 to about 6.0 microns, about 5.0 to about 5.5 microns, about 5.5 to about 12.0 microns, about 5.5 to about 11.5 microns, about 5.5 to about 11.0 microns, about 5.5 to about 10.5 microns, about 5.5 to about 10.0 microns, about 5.5 to about 9.5 microns, about 5.5 to about 9.0 microns, about 5.5 to about 8.5 microns, about 5.5 to about 8.0 microns, about 5.5 to about 7.5 microns, about 5.5 to about 7.0 microns, about 5.5 to about 6.5 microns, about 5.5 to about 6.0 microns, about 6.0 to about 12.0 microns, about 6.0 to about 11.5 microns, about 6.0 to about 11.0 microns, about 6.0 to about 10.5 microns, about 6.0 to about 10.0 microns, about 6.0 to about 9.5 microns, about 6.0 to about 9.0 microns, about 6.0 to about 8.5 microns, about 6.0 to about 8.0 microns, about 6.0 to about 7.5 microns, about 6.0 to about 7.0 microns, about 6.0 to about 6.5 microns, about 6.5 to about 12.0 microns, about 6.5 to about 11.5 microns, about 6.5 to about 11.0 microns, about 6.5 to about 10.5 microns, about 6.5 to about 10.0 microns, about 6.5 to about 9.5 microns, about 6.5 to about 9.0 microns, about 6.5 to about 8.5 microns, about 6.5 to about 8.0 microns, about 6.5 to about 7.5 microns, about 6.5 to about 7.0 microns, about 7.0 to about 12.0 microns, about 7.0 to about 11.5 microns, about 7.0 to about 11.0 microns, about 7.0 to about 10.5 microns, about 7.0 to about 10.0 microns, about 7.0 to about 9.5 microns, about 7.0 to about 9.0 microns, about 7.0 to about 8.5 microns, about 7.0 to about 8.0 microns, about 7.0 to about 7.5 microns, about 7.5 to about 12.0 microns, about 7.5 to about 11.5 microns, about 7.5 to about 11.0 microns, about 7.5 to about 10.5 microns, about 7.5 to about 10.0 microns, about 7.5 to about 9.5 microns, about 7.5 to about 9.0 microns, about 7.5 to about 8.5 microns, about 7.5 to about 8.0 microns, about 8.0 to about 12.0 microns, about 8.0 to about 11.5 microns, about 8.0 to about 11.0 microns, about 8.0 to about 10.5 microns, about 8.0 to about 10.0 microns, about 8.0 to about 9.5 microns, about 8.0 to about 9.0 microns, about 8.0 to about 8.5 microns, about 8.5 to about 12.0 microns, about 8.5 to about 11.5 microns, about 8.5 to about 11.0 microns, about 8.5 to about 10.5 microns, about 8.5 to about 10.0 microns, about 8.5 to about 9.5 microns, about 8.5 to about 9.0 microns, about 9.0 to about 12.0 microns, about 9.0 to about 11.5 microns, about 9.0 to about 11.0 microns, about 9.0 to about 10.5 microns, about 9.0 to about 10.0 microns, about 9.0 to about 9.5 microns, about 9.5 to about 12.0 microns, about 9.5 to about 11.5 microns, about 9.5 to about 11.0 microns, about 9.5 to about 10.5 microns, about 9.5 to about 10.0 microns, about 10.0 to about 12.0 microns, about 10.0 to about 11.5 microns, about 10.0 to about 11.0 microns, about 10.0 to about 10.5 microns, about 10.5 to about 12.0 microns, about 10.5 to about 11.5 microns, about 10.5 to about 11.0 microns, about 11.0 to about 12.0 microns, about 11.0 to about 11.5 microns, or about 11.5 to about 12.0 microns, and optionally the standard deviation of the population is less than 1, 2, or 3 microns. Enucleated erythroid cell diameter can be measured, e.g., using an Advia 120 hematology system, a Vi-cell ™ Cell Viability Analyzer (Beckman Coulter), or a Moxi Z cell counter (Orflo).

In some embodiments, the volume of the mean corpuscular volume of the enucleated erythroid cell is about 10 fL to about 175 fL, about 10 fL to about 160 fL, about 10 fL to about 140 fL, about 10 fL to about 120 fL, about 10 fL to about 100 fL, about 10 fL to about 95 fL, about 10 fL to about 90 fL, about 10 fL to about 85 fL, about 10 fL to about 80 fL, about 10 fL to about 75 fL, about 10 fL to about 70 fL, about 10 fL to about 65 fL, about 10 fL to about 60 fL, about 10 fL to about 55 fL, about 10 fL to about 50 fL, about 10 fL to about 45 fL, about 10 fL to about 40 fL, about 10 fL to about 35 fL, about 10 fL to about 30 fL, about 10 fL to about 25 fL, about 10 fL to about 20 fL, about 10 fL to about 15 fL, about 15 fL to about 175 fL, about 15 fL to about 160 fL, about 15 fL to about 140 fL, about 15 fL to about 120 fL, about 15 fL to about 100 fL, about 15 fL to about 95 fL, about 15 fL to about 90 fL, about 15 fL to about 85 fL, about 15 fL to about 80 fL, about 15 fL to about 75 fL, about 15 fL to about 70 fL, about 15 fL to about 65 fL, about 15 fL to about 60 fL, about 15 fL to about 55 fL, about 15 fL to about 50 fL, about 15 fL to about 45 fL, about 15 fL to about 40 fL, about 15 fL to about 35 fL, about 15 fL to about 30 fL, about 15 fL to about 25 fL, about 15 fL to about 20 fL, about 20 fL to about 175 fL, about 20 fL to about 160 fL, about 20 fL to about 140 fL, about 20 fL to about 120 fL, about 20 fL to about 100 fL, about 20 fL to about 95 fL, about 20 fL to about 90 fL, about 20 fL to about 85 fL, about 20 fL to about 80 fL, about 20 fL to about 75 fL, about 20 fL to about 70 fL, about 20 fL to about 65 fL, about 20 fL to about 60 fL, about 20 fL to about 55 fL, about 20 fL to about 50 fL, about 20 fL to about 45 fL, about 20 fL to about 40 fL, about 20 fL to about 35 fL, about 20 fL to about 30 fL, about 20 fL to about 25 fL, about 25 fL to about 175 fL, about 25 fL to about 160 fL, about 25 fL to about 140 fL, about 25 fL to about 120 fL, about 25 fL to about 100 fL, about 25 fL to about 95 fL, about 25 fL to about 90 fL, about 25 fL to about 85 fL, about 25 fL to about 80 fL, about 25 fL to about 75 fL, about 25 fL to about 70 fL, about 25 fL to about 65 fL, about 25 fL to about 60 fL, about 25 fL to about 55 fL, about 25 fL to about 50 fL, about 25 fL to about 45 fL, about 25 fL to about 40 fL, about 25 fL to about 35 fL, about 25 fL to about 30 fL, about 30 fL to about 175 fL, about 30 fL to about 160 fL, about 30 fL to about 140 fL, about 30 fL to about 120 fL, about 30 fL to about 100 fL, about 30 fL to about 95 fL, about 30 fL to about 90 fL, about 30 fL to about 85 fL, about 30 fL to about 80 fL, about 30 fL to about 75 fL, about 30 fL to about 70 fL, about 30 fL to about 65 fL, about 30 fL to about 60 fL, about 30 fL to about 55 fL, about 30 fL to about 50 fL, about 30 fL to about 45 fL, about 30 fL to about 40 fL, about 30 fL to about 35 fL, about 35 fL to about 175 fL, about 35 fL to about 160 fL, about 35 fL to about 140 fL, about 35 fL to about 120 fL, about 35 fL to about 100 fL, about 35 fL to about 95 fL, about 35 fL to about 90 fL, about 35 fL to about 85 fL, about 35 fL to about 80 fL, about 35 fL to about 75 fL, about 35 fL to about 70 fL, about 35 fL to about 65 fL, about 35 fL to about 60 fL, about 35 fL to about 55 fL, about 35 fL to about 50 fL, about 35 fL to about 45 fL, about 35 fL to about 40 fL, about 40 fL to about 175 fL, about 40 fL to about 160 fL, about 40 fL to about 140 fL, about 40 fL to about 120 fL, about 40 fL to about 100 fL, about 40 fL to about 95 fL, about 40 fL to about 90 fL, about 40 fL to about 85 fL, about 40 fL to about 80 fL, about 40 fL to about 75 fL, about 40 fL to about 70 fL, about 40 fL to about 65 fL, about 40 fL to about 60 fL, about 40 fL to about 55 fL, about 40 fL to about 50 fL, about 40 fL to about 45 fL, about 45 fL to about 175 fL, about 45 fL to about 160 fL, about 45 fL to about 140 fL, about 45 fL to about 120 fL, about 45 fL to about 100 fL, about 45 fL to about 95 fL, about 45 fL to about 90 fL, about 45 fL to about 85 fL, about 45 fL to about 80 fL, about 45 fL to about 75 fL, about 45 fL to about 70 fL, about 45 fL to about 65 fL, about 45 fL to about 60 fL, about 45 fL to about 55 fL, about 45 fL to about 50 fL, about 50 fL to about 175 fL, about 50 fL to about 160 fL, about 50 fL to about 140 fL, about 50 fL to about 120 fL, about 50 fL to about 100 fL, about 50 fL to about 95 fL, about 50 fL to about 90 fL, about 50 fL to about 85 fL, about 50 fL to about 80 fL, about 50 fL to about 75 fL, about 50 fL to about 70 fL, about 50 fL to about 65 fL, about 50 fL to about 60 fL, about 50 fL to about 55 fL, about 60 fL to about 175 fL, about 60 fL to about 160 fL, about 60 fL to about 140 fL, about 60 fL to about 120 fL, about 60 fL to about 100 fL, about 60 fL to about 95 fL, about 60 fL to about 90 fL, about 60 fL to about 85 fL, about 60 fL to about 80 fL, about 60 fL to about 75 fL, about 60 fL to about 70 fL, about 60 fL to about 65 fL, about 70 fL to about 175 fL, about 70 fL to about 160 fL, about 70 fL to about 140 fL, about 70 fL to about 120 fL, about 70 fL to about 100 fL, about 70 fL to about 95 fL, about 70 fL to about 90 fL, about 70 fL to about 85 fL, about 70 fL to about 80 fL, about 70 fL to about 75 fL, about 80 fL to about 175 fL, about 80 fL to about 160 fL, about 80 fL to about 140 fL, about 80 fL to about 120 fL, about 80 fL to about 100 fL, about 80 fL to about 95 fL, about 80 fL to about 90 fL, about 80 fL to about 85 fL, about 100 fL to about 175 fL, about 100 fL to about 160 fL, about 100 fL to about 140 fL, about 100 fL to about 120 fL, about 120 fL to about 175 fL, about 120 fL to about 160 fL, about 120 fL to about 140 fL, about 140 fL to about 175 fL, about 140 fL to about 160 fL, or about 160 fL to about 175 fL, and optionally, the standard deviation of the population is less than 50, 40, 30, 20, 10, 5, or 2 fL. The mean corpuscular volume can be measured, e.g., using a hematological analysis instrument, e.g., a Coulter counter, a Moxi Z cell counter (Orflo), or a Sysmex Hematology analyzer.

In some embodiments, the enucleated erythroid cells are human (e.g., derived from a human donor erythroid progenitor cell) enucleated erythroid cells.

In some embodiments, the enucleated erythroid cells are engineered human enucleated erythroid cells. In some examples, the engineered enucleated erythroid cells comprise a single exogenous protein (e.g., an exogenous protein present in the cytosol or present on the membrane of the engineered enucleated erythroid cell) (e.g., any of the exemplary exogenous proteins described herein or known in the art).

In other examples, the engineered enucleated erythroid cells comprise two or more exogenous proteins (e.g., any of the exemplary exogenous proteins described herein). In some examples, at least one of the two or more exogenous proteins can be present in the cytosol of the engineered enucleated erythroid cell (e.g., an enzyme, e.g., phenylalanine ammonia lyase). In some examples, at least one of the two or more exogenous proteins can be present on the membrane of the engineered enucleated erythroid cell (e.g., an Fc-binding molecule, a cytokine receptor, T-cell activating ligands, T-cell receptors, immune inhibitory molecules, MHC molecules, APC-binding molecules, autoantigens, allergens, toxins, targeting agents, receptor ligands (e.g., receptor agonists or receptor antagonists), or antibodies or antibody fragments).

Non-limiting examples of the one or more exogenous proteins that any of the engineered erythroid cells described herein can comprise are listed below in Tables A-D, in addition to the corresponding disease or condition that an engineered erythroid cell comprising the exogenous protein can be used to treat. Additional examples of exogenous proteins that can be comprised by any of the erythroid cells described herein are known in the art.

TABLE A
Exemplary Exogenous Proteins

| | | Disease or |
|---|---|---|
| Genus | Exogenous Protein | treatment |
| Enzymes | Phenylalanine ammonia | Phenylketonuria (PKU); method |
| | lyase (PAL) (e.g., | of reducing phenylalanine in the |
| | Anabaena variabilis | blood of a subject |
| | PAL) | |
| | Phenylalanine | Phenylketonuria (PKU); method |
| | hydroxylase (PAH) | of reducing phenylalanine in the |
| | | blood of a subject |
| | Asparaginase | Cancer |
| | Glutaminase | Cancer |
| | Cystathionine gamma | Homocystinuria; method of |
| | lyase (CGL) | reducing homocysteine levels in |
| | | the blood of a subject |
| | Uricase | Hyperuricemia, rheumatoid |
| | | arthritis, osteoarthritis, cerebral |
| | | stroke, ischemic heart disease, |
| | | arrhythmia, and chronic renal |
| | | disease |
| | Cystathionine beta | Homocystinuria; method of |
| | synthase (CBS) | reducing homocysteine levels in |
| | | the blood of a subject |
| | Oxalate oxidase (OxOx) | hyperoxaluria; method of |
| | | reducing the level of oxalate |
| | Oxalate decarboxylase | hyperoxaluria; method of |
| | (OxDC) | reducing the level of oxalate |

TABLE B
Exemplary Exogenous Proteins

| Genus | Exogenous Protein |
|---|---|
| Antigens | CD19, CD20, CD123, CD33, CD133, CD138, CD5, CD7, |
| | CD22, CD30, myelin basic protein, myelin proteolipid |
| | protein, myelin oligodendrocyte glycoprotein (MOG), |
| | phospholipase A2 receptor, beta-2 glycoprotein 1, a |
| | tumor antigen or neoantigen (e.g., a melanoma antigen |
| | genes-A (MAGE-A) antigen or a p53 peptide) |
| | an autoimmune disease antigen, a viral antigen (e.g., an |
| | Epstein barr virus (EBV) antigen, a human papilloma virus |
| | (HPV) antigen, and a hepatitis B virus (HBV) antigen), a |
| | bacterial antigen, or a parasite antigen; a neutrophil |
| | granule protease antigen, a NY-ESO-1/LAGE-2 antigen, a |
| | telomerase antigen, a glycoprotein 100 (gp100) antigen |

TABLE C
Exemplary Exogenous Proteins

| | | Disease or |
|---|---|---|
| Genus | Exogenous Protein | treatment |
| Immunomodulatory | cytokines, interleukins, cytokine | Cancer, |
| Molecules | receptors, Fc-binding | autoimmune |
| | molecules, T-cell activating | disorders |
| | ligands, T cell receptors, | |
| | immune inhibitory molecules, | |
| | costimulatory molecule, | |
| | coinhibitory molecules (e.g., | |
| | IL-35, IL-10, VSIG-3 or a LAG3 | |
| | agonist), MHC molecules, | |
| | APC-binding molecules, TRAIL | |
| | receptor ligands. | |
| | Exemplary immunomodulatory | |
| | molecules include, 4-1BBL, | |
| | LIGHT, anti CD28, CD80, CD86, | |
| | CD70, OX40L, GITRL, TIM4, | |
| | SLAM, CD48, CD58, CD83, | |
| | CD155, CD112, IL-15, IL-15Rα | |
| | fused to IL-15, IL-21, ICAM-1, a | |
| | ligand for LFA-1, anti-CD3, IL2, | |
| | IL15, 15Rα fused to IL-15, IL7, | |
| | IL12, IL18, IL21, IL4, IL6, IL23, | |
| | IL27, IL17, IL10, TGF-beta, | |
| | IFN-gamma, IL-1 beta, GM-CSF, | |
| | andIL-25. | |
| | Exemplary combination: IL-15Rα | |
| | fused to IL-15 and 4-1BBL | |
| | Exemplary Combination: IL-12 | |
| | and 4-1BBL | |

TABLE D
Exemplary Exogenous Proteins

| | |
|---|---|
| Antigen Presenting | an MHC class I polypeptide, an MHC class I |
| Molecule | single chain fusion protein, an MHC class II |
| | polypeptide, or an MHC class II single chain |
| | fusion protein |
| | Either unbound or bound (e.g., covalently or as |
| | a fusion protein) to an antigen |
| preproinsulin, proinsulin, | Diabetes |
| insulin | |

In some embodiments, an exogenous protein present on the membrane of the engineered enucleated erythroid cell can be a product of a click chemistry reaction (e.g., the exogenous protein may be conjugated to a protein present on the membrane of the cell (e.g., a second exogenous protein or an endogenous protein) using any of the methods described herein). In some embodiments, an exogenous protein present on the membrane of the engineered enucleated erythroid cell can the a product of a conjugation reaction using a sortase enzyme (e.g., the exogenous protein may be conjugated to a protein present on the membrane of the cell (e.g., a second exogenous protein or an endogenous protein) using any of the methods described herein). Non-limiting examples of a conjugation reaction using a sortase enzyme can be found in U.S. Pat. No. 10,260,038 and U.S. Pat. Pub. No. 2016/0082046 A1, both of which are incorporated by reference.

Some embodiments of any of the methods described herein further include click-conjugating one or more exogenous proteins to the cells (e.g., any of the cells described herein, e.g., an enucleated erythroid cell, an engineered enucleated erythroid cell, or any of the progenitor erythroid cells described herein) (e.g., using any of the methods described herein). Some embodiments of any of the methods described herein further includes hypotonically loading the cells (e.g., any of the cells described herein, e.g., an enucleated erythroid cell, an engineered enucleated erythroid cell, or any of the progenitor erythroid cells described herein) (e.g., using any of the methods described herein). Some embodiments of any of the methods described herein further include loading the cells (e.g., any of the cells described herein, e.g., an enucleated erythroid cell, an engineered enucleated erythroid cell, or any of the progenitor erythroid cells described herein) via physical manipulation (e.g., using any the methods described herein).

In some embodiments, an exogenous protein present on the membrane of the engineered enucleated erythroid cell can be a lipid-anchored protein, e.g., a GPI-anchor, an N-myristolyated protein, or a S-palmitoylated protein. In some embodiments an exogenous protein present on the membrane of the engineered enucleated erythroid cell can be a transmembrane protein (e.g., a single-pass or multi-pass transmembrane protein) or a peripheral membrane protein. In some embodiments, an exogenous protein present on the membrane of the engineered enucleated erythroid cell can be a fusion protein comprising a transmembrane domain (e.g., a fusion protein comprising the transmembrane domain of small integral membrane protein 1 (SMIM1) or glycophorin A (GPA)). In some embodiments, an exogenous protein present on the membrane of the engineered enucleated erythroid cell does not have any amino acids protruding into the extracellular space. In some embodiments, an exogenous protein present on the membrane of the engineered enucleated erythroid cell does not have any amino acids protruding into the cytosol of the engineered enucleated erythroid cell. In some embodiments, an exogenous protein present on the membrane of the engineered enucleated erythroid cell has amino acids protruding into the extracellular space and amino acids protruding into the cytosol of the engineered enucleated erythroid cells. The methods described herein can further include introducing nucleic acid encoding one or more recombinant proteins into an erythroid progenitor cell (e.g., any of the exemplary erythroid progenitor cells described herein) prior to step (a) or during step (a) (e.g., over one to three days any time during step (a), e.g., over one to three days at the beginning of step (a)). Engineered enucleated erythroid cells can be produced by introducing one or more nucleic acids (e.g., DNA expression vectors or mRNA) encoding one or more exogenous proteins (e.g., any of the exogenous proteins described herein or known in the art) into an erythroid progenitor cell (e.g., any of the erythroid progenitor cells described herein or known in the art). Exemplary methods for introducing DNA expression vectors into erthyroid progenitor cells include, but are not limited to, liposome-mediated transfer, transformation, gene guns, transfection, and transduction, e.g., viral-mediated gene transfer (e.g., performed using viral vectors including adenovirus vectors, adeno-associated viral vectors, lentiviral vectors, herpes viral vectors, and retroviral-based vectors). Additional exemplary methods for introducing DNA expression vectors into erythroid progenitor cells include the use of, e.g., naked DNA, CaPO₄ precipitation, DEAE dextran, electroporation, protoplast fusion, lipofection, and cell microinjection.

An erythroid progenitor cell can optionally be cultured before introduction of one or more nucleic acids encoding one or more exogenous proteins, under suitable conditions allowing for differentiation into engineered enucleated erythroid cells. In some embodiments, the resulting engineered enucleated erythroid cells comprise proteins associated with mature erythrocytes, e.g., hemoglobin (e.g., adult hemoglobin and/or fetal hemoglobin), glycophorin A, and exogenous proteins which can be validated and quantified by standard methods (e.g. Western blotting or FACS analysis).

In some examples, enucleated erythroid cells or erythroid progenitor cells can be transfected with mRNA encoding an exogenous protein to generate engineered enucleated erythroid cells. Messenger RNA can be derived from in vitro transcription of a cDNA plasmid construct containing a sequence encoding an exogenous protein. For example, the cDNA sequence encoding an exogenous protein may be inserted into a cloning vector containing a promoter sequence compatible with specific RNA polymerases. For example, the cloning vector ZAP Express® pBK-CMV (Stratagene, La Jolla, Calif., USA) contains T3 and T7 promoter sequences compatible with the T3 and T7 RNA polymerases, respectively. For in vitro transcription of sense mRNA, the plasmid is linearized at a restriction site downstream of the stop codon(s) corresponding to the end of the sequence encoding the exogenous protein. The mRNA is transcribed from the linear DNA template using a commercially available kit such as, for example, the RNAMaxx® High Yield Transcription Kit (from Stratagene, La Jolla, Calif., USA). In some instances, it may be desirable to generate 5′-m7GpppG-capped mRNA. As such, transcription of a linearized cDNA template may be carried out using, for example, the mMESSAGE mMACHINE High Yield Capped RNA Transcription Kit from Ambion (Austin, Tex., USA). Transcription may be carried out in a reaction volume of 20-100 μl at 37° C. for 30 min to 4 h. The transcribed mRNA is purified from the reaction mix by a brief treatment with DNase Ito eliminate the linearized DNA template followed by precipitation in 70% ethanol in the presence of lithium chloride, sodium acetate, or ammonium acetate. The integrity of the transcribed mRNA may be assessed using electrophoresis with an agarose-formaldehyde gel or commercially available Novex pre-cast TBE gels (Nove×, Invitrogen, Carlsbad, Calif., USA).

Messenger RNA encoding an exogenous protein may be introduced into enucleated erythroid cells or erythroid progenitor cells using a variety of approaches including, for example, lipofection and electroporation (van Tandeloo et al., Blood 98:49-56, 2001). For lipofection, for example, 5 μg of in vitro transcribed mRNA in Opti-MEM (Invitrogen, Carlsbad, Calif., USA) is incubated for 5-15 min at a 1:4 ratio with the cationic lipid DMRIE-C (Invitrogen).

Alternatively, a variety of other cationic lipids or cationic polymers may be used to transfect erythroid progenitor cells or enucleated erythroid cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; See, e.g., Bettinger et al., Nucleic Acids Res. 29:3882-3891, 2001). The resulting mRNA/lipid complexes are incubated with cells (1-2×10⁶ cells/mL) for 2 hours at 37° C., washed, and returned to culture. For electroporation, for example, about 5 to 20×10⁶ cells in 500 μL of Opti-MEM (Invitrogen, Carlsbad, Calif., USA) are mixed with about 20 μg of in vitro transcribed mRNA and electroporated in a 0.4-cm cuvette using, for example, an Easyject Plus device (EquiBio, Kent, United Kingdom). In some instances, it may be necessary to test various voltages, capacitances, and electroporation volumes to determine the useful conditions for transfection of a particular mRNA into an erythroid progenitor cell. In general, the electroporation parameters required to efficiently transfect cells with mRNA appear to be less detrimental to cells than those required for electroporation of DNA (van Tandeloo et al., Blood 98:49-56, 2001).

Alternatively, mRNA may be transfected into an erythroid progenitor cell or enucleated erythroid cell using a peptide-mediated RNA delivery strategy (See, e.g., Bettinger et al., Nucleic Acids Res. 29:3882-3891, 2001). For example, the cationic lipid polyethylenimine 2 kDA (Sigma-Aldrich, Saint Louis, Mo., USA) may be combined with the melittin peptide (Alta Biosciences, Birmingham, UK) to increase the efficiency of mRNA transfection, particularly in postmitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 min with the mellitin-PEI to form an RNA/peptide/lipid complex. This complex is then added to cells in serum-free culture medium for 2 to 4 h at 37° C. in a 5% CO₂ humidified environment, then removed, and the transfected cells further cultured.

In some embodiments, the engineered enucleated erythroid cells are generated by introducing a nucleic acid (e.g., any of the exemplary nucleic acids described herein) encoding one or more exogenous protein(s) (e.g., any exogenous protein or any combination of exogenous proteins described herein) into an erythroid progenitor cell. In some embodiments, the exogenous protein is encoded by a DNA, which is introduced into an erythroid progenitor cell. In some embodiments, the exogenous protein is encoded by an RNA, which is introduced into an erythroid progenitor cell.

Nucleic acid encoding one or more exogenous protein(s) may be introduced into an erythroid progenitor cell prior to terminal differentiation into an enucleated erythroid cell using a variety of DNA techniques, including, e.g., transient or stable transfections and gene therapy approaches.

Viral gene transfer may be used to transfect the cells with a nucleic acid encoding one or more exogenous protein(s). A number of viruses may be used as gene transfer vehicles including Moloney murine leukemia virus (MMLV), adenovirus, adeno-associated virus (AAV), herpes simplex virus (HSV), lentiviruses such as human immunodeficiency virus 1 (HIV1), and spumaviruses such as foamy viruses (see, e.g., Osten et al., HEP 178:177-202, 2007). Retroviruses, for example, efficiently transduce mammalian cells including human cells and integrate into chromosomes, conferring stable gene transfer.

A nucleic acid encoding one or more exogenous protein(s) can be transfected into an erythroid progenitor cell. A suitable vector is the Moloney murine leukemia virus (MMLV) vector (Malik et al., Blood 91:2664-2671, 1998). Vectors based on MMLV, an oncogenic retrovirus, are currently used in gene therapy clinical trials (Hassle et al., News Physiol. Sci. 17:87-92, 2002). For example, a DNA construct containing the cDNA encoding an exogenous protein can be generated in the MMLV vector backbone using standard molecular biology techniques. The construct is transfected into a packaging cell line such as, for example, PA317 cells and the viral supernatant is used to transfect producer cells such as, for example, PG13 cells. The PG13 viral supernatant is incubated with an erythroid progenitor cell. The expression of the exogenous protein may be monitored using FACS analysis (fluorescence-activated cell sorting), for example, with a fluorescently labeled antibody directed against the exogenous protein, if it is present on the membrane of the engineered human enucleated erythroid cell. Similar methods may be used such that an exogenous protein is present in the cytosol of an engineered human enucleated erythroid cell.

Optionally, a nucleic acid encoding a fluorescent tracking molecule such as, for example, green fluorescent protein (GFP), can be transfected into an erythroid progenitor cell using a viral-based approach (Tao et al., Stem Cells 25:670-678, 2007). Ecotopic retroviral vectors containing DNA encoding the enhanced green fluorescent protein (EGFP) or a red fluorescent protein (e.g., DsRed-Express) are packaged using a packaging cell such as, for example, the Phoenix-Eco cell line (distributed by Orbigen, San Diego, Calif.). Packaging cell lines stably express viral proteins needed for proper viral packaging including, for example, gag, pol, and env. Supernatants from the Phoenix-Eco cells into which viral particles have been shed are used to transduce erythroid progenitor cells. In some instances, transduction may be performed on a specially coated surface such as, for example, fragments of recombinant fibronectin to improve the efficiency of retroviral mediated gene transfer (e.g., RetroNectin, Takara Bio USA, Madison, Wis.). Cells are incubated in RetroNectin-coated plates with retroviral Phoenix-Eco supernatants plus suitable co-factors. Transduction may be repeated the next day. In this instance, the percentage of erythroid progenitor cells expressing EGFP or DsRed-Express may be assessed by FACS. Other reporter genes that may be used to assess transduction efficiency include, for example, beta-galactosidase, chloramphenicol acetyltransferase, and luciferase, as well as low-affinity nerve growth factor receptor (LNGFR), and the human cell surface CD24 antigen (Bierhuizen et al., Leukemia 13:605-613, 1999).

Nonviral vectors may be used to introduce a nucleic acid encoding one or more exogenous protein(s) into an erythroid progenitor cell to generate engineered enucleated erythroid cells. A number of delivery methods can be used to introduce nonviral vectors into erythroid progenitor cells including chemical and physical methods.

A nonviral vector encoding an exogenous protein may be introduced into an erythroid progenitor cell using synthetic macromolecules, such as cationic lipids and polymers (Papapetrou et al., Gene Therapy 12:S118-S130, 2005). Cationic liposomes, for example form complexes with DNA through charge interactions. The positively charged DNA/lipid complexes bind to the negative cell surface and are taken up by the cell by endocytosis. This approach may be used, for example, to transfect hematopoietic cells (see, e.g., Keller et al., Gene Therapy 6:931-938, 1999). For erythroid progenitor cells, the plasmid DNA (in a serum-free medium, such as, for example, OptiMEM (Invitrogen, Carlsbad, Calif.)) is mixed with a cationic liposome (in serum free medium), such as the commercially available transfection reagent Lipofectamine™ (Invitrogen, Carlsbad, Calif.), and allowed to incubate for at least 20 minutes to form complexes. The DNA/liposome complex is added to erythroid progenitor cells and allowed to incubate for 5-24 h, after which time transgene expression of the exogenous protein(s) may be assayed. Alternatively, other commercially available liposome tranfection agents may be used (e.g., In vivo GeneSHUTTLE™, Qbiogene, Carlsbad, Calif.).

Optionally, a cationic polymer such as, for example, polyethylenimine (PEI) may be used to efficiently transfect erythroid progenitor cells, for example hematopoietic and umbilical cord blood-derived CD34⁺ cells (see, e.g., Shin et al., Biochim. Biophys. Acta 1725:377-384, 2005). Human CD34⁺ cells are isolated from human umbilical cord blood and cultured in Iscove's modified Dulbecco's medium supplemented with 200 ng/ml stem cell factor and 20% heat-inactivated serum. Plasmid DNA encoding the exogenous protein(s) is incubated with branched or linear PEIs varying in size from 0.8 K to 750 K (Sigma Aldrich, Saint Louis, Mo., USA; Fermetas, Hanover, Md., USA). PEI is prepared as a stock solution at 4.2 mg/mL distilled water and slightly acidified to pH 5.0 using HCl. The DNA may be combined with the PEI for 30 min at room temperature at various nitrogen/phosphate ratios based on the calculation that 1 μg of DNA contains 3 nmol phosphate and 1 μL of PEI stock solution contains 10 nmol amine nitrogen. The isolated CD34⁺ cells are seeded with the DNA/cationic complex, centrifuged at 280×g for 5 minutes and incubated in culture medium for 4 or more hours until expression of the exogenous protein(s) is/are assessed.

A plasmid vector may be introduced into suitable erythroid progenitor cells using a physical method such as particle-mediated transfection, “gene gun,” biolistics, or particle bombardment technology (Papapetrou, et al., Gene Therapy 12:S118-S130, 2005). In this instance, DNA encoding the exogenous protein is absorbed onto gold particles and administered to cells by a particle gun. This approach may be used, for example, to transfect erythroid progenitor cells, e.g., hematopoietic stem cells derived from umbilical cord blood (see, e.g., Verma et al., Gene Therapy 5:692-699, 1998). As such, umbilical cord blood is isolated and diluted three-fold in phosphate buffered saline. CD34⁺ cells are purified using an anti-CD34 monoclonal antibody in combination with magnetic microbeads coated with a secondary antibody and a magnetic isolation system (e.g., Miltenyi MiniMac System, Auburn, Calif., USA). The CD34⁺ enriched cells may be cultured as described herein. For transfection, plasmid DNA encoding the exogenous protein(s) is precipitated onto a particle, e.g., gold beads, by treatment with calcium chloride and spermidine. Following washing of the DNA-coated beads with ethanol, the beads may be delivered into the cultured cells using, for example, a Biolistic PDS-1000/He System (Bio-Rad, Hercules, Calif., USA). A reporter gene such as, for example, beta-galactosidase, chloramphenicol acetyltransferase, luciferase, or green fluorescent protein may be used to assess efficiency of transfection.

Optionally, electroporation methods may be used to introduce a plasmid vector into erythroid progenitor cells. Electroporation creates transient pores in the cell membrane, allowing for the introduction of various molecules into the cells including, for example, DNA and RNA. As such, CD34⁺ cells are isolated and cultured as described herein. Immediately prior to electroporation, the cells are isolated by centrifugation for 10 min at 250×g at room temperature and resuspended at 0.2 −10×10⁶ viable cells/ml in an electroporation buffer such as, for example, X-VIVO 10 supplemented with 1.0% human serum albumin (HSA). The plasmid DNA (1-50 μg) is added to an appropriate electroporation cuvette along with 500 μL of cell suspension.

Electroporation may be done using, for example, an ECM 600 electroporator (Genetronics, San Diego, Calif., USA) with voltages ranging from 200 V to 280 V and pulse lengths ranging from 25 to 70 milliseconds. A number of alternative electroporation instruments are commercially available and may be used for this purpose (e.g., Gene Pulser Xcell™, BioRad, Hercules, Calif.; Cellject Duo, Thermo Science, Milford, Mass.). Alternatively, efficient electroporation of isolated CD34⁺ cells may be performed using the following parameters: 4 mm cuvette, 1600 μE, 550 V/cm, and 10 μg of DNA per 500 μL of cells at 1×10⁵ cells/mL (Oldak et al., Acta Biochim. Polonica 49:625-632, 2002).

Nucleofection, a form of electroporation, may also be used to transfect erythroid progenitor cells. In this instance, transfection is performed using electrical parameters in cell-type specific solutions that enable DNA (or other reagents) to be directly transported to the nucleus, thus reducing the risk of possible degradation in the cytoplasm. For example, a Human CD34 Cell Nucleofector™ Kit (from Amaxa Inc.) may be used to transfect erythroid progenitor cells. In this instance, 1−5×10⁶ cells in Human CD34 Cell Nucleofector™ Solution are mixed with 1-5 μg of DNA and transfected in the Nucleofector™ instrument using preprogrammed settings as determined by the manufacturer.

Erythroid progenitor cells may be non-virally transfected with a conventional expression vector which is unable to self-replicate in mammalian cells unless it is integrated in the genome. Alternatively, erythroid progenitor cells may be transfected with an episomal vector which may persist in the host nucleus as autonomously replicating genetic units without integration into chromosomes (Papapetrou et al., Gene Therapy 12:S118-S130, 2005). These vectors exploit genetic elements derived from viruses that are normally extrachromosomally replicating in cells upon latent infection such as, for example, EBV, human polyomavirus BK, bovine papilloma virus-1 (BPV-1), herpes simplex virus-1 (HSV), and Simian virus 40 (SV40). Mammalian artificial chromosomes may also be used for nonviral gene transfer (Vanderbyl et al., Exp. Hematol. 33:1470-1476, 2005).

Exogenous nucleic acid encoding one or more exogenous protein(s) can be assembled into expression vectors by standard molecular biology methods known in the art, e.g., restriction digestion, overlap-extension PCR, and Gibson assembly.

Exogenous nucleic acids can comprise a gene encoding an exogenous protein that is not normally present on the cell surface, e.g., of an enucleated erythroid cell, fused to a gene that encodes an endogenous or native membrane protein, such that the exogenous protein is expressed on the cell surface. For example, an exogenous gene encoding an exogenous protein can be cloned at the N terminus following the leader sequence of a type 1 membrane protein, at the C terminus of a type 2 membrane protein, or upstream of the GPI attachment site of a GPI-linked membrane protein.

Standard cloning methods can be used to introduce flexible amino acid linkers between two fused genes. For example, the flexible linker is a poly-glycine poly-serine linker such as [Gly₄Ser]₃ (SEQ ID NO: 3) commonly used in generating single-chain antibody fragments from full-length antibodies (Antibody Engineering: Methods & Protocols, B. Lo, ed., Humana Press, 2004, 576 pp.), or Ala-Gly-Ser-Thr (SEQ ID NO: 4) polypeptides such as those used to generate single-chain Arc repressors (Robinson & Sauer, Proc. Nat'l. Acad. Sci. USA 95:5929-34, 1998). In some embodiments, the flexible linker provides the exogenous protein with more flexibility and steric freedom than the equivalent construct without the flexible linker. This added flexibility is useful in applications that require binding to a target, e.g., an antibody or protein, or an enzymatic reaction of the protein for which the active site must be accessible to the substrate (e.g., the target).

In some embodiments, the methods provided include the delivery of large nucleic acids (specifically RNAs, such as mRNA) into erythroid progenitor cells by contacting the erythroid progenitor cell with the nucleic acid and introducing the nucleic acid by electroporation under conditions effective for delivery of the nucleic acid to the cell, such as those described herein. Suitable electroporators include, but are not limited to, the Bio-Rad GENE PULSER and GENE PULSER II; the Life Technologies NEON; BTX GEMINI system; and MAXCYTE electroporator. These methods do not require viral delivery or the use of viral vectors. Suitable nucleic acids include RNAs, such as mRNAs. Suitable nucleic acids also include DNAs, including transposable elements, stable episomes, plasmid DNA, or linear DNA.

Conditions for the electroporation of cell lines have been described in the literature, e.g. by Van Tendeloo et al., Blood 98(0:49-56, 2001. Suitable electroporation conditions for the methods described herein include for a Life Technologies Neon Transfection System: a pulse voltage ranging from about 500 to about 2000 V, from about 800 to about 1800 V, or from about 850 to about 1700 V; a pulse width ranging from about 5 to about 50 msec, or from about 10 to about 40 msec; and a pulse number ranging from 1 to 2 pulses, 1 to 3 pulses, 1 to 4 pulses, or 1 to 5 pulses.

Particularly suitable conditions for electroporation of erythroid progenitor cells include, e.g., for 4 days: a) pulse voltage 1300-1400, pulse width: 10-20 msec, number of pulses: 1-3; b) pulse voltage 1400, pulse width: 10 msec, number of pulses: 3; c) pulse voltage 1400, pulse width: 20 msec, number of pulses: 1 ; and d) pulse voltage 1300, pulse width: 10 msec, number of pulses: 3.

Particularly suitable conditions for electroporation of erythroid progenitor cells include, e.g., for 8-9 days: a) pulse voltage: 1400-1600, pulse width: 20, number of pulses: 1 ; b) pulse voltage: 1100-1300, pulse width: 30, number of pulses: 1; c) pulse voltage: 1000-1200, pulse width: 40, number of pulses: 1 ; d) pulse voltage: 1100-1400, pulse width: 20, number of pulses: 2; e) pulse voltage: 950-1150, pulse width: 30, number of pulses: 2; f) pulse voltage: 1300-1600, pulse width: 10, number of pulses: 3. These conditions generally lead to transfections efficiencies of at least about 60% or more (e.g. at least about 65%, 70%, 75%, 80%, 85%, 90%, 95% or at least about 97%, or more), and cell viability of at least about 70% or more (e.g. at least about 75%, 80%, 85%, 90%, 95% or at least about 97%, or more).

Particularly suitable conditions for electroporation of erythroid progenitor cells in culture under differentiation conditions include, e.g. for 12-13 days: a) pulse voltage: 1500-1700, pulse width: 20, number of pulses: 1; and b) pulse voltage: 1500-1600, pulse width: 10, number of pulses: 3. These conditions generally lead to transfections efficiencies of at least about 50% or more (e.g. at least about 55%, 60%, 65%, 70%, 75%, 80%, 85%, 90%, 95% or at least about 97%, or more), and cell viability of at least about 70% or more (e.g. at least about 75%, 80%, 85%, 90%, 95% or at least about 97%, or more).

The conditions disclosed herein with reference to the Life Technologies Neon system can easily be adjusted by one of ordinary skill in the art to fit a different electroporator and/or different electroporation set-ups with only routine experimentation and the specific electroporator described herein is not limiting for the methods disclosed.

In some embodiments, using the electroporation conditions described herein cultured erythroid progenitor cells are electroporated for a first time, then cultured for a desired period of time (optionally under differentiation conditions) and then re-electroporated a second time. In some embodiments, cultured erythroid progenitor cells are electroporated for a first time, then cultured for a desired period of time (optionally under differentiation conditions) and then re-electroporated a second, third, fourth, fifth, or sixth time. Optionally, the culturing period in between the first and second, the second and third, etc. electroporation can be varied. For example, the period in between electroporations may be adjusted as desired, e.g. the period may be 30 minutes, 1 hour, 6 hours, 12, hours, 18 hours, 24 hours, 30 hours, 36 hours, 48 hours, 3 days, 4 days, 5 days, 6 days, 7 days, 8 days, 9 days, 10 days, 11 days 12 days, 13 days 14 days, or 21 days. For example, erythroid progenitor cells may be electroporated on day 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, or 1 and 16. In another example, cells may be electroporated on day 2 and 3,2 and 4,2 and 5,2 and 6,2 and 7,2 and 8, 2 and 9, 2 and 10, 2 and 11, 2 and 12, 2 and 13, 2 and 14, 2 and 15, or 2 and 16. In yet another example, erythroid progenitor cells may be electroporated on day 3 and 4, 3 and 5, 3 and 6, 3 and 7, 3 and 8, 3 and 9, 3 and 10, 3 and 11, 3 and 12, 3 and 13, 3 and 14, 3 and 15, or 3 and 16. In yet another example, cells may be electroporated on day 4 and 5, 4 and 6, 4 and 7, 4 and 8, 4 and 9, 4 and 10, 4 and 11, 4 and 12, 4 and 13, 4 and 14, 4 and 15, or 4 and 16. In yet another example, cells may be electroporated on day 5 and 6, 5 and 7, 5 and 8, 5 and 9, 5 and 10, 5 and 11, 5 and 12, 5 and 13, 5 and 14, 5 and 15, or 5 and 16. In yet another example, erythroid progenitor cells may be electroporated on day 6 and 7, 6 and 8, 6 and 9, 6 and 10, 6 and 11, 6 and 12, 6 and 13, 6 and 14, 6 and 15, or 6 and 16. In yet another example, erythroid progenitor cells may be electroporated on day 7 and 8, 7 and 9, 7 and 10, 7 and 11, 7 and 12, 7 and 13, 7 and 14, 7 and 15, or 7 and 16. In yet another example, erythroid progenitor cells may be electroporated on day 8 and 9, 8 and 10, 8 and 11, 8 and 12, 8 and 13, 8 and 14, 8 and 15, or 8 and 16. In yet another example, erythroid progenitor cells may be electroporated on day 9 10, 9 and 11, 9 and 12, 9 and 13, 9 and 14, 9 and 15, or 9 and 16. In yet another example, erythroid progenitor cells may be electroporated on day 10 and 11, 10 and 12, 10 and 13, 10 and 14, 10 and 15, or 10 and 16. In yet another example, erythroid progenitor cells may be electroporated on day 11 and 12, 11 and 13, 11 and 14, 11 and 15, or 11 and 16. In yet another example, erythroid progenitor cells may be electroporated on day 12 and 13, 12 and 14, 12 and 15, or 12 and 16. In yet another example, erythroid progenitor cells may be electroporated on day 13 and 14, 13 and 15, or 13 and 16. In yet another example, erythroid progenitor cells may be electroporated on day 14 and 15, or 14 and 16. Optionally, the erythroid progenitor cells may be electroporated more than twice, e.g., three times, four times, five times, or six times and the interval may be selected as desired at any points of the differentiation process of the cells.

In some embodiments, using the electroporation conditions described herein, cultured erythroid progenitor cells are electroporated on day 1, 2, 3, 4, 5, 6, 7, 8, 9, 10, 11, 12, 13, 14, 15, or 16 of differentiation.

In some embodiments, the engineered enucleated erythroid cells can be click-conjugated engineered enucleated erythroid cells. A catalytic bond-forming polypeptide domain can be expressed on or in, e.g., an erythroid progenitor cell, present in the cytosol or present on the membrane. Many catalytic bond-forming polypeptides exist, including transpeptidases, sortases, and isopeptidases, including those derived from Spy0128, a protein isolated from Streptococcus pyogenes. It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Aspl 17 on SpyTag and Lys31 on SpyCatcher (Zakeri and Howarth, JACS 132:4526, 2010). The reaction is compatible with the cellular environment and highly specific for protein/peptide conjugation (Zakeri et al., Proc. Natl. Acad. Sci. U.S.A. 109:E690-E697, 2012). SpyTag and SpyCatcher have been shown to direct post-translational topological modification in elastin-like protein. For example, placement of SpyTag at the N-terminus and SpyCatcher at the C-terminus directs formation of circular elastin-like proteins (Zhang et al, J. Am. Chem. Soc. 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this disclsoure, when SpyTag and SpyCatcher are used, it is to be understood that the complementary molecule could be substituted in its place.

A catalytic bond-forming polypeptide, such as a SpyTag/SpyCatcher system, can be used to attach the exogenous protein to the surface of, e.g., an erythroid progenitor cell or an enucleated erythroid cell. The SpyTag polypeptide sequence can be expressed on the extracellular surface of the erytroid progenitor cell or the enucleated erythroid cell. The SpyTag polypeptide can be, for example, fused to the N terminus of a type-1 or type-3 transmembrane protein, e.g., glycophorin A, fused to the C terminus of a type-2 transmembrane protein, e.g., Kell, inserted in-frame at the extracellular terminus or in an extracellular loop of a multi-pass transmembrane protein, e.g., Band 3, fused to a GPI-acceptor polypeptide, e.g., CD55 or CD59, fused to a lipid-chain-anchored polypeptide, or fused to a peripheral membrane protein. An exogenous protein can be fused to SpyCatcher. The nucleic acid encoding the SpyCatcher fusion can be expressed and secreted from the same erythroid progenitor cell or enucleated erythroid cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous protein to the surface of the erythroid progenitor cell or the enucleated erythroid cell.

In one embodiment, the SpyTag polypeptide may be expressed as a fusion to the N terminus of glycophorin A under the control of the Gatal promoter in an erythroid cell. An exogenous protein fused to the SpyCatcher polypeptide sequence can be expressed under the control of the Gatal promoter in the same erythroid cell. Upon expression of both fusion polypeptides, an isopeptide bond will be formed between the SpyTag and SpyCatcher polypeptides, forming a covalent bond between the erythroid cell surface and the exogenous protein.

In another embodiment, the SpyTag polypeptide may be expressed as a fusion to the N terminus of glycophorin A under the control of the Gatal promoter in an erythroid progenitor cell or an enucleated erythroid cell. An exogenous protein fused to the SpyCatcher polypeptide sequence can be expressed in a suitable mammalian cell expression system, for example HEK293 cells. Upon expression of the SpyTag fusion polypeptide on the erythroid progenitor cell or enucleated erythroid cell, the SpyCatcher fusion polypeptide can be brought in contact with the cell. Under suitable reaction conditions, an isopeptide bond will be formed between the SpyTag and SpyCatcher polypeptides, forming a covalent bond between the erythroid progenitor cell surface or enucleated erythroid cell surface and the exogenous protein.

A catalytic bond-forming polypeptide, such as a SpyTag/SpyCatcher system, can be used to anchor an exogenous protein to the intracellular space of an erythroid progenitor cell or enucleated erythroid cell. The SpyTag polypeptide sequence can be expressed in the intracellular space of the erythroid progenitor cell or enucleated erythroid cell by a number of methods, including direct expression of the transgene, fusion to an endogenous intracellular protein such as, e.g., hemoglobin, fusion to the intracellular domain of endogenous cell surface proteins such as, e.g., Band 3, glycophorin A, Kell, or fusion to a structural component of the cytoskeleton. The SpyTag sequence is not limited to a polypeptide terminus and may be integrated within the interior sequence of an endogenous polypeptide such that polypeptide translation and localization is not perturbed. An exogenous protein can be fused to SpyCatcher. The nucleic acid sequence encoding the SpyCatcher fusion can be expressed within the same erythroid progenitor cell or enucleated erythroid cell that expresses the SpyTag fusion. Upon reaction of the SpyTag and SpyCatcher polypeptides, a covalent bond will be formed that acts to anchor the exogenous protein in the intracellular space of the erythroid progenitor cell or enucleated erythroid cell.

In one embodiment, an erythroid progenitor cell or an enucleated erythroid cell includes SpyTag fused to hemoglobin beta intracellularly. An erythroid progenitor cell may be genetically modified with a gene sequence that includes a hemoglobin promoter, beta globin gene, and a SpyTag sequence such that upon translation, intracellular beta globin is fused to SpyTag at is C terminus. In addition, the erythroid progenitor cell or enucleated erythroid cell expresses a Gatal promoter-led gene that codes for SpyCatcher driving expression of an exogenous polypeptide such that upon translation, the exogenous polypeptide is fused to SpyCatcher at its N terminus. Upon expression of both fusion proteins the SpyTag bound beta globin is linked through an isopeptide bond to the SpyCatcher bound exogenous polypeptide in the intracellular space, allowing the exogenous polypeptide to be anchored to beta globin and retained during maturation.

In another embodiment, the SpyTag polypeptide can be expressed as a fusion to the exogenous protein within an erythroid progenitor cell or an enucleated erythroid cell. The SpyCatcher polypeptide can be expressed as a fusion to the C terminus (intracellular) of glycophorin A within the same erythroid progenitor cell or enucleated erythroid cell. Upon expression of both fusion polypeptides, an isopeptide bond will be formed between the SpyTag and SpyCatcher polypeptides, forming a covalent bond between the membrane-anchored endogenous erythroid polypeptide and the exogenous protein.

Other molecular fusions may be formed between polypeptides and include direct or indirect conjugation. The polypeptides may be directly conjugated to each other or indirectly through a linker. The linker may be a peptide, a polymer, an aptamer, or a nucleic acid. The polymer may be, e.g., natural, synthetic, linear, or branched. Exogenous proteins can comprise a heterologous fusion protein that comprises a first polypeptide and a second polypeptide with the fusion protein comprising the polypeptides directly joined to each other or with intervening linker sequences and/or further sequences at one or both ends. The conjugation to the linker may be through covalent bonds or ionic bonds.

In some embodiments, the engineered enucleated erythroid cells are human enucleated erythroid cells that have been hypotonically loaded. For hypotonic loading/lysis, erythroid progenitor cells or enucleated erythroid cells are exposed to low ionic strength buffer, causing them to burst. The exogenous protein distributes within the cells. Enucleated erythroid cells or erythroid progenitor cells may be hypotonically lysed by adding 30-50 fold volume excess of 5 mM phosphate buffer (pH 8) to a pellet of isolated enucleated erythroid cells. The resulting lysed cell membranes are isolated by centrifugation. The pellet of lysed cell membranes is resuspended and incubated in the presence of the exogenous protein in a low ionic strength buffer, e.g., for 30 min. Alternatively, the lysed cell membranes may be incubated with the exogenous protein for as little as one minute or as long as several days, depending upon the best conditions determined to efficiently load the enucleated erythroid cells or erythroid progenitor cells. For hypotonic loading of a nucleic acid encoding one or more exogenous protein(s) (e.g., any of the exemplary exogenous proteins described herein or known in the art), a nucleic acid can be suspended in a hypotonic Tris-HCl solution (pH 7.0) and injected into erythroid progenitor cells. The concentration of Tris-HCl can be from about 20 mmol/l to about 150 mmol/l , depending upon the best conditions determined to efficiently load the enucleated erythroid cells.

Alternatively, erythroid progenitor cells or enucleated erythroid cells may be loaded with an exogenous protein using controlled dialysis against a hypotonic solution to swell the cells and create pores in the cell membrane (See, e.g., U.S. Pat. Nos. 4,327,710; 5,753,221; 6,495,351, and 10,046,009). For example, a pellet of cells is resuspended in 10 mM HEPES, 140 mM NaCl, 5 mM glucose pH 7.4 and dialyzed against a low ionic strength buffer containing 10 mM NaH₂P0₄, 10 mM NaHCO₃, 20 mM glucose, and 4 mM MgCl₂, pH 7.4. After 30-60 min, the cells are further dialyzed against 16 mM NaH₂P0₄, pH 7.4 solution containing the exogenous protein for an additional 30-60 min. All of these procedures may be advantageously performed at a temperature of 4° C. In some instances, it may be beneficial to load a large quantity of erythroid progenitor cells or enucleated erythroid cells by a dialysis approach and a specific apparatus designed for this purpose may be used (See, e.g., U.S. Pat. Nos. 4,327,710, 6,139,836 and 6,495,351).

### Isolating and Formulating Enucleated Erythroid Cells

In some embodiments, the methods provided herein can further include (e) isolating the population of engineered enucleated erythroid cells from the third cell culture in step (d). Engineered enucleated erythroid cells can be isolated using methods known in the art, such as but not limited to, centrifugation (e.g., density-gradient centrifugation), FACS, and MACS. In some embodiments, the methods provided herein further include (f) formulating the population of engineered enucleated erythroid cells isolated in step (e). In some examples, formulating the population of engineered enucleated erythroid cells can include mixing the isolated population of engineered enucleated erythroid cells with one of more pharmaceutically acceptable buffers or exipicents (e.g., phosphate buffered saline).

In some embodiments, the population of engineered enucleated erythroid cells is formulated into a composition (e.g., a pharmceuticaly composition) that is suitable for the storage, transportation, and/or administration of the cells to a subject. In some embodiments, the methods provided herein further include administering the formulated population of engineered enucleated erytroid cells in step (f) to a subject in need thereof (e.g., a subject previously identified or diagnosed as being in need of the one or more exogenous proteins present in an engineered enucleated erythroid cell, or a subject identified as being in need of a blood transfusion and/or an increase in erythrocytes). In some embodiments of these methods, the subject has been previously identified or diagnosed as being in need of one or more of the exogenous proteins present in the administered engineered enucleated erythroid cells. In some embodiments of these methods, the formulated population of engineered enucleated erytroid cells are administered through intravenous administration to the subject.

### Compositions

Also provided herein are populations of enucleated erythroid cells produced by any of the methods described herein. Also provided herein are compositions that include any of the populations of enucleated erythroid cells produced by any of the methods described herein. Also provided herein are formulations (e.g., pharmaceutical compositions) that are produced by any of the methods described herein. In some embodiments, the formulations are suitable for intravenous administration to a subject (e.g., any of the subjects described herein). In some embodiments of any of the compositions or formulations described herein, the composition or formulation is a dosage form of any of the enucleated erythroid cells described herein.

### Kits

Also provided herein are kits that include any of the compositions, formulations, or populations of enucleated erythroid cells provided herein. Also provided herein are kits that include one or more sterile vessels containing any of the compositions, formulations, or populations of enucleated erythroid cells described herein (e.g., a sterile conical tube, a sterile petri dish, a sterile vial (e.g., a borosilicate glass vial), and sterile plastic bags (a di-2-ethylhexyl phthalate (DEHP)-plasticized polyvinyl chloride (PVC) bag, or n-butyryl-tri(n-hexyl)-citrate (BTHC)-plasticized PVC bag). In some embodiments, any of the kits provided herein can further include instructions for administration of any of the compositions, formulations, or populations of enucleated erythroid cells described herein to a subject in need thereof.

Some embodiments of the kits described herein include a suitable single dosage form of any of the compositions, formulations, and populations of enucleated erythroid cells described herein. For example, a single dosage form of any of the compositions or formulations described herein can have a volume of, e.g., about 0.5 mL to about 2 L, about 0.5 mL to about 1800 mL, about 0.5 mL to about 1500 mL, about 0.5 mL to about 1200 mL, about 0.5 mL to about 1000 mL, about 0.5 mL to about 800 mL, about 0.5 mL to about 600 mL, about 0.5 mL to about 500 mL, about 0.5 mL to about 450 mL, about 0.5 mL to about 400 mL, about 0.5 mL to about 350 mL, about 0.5 mL to about 300 mL, about 0.5 mL to about 250 mL, about 0.5 mL to about 200 mL, about 0.5 mL to about 180 mL, about 0.5 mL to about 160 mL, about 0.5 mL to about 140 mL, about 0.5 mL to about 120 mL, about 0.5 mL to about 100 mL, about 0.5 mL to about 80 mL, about 0.5 mL to about 60 mL, about 0.5 mL to about 40 mL, about 0.5 mL to about 20 mL, about 0.5 mL to about 10 mL, about 0.5 mL to about 5 mL, about 0.5 mL to about 1.0 mL, about 1.0 mL to about 2 L, about 1.0 mL to about 1800 mL, about 1.0 mL to about 1500 mL, about 1.0 mL to about 1200 mL, about 1.0 mL to about 1000 mL, about 1.0 mL to about 800 mL, about 1.0 mL to about 600 mL, about 1.0 mL to about 500 mL, about 1.0 mL to about 450 mL, about 1.0 mL to about 400 mL, about 1.0 mL to about 350 mL, about 1.0 mL to about 300 mL, about 1.0 mL to about 250 mL, about 1.0 mL to about 200 mL, about 1.0 mL to about 180 mL, about 1.0 mL to about 160 mL, about 1.0 mL to about 140 mL, about 1.0 mL to about 120 mL, about 1.0 mL to about 100 mL, about 1.0 mL to about 80 mL, about 1.0 mL to about 60 mL, about 1.0 mL to about 40 mL, about 1.0 mL to about 20 mL, about 1.0 mL to about 10 mL, about 1.0 mL to about 5 mL, about 5 mL to about 2 L, about 5 mL to about 1800 mL, about 5 mL to about 1500 mL, about 5 mL to about 1200 mL, about 5 mL to about 1000 mL, about 5 mL to about 800 mL, about 5 mL to about 600 mL, about 5 mL to about 1800 mL, about 5 mL to about 500 mL, about 5 mL to about 450 mL, about 5 mL to about 400 mL, about 5 mL to about 350 mL, about 5 mL to about 300 mL, about 5 mL to about 250 mL, about 5 mL to about 200 mL, about 5 mL to about 180 mL, about 5 mL to about 160 mL, about 5 mL to about 140 mL, about 5 mL to about 120 mL, about 5 mL to about 100 mL, about 5 mL to about 80 mL, about 5 mL to about 60 mL, about 5 mL to about 40 mL, about 5 mL to about 20 mL, about 5 mL to about 10 mL, about 10 mL to about 2 L, about 10 mL to about 1800 mL, about 10 mL to about 1500 mL, about 10 mL to about 1200 mL, about 10 mL to about 1000 mL, about 10 mL to about 800 mL, about 10 mL to about 600 mL, about 10 mL to about 500 mL, about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 180 mL, about 10 mL to about 160 mL, about 10 mL to about 140 mL, about 10 mL to about 120 mL, about 10 mL to about 100 mL, about 10 mL to about 80 mL, about 10 mL to about 60 mL, about 10 mL to about 40 mL, about 10 mL to about 20 mL, about 20 mL to about 2 L, about 20 mL to about 1800 mL, about 20 mL to about 1500 mL, about 20 mL to about 1200 mL, about 20 mL to about 1000 mL, about 20 mL to about 800 mL, about 20 mL to about 600 mL, about 20 mL to about 500 mL, about 20 mL to about 450 mL, about 20 mL to about 400 mL, about 20 mL to about 350 mL, about 20 mL to about 300 mL, about 20 mL to about 250 mL, about 20 mL to about 200 mL, about 20 mL to about 180 mL, about 20 mL to about 160 mL, about 20 mL to about 140 mL, about 20 mL to about 120 mL, about 20 mL to about 100 mL, about 20 mL to about 80 mL, about 20 mL to about 60 mL, about 20 mL to about 40 mL, about 40 mL to about 2 L, about 40 mL to about 1800 mL, about 40 mL to about 1500 mL, about 40 mL to about 1200 mL, about 40 mL to about 1000 mL, about 40 mL to about 800 mL, about 40 mL to about 600 mL, about 40 mL to about 500 mL, about 40 mL to about 450 mL, about 40 mL to about 400 mL, about 40 mL to about 350 mL, about 40 mL to about 300 mL, about 40 mL to about 250 mL, about 40 mL to about 200 mL, about 40 mL to about 180 mL, about 40 mL to about 160 mL, about 40 mL to about 140 mL, about 40 mL to about 120 mL, about 40 mL to about 100 mL, about 40 mL to about 80 mL, about 40 mL to about 60 mL, about 60 mL to about 2 L, about 60 mL to about 1800 mL, about 60 mL to about 1500 mL, about 60 mL to about 1200 mL, about 60 mL to about 1000 mL, about 60 mL to about 800 mL, about 60 mL to about 600 mL, about 60 mL to about 500 mL, about 60 mL to about 450 mL, about 60 mL to about 400 mL, about 60 mL to about 350 mL, about 60 mL to about 300 mL, about 60 mL to about 250 mL, about 60 mL to about 200 mL, about 60 mL to about 180 mL, about 60 mL to about 160 mL, about 60 mL to about 140 mL, about 60 mL to about 120 mL, about 60 mL to about 100 mL, about 60 mL to about 80 mL, about 80 mL to about 2 L, about 80 mL to about 1800 mL, about 80 mL to about 1500 mL, about 80 mL to about 1200 mL, about 80 mL to about 1000 mL, about 80 mL to about 800 mL, about 80 mL to about 600 mL, about 80 mL to about 500 mL, about 80 mL to about 450 mL, about 80 mL to about 400 mL, about 80 mL to about 350 mL, about 80 mL to about 300 mL, about 80 mL to about 250 mL, about 80 mL to about 200 mL, about 80 mL to about 180 mL, about 80 mL to about 160 mL, about 80 mL to about 140 mL, about 80 mL to about 120 mL, about 80 mL to about 100 mL, about 100 mL to about 2 L, about 100 mL to about 1800 mL, about 100 mL to about 1500 mL, about 100 mL to about 1200 mL, about 100 mL to about 1000 mL, about 100 mL to about 800 mL, about 100 mL to about 600 mL, about 100 mL to about 500 mL, about 100 mL to about 450 mL, about 100 mL to about 400 mL, about 100 mL to about 350 mL, about 100 mL to about 300 mL, about 100 mL to about 250 mL, about 100 mL to about 200 mL, about 100 mL to about 180 mL, about 100 mL to about 160 mL, about 100 mL to about 140 mL, about 100 mL to about 120 mL, about 120 mL to about 2 L, about 120 mL to about 1800 mL, about 120 mL to about 1500 mL, about 120 mL to about 1200 mL, about 120 mL to about 1000 mL, about 120 7mL to about 800 mL, about 120 mL to about 600 mL, about 120 mL to about 500 mL, about 120 mL to about 450 mL, about 120 mL to about 400 mL, about 120 mL to about 350 mL, about 120 mL to about 300 mL, about 120 mL to about 250 mL, about 120 mL to about 200 mL, about 120 mL to about 180 mL, about 120 mL to about 160 mL, about 120 mL to about 140 mL, about 140 mL to about 2 L, about 140 mL to about 1800 mL, about 140 mL to about 1500 mL, about 140 mL to about 1200 mL, about 140 mL to about 1000 mL, about 140 mL to about 800 mL, about 140 mL to about 600 mL, about 140 mL to about 500 mL, about 140 mL to about 450 mL, about 140 mL to about 400 mL, about 140 mL to about 350 mL, about 140 mL to about 300 mL, about 140 mL to about 250 mL, about 140 mL to about 200 mL, about 140 mL to about 180 mL, about 140 mL to about 160 mL, about 160 mL to about 500 mL, about 160 mL to about 450 mL, about 160 mL to about 400 mL, about 160 mL to about 350 mL, about 160 mL to about 300 mL, about 160 mL to about 250 mL, about 160 mL to about 200 mL, about 160 mL to about 180 mL, about 180 mL to about 2 L, about 180 mL to about 1800 mL, about 180 mL to about 1500 mL, about 180 mL to about 1200 mL, about 180 mL to about 1000 mL, about 180 mL to about 800 mL, about 180 mL to about 600 mL, about 180 mL to about 500 mL, about 180 mL to about 450 mL, about 180 mL to about 400 mL, about 180 mL to about 350 mL, about 180 mL to about 300 mL, about 180 mL to about 250 mL, about 180 mL to about 200 mL, about 200 mL to about 2 L, about 200 mL to about 1800 mL, about 200 mL to about 1500 mL, about 200 mL to about 1200 mL, about 200 mL to about 1000 mL, about 200 mL to about 800 mL, about 200 mL to about 600 mL, about 200 mL to about 500 mL, about 200 mL to about 450 mL, about 200 mL to about 400 mL, about 200 mL to about 350 mL, about 200 mL to about 300 mL, about 200 mL to about 250 mL, about 250 mL to about 2 L, about 250 mL to about 1800 mL, about 250 mL to about 1500 mL, about 250 mL to about 1200 mL, about 250 mL to about 1000 mL, about 250 mL to about 800 mL, about 250 mL to about 600 mL, about 250 mL to about 500 mL, about 250 mL to about 450 mL, about 250 mL to about 400 mL, about 250 mL to about 350 mL, about 250 mL to about 300 mL, about 300 mL to about 2 L, about 300 mL to about 1800 mL, about 300 mL to about 1500 mL, about 300 mL to about 1200 mL, about 300 mL to about 1000 mL, about 300 mL to about 800 mL, about 300 mL to about 600 mL, about 300 mL to about 500 mL, about 300 mL to about 450 mL, about 300 mL to about 400 mL, about 300 mL to about 350 mL, about 350 mL to about 2 L, about 350 mL to about 1800 mL, about 350 mL to about 1500 mL, about 350 mL to about 1200 mL, about 350 mL to about 1000 mL, about 350 mL to about 800 mL, about 350 mL to about 600 mL, about 350 mL to about 500 mL, about 350 mL to about 450 mL, about 350 mL to about 400 mL, about 400 mL to about 2 L, about 400 mL to about 1800 mL, about 400 mL to about 1500 mL, about 400 mL to about 1200 mL, about 400 mL to about 1000 mL, about 400 mL to about 800 mL, about 400 mL to about 600 mL, about 400 mL to about 500 mL, about 400 mL to about 450 mL, about 450 mL to about 2 L, about 450 mL to about 1800 mL, about 450 mL to about 1500 mL, about 450 mL to about 1200 mL, about 450 mL to about 1000 mL, about 450 mL to about 800 mL, about 450 mL to about 600 mL, about 450 mL to about 500 mL, about 500 mL to about 2 L, about 500 mL to about 1800 mL, about 500 mL to about 1500 mL, about 500 mL to about 1200 mL, about 500 mL to about 1000 mL, about 500 mL to about 800 mL, about 500 mL to about 600 mL, about 600 mL to about 2 L, about 600 mL to about 1800 mL, about 600 mL to about 1500 mL, about 600 mL to about 1200 mL, about 600 mL to about 1000 mL, about 600 mL to about 800 mL, about 800 mL to about 2 L, about 800 mL to about 1800 mL, about 800 mL to about 1500 mL, about 800 mL to about 1200 mL, about 800 mL to about 1000 mL, about 1000 mL to about 2 L, about 1000 mL to about 1800 mL, about 1000 mL to about 1500 mL, about 1000 mL to about 1200 mL, about 1200 mL to about 2 L, about 1200 mL to about 1800 mL, about 1200 mL to about 1500 mL, about 1500 mL to about 2 L, about 1500 mL to about 1800 mL, or about 1800 mL to about 2 L.

### Methods of Treating a Subject

Also provided herein are methods of treating a subject in need thereof that include administering a therapeutically effective amount of any of the compositions, formulations, or populations of enucleated erythroid cells described herein. In some embodiments, the administering is performed by intravenous administration.

Some embodiments of any of the methods described herein further include administering one or more additional therapeutic agents to the subject. In some embodiments, the one or more additional therapeutic agents can be administered to the subject at the substantially the same time as any of the compositions provided herein. In some embodiments, the one or more additional therapeutic agents can be administered to the subject before or after the administration of any of the compositions described herein to the subject.

### EXAMPLES The invention is further described in the following examples, which do not limit the scope of the invention described in the claims.

### Example 1

### Perfusion Culturing of Erythroid Cells

An Alternating Tangential Flow (ATF) system was connected to a bioreactor in the late “M” phase of erythroid cell differentiation (step (d) in any of the methods described herein). Phase-specific medium was perfused into the bioreactor system, with all other conditions kept constant. A cell-specific perfusion rate (CSPR) of 0.01-0.2 nL/cell/day was used.

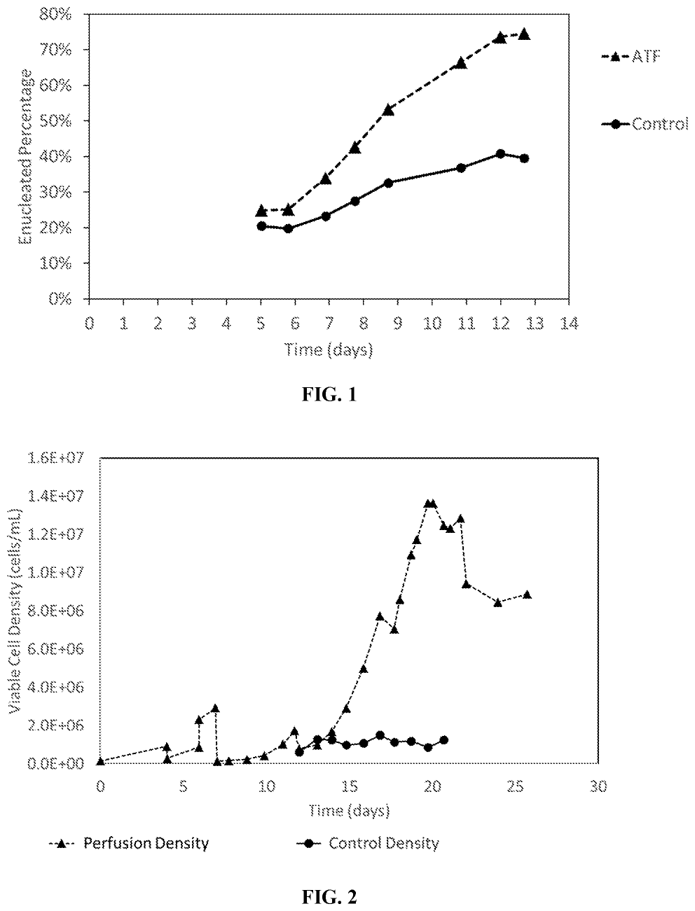

The enucleation percentages of perfusion cultured (ATF) and control erythroid cells (fed batch cultured) were measured over the course of the “M” phase. As shown in FIG. 1, perfusion cultured erythroid cells reached significantly higher enucleation rates as compared to control cells that were fed batch cultured, with between 70%-80% enucleation rate at day 13 of the culture.

The cell density of perfusion cultured erythoid cells and control erythroid cells was measured using a NovoCyte cell counter over the course of the “M” phase (step (d) in the methods described herein). As shown in FIG. 2, cells perfusion cultured reached a peak cell density of approximately 1.4×10⁷ cells/mL. In contrast, the density of control fed batch cultured cells did not exceed 2×10⁶ cells/mL.

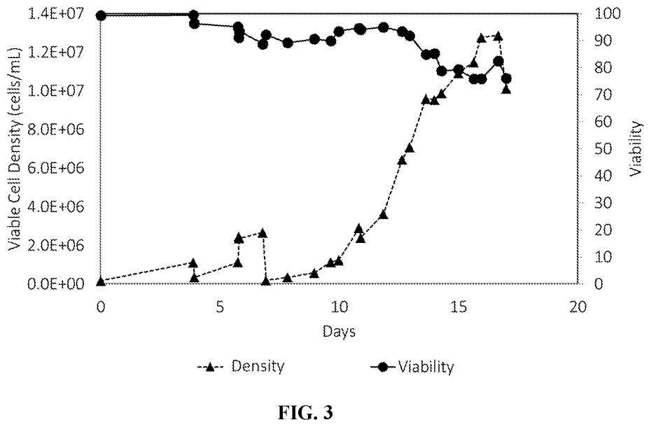

A similar ATF system set up was used to test perfusion culturing during the “D” phase of erythroid cell differentiation (corresponding to step (b) in the methods described herein). The cell density of perfusion cultured erythoid cells was measured using a NovoCyte cell counter over the course of the “D” phase. As shown in FIG. 3, perfusion cultured erythroid cells reached a peak density of approximately 1.3−1.7×10⁷ cells/mL, while maintaining high viability. At the end of “D” phase, perfusion cultured erythroid cells reached a density of 1.0×10⁷ cells/mL as compared to control fed batch cultured cells which reached a density of 1.5-3×10⁶/mL (data not shown).

These results surprisingly demonstrated that perfusion culturing of erythoroid cells during the “M” phase and/or the “D” phase resulted in increased cell densities and enucleation of differentiating erythroid progenitor cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of generating a population of enucleated erythroid cells, the method comprising:
(a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL;
(b) perfusion culturing the second cell culture for about 2 days to about 15 days;
(c) disposing a volume of the second cell culture of step (b) into a third culture medium comprised within a perfusion bioreactor to provide a third cell culture with an initial cell density of about 1×10⁵ to about 1×10⁷ cells/mL;
(d) perfusion culturing the third cell culture of step (c) for about 5 days to 20 days, wherein after step (d) the third culture medium comprises a population of enucleated erythroid cells.

2. The method of claim 1, wherein the method further comprises prior to step (a):
(i) disposing a plurality of erythroid progenitor cells in a first culture medium comprised within a vessel to provide the first cell culture with an initial cell density of about of about 0.1×10⁵ cells/mL to about 2×10⁶ cells/mL; and
(ii) batch or fed batch culturing the first cell culture for about 1 day to about 15 days.

3. The method of claim 2, wherein the vessel in step (i) is a shake flask, a shake tube, or a culture bag.

4.-21. (canceled)

22. The method of claim 2, wherein step (ii) comprises batch culturing the first cell culture.

23. The method of claim 2, wherein step (ii) comprises fed batch culturing the first cell culture.

24. The method of claim 23, wherein fed batch culturing comprises adding an additional volume of the first culture medium to the first cell culture over time.

25.-26. (canceled)

27. The method of claim 2, wherein the first culture medium comprises one or more of Flt-3 ligand, stem cell factor (SCF), IL-3, and IL-6.

28.-35. (canceled)

36. The method of claim 2, wherein the first culture medium further comprises about 1 μg/mL to about 20 μg/mL insulin.

37. (canceled)

38. The method of claim 2, wherein the first culture medium comprises about 1 mM to about 10 mM of L-glutamine, L-alanyl-L-glutamine, L-glycyl-L-glutamine, N-acetyl-L-glutamine, or a combination thereof.

39.-42. (canceled)

43. The method of claim 2, wherein the first culture medium comprises lipid.

44. The method of claim 2, wherein the first culture medium comprises about 50 μg/mL to about 400 μg/mL transferrin.

45.-60. (canceled)

61. The method of claim 1, wherein the perfusion culturing in step (b) comprises adding an additional volume of the second culture medium to the second cell culture over time.

62.-63. (canceled)

64. The method of claim 1, wherein the second culture medium comprises one or more of: transferrin, IL-3, SCF, dexamethasone, erythropoietin (EPO), and insulin.

65.-82. (canceled)

83. The method of claim 1, wherein the second culture medium comprises about 1 mM to about 10 mM of L-glutamine, L-alanyl-L-glutamine, L-glycyl-L-glutamine, N-acetyl-L-glutamine, or a combination thereof.

84.-87. (canceled)

88. The method of claim 1, wherein the second culture medium comprises lipid.

89.-101. (canceled)

102. The method of claim 1, wherein the perfusion culturing in step (d) comprises:
(i) adding an additional volume of the third culture medium to the third cell culture for a first period of time, and
(ii) adding an additional volume of a fourth culture medium to the third cell culture for a second period of time.

103.-108. (canceled)

109. The method of claim 1, wherein the third culture medium comprises one or more of: transferrin, insulin, SCF, and EPO.

110.-121. (canceled)

122. The method of claim 1, wherein the third culture medium comprises about 1 mM to about 8 mM of L-glutamine, L-alanyl-L-glutamine, L-glycyl-L-glutamine, N-acetyl-L-glutamine, or a combination thereof.

123.-125. (canceled)

126. The method of claim 1, wherein the third culture medium comprises about 0.5% v/v to about 10% v/v serum.

127. (canceled)

128. The method of claim 102, wherein the fourth culture medium comprises one or more of: transferrin, insulin, and EPO.

129.-137. (canceled)

138. The method of claim 102, wherein the fourth culture medium comprises about 1 mM to about 8 mM of L-glutamine, L-alanyl-L-glutamine, L-glycyl-L-glutamine, N-acetyl-L-glutamine, or a combination thereof.

139.-141. (canceled)

142. The method of claim 102, wherein the fourth culture medium comprises about 0.5% v/v to about 10% v/v serum.

143.-154. (canceled)

155. A method of generating a population of enucleated erythroid cells, the method comprising:
(a) disposing a volume of a first cell culture of erythroid progenitor cells into a second culture medium comprised within a vessel to provide a second cell culture with an initial cell density of about 0.1×10⁵ cells/mL to about 1×10⁷ cells/mL;
(b) batch or fed batch culturing the second cell culture for about 2 days to about 15 days;
(c) disposing a volume of the second cell culture of step (b) into a third culture medium comprised within a perfusion bioreactor to provide a third cell culture with an initial cell density of about 1×10⁵ to about 1×10⁷ cells/mL;
(d) perfusion culturing the third cell culture of step (c) for about 5 days to 20 days, wherein after step (d) the third culture medium comprises a population of enucleated erythroid cells.

156.-327. (canceled)

328. A method of generating a population of enucleated erythroid cells, the method comprising:
(a) disposing a volume of a first cell culture of erythroid progenitor cells into a second culture medium comprised within a perfusion bioreactor to provide a second cell culture with an initial cell density of about 0.1×10⁵ cells/mL to about 1×10⁷ cells/mL;
(b) perfusion culturing the second cell culture for about 2 days to about 15 days;
(c) disposing a volume of the second cell culture of step (b) into a third culture medium comprised within a vessel to provide a third cell culture with an initial cell density of about 1×10⁵ to about 1×10⁷ cells/mL;
(d) batch or fed batch culturing the third cell culture of step (c) for about 5 days to 20 days,
wherein after step (d) the third culture medium comprises a population of enucleated erythroid cells.

329.-510. (canceled)

511. The method of claim 1, wherein:
the method further comprises, prior to step (a), introducing a nucleic acid into the erythroid progenitor cells in the first cell culture, and
the method results in the production of a population of engineered enucleated erythroid cells.

512. The method of claim 511, wherein the population of engineered enucleated erythroid cells are engineered enucleated human erythroid cells.

513. (canceled)

514. The method of claim 511, wherein the engineered human enucleated erythroid cells comprise one or more exogenous protein (s).

515.-522. (canceled)

523. The method of claim 511, wherein the method further comprises:
(e) isolating the population of engineered enucleated erythroid cells from the third cell culture in step (d).

524. The method of claim 523, wherein the method further comprises:
(f) formulating the population of engineered enucleated erythroid cells isolated in step (e).

525. The method of claim 524, wherein the method further comprises:
(g) administering the formulated population of engineered enucleated erythroid cells in step (f) to a subject in need thereof.

526.-527. (canceled)

528. A population of enucleated erythroid cells produced by the method of claim 511.

529. A formulation produced by the method of claim 524.
